(19) United States
(12) Patent Application Publication
(10) Pub. No.: US 2022/0259603 A1
(43) Pub. Date: Aug. 18, 2022

(54) METHODS AND CELLS FOR MICROBIAL PRODUCTION OF PHYTOCANNABINOIDS AND PHYTOCANNABINOID PRECURSORS

(71) Applicant: Hyasynth Biologicals Inc., Montréal (CA)

(72) Inventors: Leanne BOURGEOIS, Montréal (CA); Alexander CAMPBELL, Montréal (CA); Elizabeth-Ann KRANJEC, Montréal (CA); Mindy MELGAR, Montréal (CA); Shoham MOOKERJEE, Montréal (CA); Sylvester PALYS, Montréal (CA); Alexandre THERRIEN, Montréal (CA); Curtis WALTON, Montréal (CA); Kevin WOO, Montréal (CA); Xiaohua ZHANG, Montréal (CA)

(21) Appl. No.: 17/610,278

(22) PCT Filed: May 21, 2020
(86) PCT No.: PCT/CA2020/050687

§ 371 Date: Nov. 10, 2021

Related U.S. Application Data
(60) Provisional application No. 62/851,400, filed on May 22, 2019.
(60) Provisional application No. 62/851,333, filed on May 22, 2019.
(60) Provisional application No. 62/851,839, filed on May 23, 2019.
(60) Provisional application No. 62/868,396, filed on Jun. 28, 2019.
(60) Provisional application No. 62/950,515, filed on Dec. 19, 2019.
(60) Provisional application No. 62/981,142, filed on Feb. 25, 2020.
(60) Provisional application No. 62/990,096, filed on Mar. 16, 2020.

(51) Int. Cl. C12N 15/52; C12N 15/70; C12N 15/81; C12N 9/10; C12P 17/04; C12P 7/22; C12P 7/42

(52) U.S. Cl. CPC C12N 15/52; C12N 15/70; C12N 15/81; C12P 7/42; C12P 17/04; C12P 7/22; C12N 9/1085

(57) ABSTRACT

The present disclosure relates generally to methods and cell lines for the production of phytocannabinoids, phytocannabinoid precursors or intermediates, or phytocannabinoid analogue. Methods for transformation of host cells, such as yeast cells, are described. Cells may be transformed, for example, with a polynucleotide encoding a polyketide synthase (PKS) enzyme, a polynucleotide encoding an olivetolic acid cyclase (OAC) enzyme, and/or a polynucleotide encoding a prenyltransferase (PT) enzyme; and optionally a polynucleotide encoding an acyl-CoA synthase (Alk) enzyme; a polynucleotide encoding a fatty acyl CoA activating (CsAAE) enzyme; and/or a polynucleotide encoding a THCa synthase (OXC) enzyme.

### CROSS REFERENCE TO RELATED APPLICATIONS

This application claims the benefit of and priority to U.S. Provisional Patent Application No. 62/851,400 filed May 22, 2019; U.S. Provisional Patent Application No. 62/851,333 filed May 22, 2019; U.S. Provisional Patent Application No. 62/851,839 filed May 23, 2019; U.S. Provisional Patent Application No. 62/868,396 filed Jun. 28, 2019; U.S. Provisional Patent Application No. 62/950,515 filed Dec. 19, 2019; U.S. Provisional Patent Application No. 62/981,142 filed Feb. 25, 2020; and U.S. Provisional Patent Application No. 62/990,096 filed Mar. 16, 2020, all of which are hereby incorporated by reference.

### FIELD

The present disclosure relates generally to methods and cell lines for the production of phytocannabinoids, as well as for production of precursors and intermediates in the production phytocannabinoids.

### BACKGROUND

Phytocannabinoids are a large class of compounds with over 100 different known structures that are produced in the Cannabis sativa plant. Phytocannabinoids are known to be biosynthesized in C. sativa, or may result from thermal or other decomposition from phytocannabinoids biosynthesized in C. sativa. These bio-active molecules, such as tetrahydrocannabinol (THC) and cannabidiol (CBD), can be extracted from plant material for medical and recreational purposes. However, the synthesis of plant material is costly, not readily scalable to large volumes, and requires lengthy growing periods to produce sufficient quantities of phytocannabinoids. While the C. sativa plant is also a valuable source of grain, fiber, and other material, growing C. sativa for phytocannabinoid production, particularly indoors, is costly in terms of energy and labour. Subsequent extraction, purification, and fractionation of phytocannabinoids from the C. sativa plant is also labour and energy intensive.

Phytocannabinoids are pharmacologically active molecules that contribute to the medical and psychotropic effects of C. sativa. Biosynthesis of phytocannabinoids in the C. sativa plant scales similarly to other agricultural projects. As with other agricultural projects, large scale production of phytocannabinoids by growing C. sativa requires a variety of inputs (e.g. nutrients, light, pest control, CO, etc.). The inputs required for cultivating C. sativa must be provided. In addition, cultivation of C. sativa, where allowed, is currently subject to heavy regulation, taxation, and rigorous quality control where products prepared from the plant are for commercial use, further increasing costs.

Phytocannabinoid analogues are pharmacologically active molecules that are structurally similar to phytocannabinoids. Phytocannabinoid analogues are often synthesized chemically, which can be labour intensive and costly. As a result, it may be economical to produce the phytocannabinoids and phytocannabinoid analogues in a robust and scalable, fermentable organism. Saccharomyces cerevisiae is an example of a fermentable organism that has been used to produce industrial scales of similar molecules.

The time, energy, and labour involved in growing C. sativa for production of naturally-occurring phytocannabinoids provides a motivation to produce transgenic cell lines for production of phytocannabinoids by other means. Polyketides, including olivetolic acid and its analogues are valuable precursors to phytocannabinoids.

Polyketides are precursors to many valuable secondary metabolites in plants. For example, phytocannabinoids, which are naturally produced in Cannabis sativa, other plants, and some fungi, have significant commercial value. Polyketides are a class of compounds which contain (or are derived from compounds containing) a plurality of acetoacetyl groups. Polyketide are synthesized in plants, bacteria, and fungi by polyketide synthases (PKS). Aromatic polyketides are useful in synthesis of phytocannabinoids.

It is desirable to find alternate methods for the production of phytocannabinoids, and/or for the production of compounds useful in phytocannabinoid synthesis as intermediate or precursor compounds, such as aromatic polyketides.

### SUMMARY

Numerous methods and aspects thereof are described for producing phytocannabinoids or analogues thereof. Specific summaries of particular aspects of the invention described herein are included in overview within each of the following parts:

PART 1—Prenyltransferase PT104 For Production of Prenylated Polyketides and Phytocannabinoids

PART 2—ABBA Family Prenyltransferases For Production Of Prenylated Polyketides and Phytocannabinoids

PART 3—Polyketide Synthase III and Acyl-CoA Synthases for Production of Aromatic Polyketides and Phytocannabinoids

PART 4—Dictyostelium discoideum Polyketide Synthase (DiPKS), Olivetolic Acid Cyclase (OAC), Prenyltransferases, and Mutants Thereof for Production Of Phytocannabinoids

PART 5—Prenyltransferases From Stachybotrys For The Production Of Phytocannabinoids

PART 6—PKS, NpgA, OAC and Mutants Thereof in the Production Of Polyketides and Phytocannabinoids

PART 7—Methods and Cells for Production of Phytocannabinoids or Phytocannabinoid Precursors Incorporating Aspects of PART 1 to PART 6

Other aspects and features of the present disclosure will become apparent to those ordinarily skilled in the art upon review of the following description of specific embodiments in conjunction with the accompanying figures.

### BRIEF DESCRIPTION OF THE FIGURES

Embodiments of the present disclosure will now be described, by way of example only, with reference to the attached Figures with regard to PARTS 1 to 7.

Part 1

FIG. 1 depicts a generalize scheme for the use of the PT104 to attach a prenyl moiety to aromatic polyketides to produce prenylated polyketides.

FIG. 2 depicts examples of specific aromatic polyketides in the production of phytocannabinoids.

FIG. 3 depicts structures of phytocannabinoids produced from the C—C bond formation between a polyketide precursor and geranyl pyrophosphate.

FIG. 4 outlines the native biosynthetic pathway for cannabinoid production in Cannabis sativa.

FIG. 5 outlines a biosynthetic pathway for cannabinoid synthesis as described herein.

FIG. 6 depicts the reaction involving PT104 (rdPT1) in the known synthetic pathway to grifolic acid.

FIG. 7 depicts a synthetic route for cannabigorcinic acid involving PT104.

FIG. 8 shows de-novo CBGa production by yeast strain HB887.

FIG. 9 shows de-novo simultaneous production of CBGa and CBGOa by yeast strain HB887.

Part 2

FIG. 10 depicts a generalize scheme for the use of the prenyltransferases described herein to attach a prenyl moiety to aromatic polyketides to produce prenylated polyketides.

FIG. 11 depicts a specific example in the production of cannabinoids.

FIG. 12 depicts a pathway for production of Cannabigorcinic acid in S. cerevisiae.

FIG. 13 depicts a chromatogram showing positive production of CBG.

FIG. 14 depicts a chromatogram showing positive production of CBGa.

FIG. 15 depicts a chromatogram showing positive production of CBGVa.

FIG. 16 depicts a chromatogram showing positive production of CBGO.

FIG. 17 depicts a chromatogram showing positive production of CBGOa.

FIG. 18 shows in vivo production of orsellinic Acid and CBGOa in strains produced according to Example 3.

Part 3

FIG. 19 depicts known pathways involving fatty acid-CoA for formation of different polyketides.

FIG. 20 schematically depicts pathways for cannabinoid formation by prenylation of polyketides.

FIG. 21 outlines a biosynthetic pathway for cannabinoid synthesis as described in Example 5.

FIG. 22 shows production of THCVa in S. cerevisiae using a polyketide synthase according to Examples 6 to 11.

FIG. 23 shows olivetol and olivetolic acid produced by strains according to Example 6.

FIG. 24 illustrates divarin, divarinic acid, CBGVa and THCVa produced by strains in Example 7.

FIG. 25 illustrates octavic acid produced by strains in Example 8.

FIG. 26 shows C5-alkynyl cannabigerolic acid peak area produced by strains in Example 9.

FIG. 27 illustrates C5-alkenyl cannabigerolic acid produced by strains in Example 10.

Part 4

FIG. 28 is a schematic of biosynthesis of olivetolic acid and related compounds with different alkyl group chain lengths in C. sativa.

FIG. 29 is a schematic of biosynthesis of CBGa from hexanoic acid, malonyl-CoA, and geranyl pyrophosphate in C. sativa.

FIG. 30 is a schematic of biosynthesis of downstream phytocannabinoids in acid form CBGa C. sativa.

FIG. 31 is a schematic of biosynthesis of MPBD by DiPKS.

FIG. 32 is a schematic of functional domains in DiPKS, with mutations to a C-methyl transferase that for lowering methylation of olivetol.

FIG. 33 is a schematic of biosynthesis of CBGa in a transformed yeast cell by DiPKS^{G1516R}, csOAC and PT254.

FIG. 34 is a schematic of biosynthesis of THCa in a transformed yeast cell by DiPKS^{G1516R}, csOAC, PT254 and THCa Synthase.

FIG. 35 shows production of olivetolic acid by DiPKS^{G1516R} and csOAC in a strain of S. cerevisiae.

FIG. 36 shows production of CBGa by DiPKS^{G1516R}, csOAC and PT254 in two strains of S. cerevisiae.

FIG. 37 shows production of olivetolic acid by DiPKS^{G1516R} and csOAC in a strain of S. cerevisiae and of CBGa and olivetolic acid by DiPKS^{G1516R}, csOAC and PT254 in two strains of S. cerevisiae.

FIG. 38 shows production of THCa acid by DiPKS^{G1516R}, csOAC, PT254 and THCA synthase in a strain of S. cerevisiae.

Part 5

FIG. 39 depicts a generalize scheme for the use of the PT72, PT273, or PT296 to attach a prenyl moiety to aromatic polyketides to produce prenylated polyketides.

FIG. 40 depicts examples of specific aromatic polyketides in the production of phytocannabinoids.

FIG. 41 depicts a synthetic route for cannabigorcinic acid involving PT72, PT273, or PT296.

Part 6

FIG. 42 is a schematic of biosynthesis of MPBD by DiPKS, synthesis of olivetol by DiPKS^{G1516R} and synthesis of olivetolic acid by DiPKS^{G1516R} and csOAC.

FIG. 43 shows production data for MPBD and olivetol in eight strains of S. cerevisiae.

FIG. 44 shows production data for olivetolic acid and olivetol in four strains of S. cerevisiae.

FIG. 45 shows production data for olivetolic acid and olivetol in nine strains of S. cerevisiae.

### DETAILED DESCRIPTION

Certain terms used herein are described below.

The term “cannabinoid” as used herein refers to a chemical compound that shows direct or indirect activity at a cannabinoid receptor. Non limiting examples of cannabinoids include tetrahydrocannabinol (THC), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), and cannabigerol monomethyl ether (CBGM).

The term “phytocannabinoid” as used herein refers to a cannabinoid that typically occurs in a plant species. Exemplary phytocannabinoids produced according to the invention include cannabigerol (CBG), cannabigerolic acid (CBGa), cannabigerovarin (CBGv), cannabigerovarinic acid (CBGva), cannabigerocin (CBGo), or cannabigerocinic acid (CBGoa).

Cannabinoids and phytocannabinoids may contain or may lack one or more carboxylic acid functional groups. Non limiting examples of such cannabinoids or phytocannabinoids containing carboxylic acid function groups or phytocannabinoids include tetrahydrocannabinolic acid (THCA), cannabidiolic acid (CBDA), and cannabichromenic acid (CBCA).

The term “homologue” includes homologous sequences from the same and other species and orthologous sequences from the same and other species. Different polynucleotides or polypeptides having homology may be referred to as homologues.

The term “homology” may refer to the level of similarity between two or more polynucleotide and/or polypeptide sequences in terms of percent of positional identity (i.e., sequence similarity or identity). Homology also refers to the concept of similar functional properties among different polynucleotide or polypeptides. Thus, the compositions and methods herein may further comprise homologues to the polypeptide and polynucleotide sequences described herein.

The term “orthologous,” as used herein, refers to homologous polypeptide sequences and/or polynucleotide sequences in different species that arose from a common ancestral gene during speciation.

As used herein, a “homologue” may have a significant sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% and/or 100%) to the polynucleotide sequences herein.

As used herein “sequence identity” refers to the extent to which two optimally aligned polynucleotide or peptide sequences are invariant throughout a window of alignment of components, e.g., nucleotides or amino acids. “Identity” can be readily calculated by known methods.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

The terms “fatty acid-CoA”, “fatty acyl-CoA”, or “CoA donors” as used herein may refer to compounds useful in polyketide synthesis as primer molecules which react in a condensation reaction with an extender unit (such as malonyl-CoA) to form a polyketide. Examples of fatty acid-CoA molecules (also referred to herein as primer molecules or CoA donors), useful in the synthetic routes described herein include but are not limited to: acetyl-CoA, butyryl-CoA, hexanoyl-CoA. These fatty acid-CoA molecules may be provided to host cells or may be synthesized by the host cells for biosynthesis of polyketides, as described herein.

Two nucleotide sequences can be considered to be substantially “complementary” when the two sequences hybridize to each other under stringent conditions. In some examples, two nucleotide sequences considered to be substantially complementary hybridize to each other under highly stringent conditions.

The terms “stringent hybridization conditions” and “stringent hybridization wash conditions” in the context of nucleic acid hybridization experiments, for example in Southern hybridizations and Northern hybridizations are sequence dependent, and are different under different environmental parameters. In some examples, generally, highly stringent hybridization and wash conditions are selected to be about 5° C. lower than the thermal melting point (Tm) for the specific sequence at a defined ionic strength and pH.

In some examples, polynucleotides include polynucleotides or “variants” having at least about 50%, 55%, 60%, 65%, 70%, 75%, 80%, 85%, 90%, 95%, 96%, 97%, 98%, 99% or 100% sequence identity to any of the reference sequences described herein, typically where the variant maintains at least one biological activity of the reference sequence.

As used herein, the terms “polynucleotide variant” and “variant” and the like refer to polynucleotides displaying substantial sequence identity with a reference polynucleotide sequence or polynucleotides that hybridize with a reference sequence under, for example, stringent conditions. These terms may include polynucleotides in which one or more nucleotides have been added or deleted, or replaced with different nucleotides compared to a reference polynucleotide. It will be understood that that certain alterations inclusive of mutations, additions, deletions and substitutions can be made to a reference polynucleotide whereby the altered polynucleotide retains the biological function or activity of the reference polynucleotide.

In some examples, the polynucleotides described herein may be included within “vectors” and/or “expression cassettes”.

In some embodiments, the nucleotide sequences and/or nucleic acid molecules described herein may be “operably” or “operatively” linked to a variety of promoters for expression in host cells. Thus, in some examples, the invention provides transformed host cells and transformed organisms comprising the transformed host cells, wherein the host cells and organisms are transformed with one or more nucleic acid molecules/nucleotide sequences of the invention. As used herein, “operably linked to,” when referring to a first nucleic acid sequence that is operably linked to a second nucleic acid sequence, means a situation when the first nucleic acid sequence is placed in a functional relationship with the second nucleic acid sequence. For instance, a promoter is operably associated with a coding sequence if the promoter effects the transcription or expression of the coding sequence.

In the context of a polypeptide, “operably linked to,” when referring to a first polypeptide sequence that is operably linked to a second polypeptide sequence, refers to a situation when the first polypeptide sequence is placed in a functional relationship with the second polypeptide sequence.

The term a “promoter,” as used herein, refers to a nucleotide sequence that controls or regulates the transcription of a nucleotide sequence (i.e., a coding sequence) that is operably associated with the promoter. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression.

Promoters can include, for example, constitutive, inducible, temporally regulated, developmentally regulated, chemically regulated, tissue-preferred and tissue-specific promoters for use in the preparation of recombinant nucleic acid molecules, i.e., chimeric genes.

The choice of promoter will vary depending on the temporal and spatial requirements for expression, and also depending on the host cell to be transformed. Thus, for example, where expression in response to a stimulus is desired a promoter inducible by stimuli or chemicals can be used. Where continuous expression at a relatively constant level is desired throughout the cells or tissues of an organism a constitutive promoter can be chosen.

In some examples, vectors may be used.

In some examples, the polynucleotide molecules and nucleotide sequences described herein can be used in connection with vectors.

The term “vector” refers to a composition for transferring, delivering or introducing a nucleic acid or polynucleotide into a host cell. A vector may comprise a polynucleotide molecule comprising the nucleotide sequence(s) to be transferred, delivered or introduced. Non-limiting examples of general classes of vectors include, but are not limited to, a viral vector, a plasmid vector, a phage vector, a phagemid vector, a cosmid, a fosmid, a bacteriophage, or an artificial chromosome. The selection of a vector will depend upon the preferred transformation technique and the target species for transformation.

As used herein, “expression vectors” refers to a nucleic acid molecule comprising a nucleotide sequence of interest, wherein said nucleotide sequence is operatively associated with at least a control sequence (e.g., a promoter). Thus, some examples provide expression vectors designed to express the polynucleotide sequences of described herein.

An expression vector comprising a polynucleotide sequence of interest may be “chimeric”, meaning that at least one of its components is heterologous with respect to at least one of its other components. An expression cassette may also be one that is naturally occurring but has been obtained in a recombinant form useful for heterologous expression. In some examples, however, the expression vector is heterologous with respect to the host. For example, the particular polynucleotide sequence of the expression vector does not occur naturally in the host cell and must have been introduced into the host cell or an ancestor of the host cell by a transformation event.

In some examples, an expression vector may also include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, promoters, enhancers, introns, 5′ and 3′ untranslated regions, translation leader sequences, termination signals, and polyadenylation signal sequences.

An expression vector may also include a nucleotide sequence for a selectable marker, which can be used to select a transformed host cell.

As used herein, “selectable marker” means a nucleotide sequence that when expressed imparts a distinct phenotype to the host cell expressing the marker and thus allows such transformed host cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, a sugar, a carbon source, or the like), or on whether the marker is simply a trait that one can identify through observation or testing, such as by screening. Examples of suitable selectable markers are known in the art and can be used in the expression vectors described herein.

The vector and/or expression vectors and/or polynucleotides may be introduced in to a cell.

The term “introducing,” in the context of a nucleotide sequence of interest (e.g., the nucleic acid molecules/constructs/expression vectors), refers to presenting the nucleotide sequence of interest to cell host in such a manner that the nucleotide sequence gains access to the interior of a cell. Where more than one nucleotide sequence is to be introduced these nucleotide sequences can be assembled as part of a single polynucleotide or nucleic acid construct, or as separate polynucleotide or nucleic acid constructs, and can be located on the same or different transformation vectors. Accordingly, these polynucleotides may be introduced into host cells in a single transformation event, or in separate transformation events.

As used herein, the term “contacting” refers to a process by which, for example, a compound may be delivered to a cell. The compound may be administered in a number of ways, including, but not limited to, direct introduction into a cell (i.e., intracellularly) and/or extracellular introduction into a cavity, interstitial space, or into the circulation of the organism.

The term “transformation” or “transfection” as used herein refers to the introduction of a polynucleotide or heterologous nucleic acid into a cell. Transformation of a cell may be stable or transient.

The term “transient transformation” as used herein in the context of a polynucleotide refers to a polynucleotide introduced into the cell and does not integrate into the genome of the cell.

The terms “stably introducing” or “stably introduced” in the context of a polynucleotide introduced into a cell is intended to represent that the introduced polynucleotide is stably incorporated into the genome of the cell, and thus the cell is stably transformed with the polynucleotide.

The term “host cell” includes an individual cell or cell culture which can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transformed in vivo or in vitro with a recombinant vector or a polynucleotide of the invention. A host cell which comprises a recombinant vector of the invention is a recombinant host cell.

In some examples, a host cell may be a bacterial cell, a fungal cell, a protist cell, or a plant cell. Specific examples of host cells are described below.

### Part 1

Prenyltransferase PT104 for Production of Prenylated Polyketides and Phytocannabinoids

This section relates generally to methods and cell lines for the production of phytocannabinoids using host cells transformed with a sequence encoding a PT104 prenyltransferase protein. Examples include production of a variety of cannabinoids in yeast.

### Overview

There is provided herein a method of producing a phytocannabinoid or phytocannabinoid analogue in a host cell that produces a polyketide and a prenyl donor. The method comprises transforming the host cell with a sequence encoding a prenyltransferase PT104 protein and culturing the transformed host cell to produce the phytocannabinoid or phytocannabinoid analogue.

Further, there is provided herein a method of producing a phytocannabinoid or phytocannabinoid analogue, comprising providing a host cell which produces a polyketide precursor and a prenyl donor, introducing into the host cell a polynucleotide encoding a prenyltransferase PT104 protein, and culturing the host cell under conditions sufficient for production of the prenyltransferase PT104 protein for producing the phytocannabinoid or phytocannabinoid analogue from the polyketide precursor and the prenyl donor. The PT104 protein is a protein as set forth in SEQ ID NO:1; a protein with at least 70% identity with SEQ ID NO:1; a protein that differs from SEQ ID NO:1 by one or more residues that are substituted, deleted and/or inserted; or derivatives thereof bearing prenyltransferase activity.

Additionally, there is provided herein an expression vector comprising a nucleotide sequence encoding prenyltransferase PT104 protein, wherein the nucleotide sequence comprises at least 70% identity with positions 98-1153 of SEQ ID NO:17, or wherein the prenyltransferase PT104 protein comprises at least 70% identity with SEQ ID NO:1. Host cells transformed with the expression vector are also described.

### Detailed Description of Part 1

Generally, there is described herein the production of phyotocannabinoids or phytocannabinoid analogues.

The method described herein produces a phytocannabinoid or a phytocannabinoid analogue in a host cell, which host cell comprises or is capable of producing a polyketide and a prenyl donor. The method comprises transforming the host cell with a sequence encoding a prenyltransferase PT104 protein, and subsequently culturing the transformed cell to produce said phytocannabinoid or phytocannabinoid analogue.

The PT104 protein may be one having one of the following characteristics: (a) a protein as set forth in SEQ ID NO:1; (b) a protein with at least 70% identity with SEQ ID NO:1; (c) a protein that differs from (a) by one or more residues that are substituted, deleted and/or inserted; or (d) a derivative of (a), (b), or (c).

The sequence encoding the prenyltransferase PT104 protein may have one of the following characteristics: (a) a nucleotide sequence as set forth in positions 98-1153 of SEQ ID NO:17; (b) a nucleotide sequence having at least 70% identity with the nucleotide sequence of (a); (c) a nucleotide sequence that hybridizes with the complementary strand of the nucleic acid of (a) and it may be that such a polynucleotide hybridizes with the complementary strand under conditions of high stringency; (d) a nucleotide sequence that differs from (a) by one or more nucleotides that are substituted, deleted, and/or inserted; or (e) a derivative of (a), (b), (c), or (d).

The polyketide may be one of the following:

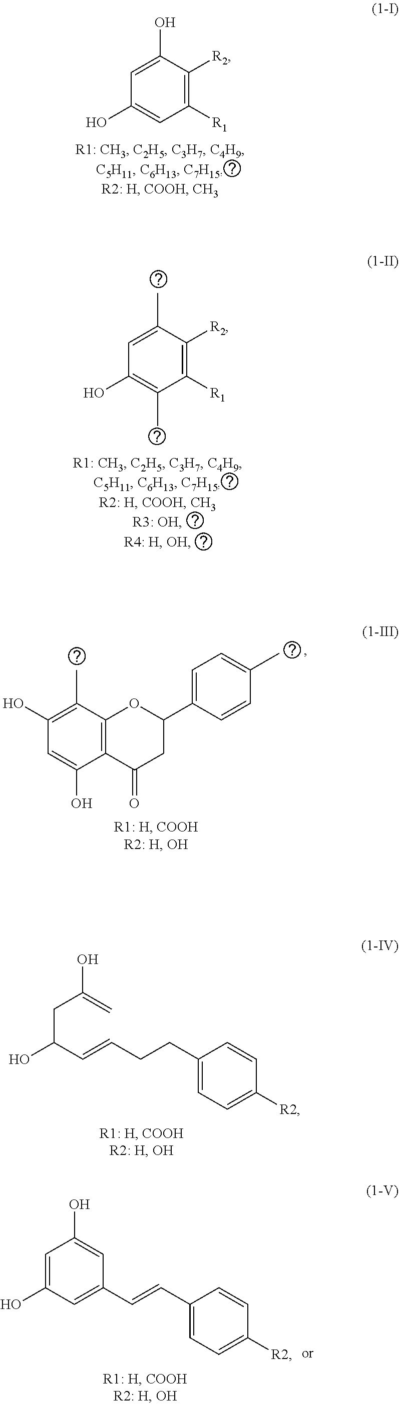

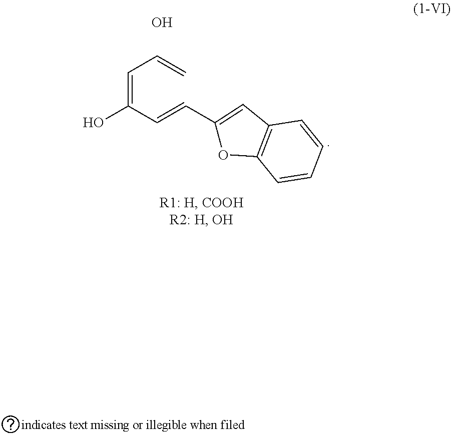

The prenyl donor may have the following structure:

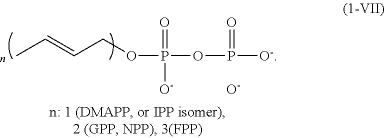

For example, the prenyl donor may be geranyl diphosphate (GPP), farnesyl diphosphate (FPP), or neryl diphosphate (NPP).

The phytocannabinoid or phytocannabinoid analogue formed may be:

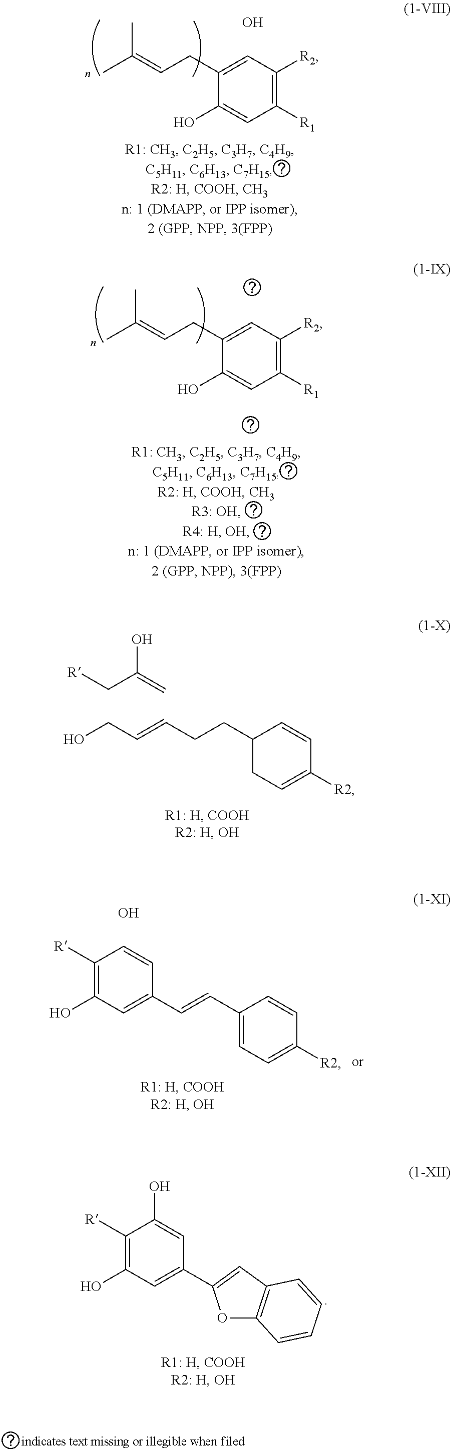

The protein encoded by the nucleotide sequence with which the host cell is transformed may have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the prenyltransferase PT104 protein of SEQ ID NO:1.

The nucleotide sequence may have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to positions 98-1153 of SEQ ID NO:17.

The polyketide prenylated in the method may be olivetol, olivetolic acid, divarin, divarinic acid, orcinol, or orsellinic acid.

The phytocannabinoid so formed may be cannabigerol (CBG), cannabigerolic acid (CBGa), cannabigerovarin (CBGv), cannabigerovarinic acid (CBGva), cannabigerocin (CBGO), or cannabigerocinic acid (CBGOa).

As exemplary embodiments, when the polyketide is olivetol then the phytocannabinoid formed is cannabigerol (CBG); when the polyketide is olivetolic acid then the phytocannabinoid formed is cannabigerolic acid (CBGa); when the polyketide is divarin then the phytocannabinoid formed is cannabigerovarin (CBGv); when the polyketide is divarinic acid then the phytocannabinoid formed is cannabigerovarinic acid (CBGva); when the polyketide is orcinol then the phytocannabinoid is cannabigerocin (CBGO); and when the polyketide is orsellinic acid then the phytocannabinoid is cannabigerocinic acid (CBGOa).

The host cell can be a bacterial cell, a fungal cell, a protist cell, or a plant cell, such as any of the exemplary cell types noted herein in Table 2. Exemplary host cell types include S. cerevisiae, E. coli, Yarrowia lipolytica, and Komagataella phaffii.

A method is described for producing a phytocannabinoid or phytocannabinoid analogue, comprising: providing a host cell which produces a polyketide precursor and a prenyl donor, introducing into the host cell a polynucleotide encoding a prenyltransferase PT104 protein, and culturing the host cell under conditions sufficient for production of the prenyltransferase PT104 protein for producing the phytocannabinoid or phytocannabinoid analogue from the polyketide precursor and the prenyl donor.

In any of the methods described herein, the host cell may have one or more additional genetic modification, such as for example: (a) a nucleic acid as set forth in any one of SEQ ID NO:2 to SEQ ID NO:14; (b) a nucleic acid having at least 70% identity with the nucleotide sequence of (a); (c) a nucleic acid that hybridizes with the complementary strand of the nucleic acid of (a); (d) a nucleic acid encoding a polypeptide with the same enzyme activity as the polypeptide encoded by any one of the nucleic acid sequences of (a); (e) a nucleotide sequence that differs from (a) by one or more nucleotides that are substituted, deleted, and/or inserted; or (f) a derivative of (a), (b), (c), (d), or (e). Such an additional genetic modification may comprise, for example, one or more of NpgA (SEQ ID NO:2), PDH (SEQ ID NO:8), Maf1 (SEQ ID NO:9), Erg20K197E (SEQ ID NO:10), tHMGr-IDI (SEQ ID NO:12), and/or PGK1p:ACC^{1S659A,S1157A} (SEQ ID NO:13).

One or more genetic modification may be made to the host cell in order to increase the available pool of terpenes and/or malonyl-coA in the cell. For example, such a genetic modification may include tHMGr-IDI (SEQ ID NO: 12); PGK1p:ACC^{1S659A,S1157A} (SEQ ID NO:13); and/or Erg20K197E (SEQ ID NO:10).

There is described herein an expression vector comprising a nucleotide sequence encoding prenyltransferase PT104 protein, wherein the nucleotide sequence comprises at least 70% identity with positions 98-1153 of SEQ ID NO:17, or wherein the prenyltransferase PT104 protein comprises at least 70% identity with SEQ ID NO:1.

In such an expression vector, the nucleotide sequence encoding the prenyltransferase PT104 protein may comprises, for example, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with positions 98-1153 of SEQ ID NO:17.

In such an expression vector the prenyltransferase PT104 protein may be one having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with SEQ ID NO:1.

A host cell is described herein that is transformed with any one of the expression vectors describe, wherein transformation occurs according to any known process. Such a host cell may additionally comprising one or more of: (a) a nucleic acid as set forth in any one of SEQ ID NO:2 to SEQ ID NO:14; (b) a nucleic acid having at least 70% identity with the nucleotide sequence of (a); (c) a nucleic acid that hybridizes with the complementary strand of the nucleic acid of (a), and this hybridization may occur under stringent conditions; (d) a nucleic acid encoding a protein with the same enzyme activity as the protein encoded by any one of the nucleic acid sequences of (a); (e) a nucleic acid that differs from (a) by one or more nucleotides that are substituted, deleted, and/or inserted; or (f) a derivative of (a), (b), (c), (d), or (e).

The host cell may be a bacterial cell, a fungal cell, a protist cell, or a plant cell, such as any cell described herein. Exemplary cells include S. cerevisiae, E. coli, Yarrowia lipolytica, and Komagataella phaffii.

The methods, vectors, and cell lines described herein may advantageously be used for the production of phytocannabinoids. By utilizing a protein having prenyltransferase activity, such as PT104 from Rhododendron dauricum, the transformation into a heterologous host cell permits the production of cannabinoids without requiring growth of a whole plant. Cannabinoids such as, but not limited to, CBGa and CBGOa, can be prepared and isolated economically and under controlled conditions. Advantageously, it has been found that PT014 functions well in host cells, such as but not limited to yeast, permitting efficient prenylation of aromatic polyketides in the pathway of phytocannabinoid synthesis.

Phytocannabinoids are a large class of compounds with over 100 different known structures that are produced in the Cannabis sativa plant. These bio-active molecules, such as tetrahydrocannabinol (THC) and cannabidiol (CBD), can be extracted from plant material for medical and recreational purposes.

Phytocannabinoids are synthesized from polyketide and terpenoid precursors which are derived from two major secondary metabolism pathways in the cell. For example, the C—C bond formation between the polyketide olivetolic acid and the allylic isoprene diphosphate geranyl pyrophosphate (GPP) produces the cannabinoid cannabigerolic acid (CBGa). This reaction type is catalyzed by enzymes known as prenyltransferases. The Cannabis plant utilizes a membrane-bound prenyltransferase to catalyze the addition of the prenyl moiety to olivetolic acid to form CBGa.

The prenyltransferase referenced herein as “PT104”, which may interchangeably be referenced as d31RdPT1, is known as a daurichromenic acid synthase, an integral membrane protein from Rhododendron dauricum, that has been characterized to convert orsellinic acid and farnesyl pyrophosphate (FPP) to grifolic acid (Saeki et al., 2018).

PT102 (rdPT1) has known utility in the synthetic pathway to grifolic acid, which is an intermediate in the production of daurichromenic acid, a small molecule with anti-HIV properties. PT104 was previously characterized to strictly prefer orsellinic acid as the polyketide precursor and farnesyl pyrophosphate as the preferred prenyl donor. However, it has been surprisingly found, as described herein, that olivetolic acid and GPP can also be taken as substrates for the truncated enzyme, which may thus advantageously be used in phytocannabionoid synthesis. As described herein, PT104 may be used to transform a host cell, for use in prenylating polyketides in the pathway to phytocannabinoid synthesis.

In one aspect, there is a method described of producing a phytocannabinoid or phytocannabinoid analogue, comprising: utilizing PT104, a recombinant prenyltransferase, to react a polyketide with a GPP to produce a phytocannabinoid or phytocannabinoid analogue.

In one aspect there is described a method of producing cannabigorcinic acid (CBGOa), comprising: providing a host cell which produces orsellinic acid; introducing a polynucleotide encoding prenyltransferase PT014 polypeptide into said host cell, culturing the host cell under conditions sufficient for PT104 polypeptide production in effective amounts to react with geranyl phyrophosphate to produce CBGOa.

In one aspect there is described a method of producing cannabigorcinic acid (CBGOa), comprising: culturing a host cell which produces orsellinic acid and comprises a polynucleotide encoding prenyltransferase PT104 polypeptide under conditions sufficient for PTase polypeptide production.

Non limiting examples of phytocannabinoids that can be prepared according to the methods describe include the following, and their acids, tetrahydrocannabinol (THC), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), and cannabigerol monomethyl ether (CBGM). Acid forms

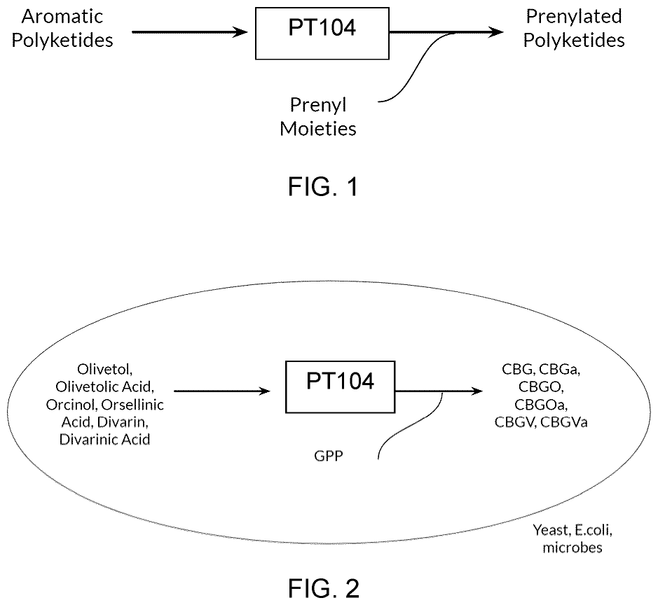

FIG. 1 depicts a generalized scheme for the use of the PT104, as described herein, to attach a prenyl moiety to aromatic polyketides to produce prenylated polyketides.

FIG. 2 depicts examples of specific aromatic polyketides used in the pathway to the production of phytocannabinoids.

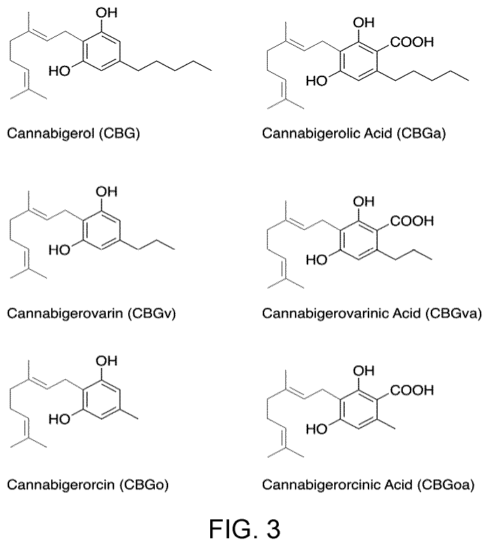

FIG. 3 depicts structures of certain phytocannabinoids produced from the C—C bond formation between a polyketide precursor and geranyl pyrophosphate.

In some example, the cannabinoid or phytocannabinoid may have one or more carboxylic acid functional groups. Non limiting examples of such cannabinoids or phytocannabinoids include tetrahydrocannabinolic acid (THCA), cannabidiolic acid (CBDA), cannabichromenic acid (CBCA), and tetrahydrocannabivarin acid (THCVa).

In some example, the cannabinoid or phytocannabinoid may lack carboxylic acid functional groups. Non limiting examples of such cannabinoids or phytocannabinoids include THC, CBD, CBG, CBC, and CBN.

In some examples of the method described herein, the phytocannabinoid produced is cannabigerol (CBG), cannabigerolic acid (CBGa), cannabigerovarin (CBGv), cannabigerovarinic acid (CBGva), cannabigerocin (CBGo), or cannabigerocinic acid (CBGoa).

In some examples of the method described herein, the polyketide is olivetol, olivetolic acid, divarin, divarinic acid, orcinol, or orsellinic acid.

In some example of the method herein, when the polyketide is olivetol the phytocannabinoid formed is cannabigerol (CBG), when the polyketide is olivetolic acid then the phytocannabinoid is cannabigerolic acid (CBGa), when the polyketide is divarin then the phytocannabinoid is cannabigerovarin (CBGv), when the polyketide is divarinic acid then the phytocannabinoid is cannabigerovarinic acid (CBGva), when the polyketide is orcinol then the phytocannabinoid is cannabigerocin (CBGo), and when the polyketide is orsellinic acid then the phytocannabinoid is cannabigerocinic acid (CBGoa).

Table 1 provides a list of polyketides, prenyl donors and resulting prenylated polyketides. The following terms are used: DMAPP for dimethylallyl diphosphate; GPP for geranyl diphosphate; FPP for farnesyl diphosphate; NPP for neryl diphosphate; and IPP for isopentenyl diphosphate.

TABLE 1
Polyketides, Prenyl Donors and Prenylated Polyketides

| | | | Prenylated Polyketide |
|---|---|---|---|
| # | Polyketide Structure | Prenyl Structure | Structure |
| 1 | 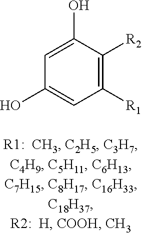 | 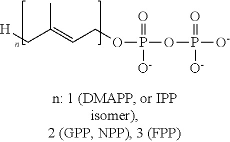 | 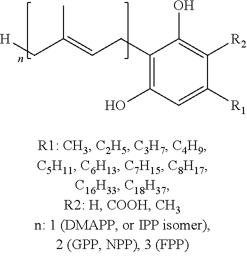 |
| 2 | 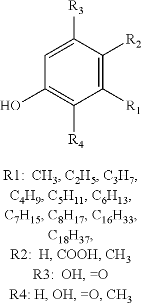 | 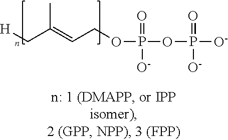 | 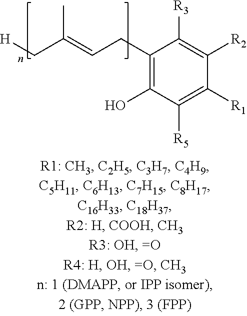 |
| 3 | 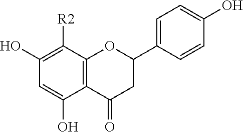 | 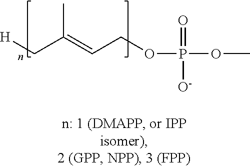 | 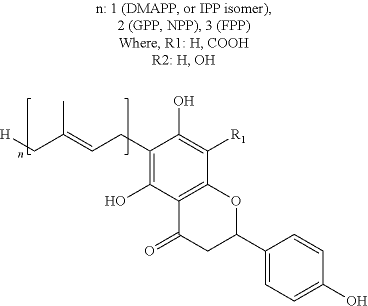 |
| | 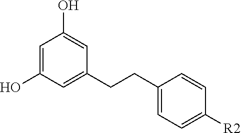 | | 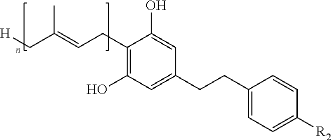 |
| | 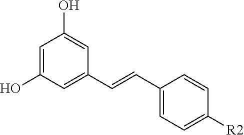 | | 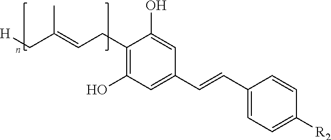 |
| | 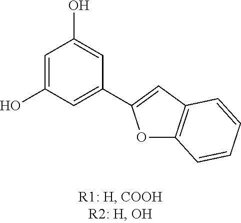 [SMILES: Oc1cc(O)cc(-c2cc3ccccc3o2)c1] | | 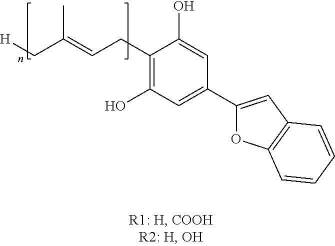 |

Table 2 lists specific examples of host cell organisms for use in one or more of the methods described herein.

TABLE 2
List of Host Cell Organisms

| Type | Organisms |
|---|---|
| Bacteria | Escherichia coli, Streptomyces coelicolor and other species., Bacillus |
| | subtilis, Mycoplasma genitalium, Synechocytis, Zymomonas mobilis, |
| | Corynebacterium glutamicum, Synechococcus sp., Salmonella typhi, |
| | Shigella flexneri, Shigella sonnei, and Shigella disenteriae, Pseudomonas |
| | putida, Pseudomonas aeruginosa, Pseudomonas mevalonii, Rhodobacter |
| | sphaeroides, Rhodobacter capsulatus, Rhodospirillum rubrum, |
| | Rhodococcus sp. |
| Fungi | Saccharomyces cerevisiae, Ogataea polymorpha, Komagataella phaffii, |
| | Kluyveromyces lactis, Neurospora crassa, Aspergillus niger, Aspergillus |
| | nidulans, Schizosaccharomyces pombe, Yarrowia lipolytica, |
| | Myceliophthora thermophila, Aspergillus oryzae, Trichoderma reesei, |
| | Chrysosporium lucknowense, Fusarium sp., Fusarium gramineum, |
| | Fusarium venenatum, Pichia finlandica, Pichia trehalophila, Pichia |
| | koclamae, Pichia membranaefaciens, Pichia opuntiae, Pichia |
| | thermotolerans, Pichia salictaria, Pichia guercuum, Pichia pijperi, Pichia |
| | stipitis, Pichia methanolica, Hansenula polymorpha. |
| Protists | Chlamydomonas reinhardtii, Dictyostelium discoideum, Chlorella sp., |
| | Haematococcus pluvialis, Arthrospira platensis, Dunaliella sp., |
| | Nannochloropsis oceanica. |
| Plants | Cannabis sativa, Arabidopsis thaliana, Theobroma cacao, maize, banana, |
| | peanut, field peas, sunflower, Nicotiana sp., tomato, canola, wheat, barley, |
| | oats, potato, soybeans, cotton, sorghum, lupin, rice. |

Table 3 lists the sequences described herein, for greater certainty. Actual sequences are provided in later tables, below.

TABLE 3
List of sequence characteristics

| | | | Length of | Position of coding |
|---|---|---|---|---|
| SEQ ID NO: | Description | DNA/Protein | sequence | sequence |
| SEQ ID NO. 1 | PT104 aa sequence | Protein | 102 | all |
| SEQ ID NO. 2 | NpgA | DNA | 3564 | 1170-2201 |
| SEQ ID NO. 3 | DiPKS-1 | DNA | 11114 | 849-10292 |
| SEQ ID NO. 4 | DiPKS-2 | DNA | 10890 | 717-10160 |
| SEQ ID NO. 5 | DiPKS-3 | DNA | 11300 | 795-10238 |
| SEQ ID NO. 6 | DiPKS-4 | DNA | 11140 | 794-10237 |
| SEQ ID NO. 7 | DiPKS-5 | DNA | 11637 | 1172-10615 |
| SEQ ID NO. 8 | PDH | DNA | 7114 | Ald6: 1444-2949 |
| | | | | ACS: 3888-5843 |
| SEQ ID NO. 9 | Maf1 | DNA | 3256 | 936-2123 |
| SEQ ID NO. 10 | Erg20K197E | DNA | 4254 | 2683-3423 |
| SEQ ID NO. 11 | Erg1p:UB14- | DNA | 3503 | 1364-2701 |
| | Erg20:deg | | | |
| SEQ ID NO. 12 | tHMGr-IDI | DNA | 4843 | tHMGR1: 877-2385 |
| | | | | IDI1: 3209-4075 |
| SEQ ID NO. 13 | PGK1p:ACC1^{S659A, S1157A} | DNA | 7673 | Pgk1p: 222-971 |
| | | | | Acc1mut: 972-7673 |
| SEQ ID NO. 14 | OAC | DNA | 2177 | 842-1150 |
| SEQ ID NO. 15 | csOAC aa sequence | Protein | 102 | all |
| SEQ ID NO. 16 | DiPKSG1516R aa | Protein | 3147 | all |
| | sequence | | | |
| SEQ ID NO. 17 | PLAS250 | DNA | 6841 | 98-1153 |
| SEQ ID NO. 18 | PLAS36 | DNA | 8980 | |

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES—PART 1

### Example 1

PT104 in Production of Prenylated Polyketides in Yeast

Introduction. Phytocannabinoids are naturally produced in Cannabis sativa, other plants, and some fungi. Over 105 phytocannabinoids are known to be biosynthesized in C. sativa, or result from thermal or other decomposition from phytocannabinoids biosynthesized in C. sativa. While the C. sativa plant is also a valuable source of grain, fiber, and other material, growing C. sativa for phytocannabinoid production, particularly indoors, is costly in terms of energy and labour. Subsequent extraction, purification, and fractionation of phytocannabinoids from the C. sativa plant is also labour and energy intensive.

Phytocannabinoids are pharmacologically active molecules that contribute to the medical and psychotropic effects of C. sativa. Biosynthesis in the C. sativa plant scales similarly to other agricultural projects. As with other agricultural projects, large scale production of phytocannabinoids by growing C. sativa requires a variety of inputs (e.g. nutrients, light, pest control, CO2, etc.). The inputs required for cultivating C. sativa must be provided. In addition, cultivation of C. sativa, where allowed, is currently subject to heavy regulation, taxes, and rigorous quality control where products prepared from the plant are for commercial use, further increasing costs. As a result, it may be economical to produce the phytocannabinoids in a robust and scalable, fermentable organism. Saccharomyces cerevisiae has been used to produce industrial scales of similar molecules.

The time, energy, and labour involved in growing C. sativa for phytocannabinoid production provides a motivation to produce transgenic cell lines for production of phytocannabinoids in yeast. One example of such efforts is provided in International patent application by Mookerjee et al. WO2018/148848.

Production of phytocannabinoids in genetically modified strains of Saccharomyces cerevisiae are described in this Example. The modified strains have been transformed with genes coding for a prenyltransferase (PT104) from Rhododendron dauricum that catalyzes the synthesis of cannabigerolic acid (CBGA) from olivetolic acid (OLA) and geranyl pyrophosphate (GPP).

In C. sativa, a prenyltransferase enzyme catalyzes the synthesis of CBGa from olivetolic acid and GPP. However, the C. sativa prenyltransferase functions poorly in S. cerevisiae, as described in U.S. Pat. No. 8,884,100.

PT104 was evaluated in this Example, to determine advantages over the C. sativa prenyltransferase when expressed in S. cerevisiae, to catalyze the synthesis of CBGA from OLA and GPP so as to create a consolidated phytocannabinoid producing strain of S. cerevisiae. The S. cerevisiae may also have one or more mutations or modification in genes and metabolic pathways that are involved in OLA and/or GPP production or consumption.

The modified S. cerevisiae strain may also express genes encoding for Dictyostelium polyketide synthase (DiPKS), a hybrid Type1 FAS-Type 3 PKS from Dictyostelium discoideum (Ghosh et al., 2008) and olivetolic acid cyclase (OAC) from C. sativa (Gagne et al., 2012). DiPKS allows for the direct production of methyl-Olivetol (meOL) from malonyl-coA, a native yeast metabolite. Certain mutants of DiPKS have been identified that lead to the direct production of olivetol (OL) from malonyl-coA (WO2018/148848). OAC has been demonstrated to assist in the production of olivetolic acid when a suitable Type 3 PKS is used.

The C. sativa cannabis pathway enzymes requires hexanoic acid for the production of OLA. However, hexanoic acid is highly toxic to S. cerevisiae and greatly diminishes its growth phenotype. As a result, when using DiPKS and OAC rather than the C. sativa pathway enzymes, hexanoic acid need not be added to the growth media, which may result in increased growth of the S. cerevisiae cultures and greater production of olivetolic acid. The S. cerevisiae may have over-expression of native acetaldehyde dehydrogenase and expression of a modified version of an acetoacetyl-CoA carboxylase or other genes, the modifications resulting in lowered mitochondrial acetaldehyde catabolism. Lowering mitochondrial acetaldehyde catabolism by diverting the acetaldehyde into acetyl-CoA production increases malonyl-CoA available for synthesizing olivetolic acid.

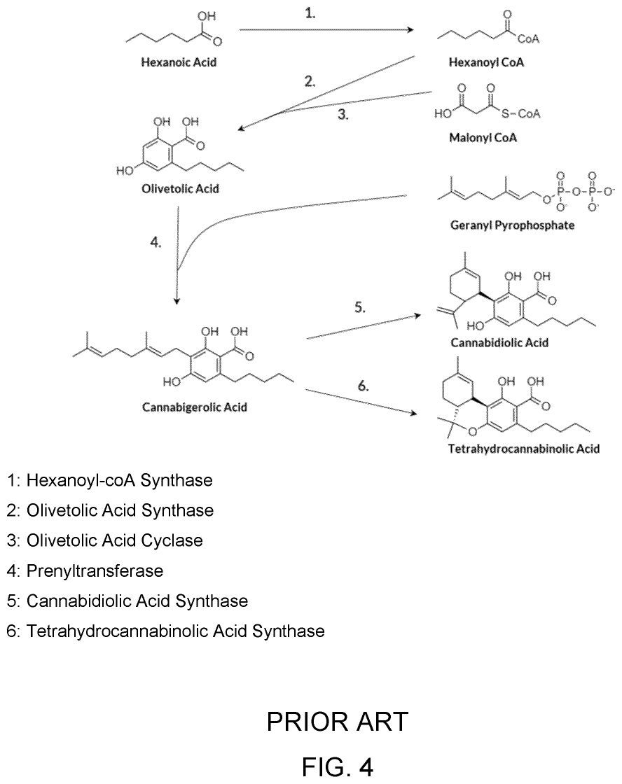

FIG. 4 outlines the native biosynthetic pathway for cannabinoid production in Cannabis sativa. Hexanoic acid is converted to hexanoyl-CoA by hexanoyl-CoA synthase (1). Hexanoyl-CoA is used, together with malonyl-CoA as an extender unit, by the olivetolic acid synthase (2) and olivetolic acid cyclase (3) enzymes. This produces olivetolic acid. Olivetolic acid and geranyl pyrophosphate (GPP) are subsequently converted into cannabigerolic acid (CBGa) by a prenyltransferase enzyme (4), such as a geranyl transferase. The prenyl group on CBGa is subsequently cyclized to produce tetrahydrocannabinollic acid (THCa) and cannabidiolic acid (CBDa) with the reactions being catalyzed by the oxidocyclases: tetrahydrocannabinolic acid (THCa) synthase (6) and cannabidiolic acid (CBGa) synthase (5) respectively.

As expression and functionality of the C. sativa pathway in S. cerevisiae is hindered by problems of toxic precursors and poor expression, this Example utilizes a novel biosynthetic route for cannabinoid production. This route was developed to overcome one or more of the above-described detrimental issues.

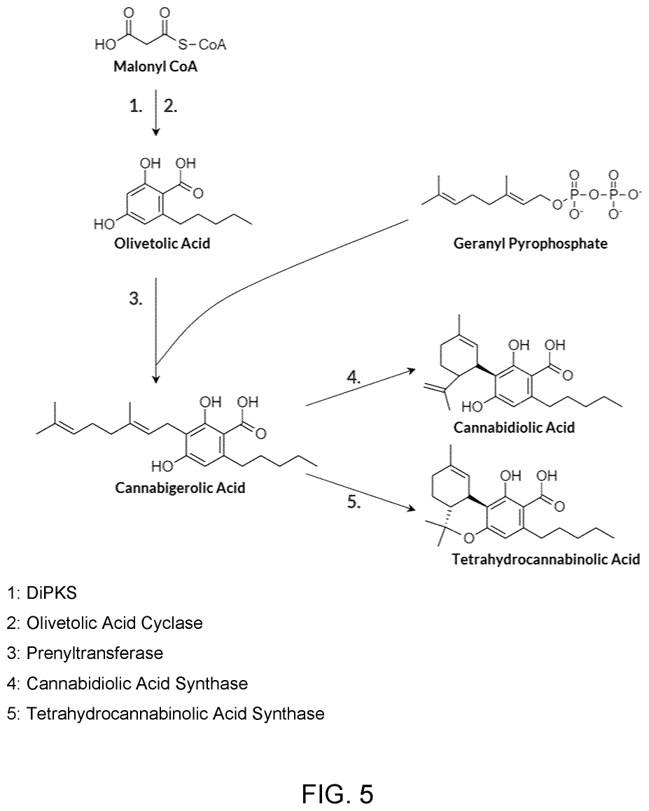

FIG. 5 outlines the pathway of cannabinoid biosynthesis as described herein. A four enzyme system is described. Dictyostelium polyketide synthase (DiPKS) (1), from D. discoideum and olivetolic acid cyclase (OAC) (2) from C, sativa are used to produce olivetolic acid directly from glucose, via acetyl CoA and malonyl CoA. Geranyl pyrophosphate (GPP) from the yeast terpenoid pathway and olivetolic acid (OLA) are subsequently converted to Cannabigerolic acid using a prenyltransferase (3), which in this example is: PT104. Cannabigerolic acid is then further cyclized to produce THCa or CBDa using C. sativa THCa synthase (5) or CBDa synthase (4) enzymes, respectively.

The prenyltransferase referenced herein as “PT104”, which may interchangeably be referenced as RdPT1, is a daurichromenic acid synthase, an integral membrane protein from Rhododendron dauricum, that has been characterized to convert orsellinic acid and farnesyl pyrophosphate (FPP) to grifolic acid (Saeki et al., 2018).

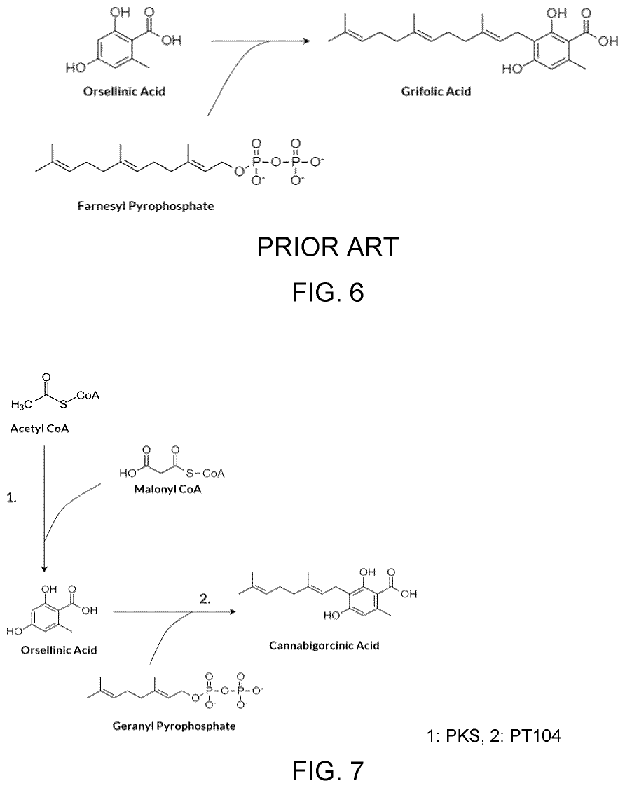

FIG. 6 outlines the function of PT104 (d31rdPT1) in the known synthetic pathway to grifolic acid. Grifolic acid is an intermediate in the production of daurichromenic acid, an anti-HIV small molecule. This enzyme was previously characterized to strictly prefer orsellinic acid as the polyketide precursor and farnesyl pyrophosphate as the preferred prenyl donor. However it has been surprisingly found, as described herein, that olivetolic acid and GPP can also be taken as substrates for this enzyme. This leads to advantages for the use of this enzyme in phytocannabionoid synthesis.

FIG. 7 illustrates synthesis of cannabigorcinic acid starting with malonyl CoA and Acetyl CoA with PKS to form orsellinic acid, which together with GPP and PT104 as described herein results in cannabigorcinic acid.

This example describes, for the first time, the in vivo production of cannabigerorcinic acid (CBGOa) and CBGa in S. cerevisiae using PT104 as the prenyltransferase.

Table 4 shows the modifications made to the base strain used in this example to allow olivetolic acid production. The modifications are named, and described with reference to a sequence (SEQ ID NO.), the integration region in the genome, and other details such as the genetic structure of the sequence.

TABLE 4
Modifications to base strain used in Example 1

| | | | Integration | | Genetic |
|---|---|---|---|---|---|
| | | SEQ ID | Region/ | | Structure of |
| # | Name | NO. | Plasmid | Description | Sequence |
| 1 | NpgA | SEQ ID | Flagfeldt Site | Phosphopantetheinyl Transferase from | Site14Up::Tef1p: |
| | | NO. 2 | 14 integration | Aspergillus niger. Accessory Protein for | NpgA:Prm9t: |
| | | | | DiPKS (see Kim et al., 2015) | Site14Down |
| 2 | DiPKS- | SEQ ID | USER Site | Type 1 FAS fused to Type 3 PKS from D. | XII-1up::Gal1p: |
| | 1 | NO. 3 | XII-1 | discoideum. Produces Olivetol from | DiPKSG1516R: |
| | | | integration | malonyl-coA | Prm9t::XII1-down |
| | | | (Jensen et | | |
| | | | al., 2014) | | |
| 3 | DiPKS- | SEQ ID | Wu site 1 | Type 1 FAS fused to Type 3 PKS from D. | Wu1up::Gal1p: |
| | 2 | NO. 4 | integration | discoideum. Produces Olivetol from | DiPKSG1516R: |
| | | | | malonyl-coA | Prm9t::Wu1down |
| 4 | DiPKS- | SEQ ID | Wu site 3 | Type 1 FAS fused to Type 3 PKS from D. | Wu3up::Gal1p: |
| | 3 | NO. 5 | integration | discoideum. Produces Olivetol from | DiPKSG1516 |
| | | | | malonyl-coA | R:Prm9t::Wu3down |
| 5 | DiPKS- | SEQ ID | Wu site 6 | Type 1 FAS fused to Type 3 PKS from D. | Wu6up::Gal1p: |
| | 4 | NO. 6 | integration | discoideum. Produces Olivetol from | DiPKSG1516R: |
| | | | | malonyl-coA | Prm9t::Wu6down |
| 6 | DiPKS- | SEQ ID | Wu site 18 | Type 1 FAS fused to Type 3 PKS from D. | Wu18up::Gal1p: |
| | 5 | NO. 7 | integration | discoideum. Produces Olivetol from | DiPKSG1516R: |
| | | | | malonyl-coA | Prm9t::Wu18down |
| 7 | PDH | SEQ ID | Flagfeldt Site | Acetaldehyde dehydrogenase (ALD6) | 19Up::Tdh3p: |
| | | NO. 8 | 19 integration | from S. cerevisiae and acetoacetyl coA | Ald6:Adh1::Tef1p: |
| | | | | synthase (AscL641P) from Salmonella | seACS1^{L641P}: |
| | | | | enterica. Will allow greater accumulation | Prm9t::19Down |
| | | | | of acetyl-coA in the cell. (Shiba et al., | |
| | | | | 2007) | |
| 8 | Maf1 | SEQ ID | Flagfeldt Site | Maf1 is a regulator of tRNA biosynthesis. | Site5Up::Tef1p: |
| | | NO. 9 | 5 integration | Overexpression in S. cerevisiae has | Maf1:Prm9t: |
| | | | | demonstrated higher monoterpene | Site5Down |
| | | | | (GPP) yields. (Liu et al, 2013) | |
| 9 | Erg20K | SEQ ID | Chromosomal | Mutant of Erg20 protein that diminishes | Tpi1t:ERG20K197E: |
| | 197E | NO. 10 | modification | FPP synthase activity creating greater | Cyc1t::Tef1p: |
| | | | | pool of GPP precursor. Negatively affects | KanMX:Tef1t |
| | | | | growth phenotype (Oswald et al., 2007). | |
| 10 | Erg1p:UB14- | SEQ ID | Flagfeldt Site | Sterol responsive promoter controlling | Site18Up::Erg1p: |
| | Erg20:deg | NO. 11 | 18 integration | Erg20 protein activity. Allows for regular | UB14deg:ERG20: |
| | | | | FPP synthase activity and uninhibited | Adh1t:Site18down |
| | | | | growth phenotype until accumulation of | |
| | | | | sterols which leads to a suppression of | |
| | | | | expression of enzyme. (Peng et al., | |
| | | | | 2018) | |
| 11 | tHMGr- | SEQ ID | USER Site X- | Overexpression of truncated HMGr1 and | X3up::Tdh3p:tHMGR1: |
| | IDI | NO. 12 | 3 integration | IDI1 proteins that have been previously | Adh1t::Tef1p:IDI1: |
| | | | | identified to be bottlenecks in the S. | Prm9t::X3down |
| | | | | cerevisiae terpenoid pathway responsible | |
| | | | | for GPP production. (Ro et al., 2006) | |
| 12 | PGK1p: | SEQ ID | Chromosomal | Mutations in the native S. cerevisiae | Pgk1:ACC1^{S659A, S1157A}: |
| | ACC1^{S659A, S1157A} | NO. 13 | modification | acetyl-coA carboxylase that removes | Acc1t |
| | | | | post-translational modification based | |
| | | | | down-regulation. Leads to greater | |
| | | | | malonyl-coA pools. The promoter of Acc1 | |
| | | | | was also changed to a constitutive | |
| | | | | promoter for higher expression. (Shi et | |
| | | | | al, 2014) | |
| 13 | OAC | SEQ ID | Flagfeldt Site | Plasmid expressing Cannabis sativa | Gal1p:csOAC:Eno2t |
| | | NO. 14 | 16 integration | Olivetolic acid cyclase (OAC) protein that | |
| | | | | allows the production of olivetolic acid. | |

TABLE 5
Plasmid Information

| # | Plasmid Name | Description | Selection |
|---|---|---|---|
| 1 | PLAS250 | pGAL_Gal1p:PT104:Cyc1t | Uracil |
| 2 | PLAS36 | pCAS_Hyg_Rnr2p:Cas9: | Hygromycin |
| | | Cyc1t::tRNATyr:HDV:gRNA:Snr52t | |

Table 6 lists the strains used in this example, providing the features of the strains including background, plasmids if any, genotype, etc.

TABLE 6
Strains Used

| Strain # | Background | Plasmids | Genotype | Notes |
|---|---|---|---|---|
| HB42 | -URA, -LEU | None | Saccharomyces cerevisiae | Base Strain |
| | | | CEN.PK2; ΔLEU2; ΔURA3; | |
| | | | Erg20K197E::KanMx | |
| HB742 | -URA, -LEU | None | Saccharomyces cerevisiae | Starting |
| | | | CEN.PK2; ΔLEU2; ΔURA3; | strain |
| | | | Erg20K197E::KanMx; ALD6; | |
| | | | ASC1L641P; NPGA; MAF1; | |
| | | | PGK1p:Acc1; tHMGR1; IDI; | |
| | | | DiPKS_G1516R X 5; | |
| | | | ACC1_S659A_S1157A; | |
| | | | UB14p:ERG20 | |
| HB801 | -URA, -LEU | None | Saccharomyces cerevisiae | Olivetolic |
| | | | CEN.PK2; ΔLEU2; ΔURA3; | acid |
| | | | Erg20K197E::KanMx; ALD6; | producing |
| | | | ASC1L641P; NPGA; MAF1; | strain |
| | | | PGK1p:Acc1; tHMGR1; IDI; | |
| | | | DiPKS_G1516R X 5; | |
| | | | ACC1_S659A_S1157A; | |
| | | | UB14p:ERG20; | |
| | | | Gal1p:csOAC | |
| HB887 | -URA, -LEU | PLAS250 | Saccharomyces cerevisiae | CBGa |
| | | | CEN.PK2; ΔLEU2; ΔURA3; | producing |
| | | | Erg20K197E::KanMx; ALD6; | strain |
| | | | ASC1L641P; NPGA; MAF1; | |
| | | | PGK1p:Acc1; tHMGR1; IDI; | |
| | | | DiPKS_G1516R X 5; | |
| | | | ACC1_S659A_S1157A; | |
| | | | UB14p:ERG20; | |
| | | | Gal1p:csOAC | |

Features and characteristics of sequences noted here are provided in Table 3.

Materials and Methods

Genetic Manipulations

HB42 was used as a base strain to develop all other strains in this example. All DNA was transformed into strains using the Gietz et al. (2014) transformation protocol. Plas 36 was used for the CRISPR-based genetic modifications described in this experiment (Ryan et al., 2016). All plasmids were synthesized by TWIST DNA Sciences.

The genome of HB42 was iteratively targeted by gRNA's and Cas9 expressed from PLAS36 to make the following genomic modifications in the order shown below in Table 7.

TABLE 7
Genomic Modifications to Base Strain BH42

| Order | Genomic Region | Modification |
|---|---|---|
| 1 | Flagfeldt Site 19 integration | PDH |
| 2 | Flagfeldt Site 14 integration | NpgA |
| 3 | Flagfeldt Site 5 integration | Maf1 |
| 4 | Chromosomal Modification | PGK1p:ACC1^{S659A, S1157A} |
| 5 | USER Site X-3 integration | tHMGR-IDI1 |
| 6 | USER Site XII-2 integration | DiPKS-1 |
| 7 | Flagfeldt Site 18 integration | Erg1p:UB14-Erg20:deg |
| 8 | Wu site 1 integration | DiPKS-2 |
| 9 | Wu site 3 integration | DiPKS-3 |
| 10 | Wu site 6 integration | DiPKS-4 |
| 11 | Wu site 18 integration | DiPKS-5 |

The result of the above modifications was a S. cerevisiae strain that could produce olivetol directly from glucose and was named “HB742”, as an internal laboratory designation for the purposes of this example.

The genome at Flagfeldt site 16 (Bai Flagfeldt et al., 2009) in HB742 was subsequently targeted using Cas9 and gRNA expressed from PLAS36 which was transformed into HB742. The donor for the recombination was SEQ ID NO:14. Successful integrations were selected on YPD+200 ug/ml Hygromycin and confirmed by colony PCR. This led to the creation of “HB801” (internal designation) with a galactose inducible csOAC encoding gene integrated into the genome of HB742. The genomic region containing SEQ ID NO:14 was also verified by sequencing to confirm the presence of the csOAC encoding gene. This allowed for the creation of an olivetolic acid producing strain, HB801 (internal designation). PLAS250 which encodes a galactose-inducible gene expressing PT104 was subsequently transformed into HB801 producing a strain that can synthesize cannabigorcinic acid directly from glucose, HB887 (internal designation).

Strain Growth and Media:

HB887 was grown on yeast minimal media with a composition of 1.7 g/L YNB without ammonium sulfate+1.96 g/L URA dropout amino acid supplements+1.5 g/L magnesium L-glutamate) with 2% w/v galactose, 2% w/v raffinose, 200 μg/l geneticin, and 200 ug/L ampicillin (Sigma-Aldrich, Canada). This would allow the strain to produce olivetolic acid and cannabigerolic acid and potentially other cannabinoids.

In another embodiment in this example, HB887 was grown in yeast minimal media with a composition of 1.7 g/L YNB without ammonium sulfate+1.96 g/L URA dropout amino acid supplements+1.5 g/L magnesium L-glutamate) with 2% w/v glucose, 200 μg/l geneticin, and 200 ug/L ampicillin (Sigma-Aldrich Canada). This is a non-inducible condition and the strain would not produce phytocannabinoids.

In another embodiment in this example, HB887 was grown in yeast minimal media with a composition of 1.7 g/L YNB without ammonium sulfate+1.96 g/L URA dropout amino acid supplements+1.5 g/L magnesium L-glutamate) with 2% w/v glucose, 200 μg/l geneticin, and 200 ug/L ampicillin+100 mg/L Orsellinic acid (Sigma-Aldrich, Canada). This is also a non-inducible condition and would not allow the strain to produce any phytocannabinoids.

HB887 was grown on yeast minimal media with a composition of 1.7 g/L YNB without ammonium sulfate+1.96 g/L URA dropout amino acid supplements+1.5 g/L magnesium L-glutamate) with 2% w/v galactose, 2% w/v raffinose, 200 μg/l geneticin, and 200 ug/L ampicillin+100 mg/L Orsellinic acid (Sigma-Aldrich Canada). This would allow HB887 to produce both CBGa and CBGOa.

Experimental Conditions

12 single colony replicates of strains were tested in this study. All strains were grown in 1 ml cultures in 96-well deepwell plates. The deepwell plates were incubated at 30° C. and shaken at 250 rpm for 96 hrs.

Metabolite extraction was performed with 300 μl of Acetonitrile added to 100 μl culture in a new 96-well deepwell plate, followed by 30 min of agitation at 950 rpm. The solutions were then centrifuged at 3750 rpm for 5 min. 200 μl of the soluble layer was removed and stored in a 96-well v-bottom microtiter plate. Samples were stored at −20° C. until analysis.

Samples were quantified using HPLC-MS analysis.

CBGa Quantification Protocol

The quantification of CBGa was performed using HPLC-MS on a Acquity UPLC-TQD MS. The chromatography and MS conditions are described below.

LC conditions: Column: Hypersil Gold PFP 100×2.1 mm, 1.9 μm particle size; Column temperature: 45° C.; Flow rate: 0.6 ml/min; Eluent A: Water 0.1% formic acid; and Eluent B: Acetontrile 0.1% formic acid.

Gradient (Time (min) and % B) is expressed as: Time=Initial; 51 (isocratic) and Time=2.50; 51 (isocratic).

ESI-MS conditions: Capillary: 3 kV; Source temperature: 150° C.; Desolvation gas temperature: 450° C.; Desolvation gas flow (nitrogen): 800 L/hr; and Cone gas flow (nitrogen): 50 L/hr.

CBGa detection parameters are as follows: Retention time: 1.19 min; Ion [M-H]⁻; Mass (m/z): 359.2; Mode: ES−, SIR; Span: 0; Dwell (s): 0.2; and Cone (V): 30.

CBGOa Quantification Protocol

CBGOa was quantified using HPLC-MS on a Waters Acquity TQD. Table 8 lists the CBGOa detection parameters.

TABLE 8
CBGOa Detection Parameters

| | |
|---|---|
| | Waters HSS |
| Column | 1 × 50 mm, 1.8 um |

LC Method

| A1 | Water + 0.1% FA | |
|---|---|---|
| B1 | ACN + 0.1% FA | |
| Flow rate | 0.3 mL/min | |
| | A1 | B2 |
| 0.00 min | 50% | 50% |
| 0.80 min | 15% | 85% |
| 1.00 min | 5% | 95% |
| 1.01 min | 50% | 50% |
| 1.80 min | 50% | 50% |
| | RT (min) | |
| CBGOa | 0.96 min | |

MS Method

| | | | Cone | Collision |
|---|---|---|---|---|
| ES+ | M/Z | Transition | Voltage (V) | (V) |
| CBGOa | 261.2 | → 161.1 | 20 | 12 |

Results:

Production of CBGa in S. cerevisiae.

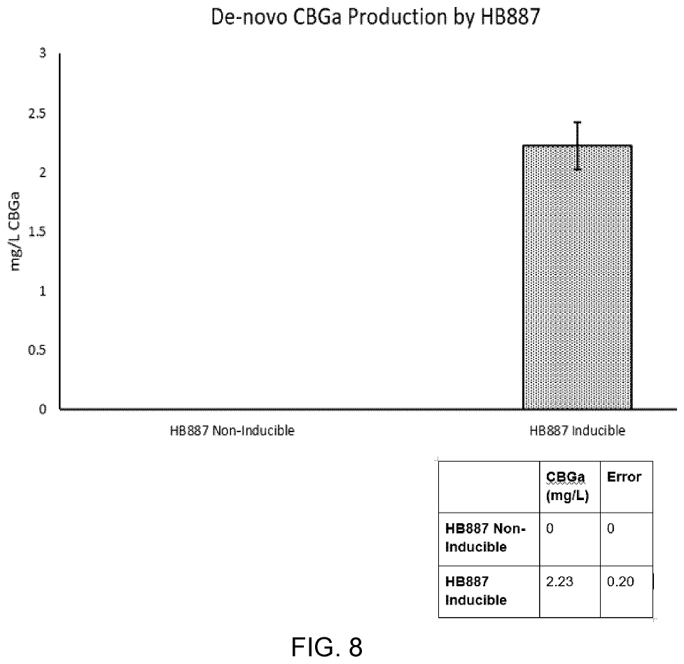

FIG. 8 illustrates the de-novo CBGa production by HB8887. These data show that CBGa was produced by HB887 directly from glucose and/or primary carbon source when it was grown under the inducible condition as opposed to its growth in the uninducible condition.

Production of CBGa and CBGOa Simultaneously in S. cerevisiae HB887.

To test the functionality of this enzyme against both of the polyketides substrates at the same time, HB887 was grown in the inducible condition along with an addition of 100 mg/L of orsellinic acid. It was observed that HB887 was producing both CBGa and CBGOa simultaneously. As this enzyme has a preference for orsellinic acid as a substrate it was more functional at producing CBGOa, however there was quantifiable CBGa production as well.

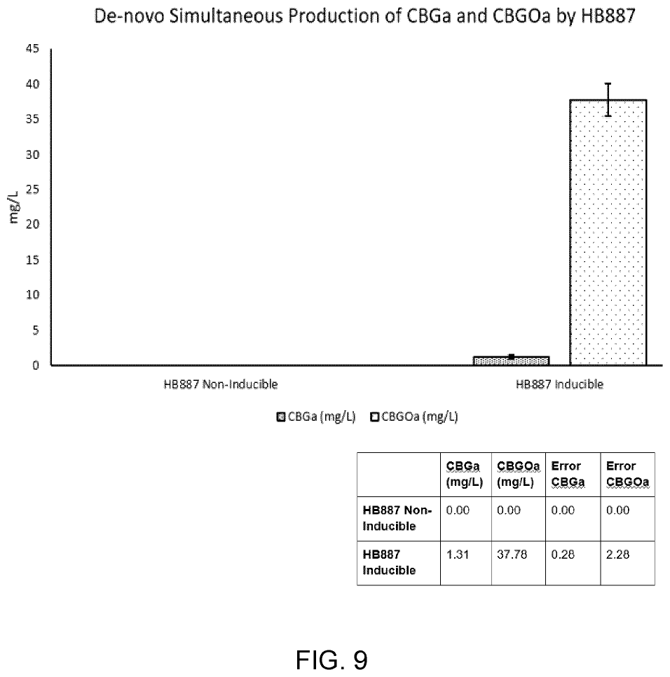

FIG. 9 illustrates the de-novo simultaneous production of CBGa and CBGOa by HB8887. These data illustrate that PT104 has the capacity to prenylate orsellinic acid and olivetolic acid.

Part 2

ABBA Family Prenyltransferases for Production of Prenylated Polyketides and Phytocannabinoids

The present disclosure relates generally to prenyltransferases, which may be of an ABBA Family type, useful in production of phytocannabinoids and phytocannabinoid precursors such as polyketides. Cells, such as yeast cells transformed with the ability to prepare such phytocannabinoids or precursors are described.

Overview

In one aspect there is provided a method of producing a phytocannabinoid or phytocannabinoid analogue comprising: providing a host cell which produces a polyketide and a prenyl donor; introducing a polynucleotide encoding prenyltransferase (PTase) polypeptide into said host cell; and culturing the host cell under conditions sufficient for PTase polypeptide production to thereby react the PTase with the polyketide and the prenyl donor to produce said phytocannabinoid or phytocannabinoid analogue.

The recombinant PTase may be one comprising or consisting of an amino acid sequence set forth in SEQ ID NOs: 59 to 97; or having at least 70% identity thereto.

Further, the recombinant PTase may be one that is encoded by polynucleotide comprising or consisting of: a nucleotide sequence set for forth in SEQ ID NOs: 20 to 58, or a nucleotide sequence having at least 70% identity thereto, or a nucleotide sequence that hybridizes with the complementary strand thereof, or a nucleotide sequence that differs therefrom by one or more nucleotides that are substituted, deleted, and/or inserted; or a derivative thereof.

An isolated polypeptide is described comprising or consisting of an amino acid sequence set forth in SEQ ID NOs: 59 to 97; or at least 50% 99% identity thereto. Further, an isolated polynucleotide is described comprising a nucleotide sequence set for forth in SEQ ID NOs: 20 to 58 or 100, or having at least 70% identity thereto or a nucleotide sequence that hybridizes with the complementary strand thereof, or which differs therefrom by one or more nucleotides that are substituted, deleted, and/or inserted; or a derivative thereof having prenyltransferase activity. Expression vectors encoding the polypeptide and host cells comprising the polynucleotide or expression vector are described.

### Detailed Description of Part 2

Generally, there is described herein the production of phyotocannabinoids or phytocannabinoid analogues.

Phytocannabinoids are a large class of compounds with over 100 different known structures that are produced in the Cannabis sativa plant. These bio-active molecules, such as tetrahydrocannabinol (THC) and cannabidiol (CBD), can be extracted from plant material for medical and recreational purposes.

Phytocannabinoids are synthesized from polyketide and terpenoid precursors which are derived from two major secondary metabolism pathways in the cell. For example, the C—C bond formation between the polyketide olivetolic acid and the allylic isoprene diphosphate geranyl pyrophosphate (GPP) produces the cannabinoid cannabigerolic acid (CBGa). This reaction type is catalyzed by enzymes known as prenyltransferases (PTases). The Cannabis plant utilizes a membrane-bound PTase to catalyze the addition of the prenyl moiety to olivetolic acid to form CBGa.

A cytosolic class of PTase that adopt an anti-parallel p/a barrel structure, known as the ABBA family PTs, may be more amenable to heterologous expression in recombinant hosts. The first reported example of this class of PTase was NphB (U.S. Pat. No. 7,361,483 B2, doi:10.1038/nature03668) which demonstrated catalytic activity for the prenylation of olivetol and olivetolic acid.

Herein, the use of nucleotide and protein sequences for ABBA PTases that demonstrate activity with aromatic acceptor substrates is reported.

In one aspect, there is a method described of producing a phytocannabinoid or phytocannabinoid analogue, comprising, reacting a recombinant prenyltransferase (PTase) with a polyketide and with a GPP to produce said phytocannabinoid or phytocannabinoid analogue.

In one aspect there is described a method of producing cannabigorcinic acid (CBGOa), comprising: providing a host cell which produces orsellinic acid; introducing a polynucleotide encoding prenyltransferase (PTase) polypeptide into said host cell, culturing the host cell under conditions sufficient for PTase polypeptide production.

In one aspect there is described a method of producing cannabigorcinic acid (CBGOa), comprising: introducing a polynucleotide encoding prenyltransferase (PTase) polypeptide into a host cell which produces orsellinic acid, culturing the host cell under conditions sufficient for PTase polypeptide production.

In one aspect there is described a method of producing cannabigorcinic acid (CBGOa), comprising: culturing a host cell which produces orsellinic acid and comprises or consists of a polynucleotide encoding prenyltransferase (PTase) polypeptide under conditions sufficient for PTase polypeptide production.

In some example of the method herein, the phytocannabinoid produced is cannabigerol (CBG), cannabigerolic acid (CBGa), cannabigerovarin (CBGv), cannabigerovarinic acid (CBGva), cannabigerocin (CBGo), or cannabigerocinic acid (CBGoa).

In some example of the method herein, the polyketide is olivetol, olivetolic acid, divarin, divarinic acid, orcinol, or orsellinic acid.

In some example of the method herein, when said polyketide is olivetol then said phytocannabinoid is cannabigerol (CBG), when said polyketide is olivetolic acid then said phytocannabinoid is cannabigerolic acid (CBGa), when said polyketide is divarin then said phytocannabinoid is cannabigerovarin (CBGv), when said polyketide is divarinic acid then said phytocannabinoid is cannabigerovarinic acid (CBGva), when said polyketide is orcinol then said phytocannabinoid is cannabigerocin (CBGo), when said polyketide is orsellinic acid then said phytocannabinoid is cannabigerocinic acid (CBGoa).

In one example, said polyketide is:

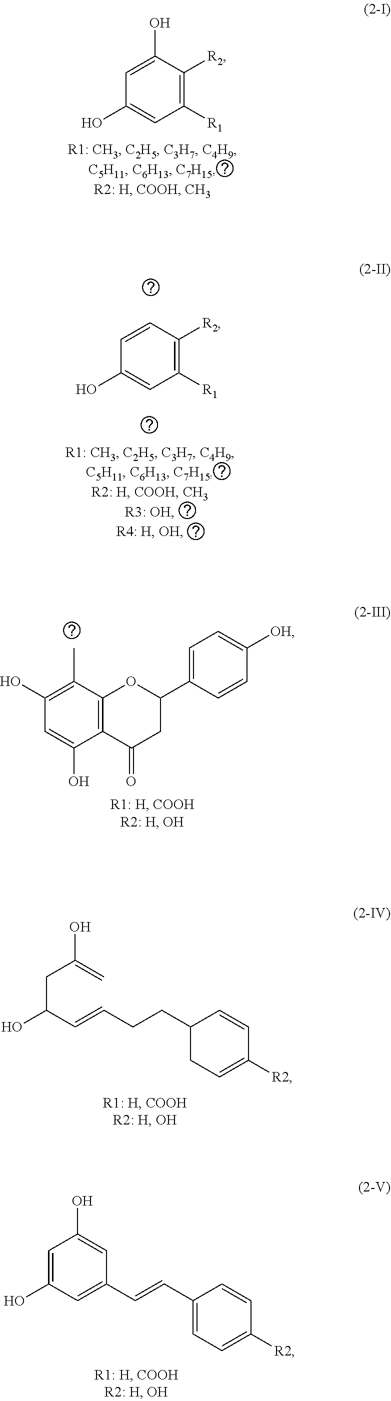

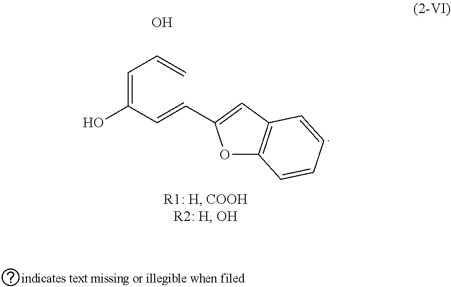

In one example, said prenyl donor is:

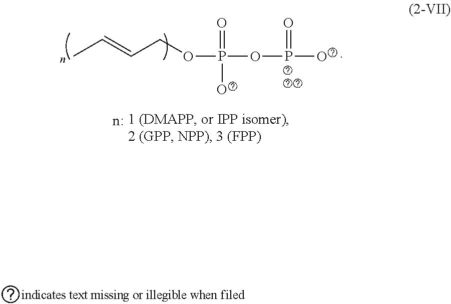

In one example, said phytocannabinoid or phytocannabinoid analogue is:

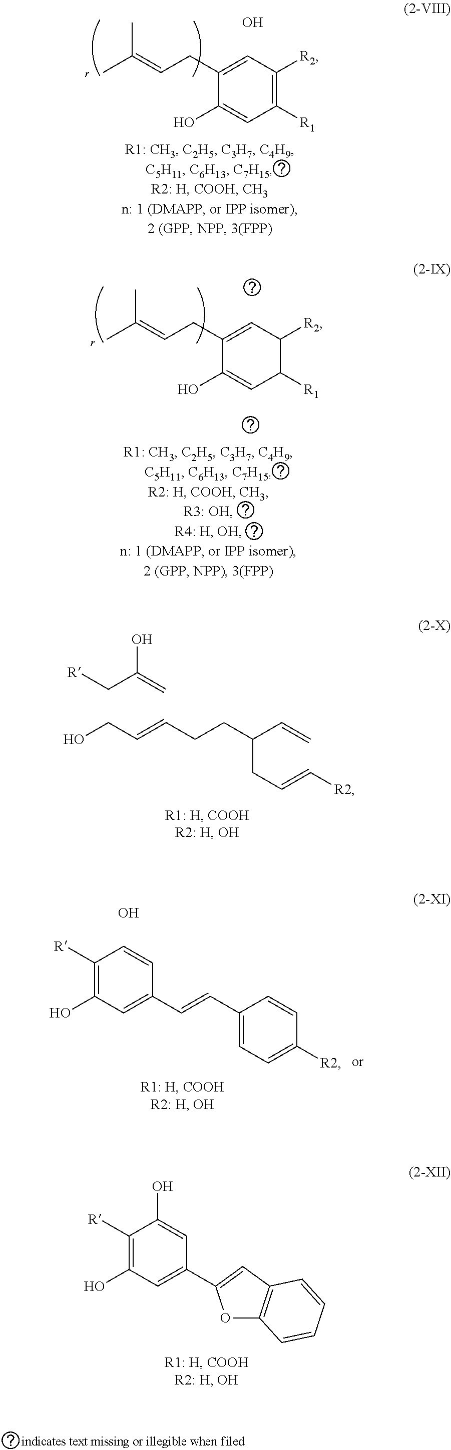

In one example, said recombinant PTase comprising or consisting of an amino acid sequence set for in SEQ ID NOs: 59 to 97; or at least 50%, at least 60%, at least 70%, at least 80%, at least 90% identity with the amino acid sequence set forth in SEQ ID NOs: 59 to 97; and/or 100% identity with the amino acid sequence set forth in SEQ ID NOs: 59 to 97.

In one example, said recombinant PTase comprises or consists of the following consensus sequence according to SEQ ID NO:118:

xxxxxxxxxxxxxxxxxxxxxxxxxxxxxxxxxxxxxxxxxxxxxxxxxx
xxxxxxxxxxxxxxxxxxxxxxxxxxxxxxxxMSxxSELDELYSAIEESA
RLLDVxCSRDKVxPVLTAYGDxxAxxxxVIAFRVxTxxRxxGELDYRFxx
xPxxxDPYxxALSNGLIxETDHPxxxxxVGSLLSDIRERxPIxSYGxxxx
IDEGVVGGFKKIWxFFPxDxMQxVSELAEIPSMPxSLADHxDxFARHGLx
DKVxLIGIDYxxKTVNVYFxxLxAExxExExxxVxSMLRELGLPEPSDQM
LxLxxKAFxIYxTxSWDSPRIERLCFxVxTxxxxDPxxLPxxxVxIEPxI
EKFxxxVxxVPYxxxGxxRRFVxYAxxxSPExGEYYKLxSYYQxxPxxLD
xMxxxxxxxxxxxxxxxxxxxxxxxxxxxxxxxx.

In one example, said recombinant PTase is encoded by polynucleotide comprising or consisting of: a) a nucleotide sequence set for forth in SEQ ID NOs: 20 to 58; b) a nucleotide sequence having at least 70% identity to the nucleic acid of a), c) a nucleotide sequence that hybridizes with the complementary strand of the nucleic acid of a), d) a nucleotide sequence that differs from a) by one or more nucleotides that are substituted, deleted, and/or inserted; or e) a derivative of a), b), c), or d). For example, in c) said polynucleotide hybridizes with the complementary strand of the nucleic acid of a) under conditions of high stringency. Further, the polynucleotide may be a nucleotide sequence that differs from a) by one or more nucleotides that are substituted, deleted, and/or inserted.

In one example, in step (b) said polynucleotide has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity.

In one example, said polyketide is olivetol, olivetolic acid, divarin, divarinic acid, orcinol, or orsellinic acid.

The host cell can be a bacterial cell, a fungal cell, a protist cell, or a plant cell, such as any of the exemplary cell types noted herein in Table 2. Exemplary host cell types include S. cerevisiae, E. coli, Yarrowia lipolytica, and Komagataella phaffii.

In one aspect there is provided an isolated polypeptide comprising or consisting of an amino acid sequence set for in SEQ ID NOs: 59 to 97; or at least 50%, 60%, 70%, 80%, or 90% identity with the amino acid sequence set forth in SEQ ID NOs: 59 to 97, or has 100% identity with the amino acid sequence set forth in SEQ ID NOs: 59 to 97.

In one aspect there is provided an isolated polynucleotide molecule comprising: a) a nucleotide sequence set for forth in SEQ ID NOs: 20 to 58; b) a nucleotide sequence having at least 70% identity to the nucleotide sequence of a), c) a nucleotide sequence that hybridizes with the complementary strand of the nucleic acid of a), d) a nucleotide sequence that differs from a) by one or more nucleotides that are substituted, deleted, and/or inserted; or e) a derivative of a), b), c), or d). For example, in c) said polynucleotide may hybridize with the complementary strand of the nucleic acid of a) under conditions of high stringency. Further, an exemplary nucleic acid may be one that differs from a) by one or more nucleotides that are substituted, deleted, and/or inserted.

In one example, b) said polynucleotide has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity.

In one aspect there is provided an expression vector comprising the isolated polynucleotide molecule described above.

In one aspect there is provided a host cell comprising the polynucleotide as described, or the expression vector.

The host cell can be a bacterial cell, a fungal cell, a protist cell, or a plant cell, such as any of the exemplary cell types noted herein in Table 2. Exemplary host cell types include S. cerevisiae, E. coli, Yarrowia lipolytica, and Komagataella phaffii.

In one example, said host cell may comprise genetic modification that increase the available pool of terpenes and malonyl-coA in the cell.

In one example, said host cell may comprise genetic modification that increase the available pool of terpenes, malonyl-coA, and a phosphopantetheinyl transferase, in the cell.

In one example, said genetic modifications comprise or consist of tHMGr-IDI (SEQ ID NO: 105) and/or PGK1p:ACC^{1S659A,S1157A} (SEQ ID NO: 106).

In one example, said genetic modifications comprise of consist of tHMGr-IDI (SEQ ID NO: 105), PGK1p:ACC^{1S659A,S1157A} (SEQ ID NO: 106), and Erg20K197E (SEQ ID NO: 104).

In one example, said genetic modifications comprise or consist of PGK1p:ACC^{1S659A,S1157A} (SEQ ID NO: 108) and OAS2 (SEQ ID NO: 99).

In one example, said host cell further comprises NpgA from Aspergillus niger.

In one example, said host cell is a from S. cerevisiae. For example, said S. cerevisiae, comprises NpgA (SEQ ID NO: 101), PDH (SEQ ID NO: 102), Maf1 (SEQ ID NO: 103), Erg20K197E (SEQ ID NO: 104), tHMGr-IDI (SEQ ID NO: 105), PGK1p:ACC^{1S659A,S1157A}(SEQ ID NO: 106), OAS2 (SEQ ID NO: 99).

In one example, said polynucleotide encoding a PTase comprises or consists of PT161 (SEQ ID NO: 100). In one example, said polynucleotide encoding a PTase comprises or consists of: a) a nucleotide sequence as set forth in PT161 (SEQ ID NO: 100); b) a nucleic acid having at least 70% identity to the nucleic acid of a), c) a nucleic acid that hybridizes with the complementary strand of the nucleic acid of a), d) a nucleic acid that differs from a) by one or more nucleotides that are substituted, deleted, and/or inserted; or e) a derivative of a), b), c), or d). Said polynucleotide may be one having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to b), while maintaining PTase activity. In c) said polynucleotide may hybridizes with the complementary strand of the nucleic acid of a) under conditions of high stringency. The nucleic acid that differs from a) by one or more nucleotides that are substituted, deleted, and/or inserted.

In one aspect there is provided a method of producing orsellinic acid in a host cell, comprising: introducing a polynucleotide encoding OAS2 from Sparassis crispa into said host cell; and culturing the host cell under conditions sufficient for OAS2 polypeptide production.

In one aspect there is provided a method of producing orsellinic acid in a host cell, comprising: culturing a host cell which comprises or consists of a polynucleotide encoding OAS2 from Sparassis crispa under conditions sufficient for OAS2 polypeptide production.

The host cell can be a bacterial cell, a fungal cell, a protist cell, or a plant cell, such as any of the exemplary cell types noted herein in Table 2. Exemplary host cell types include S. cerevisiae, E. coli, Yarrowia lipolytica, and Komagataella phaffii.

In one example, said polynucleotide encoding OAS2 from Sparassis crispa comprises or consists of: a) a nucleotide sequence set for forth in SEQ ID NO: 99; b) a nucleotide sequence having at least 70% identity to the nucleic acid of a); c) a nucleotide sequence that hybridizes with the complementary strand of the nucleic acid of a); d) a nucleotide sequence that differs from a) by one or more nucleotides that are substituted, deleted, and/or inserted; or e) a derivative of a), b), c), or d). In b) said polynucleotide may have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. In c), said polynucleotide hybridizes with the complementary strand of the nucleic acid of a) under conditions of high stringency. For example, said polynucleotide may be a nucleotide sequence that differs from a) by one or more nucleotides that are substituted, deleted, and/or inserted.

In one aspect there is provided a kit comprising: an isolated polynucleotide molecule comprising: a) a nucleotide sequence set for forth in SEQ ID NOs: 20 to 58; b) a nucleotide sequence having at least 70% identity to the nucleotide sequence of a); c) a nucleotide sequence that hybridizes with the complementary strand of the nucleic acid of a); d) a nucleotide sequence that differs from a) by one or more nucleotides that are substituted, deleted, and/or inserted; or e) a derivative of a), b), c), or d); and optionally a container and/or instructions for the use thereof.

In one example, the kit may further comprise an expression vector comprising the isolated polynucleotide molecule described above.

In one example, the kit may further comprise a host cell comprising a polynucleotide described above, or the expression vector described above. Exemplary host cell types include S. cerevisiae, E. coli, Yarrowia lipolytica, and Komagataella phaffii.

Reference is made to Table 1, above, which provides a list of polyketides, prenyl donors and prenylated polyketides which may be used or produced herein.

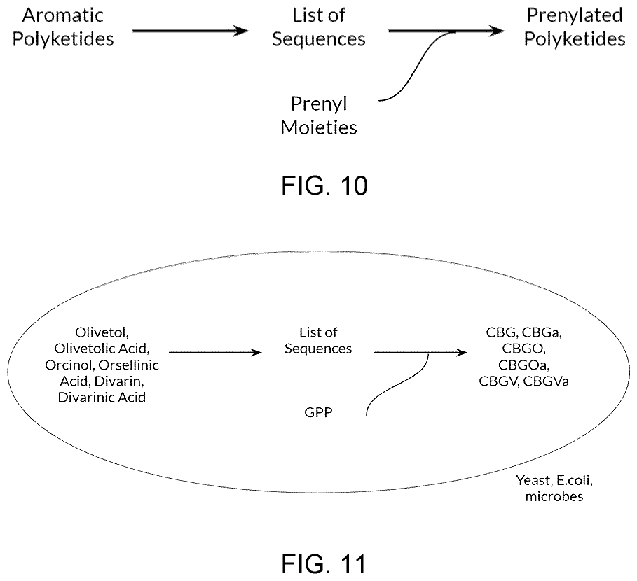

FIG. 10 depicts a generalize scheme for the use of the prenyltransferases described herein to attach a prenyl moiety to aromatic polyketides to produce prenylated polyketides.

FIG. 11 depicts a specific example in the production of cannabinoids.

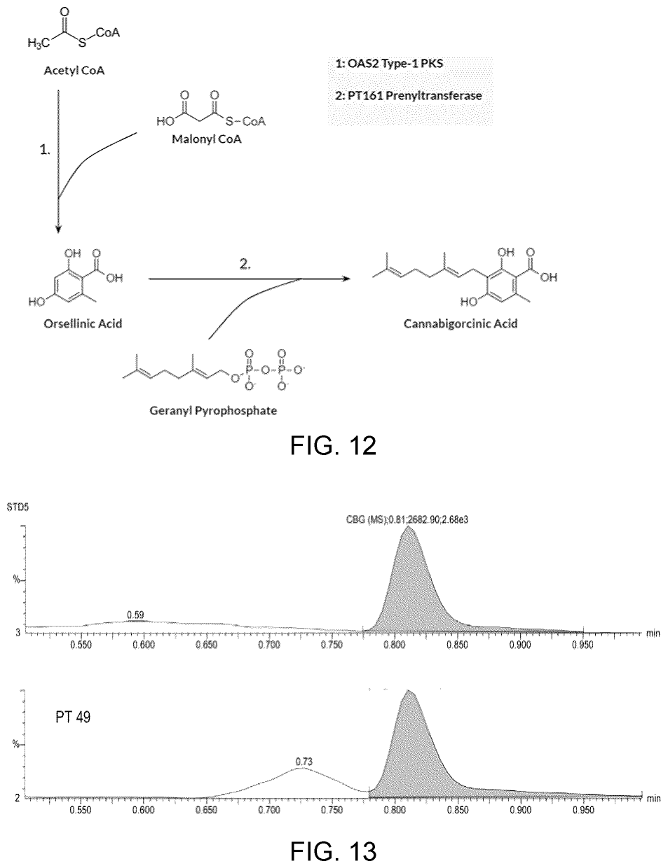

FIG. 12 depicts a pathway for production of Cannabigorcinic acid in S. cerevisiae.

As presented above, Table 2 lists additional specific examples of model organisms that may be used as host cells.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES—PART 2

### Example 2

Functional demonstration of Prenyltransferases for the production of prenylated polyketides. A cytosolic class of PTase that adopt an anti-parallel p/a barrel structure, known as the ABBA family PTs, may be more amenable to heterologous expression in recombinant hosts. The first reported example of this class of PTase was NphB (U.S. Pat. No. 7,361,483 B2, doi:10.1038/nature03668) which demonstrated catalytic activity for the prenylation of olivetol and olivetolic acid. Herein, we report the nucleotide and protein sequences for ABBA PTases that demonstrate activity with aromatic acceptor substrates.

Materials and Methods

Plasmid Construction: All plasmids were synthesized by Twist DNA sciences. SEQ ID NO. 20 to SEQ ID NO.58 were synthesized in the pET21D+ vector (SEQ ID NO.19) between base-pair 5209 and 5210.

Upon receiving the DNA from Twist DNA sciences, 100 ng of each vector was transformed into E. coli BL21 (DE3) gold chemically competent cells. The cells were plated on LB Agar plates with 75 mg/L Ampicillin as the selective agent. Successful, isolated colonies were picked by hand and inoculated into 1 ml of LB media containing 75 mg/L ampicillin in 96-well sterile deep well plates. The plates were grown for 16 hours at 37 C while being shaken at 250 RPM. After 16 hours 150 ul of each culture was transferred to a sterile microtiter plate containing 150 ul of 50% glycerol. The microtiter plates were sealed and stored at −80 C as a cell stock.

SOP for feeding assay: E. coli BL21(DE3) Gold harbouring a plasmid containing a coding sequence for the PTases stored as a cell stock were inoculated into 1 mL cultures of TB Overnight Express autoinduction media containing 75 mg/L ampicillin in sterile 96-well 2 mL deep well plates. The cultures were grown overnight at 30 degrees celsius with shaking at 950 rpm. The following day the cells were harvested by centrifugation and frozen at −20 degrees celsius. The thawed pellets were resuspended in 50 mM HEPES buffer (pH 7.5) with 10 mg/mL lysozyme, 2 U/mL benzonase, and 1× protease inhibitors. The suspension was incubated at 37 degrees celsius for 1 hour with shaking. Following lysis, the cell debris removed by centrifugation. The clarified lysate was collected and incubated with 5 mM polyketide (Olivetol, Olivetolic acid, divarinic acid, orcinol, orsellinic acid), 1.3 mM GPP in 50 mM HEPES buffer, 5 mM MgCL₂, pH 7.5, 0.4% Tween-80 to a final reaction volume of 50 uL. The reaction was incubated at 30 degrees for 24 hours.

After 24 hours 200 ul of Acetonitrile was added to the reaction and the mixture was centrifuged at 3750 RPM for 10 minutes. 150 ul of the supernatant was then transferred to another microtiter plate, sealed and stored for analysis.

Quantification and Analysis. The analysis was performed using a Waters UPLC chromatography system connected to a Waters TQD mass spectrometer. The separation was performed on an Acquity UPLC HSS C18 (30 mm×2.1 mm×1.8 um) using a reverse-phased method using Water+0.1% Formic Acid as solvent A and Methanol+0.1% Formic acid as solvent B at a flow rate of 0.8 ml/min. The gradient profile used to isolate CBG is as follows:

TABLE 9
Gradient

| | A | B |
|---|---|---|
| 0.00 min | 40% | 60% |
| 0.20 min | 40% | 60% |
| 0.55 min | 15% | 85% |
| 0.65 min | 15% | 85% |
| 0.66 min | 40% | 60% |
| 1.00 min | 40% | 60% |

The mass spectrometry is performed using an ESI source in positive mode with a cone voltage of 24V and a collision voltage of 21V for the fragmentation. The mass transitions used to characterize CBG was 317.2 to 192.9.

TABLE 10
LC-MS/MS Conditions

| CBGV-CBGO LC-MS/MS Method | | | | |
|---|---|---|---|---|
| | Acquity UPLC | | | |
| | HSS C18 (30 mm × | | | |
| Column | 2.1 mm × 1.8 um) | | | |
| LC Method | | | | |
| A1 | Water + 0.1% FA | | | |
| B1 | ACN + 0.1% FA | | | |
| Flow rate | 0.3 mL/min | | | |
| | A1 | B2 | | |
| 0.00 min | 50% | 50% | | |
| 0.80 min | 15% | 85% | | |
| 1.00 min | 5% | 95% | | |
| 1.01 min | 50% | 50% | | |
| 1.80 min | 50% | 50% | | |
| | RT (min) | | | |
| CBGO | 0.75 min | | | |
| CBGV | 0.91 min | | | |
| Ibuprofen | 0.64 min | | | |
| MS Method | | | | |
| | | | Cone | Collision |
| ES+ | M/Z | Transition | Voltage (V) | (V) |
| CBGO | 261.2 | → 161.1 | 20 | 12 |
| CBGV | 289.2 | → 164.9 | 20 | 12 |

TABLE 11
Method for CBGOa and CBGVa

| | Waters HSS | | | |
|---|---|---|---|---|
| Column | 1 × 50 mm, 1.8 um | | | |
| LC Method | | | | |
| A1 | Water + 0.1% FA | | | |
| B1 | ACN + 0.1% FA | | | |
| Flow rate | 0.3 mL/min | | | |
| | A1 | B2 | | |
| 0.00 min | 50% | 50% | | |
| 0.80 min | 15% | 85% | | |
| 1.00 min | 5% | 95% | | |
| 1.01 min | 50% | 50% | | |
| 1.80 min | 50% | 50% | | |
| | RT (min) | | | |
| CBGOa | 0.96 min | | | |
| CBGVa | 0.75 min | | | |
| MS Method | | | | |
| | | | Cone | Collision |
| ES+ | M/Z | Transition | Voltage (V) | (V) |
| CBGOa | 261.2 | → 161.1 | 20 | 12 |
| CBGVa | 303.2 | | 30 | |

Method for CBGa: LC conditions. Column: Hypersil Gold PFP 100×2.1 mm, 1.9 μm particle size. Column temperature: 45° C. Flow rate: 0.6 ml/min. Eluent A: Water 0.1% formic acid. Eluent B: Acetontrile 0.1% formic acid.

TABLE 12
Gradient

| Time (min) | % B | |
|---|---|---|
| Initial | 51 | Isocratic |
| 2.50 | 51 | |

ESI-MS conditions. Capillary: 3 kV. Source temperature: 150° C. Desolvation gas temperature: 450° C. Desolvation gas flow (nitrogen): 800 L/hr. Cone gas flow (nitrogen): 50 L/hr.

TABLE 13
Detection Parameters

| | CBGa |
|---|---|
| Retention time | 1.19 min |
| Ion | [M − H]⁻ |
| Mass (m/z) | 359.2 |
| Mode | ES−, SIR |
| Span | 0 |
| Dwell (s) | 0.2 |
| Cone (V) | 30 |

Sequences

Table 14 outlines the sequences used in this example.

TABLE 14
SEQUENCE ID NO TABLE

| SEQ ID NO: | Description | DNA/Protein | Sequence |
|---|---|---|---|
| SEQ ID NO: 19 | pET21d(+) | DNA | enclosed |
| | Empty Vector | | |
| SEQ ID NO: 20 | PT12 | DNA | enclosed |
| SEQ ID NO: 21 | PT 20 | DNA | enclosed |
| SEQ ID NO: 22 | PT 24 | DNA | enclosed |
| SEQ ID NO: 23 | PT 26 | DNA | enclosed |
| SEQ ID NO: 24 | PT 32 | DNA | enclosed |
| SEQ ID NO: 25 | PT 39 | DNA | enclosed |
| SEQ ID NO: 26 | PT 42 | DNA | enclosed |
| SEQ ID NO: 27 | PT 45 | DNA | enclosed |
| SEQ ID NO: 28 | PT 47 | DNA | enclosed |
| SEQ ID NO: 29 | PT 48 | DNA | enclosed |
| SEQ ID NO: 30 | PT 49 | DNA | enclosed |
| SEQ ID NO: 31 | PT 50 | DNA | enclosed |
| SEQ ID NO: 32 | PT 55 | DNA | enclosed |
| SEQ ID NO: 33 | PT 58 | DNA | enclosed |
| SEQ ID NO: 34 | PT 62 | DNA | enclosed |
| SEQ ID NO: 35 | PT 69 | DNA | enclosed |
| SEQ ID NO: 36 | PT 83 | DNA | enclosed |
| SEQ ID NO: 37 | PT 117 | DNA | enclosed |
| SEQ ID NO: 38 | PT 118 | DNA | enclosed |
| SEQ ID NO: 39 | PT 129 | DNA | enclosed |
| SEQ ID NO: 40 | PT 131 | DNA | enclosed |
| SEQ ID NO: 41 | PT 150 | DNA | enclosed |
| SEQ ID NO: 42 | PT 151 | DNA | enclosed |
| SEQ ID NO: 43 | PT 161 | DNA | enclosed |
| SEQ ID NO: 44 | PT 167 | DNA | enclosed |
| SEQ ID NO: 45 | PT 187 | DNA | enclosed |
| SEQ ID NO: 46 | PT 188 | DNA | enclosed |
| SEQ ID NO: 47 | PT 199 | DNA | enclosed |
| SEQ ID NO: 48 | PT 207 | DNA | enclosed |
| SEQ ID NO: 49 | PT 209 | DNA | enclosed |
| SEQ ID NO: 50 | PT 211 | DNA | enclosed |
| SEQ ID NO: 51 | PT 213 | DNA | enclosed |
| SEQ ID NO: 52 | PT 214 | DNA | enclosed |
| SEQ ID NO: 53 | PT 216 | DNA | enclosed |
| SEQ ID NO: 54 | PT 234 | DNA | enclosed |
| SEQ ID NO: 55 | PT 239 | DNA | enclosed |
| SEQ ID NO: 56 | PT 245 | DNA | enclosed |
| SEQ ID NO: 57 | PT 249 | DNA | enclosed |
| SEQ ID NO: 58 | PT 251 | DNA | enclosed |
| SEQ ID NO: 59 | PT12 | Protein | enclosed |
| SEQ ID NO: 60 | PT20 | Protein | enclosed |
| SEQ ID NO: 61 | PT24 | Protein | enclosed |
| SEQ ID NO: 62 | PT26 | Protein | enclosed |
| SEQ ID NO: 63 | PT32 | Protein | enclosed |
| SEQ ID NO: 64 | PT39 | Protein | enclosed |
| SEQ ID NO: 65 | PT42 | Protein | enclosed |
| SEQ ID NO: 66 | PT45 | Protein | enclosed |
| SEQ ID NO: 67 | PT47 | Protein | enclosed |
| SEQ ID NO: 68 | PT48 | Protein | enclosed |
| SEQ ID NO: 69 | PT49 | Protein | enclosed |
| SEQ ID NO: 70 | PT50 | Protein | enclosed |
| SEQ ID NO: 71 | PT55 | Protein | enclosed |
| SEQ ID NO: 72 | PT58 | Protein | enclosed |
| SEQ ID NO: 73 | PT62 | Protein | enclosed |
| SEQ ID NO: 74 | PT69 | Protein | enclosed |
| SEQ ID NO: 75 | PT83 | Protein | enclosed |
| SEQ ID NO: 76 | PT117 | Protein | enclosed |
| SEQ ID NO: 77 | PT118 | Protein | enclosed |
| SEQ ID NO: 78 | PT129 | Protein | enclosed |
| SEQ ID NO: 79 | PT131 | Protein | enclosed |
| SEQ ID NO: 80 | PT150 | Protein | enclosed |
| SEQ ID NO: 81 | PT151 | Protein | enclosed |
| SEQ ID NO: 82 | PT161 | Protein | enclosed |
| SEQ ID NO: 83 | PT167 | Protein | enclosed |
| SEQ ID NO: 84 | PT187 | Protein | enclosed |
| SEQ ID NO: 85 | PT188 | Protein | enclosed |
| SEQ ID NO: 86 | PT199 | Protein | enclosed |
| SEQ ID NO: 87 | PT207 | Protein | enclosed |
| SEQ ID NO: 88 | PT209 | Protein | enclosed |
| SEQ ID NO: 89 | PT211 | Protein | enclosed |
| SEQ ID NO: 90 | PT213 | Protein | enclosed |
| SEQ ID NO: 91 | PT214 | Protein | enclosed |
| SEQ ID NO: 92 | PT216 | Protein | enclosed |
| SEQ ID NO: 93 | PT234 | Protein | enclosed |
| SEQ ID NO: 94 | PT239 | Protein | enclosed |
| SEQ ID NO: 95 | PT245 | Protein | enclosed |
| SEQ ID NO: 96 | PT249 | Protein | enclosed |
| SEQ ID NO: 97 | PT251 | Protein | enclosed |

In one example, the consensus sequence for the PTs is that of SEQ ID NO:118, where X (or Xaa) residues represent “any amino acid”.

Table 15 lists the CBG peak areas from PTs.

TABLE 15
CBG peak areas from PTs

| PT# | CBG Peak Area | SD |
|---|---|---|
| PT49 | 6653 | 1786 |
| PT50 | 14865 | 1231 |
| PT48 | 1884 | 388 |
| PT151 | 1457 | 324 |
| PT211 | 628 | 361 |
| PT161 | 148 | 72 |
| PT129 | 1361 | 922 |

Table 16 lists CBGa production from PTs.

TABLE 16
CBGa production from PTs

| PT# | CBGa Peak Area | SD |
|---|---|---|
| PT42 | 42.7 | 3.1 |
| PT69 | 80.7 | 7.2 |
| PT12 | 41.3 | 5.3 |
| PT131 | 106.2 | 22.8 |
| PT117 | 67.9 | 15.4 |
| PT167 | 33.5 | 9.5 |
| PT118 | 132.3 | 8.8 |
| PT129 | 123.4 | 19.1 |
| PT188 | 78.8 | 12.5 |
| PT216 | 59.2 | 2.4 |
| PT211 | 432.4 | 52.1 |

Table 17 shows the CBGOa production from PTs.

TABLE 17
CBGOa production from PTs

| PT# | CBGOa Peak Area |
|---|---|
| PT46 | 2084.4 |
| PT24 | 2388.8 |
| PT83 | 2851.3 |
| PT26 | 2261.1 |
| PT79 | 2981.696 |
| PT82 | 3518.176 |
| PT80 | 3450.624 |
| PT167 | 3306.403 |
| PT161 | 3258.422 |

Table 18 lists the CBGVa production from PTs.

TABLE 18
CBGVa production from PTs

| PT# | CBGVa Peak Area |
|---|---|
| PT82 | 2261.838 |
| PT80 | 1149.23 |
| PT150 | 3145.72 |
| PT118 | 2004.75 |
| PT126 | 1807.25 |
| PT151 | 3412.72 |
| PT211 | 6881.75 |
| PT129 | 1741.61 |
| PT189 | 2381.57 |

Table 19 lists the CBGO production from PTs.

TABLE 19
CBGO production from PTs

| PT# | CBGO Peak Area |
|---|---|
| PT82 | 27200.37 |
| PT80 | 19279.32 |
| PT83 | 27251.37 |
| PT89 | 111341.5 |
| PT10 | 40805.17 |

### Example 3

In Vivo Production of Cannabigorcinic Acid (CBGOa)

This example describes the production of CBGOa in vivo in a Saccharomyces cerevisiae cannabinoid production strain using PT161. The strain contains genetic modifications allowing it to produce the polyketide precursor, Orsellinic acid (ORA) and the monoterpene precursor geranyl pyrophosphate (GPP). The strains in this experiment are listed in Table 20.

TABLE 20
Strains Used in Example 3

| Strain # | Background | Plasmids | Genotype | Notes |
|---|---|---|---|---|
| HB144 | -URA, -LEU | None | Saccharomyces cerevisiae | Base Strain |
| | | | CEN.PK2; ΔLEU2; ΔURA3; | |
| | | | Erg20K197E::KanMx; ALD6; | |
| | | | ASC1^{L641P}; NPGA; MAF1; | |
| | | | PGK1p:ACC1^{S659A, S1157A}; | |
| | | | tHMGR1; ID | |
| HB837 | -URA, -LEU | None | Saccharomyces cerevisiae | Orsellinic |
| | | | CEN.PK2; ΔLEU2; ΔURA3; | Acid |
| | | | Erg20K197E::KanMx; ALD6; | producing |
| | | | ASC1^{L641P}; NPGA; MAF1; | strain |
| | | | PGK1p:ACC1^{S659A, S1157A}; | |
| | | | tHMGR1; ID; OAS2:UserX-4 | |
| HB837 + | -URA, -LEU | PLAS246 | Saccharomyces cerevisiae | CBGOa |
| PLAS246 | | | CEN.PK2; ΔLEU2; ΔURA3; | producing |
| | | | Erg20K197E::KanMx; ALD6; | strain |
| | | | ASC1^{L641P}; NPGA; MAF1; | |
| | | | PGK1p:ACC1^{S659A, S1157A}; | |
| | | | tHMGR1; ID; OAS2:UserX-4 | |

A list and description of modifications to base strain is present in Table 21.

TABLE 21
Modifications to Base Strain

| | | | Integration | | |
|---|---|---|---|---|---|
| | Modification | SEQ ID | Region/ | | Genetic Structure of |
| # | name | NO. | Plasmid | Description | Sequence |
| 1 | NpgA | SEQ: | Flagfeldt Site | Phosphopantetheinyl Transferase | Site14Up::Tef1p: |
| | | 101 | 14 | from Aspergillus niger [10]. | NpgA:Prm9t: |
| | | | integration[9] | Accessory Protein for PKS's. | Site14Down |
| | | | | Necessary for OAS2 function. | |
| 2 | PDH | SEQ: | Flagfeldt Site | Acetaldehyde dehydrogenase | 19Up::Tdh3p:Ald6: |
| | | 102 | 19 integralion | (ALD6) from S. cerevisiae and | Adh1::Tef1p:seACS1 [symbol] : |
| | | | | acetoacetyl coA synthase | Prm9t::19Down |
| | | | | (AscL641P) from Salmonella | |
| | | | | enterica. Will allow greater | |
| | | | | accumulation of acetyl-coA in the | |
| | | | | cell. | |
| 3 | Maf1 | SEQ: | Flagfeldt Site | Maf1 is a regulator of tRNA | Site5Up::Tef1p: |
| | | 103 | 5 integration | biosynthesis. Overexpression in S. | Maf1:Prm9t: |
| | | | | cerevisiae has demonstrated | Site5Down |
| | | | | higher monolerpene (GPP) yields. | |
| 4 | Erg20K197E | SEQ: | Chromosomal | Mutant of Erg20 protein that | Tpi1t:ERG20K197E: |
| | | 104 | modification | diminishes FPP synthase activity | Cyc1t::Tef1p:KanMX: |
| | | | | creating greater pool of GPP | Tef1t |
| | | | | precursor. Negatively affects | |
| | | | | growth phenotype. | |
| 5 | tHMGr-IDI | SEQ: | USER Site X- | Overexpression of truncated | X3up::Tdh3p:tHMGR1: |
| | | 105 | 3 integration | HMGr1 and IDI1 proteins that have | Adh1t::Tef1p:IDI1: |
| | | | | been previously identified to be | Prm9t::X3down |
| | | | | bottlenecks in the S. cerevisiae | |
| | | | | terpenoid pathway responsible for | |
| | | | | GPP production. | |
| 6 | PGK1p: | SEQ: | Chromosomal | Mutations in the native S. | Pgk1:ACC1 [symbol] : |
| | ACC1 [symbol] | 106 | modification | cerevisiae acetyl-coA carboxylase | Acc1t |
| | | | | that removes post-translational | |
| | | | | modification based down- | |
| | | | | regulation. Leads to greater | |
| | | | | malonyl-coA pools. The promoter | |
| | | | | of Acc1 was also changed to a | |
| | | | | constitutive promoter for higher | |
| | | | | expression. | |

[symbol]

indicates data missing or illegible when filed

A list of plasmids is presented in Table 22.

TABLE 22
List of Plasmids

| # | Plasmid Name | Description | Selection |
|---|---|---|---|
| 1 | PLAS246 | pGAL_Gal1p:PT161:Cyc1t | Uracil |
| 2 | PLAS36 | pCAS_Hyg_Rnr2p:Cas9: | Hygromycin |
| | | Cyc1t::tRNATyr:HDV:gRNA:Snr52t | |

A list of Sequences is presented in Table 23.

TABLE 23
Sequences

| | | | Length of | Position of |
|---|---|---|---|---|
| SEQ ID NO: | Description | DNA/Protein | sequence | coding sequence |
| SEQ ID | Protein sequence for | Protein | 2098 | all |
| NO. 98 | OAS2 (Orsellinic acid | | | |
| | synthase) Type 1 PKS | | | |
| SEQ ID | Genomic integration of | DNA | 7717 | 728-7024 |
| NO. 99 | OAS2 into USER site X-4 | | | |
| SEQ ID | PLAS246, pGAL_URA | DNA | 6703 | 3019-3936 |
| NO. 100 | plasmid coding for gene | | | |
| | expressing PT161 | | | |
| SEQ ID | NpgA integrated in | DNA | 3564 | 1170-2201 |
| NO. 101 | Flagfelt Site 14 | | | |
| SEQ ID | PDH bypass integrated | DNA | 7114 | Ald6: 1444-2949 |
| NO. 102 | in Flagfelt Site 19 | | | ACS: 3888-5843 |
| SEQ ID | Maf1 integrated in | DNA | 3256 | 936-2123 |
| NO. 103 | Flagfelt Site 5 | | | |
| SEQ ID | Erg20K197E | DNA | 4254 | 2683-3423 |
| NO. 104 | | | | |
| SEQ ID | tHMGr-IDI integrated in | DNA | 4843 | tHMGR1: 877-2385 |
| NO. 105 | User Site X11-2 | | | IDI1: 3209-4075 |
| SEQ ID | PGK1p:ACC1^{S659A, S1157A} | DNA | 7673 | Pgk1p: 222-971 |
| NO. 106 | | | | Acc1mut: 972-7673 |
| SEQ ID | PLAS36 | DNA | 8980 | |
| NO. 107 | | | | |
| SEQ ID | PLAS414; PLAS250; | DNA or PRO | Various | |
| NO: | PT161; PT245; | | | |
| 108-117 | PLAS250; PLAS44; | | | |
| | PLAS400; PLAS411; | | | |
| | PLAS384; OAC | | | |

The orsellinic acid synthase from Sparassis crispa is a non-reducing iterative Type-1 PKS. This enzyme takes acetyl-coA, a native yeast metabolite, and iteratively adds 3 molecules of malonyl-coA to it which is then subsequently cyclizes to produce orsellinic acid. The orsellinic acid undergoes a prenylation catalyzed by PT161, in which one molecule of geranyl pyrophosphate (GPP) is condensed with one molecule of orsellinic acid, to produce cannabigorcinic acid (CBGOa). This is depicted in FIG. 12.

The S. cerevisiae strain used in this disclosure expresses a phosphopantetheinyl transferase, NpgA from Aspergillus niger. This enzyme is an accessory protein for the polyketide synthase OAS2 and is involved in the co-factor binding for OAS2.

The S. cerevisiae strain used in this disclosure contains a mutation in the ERG20 protein, ERG20K197E, that allows it to accumulate GPP inside the cell (Oswald et al., 2007), making it available for the prenylation reaction. This strain also overexpresses a truncated version of the HMGr1 protein and an ID11 protein, which are both native proteins that have been demonstrated to be bottlenecks in the S. cerevisiae terpenoid pathway (Ro et al., 2006), as a means to alleviate bottlenecks and increase the flux of carbon towards GPP accumulation in the cells. The base strain also overexpresses the MAF1 protein which is a negative regulator for tRNA biosynthesis in S. cerevisiae, as overexpression of this protein has been demonstrated to increase GPP accumulation in the cell (Liu et al., 2013).

The base strain also has multiple modifications that increase the available pool of acetyl-coA and malonyl-coA in the cell. The overexpression of the PDH bypass, which consists of the proteins ALD6 from S. cerevisiae and ACS1^{L641P} from Salmonella enterica, allows for a much greater pool of acetyl-coA in the cytosol of the yeast cell (Shiba et al., 2007). In addition, the native S. cerevisiae acetoacetyl coA carboxylase, ACC1, protein was also overexpressed by changing its promoter to a constitutive promoter. Two additional mutations, S659A and S1157A, were made in ACC1 in order to alleviate negative regulation by post-translational modification (Shi et al., 2014). This allows the yeast cell to have a much greater accumulation of malonyl-coA. The greater accumulation of acetyl-coA and malonyl-coA are necessary for orsellinic acid production in the cell.

Materials and Methods

Genetic Manipulations. HB144 was used as a base strain to develop all other strains in this experiment. All DNA was transformed into strains using the Gietz et al transformation protocol (Geitz, 2014). Plas 36 was used for the CRISPR-based genetic modifications described in this experiment (Ryan et al., 2016).

The genome at USER Site X-4 (Jensen et al., 2014) in HB144 was targeted using Cas9 and gRNA expressed from PLAS36 which was transformed into HB144. The donor for the recombination was SEQ ID NO. 99. Successful integrations were selected on YPD+200 ug/ml Hygromycin and confirmed by colony PCR. This led to the creation of HB837 with a Galactose inducible OAS2 encoding gene integrated into the genome of HB144. The genomic region containing SEQ ID NO. 99 was also verified by sequencing to confirm the presence of the OAS2 encoding gene. This allowed for the creation of an orsellinic acid producing strain, HB837. PLAS246 which encodes a galactose-inducible gene expressing PT161 was subsequently transformed into HB837 producing a strain that can synthesize cannabigorcinic acid directly from glucose.

Strain Growth and Media. HB837 was grown on Synthetic complete yeast minimal media with a composition of 1.7 g/L YNB without ammonium sulfate+1.96 g/L URA dropout amino acid supplements+76 mg/L uracil+1.5 g/L magnesium L-glutamate) with 2% w/v galactose, 2% w/v raffinose, 200 μg/l geneticin, and 200 ug/L ampicillin (Sigma-Aldrich Canada). HB837+PLAS246 was grown in the above described media lacking the Uracil component to select for the presence of PLAS246.

Experimental Conditions. Six single colony replicates of strains were tested in this study. All strains were grown in 1 ml cultures in 96-well deepwell plates. The deepwell plates were incubated at 30° C. and shaken at 250 rpm for 96 hrs.

Metabolite extraction was performed with 300 μl of Acetonitrile added to 100 μl culture in a new 96-well deepwell plate, followed by 30 min of agitation at 950 rpm. The solutions were then centrifuged at 3750 rpm for 5 min. 200 μl of the soluble layer was removed and stored in a 96-well v-bottom microtiter plate. Samples were stored at −20° C. until analysis.

Results

In the data for the in vivo production of orsellinic acid, samples were quantified using HPLC-MS analysis.

FIG. 13 depicts a chromatogram showing positive production of CBG.

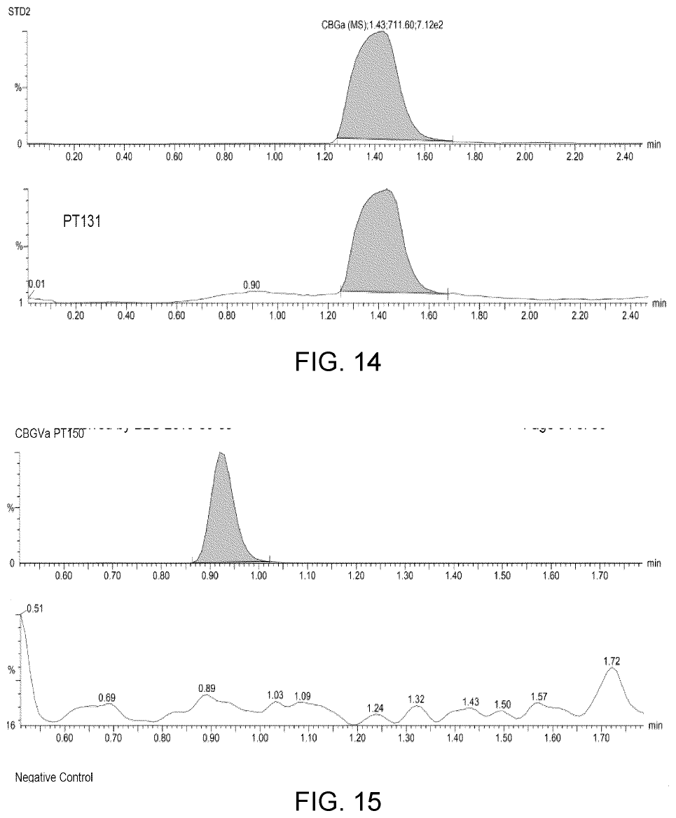

FIG. 14 depicts a chromatogram showing positive production of CBGa

FIG. 15 depicts a chromatogram showing positive production of CBGVa

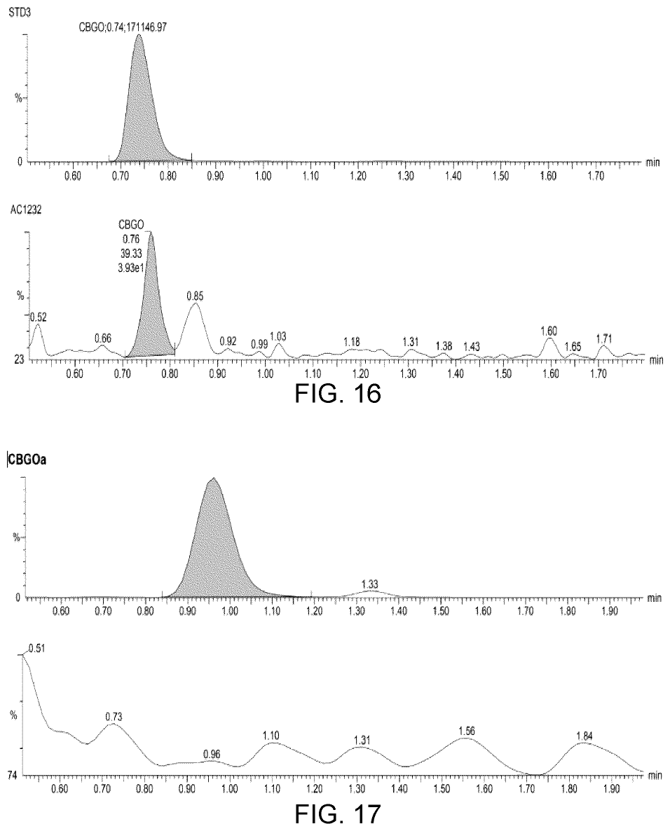

FIG. 16 depicts a chromatogram showing positive production of CBGO

FIG. 17 depicts a chromatogram showing positive production of CBGOa

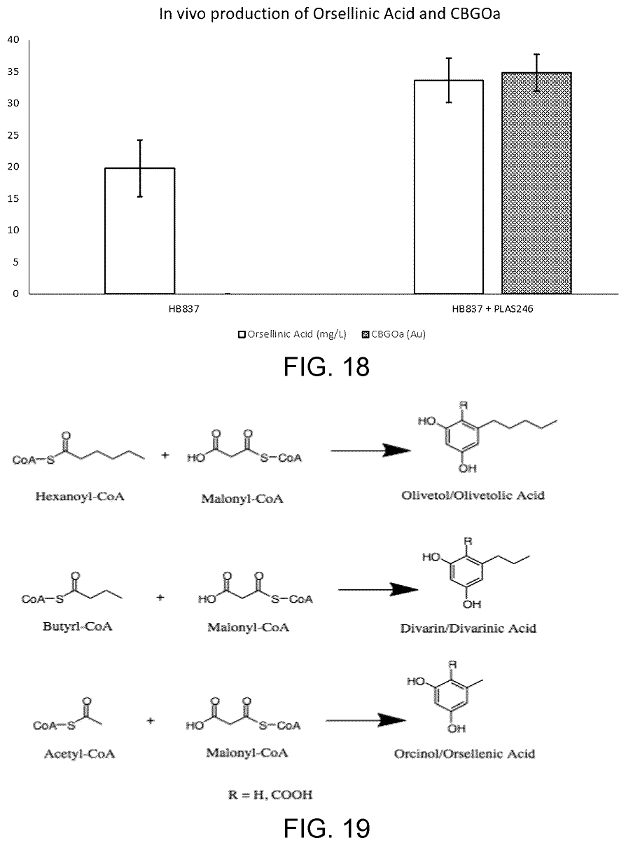

FIG. 18 illustrates increased in vivo orsellinic acid and CBGOa production, specifically: orsellinic acid (33.67±3.52 versus 19.73±4.46) and CBGOa (0.0±0.0 versus 34.86±2.91), for HB837+PLAS247, as compared with HB837 alone (mean±stdev).

part 3

Polyketide Synthase III and Acyl-CoA Synthases for Production of Aromatic Polyketides and Phytocannabinoids

This section relates generally to methods and cell lines for the production of aromatic polyketides, which can be used in phytocannabinoid synthesis utilizing a polyketide synthase III (interchangeably referenced herein as type 3 PKS or PKSIII). Examples include production of a variety of cannabinoids with PKSIII and acyl-CoA synthase enzymes in yeast, by providing different feeds. Such polyketides are useful intermediate/precursors in phytocannabinoid synthesis.

### Overview

There is provided herein a method of producing an aromatic polyketide and/or a phytocannabinoid in a host cell, comprising introducing a polynucleotide encoding a type 3 PKS protein and/or an acyl-CoA synthase protein into the host cell, and culturing the host cell under conditions sufficient for aromatic polyketide production.

Further, there is provided a method of producing a phytocannabinoid or phytocannabinoid derivative in a host cell, comprising introducing a polynucleotide encoding a type 3 PKS protein and/or an acyl-CoA synthase protein into the host cell, and culturing the cell under conditions sufficient for aromatic polyketide production, and for phytocannabinoid or phytocannabinoid derivative production therefrom.

Additionally, there is provided a method of producing an aromatic polyketide or phytocannabinoid, comprising: providing a host cell which produces from glucose, or is provided with, a fatty acid-CoA and an acetoacetyl-containing extender unit, introducing into the host cell a polynucleotide encoding a type 3 polyketide synthase (PKS) protein and/or an acyl-CoA synthase protein, and culturing the host cell under conditions sufficient for production of the aromatic polyketide, and/or the phytocannabionoid.

There is also provided a method of producing a phytocannabinoid or phytocannabinoid analogue, comprising: providing a host cell which produces from glucose, or is provided with, a fatty acid-CoA and an acetoacetyl-containing extender unit, and which prenylates aromatic polyketides with a prenyl donor, introducing into the host cell a polynucleotide encoding a type 3 polyketide synthase (PKS) protein, and culturing the host cell under conditions sufficient for production of the type 3 PKS protein for producing the aromatic polyketide for prenylation with the prenyl donor to form the phytocannabinoid or phytocannabinoid analogue.

Further, there is provided herein an expression vector comprising a nucleotide sequence encoding a type 3 PKS protein, wherein: the nucleotide sequence comprises at least 70% identity with a nucleotide sequence as set forth in any one of SEQ ID NO: 120 to 137, SEQ ID NO: 156 to 207, SEQ ID NO: 261 to 265, or a nucleotide encoding any one of SEQ ID NO:314 to 343 (PKS80 to PKS109); the type 3 PKS protein comprises at least 70% identity with any one of SEQ ID NO: -138 to 155, SEQ ID NO: 208 to 259, SEQ ID NO: 266 to 270, or SEQ ID NO:314 to 343 (PKS80 to PKS109); or the type 3 PKS protein comprises or consists of the consensus sequence as set forth in SEQ ID NO: 260. The acyl-CoA synthase protein may comprise or consist of a protein as set forth in any one of SEQ ID NO: 284 to 313 (Alk1 to Alk30), or a protein with at least 70% identity with any one of SEQ ID NO: 284 to 313 (Alk1 to Alk30). Host cells transformed with the expression vector are also provided herein.

PKSIII (or type 3 PKS) activity in yeast as well as production of novel polyketides and cannabinoids is described herein. Further, production of tetrahydrocannabivarin acid (THCVa) can be achieved by providing butyric acid to a described polyketide synthase. Further, improvements in THCVa titres by expressing a set of novel PKSIII and acyl-CoA enzymes in yeast are described. It is established in these Examples that the expression of many of these enzymes greatly improves phytocannabinoid titres.

In one exemplary embodiment, a method is described in which a host cell comprises a polynucleotide encoding at least one type 3 PKS protein selected from the group consisting of PKS80-PKS109, at least one acyl-CoA synthase protein selected from the group consisting of Alk-Alk30, and optionally a polynucleotide encoding CSAAE1, PC20, PKS73, PT254, and/or OXC155.

### Detailed Description of Part 3

Generally, there is described herein the production of polyketides in recombinant organisms, which are within the synthetic pathway to formation of phytocannabinoids or phytocannabinoid analogues.

Phytocannabinoids are a large class of compounds with over 100 different known structures that are produced in the Cannabis sativa plant. These bio-active molecules, such as tetrahydrocannabinol (THC) and cannabidiol (CBD), can be extracted from plant material for medical and recreational purposes. However, the synthesis of plant material is costly, not readily scalable to large volumes, and requires lengthy growing periods to produce sufficient quantities of phytocannabinoids.

Early stages of the cannabinoid synthetic pathway proceed via the generation of olivetolic acid by the type III PKS olivetolic acid synthase (OAS) and cyclase olivetolic acid cyclase (OAC) (Taura et al., 2009). This reaction uses a hexanoyl-CoA starter as well as three units of malonyl-CoA. Olivetolic acid is the backbone of most classical cannabinoids and can be prenylated to form CBGA, which is ultimately converted to CBDA or THCA by an oxidocyclase. Production of olivetolic acid in S. cerevisiae is challenging as OAS generates significant by-products such as HTAL, PDAL and olivetol (Gagne et al., 2012).

Phytocannabinoids may be synthesized from polyketides such as olivetolic acid by prenylation of the polyketide, ie—the formation of a C—C bond between the polyketide and an allylic isoprene, such as diphosphate geranyl pyrophosphate (GPP). Prenylation of olivetolic acid by GPP produces the cannabinoid cannabigerolic acid (CBGa). This reaction type is catalyzed by enzymes known as prenyltransferases. The Cannabis plant utilizes a membrane-bound prenyltransferase to catalyze the addition of the prenyl moiety to olivetolic acid to form CBGa.

In one aspect, there is a method described of producing polyketides in a recombinant organism, which polyketide may be used by the organism in a pathway to synthesis of a phytocannabinoid or phytocannabinoid analogue.

A method is described herein for producing a phytocannabinoid or an aromatic polyketide in a host cell, comprising introducing a polynucleotide encoding a type 3 PKS protein and/or an acyl-CoA synthase protein into the host cell, and culturing the cell under conditions sufficient for aromatic polyketide production, and optionally under conditions sufficient for phytocannabinoid production therefrom.

The host cell may produce the aromatic polyketide from a fatty acid-CoA and an acetoacetyl-containing extender unit, which may be either synthesized by the cell, for example via metabolism of a sugar such as glucose. Alternatively, these compounds may be provided to the host cell.

A further method of producing an aromatic polyketide is described herein, comprising: providing a host cell which produces from glucose, or is provided with, a fatty acid-CoA and an acetoacetyl-containing extender unit; introducing into the host cell a polynucleotide encoding a type 3 polyketide synthase (PKS) protein; and culturing the host cell under conditions sufficient for production of the aromatic polyketide protein for producing the aromatic polyketide from the fatty acid-CoA and the extender unit.

Further, the host cell may produce the aromatic polyketide using the acyl-CoA synthase.

Additionally, a method of producing a phytocannabinoid or phytocannabinoid analogue is described herein. The method comprises providing a host cell which produces from glucose, or is provided with, a fatty acid-CoA and an acetoacetyl-containing extender unit, and which prenylates aromatic polyketides with a prenyl donor; introducing into the host cell a polynucleotide encoding a type 3 polyketide synthase (PKS) protein; and culturing the host cell under conditions sufficient for production of the type 3 PKS protein for producing the aromatic polyketide for prenylation with the prenyl donor to form the phytocannabinoid or phytocannabinoid analogue.

Introducing the polynucleotide into the host cell may comprise transformation of the host cell using any acceptable transformation methodology.

The type 3 PKS protein is one that is not native to C. sativa. For example, the type 3 PKS protein may comprise or consist of: (a) a protein as set forth in any one of SEQ ID NO: -138-155, SEQ ID NO: -208-259, SEQ ID NO: 266-270, or SEQ ID NO:314-343 (PKS80 to PKS109); (b) a protein with at least 70% identity with any one of SEQ ID NO: 138-155, SEQ ID NO: -208-259, SEQ ID NO: 266-270, or SEQ ID NO:314-343 (PKS80 to PKS109); (c) a protein that differs from (a) by one or more residues that are substituted, deleted and/or inserted; or (d) a derivative of (a), (b), or (c).

The acyl-CoA synthase protein may comprise or consists of (a) a protein as set forth in any one of SEQ ID NO: 284-313 (Alk1 to Alk30); (b) a protein with at least 70% identity with any one of SEQ ID NO: 284-313 (Alk1 to Alk30); (c) a protein that differs from (a) by one or more residues that are substituted, deleted and/or inserted; or (d) a derivative of (a), (b), or (c).

The nucleotide sequence encoding the type 3 PKS protein is also one that is not native to C. sativa. For example, it may be a sequence that comprises or consisting of: (a) a nucleotide sequence as set forth in any one of SEQ ID NO: -120-137, SEQ ID NO: 156-207, SEQ ID NO: 261-265, or a nucleotide encoding any one of SEQ ID NO:314-343 (PKS80 to PKS109); (b) a nucleotide sequence having at least 70% identity with the nucleotide sequence of (a); (c) a nucleotide that hybridizes with the complementary strand of the nucleotide sequence of (a); (d) a nucleotide sequence that differs from (a) by one or more nucleotides that are substituted, deleted, and/or inserted; or (e) a derivative of (a), (b), (c), or (d). In the event a complementary strand is used, the nucleotide may be one that hybridizes with the complementary strand of the nucleotide sequence of (a) under conditions of high stringency.

The protein may have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any one of SEQ ID NO: -138-155, SEQ ID NO: -208-259, SEQ ID NO: 266-270, or SEQ ID NO:314-343 (PKS80 to PKS109). The type 3 PKS protein may comprises or consists of the consensus sequence as set forth in SEQ ID NO: 260, reflecting consensus based on sequences SEQ ID NO: -138-155, SEQ ID NO: -208-259, and SEQ ID NO: -266-270.

The nucleotide sequence may be at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleotide as set forth in any one of SEQ ID NO: -120-137, SEQ ID NO: -156-207, or SEQ ID NO: -261-265.

The nucleotide sequence encoding the acyl-CoA synthases protein may comprise or consisting of: (a) a nucleotide sequence encoding a protein as set forth in any one of SEQ ID NO: 284-313 (Alk1 to 30); (b) a nucleotide sequence having at least 70% identity with the nucleotide sequence of (a); (c) a nucleotide that hybridizes with the complementary strand of the nucleotide sequence of (a); (d) a nucleotide sequence that differs from (a) by one or more nucleotides that are substituted, deleted, and/or inserted; or (e) a derivative of (a), (b), (c), or (d).

The acetoacetyl-containing extender unit used in the method may comprise malonyl-CoA.

The host cell may comprise one or more genetic modifications that increase the available malonyl-CoA in the cell.

The aromatic polyketide may be any of those described herein as formula 3-I to 3-VI. For example, the aromatic polyketide may be olivetol, olivetolic acid, divarin, divarinic acid, orcinol, or orsellinic acid.

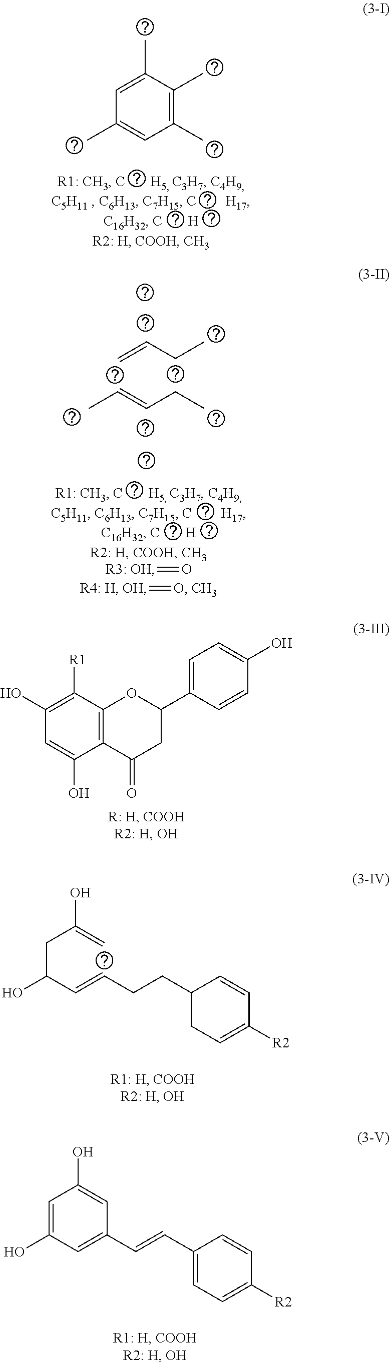

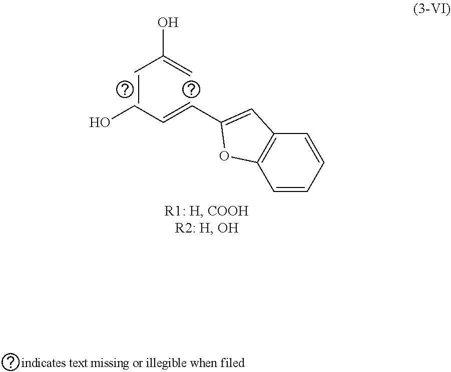

In the methods wherein the host cell produces a phytocannabinoid or phytocannabinoid analogue, this may be done by prenylation of the aromatic polyketide with a prenyl donor. The prenyl donor may be described as shown in formula 3-VII.

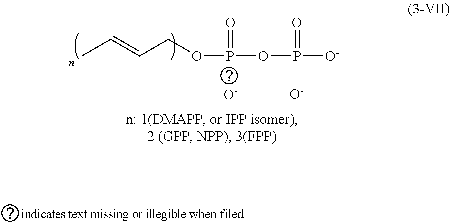

The phytocannabinoid or phytocannabinoid analogue formed may be any of formula 3-VIII to 3-XII.

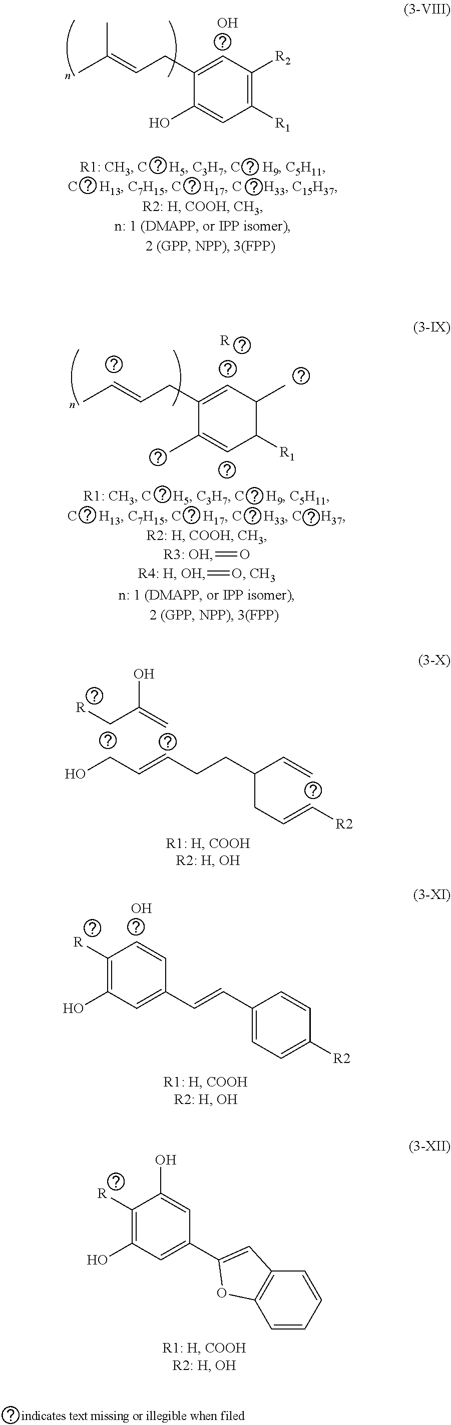

The phytocannabinoid so formed may be cannabigerol (CBG), cannabigerolic acid (CBGa), cannabigerovarin (CBGv), cannabigerovarinic acid (CBGva), cannabigerocin (CBGO), or cannabigerocinic acid (CBGOa). For example, when the aromatic polyketide is olivetol the phytocannabinoid is cannabigerol (CBG), when the aromatic polyketide is olivetolic acid the phytocannabinoid is cannabigerolic acid (CBGa), when said aromatic polyketide is divarin the phytocannabinoid is cannabigerovarin (CBGv), when the aromatic polyketide is divarinic acid the phytocannabinoid is cannabigerovarinic acid (CBGva), when the polyketide is orcinol the phytocannabinoid is cannabigerocin (CBGO), or when the aromatic polyketide is orsellinic acid the phytocannabinoid is cannabigerocinic acid (CBGOa).

The host cell may be a bacterial cell, a fungal cell, a protist cell, or a plant cell, and may for example, be any one of the cell types described hereinbelow. For example, the host cell is S. cerevisiae, E. coli, Yarrowia lipolytica, or Komagataella phaffii.

An expression vector is described herein comprising a nucleotide sequence encoding a type 3 PKS protein, wherein: the nucleotide sequence comprises at least 70% identity with a nucleotide sequence as set forth in any one of SEQ ID NO: -120-137, SEQ ID NO: 156-207, or SEQ ID NO: -261-265; the type 3 PKS protein comprises at least 70% identity with any one of SEQ ID NO: -138-155, SEQ ID NO: 208-259, SEQ ID NO: 266-270, or SEQ ID NO:314-343 (PKS80 to PKS109); or the type 3 PKS protein comprises or consists of the consensus sequence as set forth in SEQ ID NO: 260, as based on the consensus of sequences SEQ ID NO: -138-155, SEQ ID NO: -208-259, and SEQ ID NO: 266-270. It is understood that the expression “at least 70% identity” encompasses identities of 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% with the specified sequence. The expression vector may comprise or consist of a nucleic acid sequence encoding the type 3 PKS protein according to SEQ ID NO: 260. A host cell transformed with this expression vector is also described, wherein the host cell is a bacterial cell, a fungal cell, a protist cell, or a plant cell, for example of any of the types described herein below, with exemplary (but non-limiting) cell types being: S. cerevisiae, E. coli, Yarrowia lipolytica, or Komagataella phaffii.

In some example of the method herein, the phytocannabinoid produced is cannabigerol (CBG), cannabigerolic acid (CBGa), cannabigerovarin (CBGv), cannabigerovarinic acid (CBGva), cannabigerocin (CBGo), or cannabigerocinic acid (CBGoa).

In some example of the method herein, the polyketide is olivetol, olivetolic acid, divarin, divarinic acid, orcinol, or orsellinic acid.

In some examples of the downstream use of the polyketides produced in recombinant organisms as described herein, the polyketide may go on to phytocannabinoid synthesis. For example, the polyketide is olivetol then the phytocannabinoid is cannabigerol (CBG), when the polyketide is olivetolic acid then the phytocannabinoid is cannabigerolic acid (CBGa), when the polyketide is divarin then the phytocannabinoid is cannabigerovarin (CBGv), when the polyketide is divarinic acid then the phytocannabinoid is cannabigerovarinic acid (CBGva), when the polyketide is orcinol then the phytocannabinoid is cannabigerocin (CBGo), and when the polyketide is orsellinic acid then the phytocannabinoid produced is cannabigerocinic acid (CBGoa).

In the method described herein, the host cell may comprise a polynucleotide encoding at least one type 3 PKS protein selected from the group consisting of PKS80-PKS109, at least one acyl-CoA synthase protein selected from the group consisting of Alk1-Alk30, and optionally a polynucleotide encoding CSAAE1, PC20, PKS73, PT254, and/or OXC155.

In one example, the host cell is fed butyric acid and produces THCVa.

An expression vector is described comprising a nucleotide sequence encoding a type 3 PKS protein and/or an acyl-CoA synthase protein, wherein the type 3 PKS encoding nucleotide sequence comprises at least 70% identity with a nucleotide sequence as set forth in any one of SEQ ID NO: -120-137, SEQ ID NO: 156-207, SEQ ID NO: 261-265, or a nucleotide encoding any one of SEQ ID NO:314-343 (PKS80 to PKS109); the type 3 PKS protein comprises at least 70% identity with any one of SEQ ID NO: 138-155, SEQ ID NO: 208-259, SEQ ID NO: 266-270, or SEQ ID NO:314-343 (PKS80 to PKS109); or the type 3 PKS protein comprises or consists of the consensus sequence as set forth in SEQ ID NO: 260; and/or the acyl-CoA synthase protein encoding nucleotide sequence comprises at least 70% identity with a nucleotide sequence encoding a protein as set forth in any one of SEQ ID NO: 284-313 (Alk1-Alk30); or the an acyl-CoA synthase protein comprises at least 70% identity with any one of SEQ ID NO: 284-313 (Alk1-Alk30).

The protein(s) encoded by the expression vector may have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with any one of SEQ ID NO: -138-155, SEQ ID NO: 208-259, SEQ ID NO: 266-270, or SEQ ID NO:314-343 (PKS80 to PKS109).

Further, the expression vector may comprise the nucleotide sequence which has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with any one of SEQ ID NO: -120-137, SEQ ID NO: 156-207, or SEQ ID NO: -261-265.

A host cell transformed with the expression vector above is described herein, which may be a bacterial cell, a fungal cell, a protist cell, or a plant cell. Table 2 described a variety of host cell types within these categories. Exemplary host cells include S. cerevisiae, E. coli, Yarrowia lipolytica, or Komagataella phaffii.

Reference is made to Table 1, above, which provides a list of polyketides, prenyl donors and prenylated polyketides which may be used or produced in the methods described.

These polyketides, together with prenyl donors and resulting prenylated polyketides are listed so as to illustrate the phytocannabinoids that may be synthesized as a result. The following terms are used: DMAPP for dimethylallyl diphosphate; GPP for geranyl diphosphate; FPP for farnesyl diphosphate; NPP for neryl diphosphate; and IPP for isopentenyl diphosphate.

As provided above in Table 2 there are numerous specific examples of host cell organisms possible for use in one or more of the methods described herein.

Table 24 lists possible CoA donors (or “primers”) for use in the polyketide synthase reaction of type 3 PKS, together with extender units containing acetoacetyl moieties (such as malonyl-CoA) to thereby form a polyketide intermediate in host cell formation of phytocannabinoids.

TABLE 24
CoA donors for Type 3 PKS reaction

| Structure | R-Group, if any |
|---|---|
| 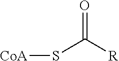 | R = CH₃, C₂H₅, C₃H₇, C₄H₉, C₅H₁₁, C₆H₁₃, C₇H₁₅, C₈H₁₇, C₁₆H₃₃, C₁₈H₃₇, |
| 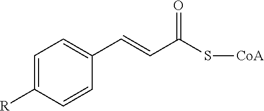 | R = H, OH |
| 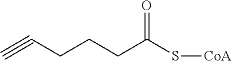 | |
| 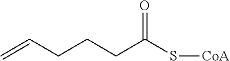 | |
| 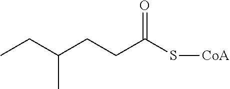 | |
| 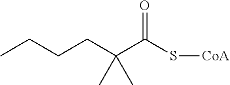 | |
| 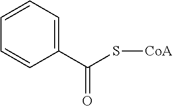 | |
| 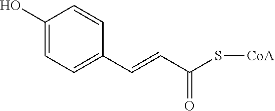 | |
| 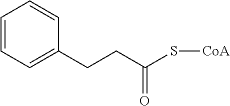 | |

Table 25 lists the sequences described herein, for greater certainty. Actual sequences are provided in later tables, below. The Type 3 PKS protein is one that is not native to C. sativa.

TABLE 25
List of sequence characteristics

| SEQ ID NO: | Description | DNA/Protein |
|---|---|---|
| SEQ ID NO: 119 | pET21d(+) | DNA |
| | Empty Vector | |
| SEQ ID NO. 120 | PKS8 | DNA |
| SEQ ID NO. 121 | PKS10 | DNA |
| SEQ ID NO. 122 | PKS17 | DNA |
| SEQ ID NO. 123 | PKS20 | DNA |
| SEQ ID NO. 124 | PKS22 | DNA |
| SEQ ID NO. 125 | PKS25 | DNA |
| SEQ ID NO. 126 | PKS26 | DNA |
| SEQ ID NO. 127 | PKS27 | DNA |
| SEQ ID NO. 128 | PKS31 | DNA |
| SEQ ID NO. 129 | PKS33 | DNA |
| SEQ ID NO. 130 | PKS47 | DNA |
| SEQ ID NO. 131 | PKS48 | DNA |
| SEQ ID NO. 132 | PKS49 | DNA |
| SEQ ID NO. 133 | PKS54 | DNA |
| SEQ ID NO. 134 | PKS56 | DNA |
| SEQ ID NO. 135 | PKS57 | DNA |
| SEQ ID NO. 136 | PKS58 | DNA |
| SEQ ID NO. 137 | PKS61 | DNA |
| SEQ ID NO. 138 | PKS8 | Protein |
| SEQ ID NO. 139 | PKS10 | Protein |
| SEQ ID NO. 140 | PKS17 | Protein |
| SEQ ID NO. 141 | PKS20 | Protein |
| SEQ ID NO. 142 | PKS22 | Protein |
| SEQ ID NO. 143 | PKS25 | Protein |
| SEQ ID NO. 144 | PKS26 | Protein |
| SEQ ID NO. 145 | PKS27 | Protein |
| SEQ ID NO. 146 | PKS31 | Protein |
| SEQ ID NO. 147 | PKS33 | Protein |
| SEQ ID NO. 148 | PKS47 | Protein |
| SEQ ID NO. 149 | PKS48 | Protein |
| SEQ ID NO. 150 | PKS49 | Protein |
| SEQ ID NO. 151 | PKS54 | Protein |
| SEQ ID NO. 152 | PKS56 | Protein |
| SEQ ID NO. 153 | PKS57 | Protein |
| SEQ ID NO. 154 | PKS58 | Protein |
| SEQ ID NO. 155 | PKS61 | Protein |
| SEQ ID NO. 156 | PKS02 | DNA |
| SEQ ID NO. 157 | PKS03 | DNA |
| SEQ ID NO. 158 | PKS04 | DNA |
| SEQ ID NO. 159 | PKS05 | DNA |
| SEQ ID NO. 160 | PKS06 | DNA |
| SEQ ID NO. 161 | PKS07 | DNA |
| SEQ ID NO. 162 | PKS09 | DNA |
| SEQ ID NO. 163 | PKS11 | DNA |
| SEQ ID NO. 164 | PKS12 | DNA |
| SEQ ID NO. 165 | PKS13 | DNA |
| SEQ ID NO. 166 | PKS14 | DNA |
| SEQ ID NO. 167 | PKS15 | DNA |
| SEQ ID NO. 168 | PKS16 | DNA |
| SEQ ID NO. 169 | PKS18 | DNA |
| SEQ ID NO. 170 | PKS19 | DNA |
| SEQ ID NO. 171 | PKS21 | DNA |
| SEQ ID NO. 172 | PKS23 | DNA |
| SEQ ID NO. 173 | PKS24 | DNA |
| SEQ ID NO. 174 | PKS28 | DNA |
| SEQ ID NO. 175 | PKS29 | DNA |
| SEQ ID NO. 176 | PKS30 | DNA |
| SEQ ID NO. 177 | PKS32 | DNA |
| SEQ ID NO. 178 | PKS34 | DNA |
| SEQ ID NO. 179 | PKS35 | DNA |
| SEQ ID NO. 180 | PKS36 | DNA |
| SEQ ID NO. 181 | PKS37 | DNA |
| SEQ ID NO. 182 | PKS38 | DNA |
| SEQ ID NO. 183 | PKS39 | DNA |
| SEQ ID NO. 184 | PKS40 | DNA |
| SEQ ID NO. 185 | PKS41 | DNA |
| SEQ ID NO. 186 | PKS42 | DNA |
| SEQ ID NO. 187 | PKS43 | DNA |
| SEQ ID NO. 188 | PKS44 | DNA |
| SEQ ID NO. 189 | PKS45 | DNA |
| SEQ ID NO. 190 | PKS46 | DNA |
| SEQ ID NO. 191 | PKS50 | DNA |
| SEQ ID NO. 192 | PKS51 | DNA |
| SEQ ID NO. 193 | PKS52 | DNA |
| SEQ ID NO. 194 | PKS53 | DNA |
| SEQ ID NO. 195 | PKS55 | DNA |
| SEQ ID NO. 196 | PKS59 | DNA |
| SEQ ID NO. 197 | PKS60 | DNA |
| SEQ ID NO. 198 | PKS62 | DNA |
| SEQ ID NO. 199 | PKS63 | DNA |
| SEQ ID NO. 200 | PKS64 | DNA |
| SEQ ID NO. 201 | PKS65 | DNA |
| SEQ ID NO. 202 | PKS66 | DNA |
| SEQ ID NO. 203 | PKS67 | DNA |
| SEQ ID NO. 204 | PKS68 | DNA |
| SEQ ID NO. 205 | PKS69 | DNA |
| SEQ ID NO. 206 | PKS70 | DNA |
| SEQ ID NO. 207 | PKS71 | DNA |
| SEQ ID NO. 208 | PKS02 | Protein |
| SEQ ID NO. 209 | PKS03 | Protein |
| SEQ ID NO. 210 | PKS04 | Protein |
| SEQ ID NO. 211 | PKS05 | Protein |
| SEQ ID NO. 212 | PKS06 | Protein |
| SEQ ID NO. 213 | PKS07 | Protein |
| SEQ ID NO. 214 | PKS09 | Protein |
| SEQ ID NO. 215 | PKS11 | Protein |
| SEQ ID NO. 216 | PKS12 | Protein |
| SEQ ID NO. 217 | PKS13 | Protein |
| SEQ ID NO. 218 | PKS14 | Protein |
| SEQ ID NO. 219 | PKS15 | Protein |
| SEQ ID NO. 220 | PKS16 | Protein |
| SEQ ID NO. 221 | PKS18 | Protein |
| SEQ ID NO. 222 | PKS19 | Protein |
| SEQ ID NO. 223 | PKS21 | Protein |
| SEQ ID NO. 224 | PKS23 | Protein |
| SEQ ID NO. 225 | PKS24 | Protein |
| SEQ ID NO. 226 | PKS28 | Protein |
| SEQ ID NO. 227 | PKS29 | Protein |
| SEQ ID NO. 228 | PKS30 | Protein |
| SEQ ID NO. 229 | PKS32 | Protein |
| SEQ ID NO. 230 | PKS34 | Protein |
| SEQ ID NO. 231 | PKS35 | Protein |
| SEQ ID NO. 232 | PKS36 | Protein |
| SEQ ID NO. 233 | PKS37 | Protein |
| SEQ ID NO. 234 | PKS38 | Protein |
| SEQ ID NO. 235 | PKS39 | Protein |
| SEQ ID NO. 236 | PKS40 | Protein |
| SEQ ID NO. 237 | PKS41 | Protein |
| SEQ ID NO. 238 | PKS42 | Protein |
| SEQ ID NO. 239 | PKS43 | Protein |
| SEQ ID NO. 240 | PKS44 | Protein |
| SEQ ID NO. 241 | PKS45 | Protein |
| SEQ ID NO. 242 | PKS46 | Protein |
| SEQ ID NO. 243 | PKS50 | Protein |
| SEQ ID NO. 244 | PKS51 | Protein |
| SEQ ID NO. 245 | PKS52 | Protein |
| SEQ ID NO. 246 | PKS53 | Protein |
| SEQ ID NO. 247 | PKS55 | Protein |
| SEQ ID NO. 248 | PKS59 | Protein |
| SEQ ID NO. 249 | PKS60 | Protein |
| SEQ ID NO. 250 | PKS62 | Protein |
| SEQ ID NO. 251 | PKS63 | Protein |
| SEQ ID NO. 252 | PKS64 | Protein |
| SEQ ID NO. 253 | PKS65 | Protein |
| SEQ ID NO. 254 | PKS66 | Protein |
| SEQ ID NO. 255 | PKS67 | Protein |
| SEQ ID NO. 256 | PKS68 | Protein |
| SEQ ID NO. 257 | PKS69 | Protein |
| SEQ ID NO. 258 | PKS70 | Protein |
| SEQ ID NO. 259 | PKS71 | Protein |
| SEQ ID NO. 260 | Consensus | Protein |
| SEQ ID NO. 261 | PKS72 | DNA |
| SEQ ID NO. 262 | PKS73 | DNA |
| SEQ ID NO. 263 | PKS74 | DNA |
| SEQ ID NO. 264 | PKS75 | DNA |
| SEQ ID NO. 265 | PKS76 | DNA |
| SEQ ID NO. 266 | PKS72 | Protein |
| SEQ ID NO. 267 | PKS73 | Protein |
| SEQ ID NO. 268 | PKS74 | Protein |
| SEQ ID NO. 269 | PKS75 | Protein |
| SEQ ID NO. 270 | PKS76 | Protein |

In one embodiment, a consensus sequence for Type 3 PKS, based on sequences SEQ ID NO: -138 to 155, SEQ ID NO: -208 to 259, and SEQ ID NO: -266 to 270 is:

(SEQ ID NO: 260)
xxxxxxxxxxxxxxxxxxxxxxxxxxxxxxxxQrAExGxxxxATILAIGT
AxPxNxIxQSDYxDYYFRITxxSExxTELKEKFKRxICDKSxIKKRYxxx
xxMxLxxExxxxxxxxxxxxxxxxxxxxxxxxxxxxxExLKENPNMxxYx
xxxxxxxxxxxxxxxPSLDxRxDIxVxEVPKLxKEAAxKAIKExxWGQxx
xSxxKITHLVFxTxTGxVxMPGxDYQLxKxLGxLrPSVKRVMMYxMGCFA
GgTxLRLAKDLAENNxxxxKGAxxRVLVVCSEIxTAxVxFRxPSDxxxxx
LDSLxVGxALFGDGxAAAVIVGADPxxxxxxExxxRPLFELVxxxQxILP
DSExaIxxxxxLRExGLxFxLxxKxVPxxxxxLISkNIEkxLxExxxxLx
xxxxgxxxxxxxISxxDWNxxxxxxLFWIVHPGGxAILDxVExkLGLxxE
KMRATRxVLSEYGNMSSAxVLEVLDEMRKKsxxxEGxxxxGExxxxxGxE
WGVLxxFGPGLTVExVVLxSVxxxxxxxxxxxxxxxxxxxxxxxxxxxxx
xxxxxxxxxxxxxxxxxxxxxxxxxxxxxxxxxxxxxxxxxxxxxxxxxx
xxxxxxxxxx.

Amino acid sequences in agreement with the consensus sequence, and nucleotide sequences encoding such amino acid sequences are encompassed herein.

The method of the invention can be conveniently practiced by providing the compounds and/or compositions in the form of a kit, which may be used in a method to transform a host cell. Such kits may contain or be associated with instructions for use thereof.

### EXAMPLES—PART 3

To gain a better understanding of the invention described herein, the following examples are set forth. It should be understood that these examples are for illustrative purposes only. Therefore, they should not limit the scope of this invention in anyway.

### Example 4

Functional Demonstration of Production of Polyketides in a Transformed Host Cell.

### INTRODUCTION

Phytocannabinoids, such as tetrahydrocannabinol (THC) and cannabidiol (CBD), can be extracted from plant material for medical and psychotropic purposes. However, the synthesis of plant material is costly, not readily scalable to large volumes, and requires a lengthy grow periods to produce sufficient quantities of phytocannabinoids. An organism capable of fermentation, such as Saccharomyces cerevisiae, that is capable of producing cannabinoids would provide an economical route to producing these compounds on an industrial scale.

The early stages of the cannabinoid pathway proceeds via the generation of olivetolic acid by the type III PKS olivetolic acid synthase (OAS) and cyclase olivetolic acid cyclase (OAC). This reaction uses a hexanoyl-CoA starter as well as three units of malonyl-CoA. Olivetolic acid is the backbone of most classical cannabinoids and can be prenylated to form CBGA, which is ultimately converted to CBDA or THCA by an oxidocyclase. Production of olivetolic acid in S. cerevisiae is challenging as OAS generates significant by-products such as HTAL, PDAL and olivetol.

These by-products can be reduced in a recombinant organism by the introduction of olivetolic acid cyclase (OAC) but even with this enzyme by-products can account for up to 80% of the total carbon in the reaction.

In this example, it is reported for the first time that the addition to a host organism of a type III polyketide synthase (PKS) renders the organism capable of producing olivetolic acid and olivetol from hexanoyl-CoA and malonyl-CoA. The addition of a type 3 PKS enzyme to a host cell may be used to improve cannabinoid production in hosts such as S. cerevisiae and E. coli, or any other appropriate host microorganism.

In addition, these type 3 PKS enzymes may be used to access resorcinol/resorcylic acids with variant alkyl tails such as orcinol, orsellinic acid, divarin, and divarinic acid. These polyketides so formed can be prenylated and used to produce cannabinoids such as cannabivarins and cannabiorcinols, in downstream metabolic reactions, optionally within the host organism.

FIG. 19 depicts pathways for formation of different polyketides (also referred to herein as resorcinols or resorcyclic acids) polyketides from a fatty acid-CoA with (3×) malonyl-CoA, as the acetoacetyl-containing extender unit, as a consequences of the type 3 polyketide synthase (type 3 PKS) reaction. Hexanoyl-CoA and (3×) malonyl-CoA form olivetol/olivetolic acid; butyrl-CoA and (3×) malonyl-CoA form divarin/divarinic acid; and acetyl-CoA together with (3×) malonyl-CoA form orcinol/orsellenic acid.

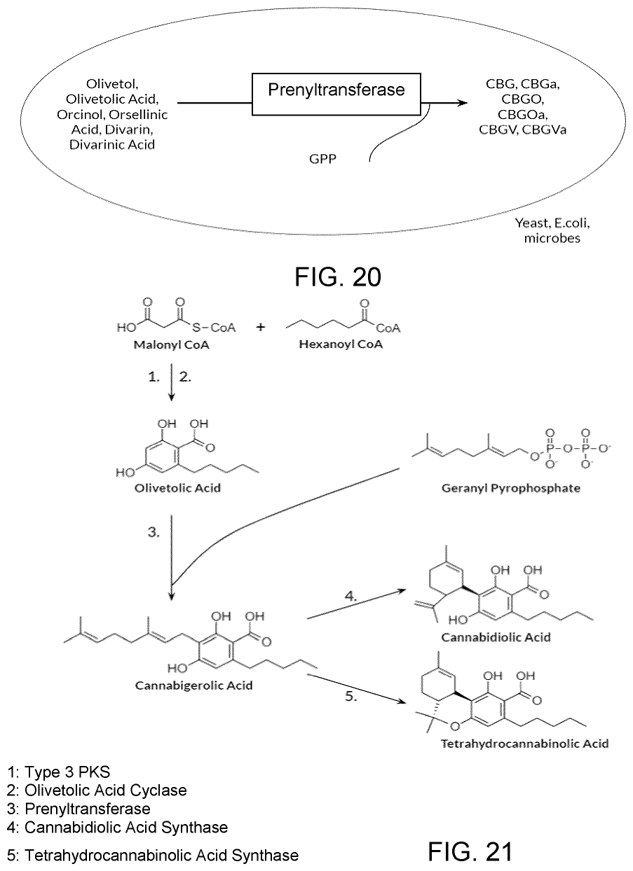

FIG. 20 depicts pathways for prenylation of polyketides with GPP, useful in the formation of certain phytocannabinoids. Please refer to FIG. 3 above, which shows structures of select phytocannabinoids of interest.

Materials and Methods

Plasmid Construction. All plasmids were synthesized by Twist DNA sciences. The sequences for PKS2 to PKS71 (see correspondence to SEQ ID Nos in Table 25) were synthesized in the pET21D+ vector (SEQ ID NO:119) between base-pair 5209 and 5210.

Upon receiving the DNA from Twist DNA sciences, 100 ng of each vector was transformed into E. coli BL21 (DE3) gold chemically competent cells. The cells were plated on LB Agar plates with 75 mg/L Ampicillin as the selective agent. Successful, isolated colonies were picked by hand and inoculated into 1 ml of LB media containing 75 mg/L ampicillin in 96-well sterile deep well plates. The plates were grown for 16 hours at 37° C. while being shaken at 250 RPM. After 16 hours 150 μl of each culture was transferred to a sterile microtiter plate containing 150 μl of 50% glycerol. The microtiter plates were sealed and stored at −80° C. as a cell stock.

SOP for feeding assay. E. coli BL21(DE3) Gold harbouring a plasmid containing a coding sequence for a type 3 PKS stored as a cell stock were inoculated into 1 mL cultures of TB Overnight Express autoinduction media containing 75 mg/L ampicillin in sterile 96-well 2 mL deep well plates. The cultures were grown overnight at 30° C. with shaking at 950 rpm. The following day the cells were harvested by centrifugation and frozen at −20° C. The thawed pellets were resuspended in 50 mM HEPES buffer (pH 7.5) with 10 mg/mL lysozyme, 2 U/mL benzonase, and 1× protease inhibitors. The suspension was incubated at 37° C. for 1 hour with shaking.

Following lysis, 20 μL of water was added to the cell lysate and centrifuged at max speed for 15 minutes. A total of 30 μL clear lysate was added to 20 μL of 50 mM HEPES buffer (pH 7.5) mixture containing a final concentration of 500 μM hexanoyl-CoA starter unit (the starter unity may be, for example: acetyl-CoA, butyryl-CoA, or hexanoyl-CoA), 1 mM malonyl-CoA extender unit, and 0.4% tween. The plate is sealed with a plate sealer and the reaction mixture is incubated at 30° C. without shaking in an incubator for 24 hours.

After 24 hours 200 μl of Acetonitrile was added to the reaction and the mixture was centrifuged at 3750 RPM for 10 minutes. 150 μl of the supernatant was then transferred to another microtiter plate, sealed and stored for analysis.

Quantification and Analysis. The analysis was performed using a Waters UPLC chromatography system connected to a Waters TQD mass spectrometer. The separation was performed on an Waters HSS column (1×50 mm, 1.8 um) using a reverse-phased method using water+0.1% formic acid as solvent A and acetonitrile (ACN)+0.1% formic acid as solvent B at a flow rate of 0.2 mL/min. Retention times (RT) for olivetol was 1.40 min and for olivetolic acid was 1.28 min.

Table 26 shows the column gradient profile used to isolate polyketide product.

TABLE 26
Gradient Profile

| | A | B |
|---|---|---|
| 0.00 min | 70% | 30% |
| 1.2 min | 50% | 50% |
| 1.70 min | 30% | 70% |
| 1.71 min | 70% | 30% |

The fractions assessed for olivetol or olivetolic acid were directed to mass spectrometry, performed using an ESI source in positive mode with a cone voltage of 24V and a collision voltage of 21V for the fragmentation.

Table 27 provides the parameters pertaining to the MS method for detection and quantification of products: olivetol and olivetolic acid.

TABLE 27
MS Parameters for Product Detection

| ES+ | M/Z | Transition | Cone Voltage (V) | Collision (V) |
|---|---|---|---|---|
| Olivetol | 181.1 | → 71 | 26 | 15 |
| Olivetolic Acid | 223.01 | → 171 | 35 | 20 |

Results and Discussion

E. coli cells transformed with Type 3 PKS and provided with hexanoyl-CoA and malonyl-CoA were able to form polyketide products.

Table 28 depicts olivetol and olivetolic acid concentrations found to be produced by a select subset of the transformed host cells upon culturing as described herein. The production of olivetol and olivetolic acid by feeding hexanoyl-CoA and malonyl-CoA to the transformed E. coli cells was evaluated in the cell lysate.

TABLE 28
Olivetol and Olivetolic Acid in Transformed E. coli Lysate

| | Olivetol Concentration | Olivetolic Acid |
|---|---|---|
| PKS # | (ug/L) | Concentration (ug/L) |
| Empty Vector | 0 | 0 |
| (Negative) | | |
| PKS8 | 0 | 1 |
| PKS10 | 0 | 10 |
| PKS17 | 0 | 6 |
| PKS20 | 0 | 15 |
| PKS22 | 0 | 2 |
| PKS25 | 0 | 1 |
| PKS26 | 0 | 1 |
| PKS27 | 0 | 2 |
| PKS31 | 0 | 1 |
| PKS33 | 0 | 1 |
| PKS47 | 2 | 1 |
| PKS48 | 0 | 1 |
| PKS49 | 4 | 1 |
| PKS54 | 0 | 2 |
| PKS56 | 0 | 1 |
| PKS57 | 0 | 1 |
| PKS58 | 0 | 5 |
| PKS61 | 0 | 14 |

These results are extremely promising for the Type 3 PKS sequences evaluated in this cell type. Cells not shown in Table 28 did not produce detectable quantities of polyketide under the experimental conditions described. However, with minor adjustments to conditions, and/or in different host cells, the other Type 3 PKS sequences may produce polyketide product from a fatty acid-CoA and extender unit comprising an acetoacetyl moiety (such as malonyl-CoA) starting materials.

### Example 5

Production of Cannabigerolic Acid (CBGa) in Recombinant Yeast Transformed with Type 3 PKS

This examples describes the production of cannabigerolic acid (CBGa) in vivo in a Saccharomyces cerevisiae strain that is capable of prenylating polyketides. The strain is one that is genetically modified with Type 3 PKS to produce the polyketide precursor of CBGa: olivetolic acid. Further, the strain is one capable of producing the monoterpene precursor geranyl pyrophosphate (GPP) as the prenyl moiety for the prenyltransferase reaction that leads to CBGa production. Please refer to FIG. 4 for a schematic overview of the native biosynthetic pathway for cannabinoid production in Cannabis sativa, in which the production of cannabigerolic acid, as well as cannabidiolic acid and tetrahydrocannabinolic acid is shown.

FIG. 21 illustrates an overview of a possible metabolic pathway in a yeast cell transformed with Type 3 PKS in the production of cannabigerolic acid, according to this example, as well as downstream formation of cannabidiolic acid and tetrahydrocannabinolic acid. Type 3 PKS (1) as described herein, and olivetolic acid cyclase (OAC) from C. sativa (2) are used to produce olivetolic acid via hexanoyl-CoA and malonyl-CoA. Geranyl pyrophosphate (GPP) from the yeast terpenoid pathway and olivetolic acid (OLA) are subsequently converted to cannabigerolic acid using a prenyltransferase (3). Cannabigerolic acid is then further cyclized to produce THCa or CBDa using C. sativa Tetrahydrocannabinolic Acid (THCa) synthase (5) or cannabidiolic acid (CBDa) synthase (4) enzymes, respectively.

In this Example, the base strain used may be HB144 Saccharomyces cerevisiae having genotype CEN.PK2; ΔLEU2; ΔURA3; Erg20K197E::KanMx;ALD6; ASC1L641P; NPGA; MAF1; PGK1p:ACC1S659A, S1157A; tHMGR1;ID.

The base strain may be transformed with one or more vectors, such as a plasmid containing at least the nucleotide sequence encoding a Type 3 PKS according to any one of SEQ ID NO: 120 to SEQ ID NO: 137.

The modified S. cerevisiae strain used as disclosed herein under conditions conducive to cannabinoid formation. A 6-carbon fatty acid-CoA substrate, hexanoyl-CoA, and an extender unit containing an acetoacetyl moiety (such as malonyl-CoA) may be provided, or the transformed cells may produce same intracellularly from a sugar substrate. The cells are cultured and maintained under conditions conducive to cannabinoid CBGa production.

The base strain may contain one or more genetic modifications that increase the available pool of hexanoyl-CoA and malonyl-CoA in the cell. For example, the native S. cerevisiae acetoacetyl-CoA carboxylase, ACC1, protein may also be overexpressed by changing its promoter to a constitutive promoter, and may have additional mutations, such as S659A and S1 157A in ACC1 in order to alleviate negative regulation by post-translational modification (Shi et al., 2014), which can thereby permit the cell to have a greater accumulation of malonyl-CoA. A greater accumulation of malonyl-CoA provides additional substrate to the type 3 PKS enzyme, and thus can enhance olivetolic acid production in the cell.

Genetic manipulations of the base strain HB144, may be conducted in a known manner, to develop transformed yeast cells. DNA may be transformed into the base strain using the Gietz et al. transformation protocol (Gietz, 2014). Plas 36 may be used for CRISPR-based genetic modifications (Ryan et al., 2016). Sequences according to any one of SEQ ID NO:120 to SEQ ID No:137 can thus be inserted into the host yeast cell to create a strain containing type 3 PKS that can synthesize CBGa either directly from glucose, or from other primer and/or extender units provided to the cell, with enhanced polyketide synthesis.

Host cells, such as yeast cells, transformed in this way may be used to produce phytocannabinoids or phytocannabinoid derivatives.

### Examples 6 to 11

Methods and Cell Lines for the Production of Polyketides

Introduction. Rationale, background, and common methodologies for Examples 6 to 11 are described herein below. In Examples 4 and 5, above, polyketide synthases are described that can produce olivetol when expressed in E. coli. In Examples 6 to 11, a PKSIII library is provided, which is also active in S. cerevisiae, and can produce olivetol and olivetolic acid when fed hexanoic acid and expressed with an appropriate acyl-CoA synthase and polyketide cyclase.

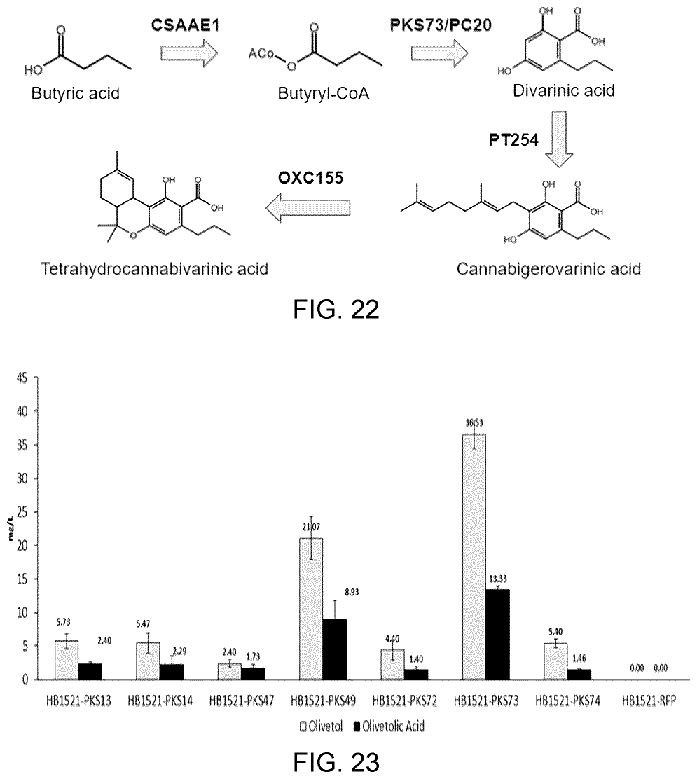

Due to the promiscuous nature of PKSIII enzymes, other starter units can also be accepted in place of hexanoyl-CoA yielding a variety of carbon tails in the resultant polyketides. As an example, it is shown here that the production of THCVa by feeding butyric acid to a novel polyketide synthase co-expressed with the appropriate C. sativa enzymes (FIG. 22). This process is analogous to the production of THCa using hexanoic acid.

FIG. 22 is a schematic illustration of the production of THCVa in S. cerevisiae using a polyketide synthase as described herein.

The polyketide synthases described in Examples 4 and 5 are also capable of forming products using other fatty acid feeds. In the current examples, a polyketide library is described that can accept octanoic acid, hexenoic and hexynoic acid (structures in Table 29). When co-expressed with an acyl-CoA synthase and polyketide cyclase it is shown herein how that these enzymes produce the corresponding polyketide acid. Prenyltransferases from C. sativa (PT254), stachybotrys (PT72+273), or R. dauricum (PT104) can then be used to convert these products to the corresponding cannabinoids. Herein is shown the production of C7-alkyl resorcylic acid, C5-alkenyl cannabigerolic acid and C5-alkynyl resorcylic acid. Structures of polyketides and cannabinoid products generated by providing octanoic, hexenoic or hexynoic acid, in Examples 6 to 11 are shown below.

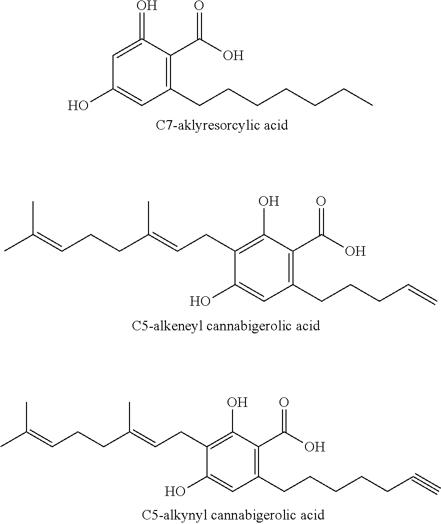

TABLE 29
Structure And Concentration Of Fatty Acids Fed For In Vivo Assays

| | | Concentration of acid | | |
|---|---|---|---|---|
| Fed assay | Acid structure | in assay | | Experiment Fed |
| Butyric acid | 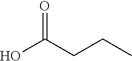 | 5 | mM | Example 7 and Example 11 |
| Hexanoic acid | 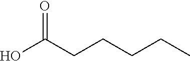 | 1 | mM | Example 6 |
| Octanoic acid | 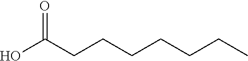 | 0.3 | mM | Example 8 |
| Hexynoic acid | 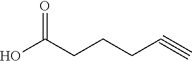 | 1 | mM | Example 9 |
| Hexenoic acid | 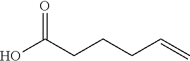 | 1 | mM | Example 10 |

An additional set of polyketide and acyl-CoA synthases are provided, and these Examples show that they can be used to improve THCVa titres. An expanded set of polyketide synthases (PKS80 to PKS109) and acyl-CoA synthases (Alk1 to Alk30) are provided. These synthases are transformed these into strains engineered to produce THCVa. It is established in these Examples that the expression of many of these enzymes greatly improved final cannabinoid titres.

Table 30 lists the modifications to the base strains used in Examples 6 to 11, as well as providing sequences.

TABLE 30
Modifications to base strains used in Examples 6 to 11

| | | Integration | | Genetic |
|---|---|---|---|---|
| Modification | SEQ | Region/ | | Structure of |
| name | ID NO. | Plasmid | Description | Sequence |
| (#1) | SEQ ID | USER site | CSAAE (Stout et al., 2012) is a C. sativa | XI-2up::pGAL-CSAAE1- |
| CSAAE1 | NO. 271 | XI-2 | enzyme that catalyzes the formation of | cyc::XI-2up |
| | | integration | fatty acyl-Coa's from free fatty acids. It | |
| | | | has demonstrated activity on hexanoic | |
| | | | and butyric acid | |
| (#2) | SEQ ID | Flagfeldt site | PC20 (Gagne et al., 2012) is a C. sativa | Fgf16::pGAL-PC20- |
| PC20 | NO. 272 | 16 integration | enzyme that is required for olivetolic acid | cyc::FgF16 |
| | | | formation | |
| (#3a) | SEQ ID | Flagfeldt site | PT254 (Luo et al., 2019) is a C. sativa | Fgf20::pGAL- |
| PT254 | NO. 279 | 20 integration | enzyme that prenylates olivetolic acid to | PT254-cyc::Fgf20 |
| | | | form cannabigerolic acid. It contains a 76 | |
| | | | amino acid truncation at the N-terminal | |
| (#3b) | SEQ ID | X-4 | PT72 is a Stachybotrys bisby enzyme that | Fgf20::pGAL-PT72- |
| PT72 | NO. 280 | integration | prenylates olivetolic acid to form | cyc::Fgf20 |
| | | site | cannabigerolic acid | |
| (#3c) | SEQ ID | X-4 | PT104 is a Rhododendron dauricum | Fgf20::pGAL- |
| PT104 | NO. 281 | integration | enzyme that prenylates olivetolic acid to | PT104-cyc::Fgf20 |
| | | site | form cannabigerolic acid. Contains a 34 | |
| | | | amino acid truncation at the N-terminal. | |
| (#3d) | SEQ ID | X-4 | PT273 is a Stachybotrys chlorohalonata | Fgf20::pGAL- |
| PT273 | NO. 282 | integration | enzyme that prenylates olivetolic acid to | PT274-cyc::Fgf20 |
| | | site | form cannabigerolic acid | |
| (#11) | SEQ ID | Apel-3 | OXC155 is a modified THCa synthase | Apel-3::OXC155- |
| OXC155 | NO. 273 | integration | from C. sativa. A 5′ OST-proAF tag has | cyc::Apel-3 |
| | | | been added to this gene. This enzyme will | |
| | | | produce THCa from a CBGa precursor | |

TABLE 31
Plasmids used in Examples 6-11

| # | Plasmid Name | Description | Selection | Backbone |
|---|---|---|---|---|
| 1 | PLAS400 | Gal1p:RFP:Cyc1t | Uracil | pYES-URA |
| 2 | PLAS434 | Gal1p:PKS13:Cyc1t | Uracil | pYES-URA |
| 3 | PLAS435 | Gal1p:PKS14:Cyc1t | Uracil | pYES-URA |
| 4 | PLAS436 | Gal1p:PKS47:Cyc1t | Uracil | pYES-URA |
| 5 | PLAS437 | Gal1p:PKS49:Cyc1t | Uracil | pYES-URA |
| 6 | PLAS438 | Gal1p:PKS72:Cyc1t | Uracil | pYES-URA |
| 7 | PLAS439 | Gal1p:PKS73:Cyc1t | Uracil | pYES-URA |
| 8 | PLAS440 | Gal1p:PKS74:Cyc1t | Uracil | pYES-URA |
| 9 | PLAS441 | Gal1p:PKS45:Cyc1t | Uracil | pYES-URA |
| 10 | PLAS442 | Gal1p:PKS65:Cyc1t | Uracil | pYES-URA |
| 11 | PLAS469 | pGAL:Alk27:Cyct | Uracil | pYES-URA |
| 12 | PLAS492 | pGAL:PKS92:Cyct | Uracil | pYES-URA |
| 13 | PLAS493 | pGAL:PKS100:Cyct | Uracil | pYES-URA |
| 14 | PLAS501 | pGAL:PKS108:Cyct | Uracil | pYES-URA |
| 15 | PLAS462 | pGAL:Alk20:Cyct | Uracil | pYES-URA |
| 16 | PLAS470 | pGAL:Alk28:Cyct | Uracil | pYES-URA |
| 17 | PLAS478 | pGAL:PKS85:Cyct | Uracil | pYES-URA |
| 18 | PLAS486 | pGAL:PKS93:Cyct | Uracil | pYES-URA |
| 19 | PLAS494 | pGAL:PKS101:Cyct | Uracil | pYES-URA |
| 20 | PLAS502 | pGAL:PKS109:Cyct | Uracil | pYES-URA |
| 21 | PLAS463 | pGAL:Alk21:Cyct | Uracil | pYES-URA |
| 22 | PLAS471 | pGAL:Alk29:Cyct | Uracil | pYES-URA |
| 23 | PLAS479 | pGAL:PKS86:Cyct | Uracil | pYES-URA |
| 24 | PLAS487 | pGAL:PKS94:Cyct | Uracil | pYES-URA |
| 25 | PLAS495 | pGAL:PKS102:Cyct | Uracil | pYES-URA |
| 26 | PLAS464 | pGAL:Alk22:Cyct | Uracil | pYES-URA |
| 27 | PLAS472 | pGAL:Alk30:Cyct | Uracil | pYES-URA |
| 28 | PLAS480 | pGAL:PKS87:Cyct | Uracil | pYES-URA |
| 29 | PLAS467 | pGAL:Alk25:Cyct | Uracil | pYES-URA |
| 30 | PLAS475 | pGAL:PKS82:Cyct | Uracil | pYES-URA |
| 31 | PLAS483 | pGAL:PKS90:Cyct | Uracil | pYES-URA |
| 32 | PLAS491 | pGAL:PKS98:Cyct | Uracil | pYES-URA |
| 33 | PLAS499 | pGAL:PKS106:Cyct | Uracil | pYES-URA |
| 34 | PLAS468 | pGAL:Alk26:Cyct | Uracil | pYES-URA |
| 35 | PLAS476 | pGAL:PKS83:Cyct | Uracil | pYES-URA |
| 36 | PLAS484 | pGAL:PKS91:Cyct | Uracil | pYES-URA |
| 37 | PLAS492 | pGAL:PKS99:Cyct | Uracil | pYES-URA |
| 38 | PLAS500 | pGAL:PKS107:Cyct | Uracil | pYES-URA |
| 39 | PLAS443 | pGAL:Alk1:Cyct | Uracil | pYES-URA |
| 40 | PLAS444 | pGAL:Alk2:Cyct | Uracil | pYES-URA |
| 41 | PLAS445 | pGAL:Alk3:Cyct | Uracil | pYES-URA |
| 42 | PLAS446 | pGAL:Alk4:Cyct | Uracil | pYES-URA |
| 43 | PLAS447 | pGAL:Alk5:Cyct | Uracil | pYES-URA |
| 44 | PLAS448 | pGAL:Alk6:Cyct | Uracil | pYES-URA |
| 45 | PLAS449 | pGAL:Alk7:Cyct | Uracil | pYES-URA |
| 46 | PLAS450 | pGAL:Alk8:Cyct | Uracil | pYES-URA |
| 47 | PLAS451 | pGAL:Alk9:Cyct | Uracil | pYES-URA |
| 48 | PLAS452 | pGAL:Alk10:Cyct | Uracil | pYES-URA |
| 49 | PLAS453 | pGAL:Alk11 :Cyct | Uracil | pYES-URA |
| 50 | PLAS454 | pGAL:Alk12:Cyct | Uracil | pYES-URA |
| 51 | PLAS455 | pGAL:Alk13:Cyct | Uracil | pYES-URA |
| 52 | PLAS456 | pGAL:Alk14:Cyct | Uracil | pYES-URA |
| 53 | PLAS457 | pGAL:Alk15:Cyct | Uracil | pYES-URA |
| 54 | PLAS458 | pGAL:Alk16:Cyct | Uracil | pYES-URA |
| 55 | PLAS459 | pGAL:Alk17:Cyct | Uracil | pYES-URA |
| 56 | PLAS460 | pGAL:Alk18:Cyct | Uracil | pYES-URA |
| 57 | PLAS461 | pGAL:Alk19:Cyct | Uracil | pYES-URA |

TABLE 32
Strains used in Examples 6 to 11

| Strain # | Background | Plasmids | Genotype | Notes |
|---|---|---|---|---|
| HB144 | -URA, -LEU | None | Saccharomyces cerevisiae | Base strain |
| | | | CEN.PK2; ΔLEU2; ΔURA3; | |
| | | | Erg20K197E::KanMx; | |
| | | | ALD6; ASC1L641P; NPGA; | |
| | | | MAF1; PGK1p:Acc1 | |
| HB1629 | -URA, -LEU | None | Saccharomyces cerevisiae | Host strain |
| | | | CEN.PK2; ΔLEU2; ΔURA3; | expressing PT273 |
| | | | Erg20K197E::KanMx; ALD6; | used in fatty acid |
| | | | ASC1L641P; NPGA; MAF1; | feeding assay for |
| | | | PGK1p:Acc1; tHMGR1; IDI; | the production of |
| | | | CsAAE1; PC20; PT273 | alkyl variant |
| | | | | cannabinoids |
| HB1630 | -URA, -LEU | None | Saccharomyces cerevisiae | Host strain |
| | | | CEN.PK2; ΔLEU2; ΔURA3; | expressing PT72 |
| | | | Erg20K197E::KanMx; ALD6; | used in fatty acid |
| | | | ASC1L641P; NPGA; MAF1; | feeding assay for |
| | | | PGK1p:Acc1; tHMGR1; IDI; | the production of |
| | | | CsAAE1; PC20; PT72 | alkyl variant |
| | | | | cannabinoids |
| HB1631 | -URA, -LEU | None | Saccharomyces cerevisiae | Host strain |
| | | | CEN.PK2; ΔLEU2; ΔURA3; | expressing PT104 |
| | | | Erg20K197E::KanMx; ALD6; | used in fatty acid |
| | | | ASC1L641P; NPGA; MAF1; | feeding assay for |
| | | | PGK1p:Acc1; tHMGR1; IDI; | the production of |
| | | | CsAAE1; PC20; PT104 | alkyl variant |
| | | | | cannabinoids |
| HB1632 | -URA, -LEU | None | Saccharomyces cerevisiae | Host strain |
| | | | CEN.PK2; ΔLEU2; ΔURA3; | expressing PT254 |
| | | | Erg20K197E::KanMx; ALD6; | used in fatty acid |
| | | | ASC1L641P; NPGA; MAF1; | feeding assay for |
| | | | PGK1p:Acc1; tHMGR1; IDI; | the production of |
| | | | CsAAE1; PC20; PT254 | alkyl variant |
| | | | | cannabinoids |
| HB1629- | -URA, -LEU | PLAS434 | Saccharomyces cerevisiae | Expresses PKS13 |
| PKS13 | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; IDI | |
| | | | CsAAE1; PC20; PT273 | |
| HB1629- | -URA, -LEU | PLAS435 | Saccharomyces cerevisiae | Expresses PKS14 |
| PKS14 | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; IDI | |
| | | | CsAAE1; PC20; PT273 | |
| HB1629- | -URA, -LEU | PLAS436 | Saccharomyces cerevisiae | Expresses PKS47 |
| PKS47 | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; IDI | |
| | | | CsAAE1; PC20; PT273 | |
| HB1629- | -URA, -LEU | PLAS437 | Saccharomyces cerevisiae | Expresses PKS49 |
| PKS49 | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; IDI | |
| | | | e CsAAE1; PC20; PT273 | |
| HB1629- | -URA, -LEU | PLAS442 | Saccharomyces cerevisiae | Expresses PKS65 |
| PKS65 | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; IDI | |
| | | | CsAAE1; PC20; PT273 | |
| H1B1630- | -URA, -LEU | PLAS441 | Saccharomyces cerevisiae | Expresses PKS45 |
| PKS45 | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; IDI | |
| | | | CsAAE1; PC20; PT72 | |
| HB1631- | -URA, -LEU | PLAS434 | Saccharomyces cerevisiae | Expresses PKS13 |
| PKS13 | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; IDI | |
| | | | CsAAE1; PC20; PT104 | |
| HB1631- | -URA, -LEU | PLAS435 | Saccharomyces cerevisiae | Expresses PKS14 |
| PKS14 | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; IDI | |
| | | | CsAAE1; PC20; PT104 | |
| HB1631- | -URA, -LEU | PLAS441 | Saccharomyces cerevisiae | Expresses PKS45 |
| PKS45 | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; IDI | |
| | | | CsAAE1; PC20; PT104 | |
| HB1632- | -URA, -LEU | PLAS434 | Saccharomyces cerevisiae | Expresses PKS13 |
| PKS13 | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; IDI | |
| | | | CsAAE1; PC20; PT254 | |
| HB1632- | -URA, -LEU | PLAS435 | Saccharomyces cerevisiae | Expresses PKS14 |
| PKS14 | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; IDI | |
| | | | CsAAEI; PT254 | |
| HB1632- | -URA, -LEU | PLAS439 | Saccharomyces cerevisiae | Expresses PKS73 |
| PKS73 | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; IDI | |
| | | | CsAAEI; PT254 | |
| HB1521 | -URA, -LEU | None | Saccharomyces cerevisiae | Parent strain for in |
| | | | CEN.PK2; ΔLEU2; ΔURA3; | hexanoic acid |
| | | | Erg20K197E::KanMx; ALD6; | feeding assay |
| | | | ASC1L641P; NPGA; MAF1; | |
| | | | PGK1p:Acc1; tHMGR1; IDI | |
| | | | CSAAE1; PC20 | |
| HB1521- | -URA, -LEU | PLAS434 | Saccharomyces cerevisiae | Produces olivetol |
| PKS13 | | | CEN.PK2; ΔLEU2; ΔURA3; | and olivetolic acid |
| | | | Erg20K197E::KanMx; ALD6; | when fed with |
| | | | ASC1L641P; NPGA; MAF1; | hexanoic acid |
| | | | PGK1p:Acc1; tHMGR1; IDI | |
| | | | CSAAE1; PC20 | |
| HB1521- | -URA, -LEU | PLAS435 | Saccharomyces cerevisiae | Produces olivetol |
| PKS14 | | | CEN.PK2; ΔLEU2; ΔURA3; | and olivetolic acid |
| | | | Erg20K197E::KanMx; ALD6; | when fed with |
| | | | ASC1L641P; NPGA; MAF1; | hexanoic acid |
| | | | PGK1p:Acc1; tHMGR1; IDI | |
| | | | CSAAE1; PC20 | |
| HB1521- | -URA, -LEU | PLAS436 | Saccharomyces cerevisiae | Produces olivetol |
| PKS47 | | | CEN.PK2; ΔLEU2; ΔURA3; | and olivetolic acid |
| | | | Erg20K197E::KanMx; ALD6; | when fed with |
| | | | ASC1L641P; NPGA; MAF1; | hexanoic acid |
| | | | PGK1p:Acc1; tHMGR1; IDI | |
| | | | CSAAE1; PC20 | |
| HB1521- | -URA, -LEU | PLAS437 | Saccharomyces cerevisiae | Produces olivetol |
| PKS49 | | | CEN.PK2; ΔLEU2; ΔURA3; | and olivetolic acid |
| | | | Erg20K197E::KanMx; ALD6; | when fed with |
| | | | ASC1L641P; NPGA; MAF1; | hexanoic acid |
| | | | PGK1p:Acc1; tHMGR1; IDI | |
| | | | CSAAE1; PC20 | |
| HB1521- | -URA, -LEU | PLAS438 | Saccharomyces cerevisiae | Produces olivetol |
| PKS72 | | | CEN.PK2; ΔLEU2; ΔURA3; | and olivetolic acid |
| | | | Erg20K197E::KanMx; ALD6; | when fed with |
| | | | ASC1L641P; NPGA; MAF1; | hexanoic acid |
| | | | PGK1p:Acc1; tHMGR1; IDI | |
| | | | CSAAE1; PC20 | |
| HB1521- | -URA, -LEU | PLAS439 | Saccharomyces cerevisiae | Produces olivetol |
| PKS73 | | | CEN.PK2; ΔLEU2; ΔURA3; | and olivetolic acid |
| | | | Erg20K197E::KanMx; ALD6; | when fed with |
| | | | ASC1L641P; NPGA; MAF1; | hexanoic acid |
| | | | PGK1p:Acc1; tHMGR1; IDI | |
| | | | CSAAE1; PC20 | |
| HB1521- | -URA, -LEU | PLAS440 | Saccharomyces cerevisiae | Produces olivetol |
| PKS74 | | | CEN.PK2; ΔLEU2; ΔURA3; | and olivetolic acid |
| | | | Erg20K197E::KanMx; ALD6; | when fed with |
| | | | ASC1L641P; NPGA; MAF1; | hexanoic acid |
| | | | PGK1p:Acc1; tHMGR1; IDI | |
| | | | CSAAE1; PC20 | |
| HB1521- | -URA, -LEU | PLAS400 | Saccharomyces cerevisiae | Negative control for |
| RFP | | | CEN.PK2; ΔLEU2; ΔURA3; | hexanoic acid |
| | | | Erg20K197E::KanMx; ALD6; | feeding |
| | | | ASC1L641P; NPGA; MAF1; | |
| | | | PGK1p:Acc1; tHMGR1; IDI | |
| | | | CSAAE1; PC20 | |
| HB1775- | -URA, -LEU | PLAS400 | Saccharomyces cerevisiae | Produces THCVa |
| RFP | | | CEN.PK2; ΔLEU2; ΔURA3; | when fed butyric |
| | | | Erg20K197E::KanMx; ALD6; | acid |
| | | | ASC1L641P; NPGA; MAF1; | |
| | | | PGK1p:Acc1; tHMGR1; IDI | |
| | | | CSAAE1; PC20; PT254; OXC155 | |
| HB144- | -URA, -LEU | PLAS400 | Saccharomyces cerevisiae | Produces THCVa |
| RFP | | | CEN.PK2; ΔLEU2; ΔURA3; | when fed hexanoic |
| | | | Erg20K197E::KanMx; ALD6; | acid |
| | | | ASC1L641P; NPGA; MAF1; | |
| | | | PGK1p:Acc1 | |
| HB1775- | URA, -LEU | PLAS469 | Saccharomyces cerevisiae | Alk27 |
| Alk27 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS492 | Saccharomyces cerevisiae | PKS92 |
| PKS92 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS493 | Saccharomyces cerevisiae | PKS100 |
| PKS100 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS501 | Saccharomyces cerevisiae | PKS108 |
| PKS108 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS462 | Saccharomyces cerevisiae | Alk20 |
| Alk20 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS470 | Saccharomyces cerevisiae | Alk28 |
| Alk28 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS478 | Saccharomyces cerevisiae | PKS85 |
| PKS85 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS486 | Saccharomyces cerevisiae | PKS93 |
| PKS93 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS494 | Saccharomyces cerevisiae | PKS101 |
| PKS101 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS502 | Saccharomyces cerevisiae | PKS109 |
| PKS109 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS463 | Saccharomyces cerevisiae | Alk21 |
| Alk21 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS471 | Saccharomyces cerevisiae | Alk29 |
| Alk29 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS479 | Saccharomyces cerevisiae | PKS86 |
| PKS86 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS487 | Saccharomyces cerevisiae | PKS94 |
| PKS94 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS495 | Saccharomyces cerevisiae | PKS102 |
| PKS102 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS464 | Saccharomyces cerevisiae | Alk22 |
| Alk22 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS472 | Saccharomyces cerevisiae | Alk30 |
| Alk30 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS480 | Saccharomyces cerevisiae | PKS87 |
| PKS87 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS467 | Saccharomyces cerevisiae | Alk25 |
| Alk25 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS475 | Saccharomyces cerevisiae | PKS82 |
| PKS82 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS483 | Saccharomyces cerevisiae | PKS90 |
| PKS90 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS491 | Saccharomyces cerevisiae | PKS98 |
| PKS98 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS499 | Saccharomyces cerevisiae | PKS106 |
| PKS106 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS468 | Saccharomyces cerevisiae | Alk26 |
| Alk26 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS476 | Saccharomyces cerevisiae | PKS83 |
| PKS83 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS484 | Saccharomyces cerevisiae | PKS91 |
| PKS91 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS492 | Saccharomyces cerevisiae | PKS99 |
| PKS99 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS500 | Saccharomyces cerevisiae | PKS107 |
| PKS107 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS443 | Saccharomyces cerevisiae | Alk1 |
| Alk1 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS444 | Saccharomyces cerevisiae | Alk2 |
| Alk2 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS445 | Saccharomyces cerevisiae | Alk3 |
| Alk3 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS446 | Saccharomyces cerevisiae | Alk4 |
| Alk4 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS447 | Saccharomyces cerevisiae | Alk5 |
| Alk5 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS448 | Saccharomyces cerevisiae | Alk6 |
| Alk6 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS449 | Saccharomyces cerevisiae | Alk7 |
| Alk7 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS450 | Saccharomyces cerevisiae | Alk8 |
| Alk8 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS451 | Saccharomyces cerevisiae | Alk9 |
| Alk9 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS452 | Saccharomyces cerevisiae | Alk10 |
| Alk10 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS453 | Saccharomyces cerevisiae | Alk11 |
| Alk11 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS454 | Saccharomyces cerevisiae | Alk12 |
| Alk12 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS455 | Saccharomyces cerevisiae | Alk13 |
| Alk13 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS456 | Saccharomyces cerevisiae | Alk14 |
| Alk14 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS457 | Saccharomyces cerevisiae | Alk15 |
| Alk15 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS458 | Saccharomyces cerevisiae | Alk16 |
| Alk16 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS459 | Saccharomyces cerevisiae | Alk17 |
| Alk17 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS460 | Saccharomyces cerevisiae | Alk18 |
| Alk18 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |
| HB1775- | URA, -LEU | PLAS461 | Saccharomyces cerevisiae | Alk19 |
| Alk19 | | | CEN.PK2; ΔLEU2; ΔURA3; | overexpression |
| | | | Erg20K197E::KanMx; ALD6; | |
| | | | ASC1L641P; NPGA; MAF1; | |
| | | | PGK1p:Acc1; tHMGR1; IDI | |
| | | | CSAAE1; PC20; PT254; OXC155 | |

Table 33 shows genes and proteins used in these Examples. Note that sequences for PKS13-76 are provided above.

TABLE 33
Genes And Proteins Used

| | | | Length of | Position of coding |
|---|---|---|---|---|
| SEQ ID NO: | Description | DNA/Protein | sequence | sequence |
| SEQ ID NO. 271 | CsAAE1 | Gene | 3858 | 856-3021 |
| SEQ ID NO. 272 | PC20 | Gene | 2051 | 842-1150 |
| SEQ ID NO. 273 | OXC155 | Gene | 4684 | 1505-3355 |
| SEQ ID NO. 274 | PDH | Gene | 7114 | Ald6: 1444-2949 |
| | | | | ACS: 3888-5843 |
| SEQ ID NO. 275 | MAF1 | Gene | 3256 | 36-2123 |
| SEQ ID NO. 276 | ERG20K197E | Gene | 4254 | 2842-3900 |
| SEQ ID NO. 277 | tHMGR1-IDI | Gene | 4843 | tHMGR1: 885-2393 |
| | | | | IDI1: 3209-4075 |
| SEQ ID NO. 278 | NPGA | Gene | 3564 | 1170-2201 |
| SEQ ID NO. 279 | PT254 | Gene | 2395 | 728-1699 |
| SEQ ID NO. 280 | PT72 | Gene | 2425 | 728-1729 |
| SEQ ID NO. 281 | PT104 | Gene | 2479 | 728-1783 |
| SEQ ID NO. 282 | PT273 | Gene | 2413 | 728-1717 |
| SEQ ID NO. 283 | RFP | Protein | 243 | all |
| SEQ ID NO. 284 | Alk1 | Protein | 721 | all |
| SEQ ID NO. 285 | Alk2 | Protein | 721 | all |
| SEQ ID NO. 286 | Alk3 | Protein | 537 | all |
| SEQ ID NO. 287 | Alk4 | Protein | 720 | all |
| SEQ ID NO. 288 | Alk5 | Protein | 640 | all |
| SEQ ID NO. 289 | Alk6 | Protein | 726 | all |
| SEQ ID NO. 290 | Alk7 | Protein | 722 | all |
| SEQ ID NO. 291 | Alk8 | Protein | 722 | all |
| SEQ ID NO. 292 | Alk9 | Protein | 744 | all |
| SEQ ID NO. 293 | Alk10 | Protein | 415 | all |
| SEQ ID NO. 294 | Alk11 | Protein | 721 | all |
| SEQ ID NO. 295 | Alk12 | Protein | 727 | all |
| SEQ ID NO. 296 | Alk13 | Protein | 729 | all |
| SEQ ID NO. 297 | Alk14 | Protein | 731 | all |
| SEQ ID NO. 298 | Alk15 | Protein | 686 | all |
| SEQ ID NO. 299 | Alk16 | Protein | 727 | all |
| SEQ ID NO. 300 | Alk17 | Protein | 732 | all |
| SEQ ID NO. 301 | Alk18 | Protein | 689 | all |
| SEQ ID NO. 302 | Alk19 | Protein | 544 | all |
| SEQ ID NO. 303 | Alk20 | Protein | 546 | all |
| SEQ ID NO. 304 | Alk21 | Protein | 577 | all |
| SEQ ID NO. 305 | Alk22 | Protein | 586 | all |
| SEQ ID NO. 306 | Alk23 | Protein | 572 | all |
| SEQ ID NO. 307 | Alk24 | Protein | 577 | all |
| SEQ ID NO. 308 | Alk25 | Protein | 577 | all |
| SEQ ID NO. 309 | Alk26 | Protein | 446 | all |
| SEQ ID NO. 310 | Alk27 | Protein | 446 | all |
| SEQ ID NO. 311 | Alk28 | Protein | 448 | all |
| SEQ ID NO. 312 | Alk29 | Protein | 447 | all |
| SEQ ID NO. 313 | Alk30 | Protein | 627 | all |
| SEQ ID NO. 314 | PKS80 | Protein | 345 | all |
| SEQ ID NO. 315 | PKS81 | Protein | 427 | all |
| SEQ ID NO. 316 | PKS82 | Protein | 330 | all |
| SEQ ID NO. 317 | PKS83 | Protein | 363 | all |
| SEQ ID NO. 318 | PKS84 | Protein | 450 | all |
| SEQ ID NO. 319 | PKS85 | Protein | 356 | all |
| SEQ ID NO. 320 | PKS86 | Protein | 367 | all |
| SEQ ID NO. 321 | PKS87 | Protein | 392 | all |
| SEQ ID NO. 322 | PKS88 | Protein | 345 | all |
| SEQ ID NO. 323 | PKS89 | Protein | 392 | all |
| SEQ ID NO. 324 | PKS90 | Protein | 408 | all |
| SEQ ID NO. 325 | PKS91 | Protein | 392 | all |
| SEQ ID NO. 326 | PKS92 | Protein | 343 | all |
| SEQ ID NO. 327 | PKS93 | Protein | 392 | all |
| SEQ ID NO. 328 | PKS94 | Protein | 392 | all |
| SEQ ID NO. 329 | PKS95 | Protein | 329 | all |
| SEQ ID NO. 330 | PKS96 | Protein | 372 | all |
| SEQ ID NO. 331 | PKS97 | Protein | 365 | all |
| SEQ ID NO. 332 | PKS98 | Protein | 392 | all |
| SEQ ID NO. 333 | PKS99 | Protein | 366 | all |
| SEQ ID NO. 334 | PKS100 | Protein | 398 | all |
| SEQ ID NO. 335 | PKS101 | Protein | 401 | all |
| SEQ ID NO. 336 | PKS102 | Protein | 379 | all |
| SEQ ID NO. 337 | PKS103 | Protein | 395 | all |
| SEQ ID NO. 338 | PKS104 | Protein | 390 | all |
| SEQ ID NO. 339 | PKS105 | Protein | 376 | all |
| SEQ ID NO. 340 | PKS106 | Protein | 437 | all |
| SEQ ID NO. 341 | PKS107 | Protein | 377 | all |
| SEQ ID NO. 342 | PKS108 | Protein | 419 | all |
| SEQ ID NO. 343 | PKS109 | Protein | 392 | all |
| SEQ ID NO. 344 | PLAS443 | DNA | 7948 | 3019-5181 |
| SEQ ID NO. 345 | PLAS444 | DNA | 7948 | 3019-5181 |
| SEQ ID NO. 346 | PLAS445 | DNA | 7396 | 3019-4629 |
| SEQ ID NO. 347 | PLAS446 | DNA | 7945 | 3019-5178 |
| SEQ ID NO. 348 | PLAS447 | DNA | 7705 | 3019-4938 |
| SEQ ID NO. 349 | PLAS448 | DNA | 7963 | 3019-5196 |
| SEQ ID NO. 350 | PLAS449 | DNA | 7951 | 3019-5184 |
| SEQ ID NO. 351 | PLAS450 | DNA | 7951 | 3019-5184 |
| SEQ ID NO. 352 | PLAS451 | DNA | 8017 | 3019-5250 |
| SEQ ID NO. 353 | PLAS452 | DNA | 7030 | 3019-4263 |
| SEQ ID NO. 354 | PLAS453 | DNA | 7948 | 3019-5181 |
| SEQ ID NO. 355 | PLAS454 | DNA | 7966 | 3019-5199 |
| SEQ ID NO. 356 | PLAS455 | DNA | 7972 | 3019-5205 |
| SEQ ID NO. 357 | PLAS456 | DNA | 7978 | 3019-5211 |
| SEQ ID NO. 358 | PLAS457 | DNA | 7843 | 3019-5076 |
| SEQ ID NO. 359 | PLAS458 | DNA | 7966 | 3019-5199 |
| SEQ ID NO. 360 | PLAS459 | DNA | 7981 | 3019-5214 |
| SEQ ID NO. 361 | PLAS460 | DNA | 7981 | 3019-5085 |
| SEQ ID NO. 362 | PLAS461 | DNA | 7417 | 3019-4650 |
| SEQ ID NO. 363 | PLAS462 | DNA | 7429 | 3019-4662 |
| SEQ ID NO. 364 | PLAS463 | DNA | 7522 | 3019-4755 |
| SEQ ID NO. 365 | PLAS464 | DNA | 7549 | 3019-4782 |
| SEQ ID NO. 366 | PLAS465 | DNA | 7507 | 3019-4740 |
| SEQ ID NO. 367 | PLAS466 | DNA | 7522 | 3019-4755 |
| SEQ ID NO. 368 | PLAS467 | DNA | 7522 | 3019-4755 |
| SEQ ID NO. 369 | PLAS468 | DNA | 7129 | 3019-4362 |
| SEQ ID NO. 370 | PLAS469 | DNA | 7126 | 3019-4359 |
| SEQ ID NO. 371 | PLAS470 | DNA | 7135 | 3019-4368 |
| SEQ ID NO. 372 | PLAS471 | DNA | 7132 | 3019-4365 |
| SEQ ID NO. 373 | PLAS472 | DNA | 7669 | 3019-4902 |
| SEQ ID NO. 374 | PLAS473 | DNA | 6823 | 3019-4056 |
| SEQ ID NO. 375 | PLAS474 | DNA | 7069 | 3019-4302 |
| SEQ ID NO. 376 | PLAS475 | DNA | 6778 | 3019-4011 |
| SEQ ID NO. 377 | PLAS476 | DNA | 6877 | 3019-4110 |
| SEQ ID NO. 378 | PLAS477 | DNA | 7138 | 3019-4371 |
| SEQ ID NO. 379 | PLAS478 | DNA | 6856 | 3019-4089 |
| SEQ ID NO. 380 | PLAS479 | DNA | 6889 | 3019-4122 |
| SEQ ID NO. 381 | PLAS480 | DNA | 6964 | 3019-4197 |
| SEQ ID NO. 382 | PLAS481 | DNA | 6823 | 3019-4056 |
| SEQ ID NO. 383 | PLAS482 | DNA | 6964 | 3019-4197 |
| SEQ ID NO. 384 | PLAS483 | DNA | 7012 | 3019-4245 |
| SEQ ID NO. 385 | PLAS484 | DNA | 6964 | 3019-4197 |
| SEQ ID NO. 386 | PLAS485 | DNA | 6817 | 3019-4050 |
| SEQ ID NO. 387 | PLAS486 | DNA | 6964 | 3019-4197 |
| SEQ ID NO. 388 | PLAS487 | DNA | 6964 | 3019-4197 |
| SEQ ID NO. 389 | PLAS488 | DNA | 6775 | 3019-4008 |
| SEQ ID NO. 390 | PLAS489 | DNA | 6904 | 3019-4137 |
| SEQ ID NO. 391 | PLAS490 | DNA | 6883 | 3019-4116 |
| SEQ ID NO. 392 | PLAS491 | DNA | 6964 | 3019-4197 |
| SEQ ID NO. 393 | PLAS492 | DNA | 6886 | 3019-4119 |
| SEQ ID NO. 394 | PLAS493 | DNA | 6982 | 3019-4215 |
| SEQ ID NO. 395 | PLAS494 | DNA | 6991 | 3019-4224 |
| SEQ ID NO. 396 | PLAS495 | DNA | 6925 | 3019-4158 |
| SEQ ID NO. 397 | PLAS496 | DNA | 6973 | 3019-4206 |
| SEQ ID NO. 398 | PLAS497 | DNA | 6922 | 3019-4155 |
| SEQ ID NO. 399 | PLAS498 | DNA | 6916 | 3019-4149 |
| SEQ ID NO. 400 | PLAS499 | DNA | 7099 | 3019-4332 |
| SEQ ID NO. 401 | PLAS500 | DNA | 6919 | 3019-4152 |
| SEQ ID NO. 402 | PLAS501 | DNA | 7045 | 3019-4278 |
| SEQ ID NO. 403 | PLAS502 | DNA | 6964 | 3019-4197 |
| SEQ ID NO. 404 | PLAS400 | DNA | 6484 | 3019-3717 |
| SEQ ID NO. 405 | CSAAE1 | Protein | 721 | all |
| SEQ ID NO. 406 | PC20 | Protein | 102 | all |
| SEQ ID NO. 407 | PT72 | Protein | 333 | all |
| SEQ ID NO. 408 | PT104 | Protein | 351 | all |
| SEQ ID NO. 409 | PT254 | Protein | 323 | all |
| SEQ ID NO. 410 | PT296 | Protein | 329 | all |
| SEQ ID NO. 411 | OXC53 | Protein | 616 | all |

Genetic Manipulations:

HB144 was used as a base strain to develop all other strains in this experiment. All DNA was transformed into strains using the Gietz et al transformation protocol (Saeki et al., 2018). Plas 36 was used for the CRISPR-based genetic modifications described herein (Geitz 2014).

The genome of HB42 was iteratively targeted by gRNA's and Cas9 expressed from PLAS36 to make the following genomic modifications in the order of Table 34 below.

TABLE 34
Genomic Modifications

| Order | Genomic Region | Modification |
|---|---|---|
| 1 | USER site XI-2 | CSAAE1 |
| | integration | |
| 2 | Flagfeldt Site 16 | PC20 |
| | integration | |
| 3 | Flagfeldt Site 20 | PT254 |
| | integration | |
| 3 | X-4 site integration | PT72 |
| 3 | X-4 site integration | PT104 |
| 3 | X-4 site integration | PT273 |
| 4 | Apel-3 site integration | OXC155 |

Experimental Conditions. 3 single colony replicates of strains were tested in this study. Following a 48 hour preculture, all strains were grown in 1 ml media in 96-well deepwell plates. The deepwell plates were incubated at 30° C. and shaken at 950 rpm for 96 hrs. Metabolite extraction was performed by adding 300 μl of 100% acetonitrile to 100 μl of culture in a new 96-well deepwell plate. The solutions were then centrifuged at 3750 rpm for 5 min. 200 μl of the soluble layer was removed and stored in a 96-well v-bottom microtiter plate. Samples were stored at −20° C. until analysis. Samples were quantified using HPLC-MS analysis.

Quantification Protocols

Olivetol/Olivetolic Acid

The quantification of olivetol, olivetolic acid was performed using HPLC-MS on a Acquity UPLC-TQD MS. The chromatography and MS conditions are described below.

Column: Waters Acquity UPLC C18 column 1×50 mm, 1.8 um. Column temperature: 45. Flow rate: 0.35 mL/min. Eluent A: H2O 0.1% Formic Acid. Eluent B: ACN 0.1% Formic Acid.

TABLE 35
Gradient

| Time (min) | % B | Flow rate (ml/min) |
|---|---|---|
| 0 | 80 | 0.35 |
| 0.55 | 10 | 0.35 |
| 0.56 | 80 | 0.35 |
| 1.00 | 80 | 0.35 |

ESI-MS conditions: Capillary: 4 kV. Source temperature: 150° C. Desolvation gas temperature: 400° C. Drying gas flow (nitrogen): 500 L/hr. Collision gas flow (argon): 0.10 mL/min.

MRM Transitions: Olivetol (positive ionisation): m/z 181.1→m/z 71. Olivetolic acid (negative ionisation): m/z 223→179.

Divarin, divarinic acid, CBGa, THCa. The quantification of divarin, divarinic acid, CBGVa and THCVa was performed using HPLC-MS on a Acquity UPLC-TQD MS. The chromatography and MS conditions are described below.

LC conditions: Column: Waters Acquity UPLC C18 column 1×50 mm, 1.8 um. Column temperature: 45. Flow rate: 0.35 mL/min. Eluent A: H2O 0.1% Formic Acid. Eluent B: ACN 0.1% Formic Acid.

TABLE 36
Gradient

| Time (min) | % B | Flow rate (ml/min) |
|---|---|---|
| 0 | 90 | 0.35 |
| 1.20 | 10 | 0.35 |
| 1.21 | 90 | 0.35 |
| 2.00 | 90 | 0.35 |

ESI-MS conditions: Capillary: 4 kV. Source temperature: 150° C. Desolvation gas temperature: 400° C. Drying gas flow (nitrogen): 500 L/hr. Collision gas flow (argon): 0.10 mL/min.

MRM Transitions: Divarin (positive ionisation): m/z 153.0→m/z 153.0. Divarinic acid (negative ionisation): m/z 195.1→m/z 151.0. CBGVa (negative ionisation): m/z 331.2→313.2. THCVa (negative ionisation): m/z 329.2→m/z 285.2. CBGa (negative ionisation): m/z 359.2→341.2. THCa (negative ionisation): m/z 357.2→313.2.

c7-alkylresorcylic acid, c5-alkynyl cannabigerolic acid, c5-alkenyl cannabigerolic acid. The quantification for C7-alkylresorcylic acid, cannabigryolic acid and cannabigenerolic acid utilized an Agilent 6560 ion mobility-QTOF. Chromatography and MS conditions are described below. Exact masses of observed products are provided below.

LC conditions: Column: Acquity UPLC BEH 018 1.7 micron 2.1×5 mm. Column temperature: 45° C. Flow rate: 0.3 ml/min. Eluent A: Water 100%. Eluent B: Acetonitrile 100%.

TABLE 37
Gradient

| Time (min) | % B | Flow rate (ml/min) |
|---|---|---|
| 0.00 | 30 | 0.300 |
| 3.50 | 95 | 0.300 |
| 3.60 | 95 | 0.450 |
| 4.60 | 95 | 0.450 |
| 4.70 | 30 | 0.450 |
| 7.00 | 30 | 0.300 |

ESI-MS conditions: Capillary: 3.5 kV. Source temperature: 150° C. Desolvation gas temperature: 300° C. Drying gas flow (nitrogen): 600 L/hr. Sheath gas flow (nitrogen): 660 L/h r.

TABLE 38
Monoisotopic Masses Of Analyzed Minor Cannabinoids
And Their Polyketide Precursors

| | M/z of | | M/z of | |
|---|---|---|---|---|
| | polyketide | M/z of prenylated | polyketide | M/z of prenylated |
| Fed acid | alcohol | polyketide alcohol | acid | polyketide acid |
| Octanoic acid 0.3 mM | 208.1463 | 388.2614 | 252.1362 | 388.2614 |
| hexynoic acid 1 mM | 176.0837 | 356.1988 | 220.0735 | 356.1988 |
| hexenoic acid 1 mM | 178.0994 | 358.2144 | 222.0892 | 358.2144 |

### Example 6

Production of Olivetol and Olivetolic Acid in S. cerevisiae by Hexanoic Acid Feeding

This Example Involves In vivo production of olivetol and olivetolic acid in S. cerevisiae by hexanoic acid feeding. Here we show that co-expressing our type III PKS library with CSAAE1 and PC20 and feeding hexanoic acid results in the production of olivetol and olivetolic acid. These data illustrate that these enzymes also function in S. cerevisiae and can be used to produce olivetolic acid as well as olivetol.

Strain Growth and Media. Strains were grown in 500 ul pre-cultures for 48 hours in a 96 well plate. The preculture media consisted of yeast minimal media with a composition of 1.7 g/L YNB without ammonium sulfate+1.96 g/L URA dropout amino acid supplements+0.375 g/L monosodium glutamate and 10 g/L glucose. After 48 hours 50 ul of culture was transferred to a fresh 96 well plate containing 450 ul of culture media culture consisting of 1.7 g/L YNB without ammonium sulfate+1.96 g/L URA dropout amino acid supplements+1.5 g/L monosodium glutamate, 20 g/L raffinose and 20 g/L galactose+1.5 mM hexanoic acid. Strains were grown for an additional 96 hours and then extracted in acetonitrile.

Results

HB1521 was transformed with plasmids expressing either PKS(1-76) or an RFP negative were grown in the presence of 1 mM hexanoic acid. HB1521 has genomic copies CSAAE1 and PC20 from C. sativa and should produce olivetol and olivetolic acid in the presence of an appropriate polyketide synthase. Olivetol and olivetolic acid produced by these strains is shown in FIG. 23, the values for which are provided in Table 39.

TABLE 39
Olivetol and olivetolic acid produced by strains in Example 6

| Strain Name | Olivetol (mg/L) | Olivetolic acid (mg/) |
|---|---|---|
| HB1521-PKS13 | 5.73 | 2.40 |
| HB1521-PKS14 | 5.46 | 2.29 |
| HB1521-PKS47 | 2.4 | 1.73 |
| HB1521-PKS49 | 21.06 | 8.93 |
| HB1521-PKS72 | 4.40 | 1.40 |
| HB1521-PKS73 | 36.53 | 13.33 |
| HB1521-PKS74 | 5.40 | 0.69 |
| HB1521-RFP | 0 | 0 |

### Example 7

In Vivo Production of THCVa

This Example involves in vivo production of THCVa using PKS73. This shows a unique route to THCVa using PKS73 in place of the C. sativa polyketide synthase. Feeding HB1775—a strain expressing CSAAE1, PC20, PT254, PKS73, and OXC155 with butyric acid results in THCVa production.

Strain Growth and Media. Strains were grown in 500 ul pre-cultures for 48 hours in a 96 well plate. The preculture media consisted of yeast minimal media with a composition of 1.7 g/L YNB without ammonium sulfate+1.96 g/L URA dropout amino acid supplements+0.375 g/L monosodium glutamate and 10 g/L glucose. After 48 hours 50 ul of culture was transferred to a fresh 96 well plate containing 450 ul of culture media culture consisting of 1.7 g/L YNB without ammonium sulfate+1.96 g/L URA dropout amino acid supplements+1.5 g/L monosodium glutamate, 20 g/L raffinose and 20 g/L galactose+5 mM butyric acid. Strains were grown for an additional 96 hours and then extracted in acetonitrile.

Results

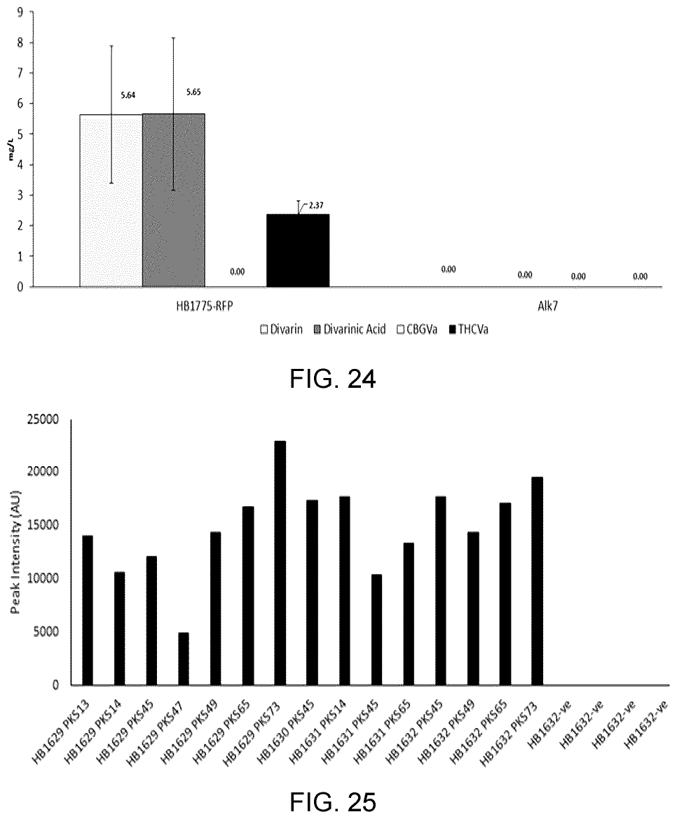

HB1775-RFP and HB144-RFP were grown in the presence in of 5 mM butyric acid. HB1775 has the genomic copies of CSAAE1, PC20, PT254 and OXC155 and PKS73, which should function as a complete pathway to THCVa. Divarin, divarinic acid, CBGVa and THCVa titres are shown in FIG. 24 and Table 40.

FIG. 24 shows divarin, divarinic acid, CBGVa and THCVa produced by strains in Example 7.

TABLE 40
Divarin, divarinic acid, CBGVa and THCVa
produced bv strains in Example 7

| | Divarin | Divarinic acid | CBGVa | THCVa |
|---|---|---|---|---|
| Strain name | (mg/L) | (mg/L) | (mg/L) | (mg/L) |
| HB1775-RFP | 5.64 | 5.65 | 0 | 2.37 |
| HB144-RFP | 0 | 0 | 0 | 0 |

### Example 8

In Vivo Production of C7-Resorcylic Acid

In this Example, in vivo production of C7-resorcylic acid. Here we show that co-expressing our type III PKS library with CSAAE1 and PC20 and feeding octanoic acid results in the production of C7-alkylresorcylic acid. These data emphasize that a wide variety of molecules can be produced.

Strain Growth and Media. Strains were grown in 500 ul pre-cultures for 48 hours in a 96 well plate. The preculture media consisted of yeast minimal media with a composition of 1.7 g/L YNB without ammonium sulfate+1.96 g/L URA dropout amino acid supplements+0.375 g/L monosodium glutamate and 10 g/L glucose. After 48 hours 50 ul of culture was transferred to a fresh 96 well plate containing 450 ul of culture media culture consisting of 1.7 g/L YNB without ammonium sulfate+1.96 g/L URA dropout amino acid supplements+1.5 g/L monosodium glutamate, 20 g/L raffinose and 20 g/L galactose+0.3 mM octanoic acid. Strains were grown for an additional 96 hours and then extracted in acetonitrile.

Results

HB1629, HB1630, HB1631, HB1632 were transformed with plasmids expressing either PKS(1-76) or an RFP negative were grown in the presence of 0.3 mM octanoic acid. C7-alkylresorcyclic acid produced by these strains is shown in FIG. 25 and Table 41. FIG. 25 shows the octavic acid produced by strains in Example 8.

TABLE 41
Octavic acid produced by strains in Example 8

| Sample | Octavic acid average peak area |
|---|---|
| HB1629 PKS13 | 14032.26184 |
| HB1629 PKS14 | 10585.66787 |
| HB1629 PKS45 | 12065.22438 |
| HB1629 PKS47 | 4928.055321 |
| HB1629 PKS49 | 14412.86157 |
| HB1629 PKS65 | 16777.29025 |
| HB1629 PKS73 | 22888.1585 |
| HB1630 PKS45 | 17342.71935 |
| HB1631 PKS14 | 17661.76765 |
| HB1631 PKS45 | 10364.85643 |
| HB1631 PKS65 | 13347.65607 |
| HB1632 PKS45 | 17692.8092 |
| HB1632 PKS49 | 14371.08001 |
| HB1632 PKS65 | 17148.85877 |
| HB1632 PKS73 | 19542.02565 |
| HB1629-ve | 0 |
| HB1630-ve | 0 |
| HB1631-ve | 0 |
| HB1632-ve | 0 |

### Example 9

In Vivo Production of C5-Alkynyl Cannabigerolic Acid

In this Example, in vivo production of C5-alkynyl cannabigerolic acid. Here we show that co-expressing our type III PKS library with CSAAE1, PC20, PT72/254/273 and feeding hexynoic acid results in the production of C5-alkynyl cannabigerolic acid. These data illustrate that a wide variety of molecules can be produced.

Strain Growth and Media. Strains were grown in 500 ul pre-cultures for 48 hours in a 96 well plate. The preculture media consisted of yeast minimal media with a composition of 1.7 g/L YNB without ammonium sulfate+1.96 g/L URA dropout amino acid supplements+0.375 g/L monosodium glutamate and 10 g/L glucose. After 48 hours 50 ul of culture was transferred to a fresh 96 well plate containing 450 ul of culture media culture consisting of 1.7 g/L YNB without ammonium sulfate+1.96 g/L URA dropout amino acid supplements+1.5 g/L monosodium glutamate, 20 g/L raffinose and 20 g/L galactose+1 mM hexynoic acid. Strains were grown for an additional 96 hours and then extracted in acetonitrile.

Results

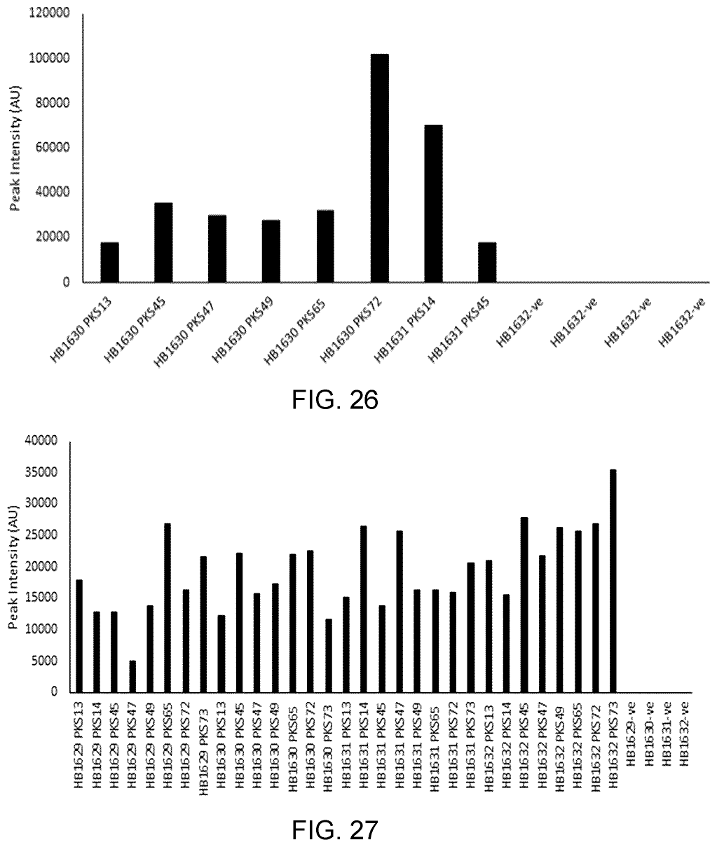

HB1629, HB1630, HB1631, HB1632 were transformed with plasmids expressing either PKS(1-76) or an RFP negative were grown in the presence of 1 mM hexynoic acid. C-alkynyl cannabigerolic acid produced by these strains is shown in FIG. 26 and Table 42.

FIG. 26 shows C5-alkynyl cannabigerolic acid peak area produced by strains in Example 9.

TABLE 4442
C5-alkynyl cannabigerolic peak area
produced by strains in Example 9

| Sample | C5-alkynyl cannabigerolic acid (AU) |
|---|---|
| HB1630 PKS13 | 17816.59 |
| HB1630 PKS45 | 35389.59 |
| HB1630 PKS47 | 29788.67 |
| HB1630 PKS49 | 27621.36 |
| HB1630 PKS65 | 32076.54 |
| HB1630 PKS72 | 101523.4 |
| HB1631 PKS14 | 70359.28 |
| HB1631 PKS45 | 17829.34 |
| HB1630-ve | 0 |
| HB1631-ve | 0 |

### Example 10

In Vivo Production of C5-Alkenyl Cannabicierolic Acid

In this Example, in vivo production of C5-alkenyl cannabigerolic acid. Here we show that co-expressing our type III PKS library with CSAAE1, PC20, PT72/254/273 and feeding hexenoic acid results in the production of C5-alkenyl cannabigerolic acid. These data serve to illustrate the wide variety of molecules that can be produced.

Strain Growth and Media. Strains were grown in 500 ul pre-cultures for 48 hours in a 96 well plate. The preculture media consisted of yeast minimal media with a composition of 1.7 g/L YNB without ammonium sulfate+1.96 g/L URA dropout amino acid supplements+0.375 g/L monosodium glutamate, 10 g/L glucose. After 48 hours 50 ul of culture was transferred to a fresh 96 well plate containing 450 ul of culture media culture consisting of 1.7 g/L YNB without ammonium sulfate+1.96 g/L URA dropout amino acid supplements+1.5 g/L monosodium glutamate, 20 g/L raffinose and 20 g/L galactose+1 mM hexenoic acid. Strains were grown for an additional 96 hours and then extracted in acetonitrile.

Results

HB1629, HB1630, HB1631, HB1632 was transformed with plasmids expressing either PKS(1-76) or an RFP negative were grown in the presence of 1 mM hexenoic acid. 05-alkenyl cannabigerolic acid produced by these strains is shown in FIG. 27 and Table 43.

FIG. 27 shows C5-alkenyl cannabigerolic acid made by strains in Example 10.

TABLE 43
C5-alkenyl cannabigerolic acid produced by strains in Example 10

| | C5- alkenyl cannabigerolic |
|---|---|
| Sample | acid peak area (AU) |
| HB1629 PKS13 | 17836.53 |
| HB1629 PKS14 | 12757.36 |
| HB1629 PKS45 | 12904.15 |
| HB1629 PKS47 | 5061.72 |
| HB1629 PKS49 | 13877.61 |
| HB1629 PKS65 | 26850.13 |
| HB1629 PKS72 | 16371.23 |
| HB1629 PKS73 | 21520.13 |
| HB1630 PKS13 | 12289.22 |
| HB1630 PKS45 | 22231.78 |
| HB1630 PKS47 | 15682.69 |
| HB1630 PKS49 | 17322.95 |
| HB1630 PKS65 | 21954.95 |
| HB1630 PKS72 | 22550.53 |
| HB1630 PKS73 | 11677.2 |
| HB1631 PKS13 | 15231.79 |
| HB1631 PKS14 | 26376.23 |
| HB1631 PKS45 | 13862.53 |
| HB1631 PKS47 | 25769.12 |
| HB1631 PKS49 | 16337.47 |
| HB1631 PKS65 | 16384.54 |
| HB1631 PKS72 | 15954.29 |
| HB1631 PKS73 | 20696.74 |
| HB1632 PKS13 | 21085.44 |
| HB1632 PKS14 | 15475.68 |
| HB1632 PKS45 | 27779.46 |
| HB1632 PKS47 | 21877.01 |
| HB1632 PKS49 | 26224.24 |
| HB1632 PKS65 | 25713.67 |
| HB1632 PKS72 | 26775.86 |
| HB1632 PKS73 | 35372.72 |
| HB1629-ve | 0 |
| HB1630-ve | 0 |
| HB1631-ve | 0 |
| HB1632-ve | 0 |

### Example 11

Overexpression of Additional Polyketide and Acyl-CoA Synthases in HB1775.

In this Example, overexpression of polyketide and acyl-CoA synthases in HB1775. In this example we transformed HB1775 with either an additional PKS (PKS80-109) or acyl-CoA synthase (Alk1-Alk30). HB1775 contains integrated copies of CSAAE1, PC20, PKS73, PT254, OXC155 and produces THCVa when fed with butyric acid. It is illustrated that overexpression of many of these enzymes in HB1775 results in an increase in THCVa titres vs the HB1775-RFP control.

Strain Growth and Media. Strains were grown in 500 ul pre-cultures for 48 hours in a 96 well plate. The preculture media consisted of yeast minimal media with a composition of 1.7 g/L YNB without ammonium sulfate+1.96 g/L URA dropout amino acid supplements+0.375 g/L monosodium glutamate and g/L glucose. After 48 hours 50 ul of culture was transferred to a fresh 96 well plate containing 450 ul of culture media culture consisting of 1.7 g/L YNB without ammonium sulfate+1.96 g/L URA dropout amino acid supplements+1.5 g/L monosodium glutamate, 20 g/L raffinose and 20 g/L galactose+5 mM butyric acid. Strains were grown for an additional 96 hours and then extracted in acetonitrile.

Results

HB1775 was transformed with either a PKS(PKS8Y-109), acyl-CoA synthase(Alk1-Alk30) or RFP. The resulting strains were grown in the presence of 5 mM butyric acid. Overexpression of many of these enzymes resulted in improved CBGVa and THCVa titres vs the control. Divarin, divarininic acid, CBGVa and THCVa titres for strains in this are shown below in Table 44.

Overexpressions for Alk24, Alk25, PKS84, PKS95, PKS13 PKS8, PKS88, PKS96 PKS104, PKS81, PKS89, PKS97, PKS105 are not listed in this data set.

TABLE 44
Divarin, divarininic acid, CBGVa and THCVa titres from strains in Example 11

| Gene | | Divarinic Acid | CBGVa | THCVa | More THCVa |
|---|---|---|---|---|---|
| Overexpression | Divarin(mg/L) | (mg/L) | (mg/L) | (mg/L) | than control? |
| HB1775-RFP | 1.41 | 1.413333 | 0 | 0.593333 | NA |
| HB1775-Alk27 | 2.796667 | 3.526667 | 0 | 1.45 | Yes |
| HB1775-PKS92 | 1.473333 | 1.656667 | 0 | 0.786667 | Yes |
| HB1775-PKS100 | 1.89 | 1.766667 | 0 | 0.776667 | Yes |
| HB1775-PKS108 | 1.276667 | 1.396667 | 0.206667 | 0.63 | Yes |
| HB1775-Alk20 | 1.843333 | 2.586667 | 0 | 1.096667 | Yes |
| HB1775-Alk28 | 2.236667 | 2.9 | 0 | 1.07 | Yes |
| HB1775-PKS85 | 1.606667 | 1.726667 | 0.2 | 0.816667 | Yes |
| HB1775-PKS93 | 1.183333 | 1.106667 | 0 | 0.536667 | No |
| HB1775-PKS101 | 1.756667 | 1.613333 | 0 | 0.876667 | Yes |
| HB1775-PKS109 | 1.21 | 1.243333 | 0 | 0.7 | Yes |
| HB1775-Alk21 | 1.59 | 1.62 | 0 | 0.776667 | Yes |
| HB1775-Alk29 | 1.333333 | 1.443333 | 0 | 0.826667 | Yes |
| HB1775-PKS86 | 1.726667 | 1.873333 | 0 | 1.07 | Yes |
| HB1775-PKS94 | 1.4 | 1.34 | 0 | 0.813333 | Yes |
| HB1775-PKS102 | 1.353333 | 1.286667 | 0.1 | 0.763333 | Yes |
| HB1775-Alk22 | 1.423333 | 1.483333 | 0 | 0.813333 | Yes |
| HB1775-Alk30 | 1.533333 | 1.7 | 0 | 0.73 | Yes |
| HB1775-PKS87 | 1.163333 | 1.26 | 0 | 0.456667 | No |
| HB1775-Alk25 | 1.796667 | 1.863333 | 0 | 0.906667 | Yes |
| HB1775-PKS82 | 1.636667 | 1.623333 | 0 | 0.926667 | Yes |
| HB1775-PKS90 | 1.73 | 1.893333 | 0 | 1.03 | Yes |
| HB1775-PKS98 | 1.356667 | 1.36 | 0 | 0.776667 | Yes |
| HB1775-PKS106 | 1.75 | 1.7 | 0 | 0.97 | Yes |
| HB1775-Alk26 | 3.103333 | 3.636667 | 0 | 1.33 | Yes |
| HB1775-PKS83 | 1.48 | 1.636667 | 0 | 0.8 | Yes |
| HB1775-PKS91 | 1.34 | 1.196667 | 0.17 | 0.51 | No |
| HB1775-PKS99 | 1.576667 | 1.64 | 0 | 0.856667 | Yes |
| HB1775-PKS107 | 1.53 | 1.59 | 0 | 0.883333 | Yes |
| HB1775-PKS73 | 3.506667 | 3.523333 | 0 | 1.653333 | Yes |
| HB1775-Alk1 | 1.563333 | 1.54 | 0.1 | 0.766667 | Yes |
| HB1775-Alk2 | 1.866667 | 2.086667 | 1.143333 | 0.85 | Yes |
| HB1775-Alk3 | 1.886667 | 2.063333 | 0 | 1.09 | Yes |
| HB1775-Alk4 | 1.97 | 2.09 | 0.843333 | 0.873333 | Yes |
| HB1775-Alk5 | 1.636667 | 1.61 | 0 | 0.89 | Yes |
| HB1775-Alk6 | 2.096667 | 2.29 | 0.93 | 1.053333 | Yes |
| HB1775-Alk7 | 2.34 | 2.746667 | 0.725 | 1.12 | Yes |
| HB1775-Alk8 | 1.756667 | 1.776667 | 0.11 | 1.04 | Yes |
| HB1775-Alk9 | 0.933333 | 1.086667 | 0 | 0.376667 | No |
| HB1775-Alk10 | 0.77 | 0.766667 | 0 | 0.35 | No |
| HB1775-Alk11 | 0.72 | 0.75 | 0.145 | 0.233333 | No |
| HB1775-Alk12 | 0.7 | 0.693333 | 0.22 | 0.26 | No |
| HB1775-Alk13 | 0.693333 | 0.943333 | 0.1 | 0.213333 | No |
| HB1775-Alk14 | 1.096667 | 1.363333 | 0 | 0.513333 | No |
| HB1775-Alk15 | 0.726667 | 0.61 | 0 | 0.063333 | No |
| HB1775-Alk16 | 0.866667 | 0.923333 | 0.2 | 0.36 | No |
| HB1775-Alk17 | 0.963333 | 1.25 | 0.14 | 0.33 | No |
| HB1775-Alk18 | 1.14 | 1.433333 | 0 | 0.5 | No |
| HB1775-Alk19 | 1.01333333 | 1.32333333 | 0 | 0.47 | No |

Part 4

Dictyostelium discoideum Polyketide Synthase (DiPKS), Olivetolic Acid Cyclase (OAC), Prenyltransferases, and Mutants Thereof for Production of Phytocannabinoids

The present disclosure relates generally to methods of production of phytocannabinoids in a host cell involving Dictyostelium discoideum polyketide synthase (DiPKS), olivetolic acid cyclase (OAC), prenyltransferases, and/or mutants of these.

Overview

It is an object of the present disclosure to obviate or mitigate at least one disadvantage of previous approaches to producing phytocannabinoids in a host cell, and of previous approaches to producing phytocannabinoid analogues.

In a first aspect, herein provided is a method and cell line for producing polyketides in recombinants organisms. The method applies, and the cell line includes, a host cell transformed with a polyketide synthase CDS, an olivetolic acid cyclase CDS and a prenyltransferase CDS. The polyketide synthase and the olivetolic acid cyclase catalyze synthesis of olivetolic acid from malonyl CoA. The olivetolic acid cyclase may include Cannabis sativa OAC. The polyketide synthase may include Dictyostelium discoideum polyketide synthase with a G1516R substitution. The prenyltransferase catalyzes synthesis of cannabigerolic acid or a cannabigerolic acid analogue, and may include PT254 from C. sativa. The host cell may include a tetrahydrocannabinolic acid synthase CDS, and the corresponding tetrahydrocannabinolic acid synthase catalyzes synthesis of A9-tetrahydrocannabinolic acid from cannabigerolic acid. The host cell may include a yeast cell, a bacterial cell, a protest cell or a plant cell.

A method of producing phytocannabinoids or phytocannabinoid analogues is described, comprising: providing a host cell comprising a first polynucleotide coding for a polyketide synthase enzyme, a second polynucleotide coding for an olivetolic acid cyclase enzyme and a third polynucleotide coding for a prenyltransferase enzyme and propagating the host cell for providing a host cell culture. The polyketide synthase enzyme and the olivetolic acid cyclase enzyme are for producing at least one precursor chemical from malonyl-CoA, the at least one precursor chemical according to formula 4-I:

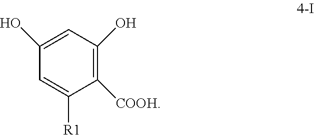

On formula 4-1, R1 is an alkyl group with a chain length of 1, 2, 3, 4, 5, 6, 7, 8, 16 or 18 carbons. The prenyltransferase enzyme is for prenylating the at least one precursor chemical with a prenyl group, providing at least one species of phytocannabinoid or phytocannabinoid analogue. The prenyl group is selected from the group consisting of dimethylallyl pyrophostphate, isopentenyl pyrophosphate, geranyl pyrophosphate, neryl pyrophosphate, farnesyl pyrophosphate and any isomer of the foregoing.

The at least one species of phytocannabinoid or phytocannabinoid analogue may have a structure according to formula 4-II:

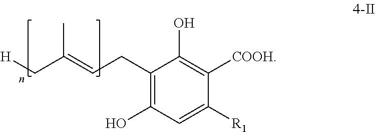

On formula 4-II, R1 is an alkyl group with a chain length of 1, 2, 3, 4, 5, 6, 7, 8, 16 or 18 carbons, and n is an integer with a value of 1, 2 or 3. The method involves propagating the host cell for providing a host cell culture capable of producing phytocannabinoids or analogues thereof.

An expression vector is described, comprising a first polynucleotide coding for a polyketide synthase enzyme; a second polynucleotide coding for an olivetolic acid cyclase enzyme; and a third polynucleotide coding for a prenyltransferase enzyme.

Further, a host cell is described for producing phytocannabinoids or analogues thereof, wherein the cell comprises a first polynucleotide coding for a polyketide synthase enzyme; a second polynucleotide coding for an olivetolic acid cyclase enzyme; and a third polynucleotide coding for a prenyltransferase enzyme.

A method of transforming a host cell for production of phytocannabinoids or phytocannabinoid analogues is also described. The method comprises introducing a first polynucleotide coding for a polyketide synthase enzyme into the host cell line; introducing a second polynucleotide coding for an olivetolic acid cyclase enzyme into the host cell; and introducing a third polynucleotide coding for a prenyltransferase enzyme into the host cell.

### Detailed Description of Part 4

Generally, the present disclosure provides methods and yeast cell lines for producing phytocannabinoids that are naturally biosynthesized in the Cannabis sativa plant and phytocannabinoid analogues with differing side chain lengths. The phytocannabinoids and phytocannabinoid analogues are produced in transgenic yeast. The methods and cell lines provided herein include application of genes for enzymes absent from the C. sativa plant. Application of genes other than the complete set of genes in the C. sativa plant that code for enzymes in the biosynthetic pathway resulting in phytocannabinoids may provide one or more benefits including biosynthesis of phytocannabinoid analogues, biosynthesis of phytocannabinoids without input of hexanoic acid, which is toxic to Saccharomyces cerevisiae and other species of yeast, and improved yield.

In a further aspect, herein provided is a method of producing phytocannabinoids or phytocannabinoid analogues, the method comprising: providing a host cell comprising a first polynucleotide coding for a polyketide synthase enzyme, a second polynucleotide coding for an olivetolic acid cyclase enzyme and a third polynucleotide coding for a prenyltransferase enzyme and propagating the host cell for providing a host cell culture. The polyketide synthase enzyme and the olivetolic acid cyclase enzyme are for producing at least one precursor chemical from malonyl-CoA, the at least one precursor chemical according to formula 4-I:

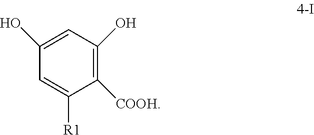

On formula 4-I, R1 is an alkyl group with a chain length of 1, 2, 3, 4, 5, 6, 7, 8, 16 or 18 carbons. The prenyltransferase enzyme is for prenylating the at least one precursor chemical with a prenyl group, providing at least one species of phytocannabinoid or phytocannabinoid analogue. The prenyl group is selected from the group consisting of dimethylallyl pyrophostphate, isopentenyl pyrophosphate, geranyl pyrophosphate, neryl pyrophosphate, farnesyl pyrophosphate and any isomer of the foregoing.

The at least one species of phytocannabinoid or phytocannabinoid analogue may have a structure according to formula 4-II:

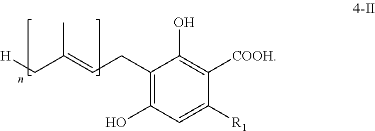

On formula 4-II, R1 is an alkyl group with a chain length of 1, 2, 3, 4, 5, 6, 7, 8, 16 or 18 carbons, and n is an integer with a value of 1, 2 or 3. The method involves propagating the host cell for providing a host cell culture capable of producing phytocannabinoids or analogues thereof.

In some embodiments, the polyketide synthase comprises a DiPKS^{G1516R} polyketide synthase enzyme, modified relative to DiPKS found from D. discoideum. In some embodiments, the first polynucleotide comprises a coding sequence for DiPKS^{G1516R} with a primary structure having between 80% and 100% amino acid residue sequence homology with a protein coded for by a reading frame defined by a coding sequence selected from the group consisting of bases 849 to 10292 of SEQ ID NO:427, bases 717 to 10160 of SEQ ID NO:428, bases 795 to 10238 of SEQ ID NO: 429, bases 794 to 10237 of SEQ ID NO:430, bases 1172 to 10615 of SEQ ID NO:431. In some embodiments, the first polynucleotide has between 80% and 100% base sequence homology with a reading frame defined by a coding sequence selected from the group consisting of bases 849 to 10292 of SEQ ID NO: 427, bases 717 to 10160 of SEQ ID NO: 428, bases 795 to 10238 of SEQ ID NO: 429, bases 794 to 10237 of SEQ ID NO:430, bases 1172 to 10615 of SEQ ID NO:431. In some embodiments, the host cell comprises a phosphopantetheinyl transferase polynucleotide coding for a phosphopantetheinyl transferase enzyme for increasing the activity of DiPKS^{G1516R}

In some embodiments, the phosphopantetheinyl transferase comprises NpgA phosphopantetheinyl transferase enzyme from A. nidulans. In some embodiments, the at least one precursor chemical comprises olivetolic acid, with a pentyl group at R1 and the at least one species of phytocannabinoid or phytocannabinoid analogue comprises a pentyl-phytocannabinoid. In some embodiments, the olivetolic acid cyclase enzyme comprises csOAC from C. sativa. In some embodiments, the second polynucleotide comprises a coding sequence for csOAC with a primary structure having between 80% and 100% amino acid residue sequence homology with a protein coded for by a reading frame defined by bases 842 to 1150 of SEQ ID NO: 415. In some embodiments, the second polynucleotide has between 80% and 100% base sequence homology with bases 842 to 1150 of SEQ ID NO: 415.

In some embodiments, the third polynucleotide codes for prenyltransferase enzyme PT254 from Cannabis sativa. In some embodiments, the third polynucleotide comprises a coding sequence for PT254 with a primary structure having between 80% and 100% amino acid residue sequence homology with a protein coded for by a reading frame defined by bases 1162 to 2133 of SEQ ID NO: 416. In some embodiments, the third polynucleotide has between 80% and 100% base sequence homology with bases 1162 to 2133 of SEQ ID NO:416.

In some embodiments, the third polynucleotide comprises a coding sequence for PT254^{R2S} with a primary structure having between 80% and 100% amino acid residue sequence homology with a protein coded for by a reading frame defined by bases 1162 to 2133 of SEQ ID NO: 417. In some embodiments, the third polynucleotide has between 80% and 100% base sequence homology with bases 1162 to 2133 of SEQ ID NO: 417.

In some embodiments, the method includes a downstream phytocannabinoid polynucleotide including a coding sequence for THCa synthase from C. sativa. In some embodiments, the downstream phytocannabinoid polynucleotide includes a coding sequence for THCa synthase with a primary structure having between 80% and 100% amino acid residue sequence homology with a protein coded for by a reading frame defined by bases 587 to 2140 of SEQ ID NO: 425.

In some embodiments, the downstream phytocannabinoid polynucleotide has between 80% and 100% base sequence homology with bases 587 to 2140 of SEQ ID NO: 425. In some embodiments, the host cell comprises a genetic modification to increase available geranylpyrophosphate. In some embodiments, the genetic modification comprises a partial inactivation of the farnesyl synthase functionality of the Erg20 enzyme.

In some embodiments, the host cell comprises an Erg20^{K197E} polynucleotide including a coding sequence for Erg20^{K197E}. In some embodiments, the host cell comprises a genetic modification to increase available malonyl-CoA. In some embodiments, the host cell comprises a yeast cell and the genetic modification comprises increased expression of Maf1. In some embodiments, the genetic modification comprises a modification for increasing cytosolic expression of an aldehyde dehydrogenase and an acetyl-CoA synthase.

In some embodiments, the host cell comprises a yeast cell and the genetic modification comprises a modification for expressing for Acs^{L641P} from S. enterica and Ald6 from S. cerevisiae. In some embodiments, the genetic modification comprises a modification for increasing malonyl-CoA synthase activity. In some embodiments, the host cell comprises a yeast cell and the genetic modification comprises a modification for expressing Acc1^{S659A; S1157A} from S. cerevisiae. In some embodiments, the host cell comprises a yeast cell comprising an Acc1 polynucleotide including the coding sequence for Acc1 from S. cerevisiae under regulation of a constitutive promoter. In some embodiments, the constitutive promoter comprises a PGK1 promoter from S. cerevisiae.

The host cell can be a bacterial cell, a fungal cell, a protist cell, or a plant cell, such as any of the exemplary cell types noted herein in Table 2. Exemplary host cell types include S. cerevisiae, E. coli, Yarrowia lipolytica, and Komagataella phaffii.

In some embodiments, the method includes extracting the at least one species of phytocannabinoid or phytocannabinoid analogue from the host cell culture.

In a further aspect, herein provided is a host cell for producing phytocannabinoids or phytocannabinoid analogues, the host cell comprising: a first polynucleotide coding for a polyketide synthase enzyme; a second polynucleotide coding for an olivetolic acid cyclase enzyme; and a third polynucleotide coding for a prenyltransferase enzyme.

In some embodiments, the host cell includes features of one or more of the host cell, the first polynucleotide, the second polynucleotide, the third nucleotide, the Erg20^{K197E} polynucleotide, the Acc1 polynucleotide, or the downstream phytocannabinoid polynucleotide as described in relation to the method of producing phytocannabinoids or phytocannabinoid analogues above.

In a further aspect, herein provided is a method of transforming a host cell for production of phytocannabinoids or phytocannabinoid analogues, the method comprising: introducing a first polynucleotide coding for a polyketide synthase enzyme into the host cell line; introducing a second polynucleotide coding for an olivetolic acid cyclase enzyme into the host cell; and introducing a third polynucleotide coding for a prenyltransferase enzyme into the host cell.

In some embodiments, the method includes application of a host cell including the features of one or more of the host cell, the first polynucleotide, the second polynucleotide, the third nucleotide, the Erg20^{K197E} polynucleotide, the Acc1 polynucleotide, or the downstream phytocannabinoid polynucleotide as described in relation to the method of producing phytocannabinoids or phytocannabinoid analogues above.

Many of the 120 phytocannabinoids found in Cannabis sativa may be synthesized in a host cell, and it may be desirable to improve production in host cells. Similarly, an approach that allows for production of phytocannabinoid analogues without the need for labour-intensive chemical synthesis may be desirable.

In C. sativa, a type 3 polyketide synthase called olivetolic acid synthase (“csOAS”) catalyzes synthesis of olivetolic acid from hexanoyl-CoA and malonyl-CoA in the presence of olivetolic acid cyclase (“csOAC”). Both csOAS and csOAC have been previously characterised as part of the C. sativa phytocannabinoid biosynthesis pathway (Gagne et al., 2012).

In C. sativa, a prenyltransferase enzyme catalyzes synthesis of cannabigerolic acid (“CBGa”) from olivetolic acid and geranyl pyrophosphate (“GPP”). One of the prenyltransferase enzymes identified in C. sativa is called d76csPT4 “PT254”. PT254 is a membrane bound enzyme with demonstrated high turnover for converting olivetolic acid to CBGa in the presence of GPP (Luo et al., 2019).

Polyketide synthase enzymes are present across all kingdoms. Dictyostelium discoideum is a species of slime mold that expresses a polyketide synthase called “DiPKS”. Wild type DiPKS is a fusion protein consisting of both a type I fatty acid synthase (“FAS”) and a polyketide synthase, and is referred to as a hybrid “FAS-PKS” protein. Wild-type DiPKS catalyzes synthesis of 4-methyl-5-pentylbenzene-1,3 diol (“MPBD”) from malonyl-CoA. The reaction has a 6:1 stoichiometric ratio of malonyl-CoA to MPBD.

A mutant form of DiPKS in which glycine 1516 is replaced by arginine (“DiPKS^{G1516R}”) disrupts a methylation moiety of DiPKS. DiPKS^{G1516R} does not synthesize MPBD. In the presence of malonyl-CoA from a glucose source, DiPKS^{G1516R} catalyzes synthesis of only olivetol, and not MPBD (Mookerjee et al., 2018 #1; Mookerjee et al., 2018 #2).

NpgA is a 4′-phosphopantethienyl transferase from Aspergillus nidulans. Expression of NpgA alongside DiPKS provides the A. nidulans phosphopantetheinyl transferase for greater catalysis of loading the phosphopantetheine group onto the ACP domain of DiPKS. NpgA also supports catalysis by DiPKS^{G1516R}

The methods and cells lines provided herein may apply and include transgenic Saccharomyces cerevisiae that have been transformed with nucleotide sequences coding for DiPKS^{G1516R}, NpgA, csOAC and PT254. Co-expression of DiPKS^{G1516R}, NpgA and csOAC in S. cerevisiae resulted in production of olivetolic acid in vivo from galactose. Co-expression of DiPKS^{G1516R}, NpgA, csOAC and PT254 in S. cerevisiae resulted in production of CBGa in vivo from galactose. Co-expression of DiPKS^{G1516R}, NpgA, csOAC, PT254 and A9-tetrahydrocannabinolic acid synthase (“THCa Synthase”) in S. cerevisiae resulted in production of A9-tetrahydrocannabinolic acid (“THCa”) in vivo from galactose.

Use of DiPKS^{G1516R} may provide advantages over csOAS for expression in S. cerevisiae to catalyze synthesis of olivetolic acid. csOAS catalyzes synthesis of olivetol from malonyl-CoA and hexanoyl-CoA. The reaction has a 3:1:1 stoichiometric ratio of malonyl-CoA to hexanoyl-CoA to olivetol. Olivetol synthesized during this reaction is carboxylated when the reaction is completed in the presence of csOAC, resulting in olivetolic acid. Hexanoic acid is toxic to S. cerevisiae. When applying csOAS and csOAC, hexanoyl-CoA is a necessary precursor for synthesis of olivetolic acid and the presence of hexanoic acid may inhibit proliferation of S. cerevisiae. When using DiPKS^{G1516R} and csOAC to produce olivetolic acid rather than csOAS and csOAC, the hexanoic acid need not be added to the growth media. The absence of hexanoic acid in growth media may result in increased growth of the S. cerevisiae cultures and greater yield of olivetolic acid compared with S. cerevisiae cultures fed with csOAS.

The S. cerevisiae may have one or more mutations in Erg20, Maf1 or other genes for enzymes or other proteins that support metabolic pathways that deplete GPP, the one or more mutations being for increasing available malonyl-CoA, GPP or both. Alternatively to S. cerevisiae, other species of yeast, including Yarrowia lipolytica, Kluyveromyces marxianus, Kluyveromyces lactis, Rhodosporidium toruloides, Cryptococcus curvatus, Trichosporon pullulan and Lipomyces lipoferetc, may be applied.

Synthesis of olivetolic acid may be facilitated by increased levels of malonyl-CoA in the cytosol. The S. cerevisiae may have overexpression of native acetaldehyde dehydrogenase and expression of a mutant acetyl-CoA synthase or other gene, the mutations resulting in lowered mitochondrial acetaldehyde catabolism. Lowering mitochondrial acetaldehyde catabolism by diverting the acetaldehyde into acetyl-CoA production increases malonyl-CoA available for synthesizing olivetol. Acc1 is the native yeast malonyl CoA synthase. The S. cerevisiae may have over-expression of Acc1 or modification of Acc1 for increased activity and increased available malonyl-CoA. The S. cerevisiae may include modified expression of Maf1 or other regulators of tRNA biosynthesis. Overexpressing native Maf1 has been shown to reduce loss of isopentenyl pyrophosphate (“IPP”) to tRNA biosynthesis and thereby improve monoterpene yields in yeast. IPP is an intermediate in the mevalonate pathway.

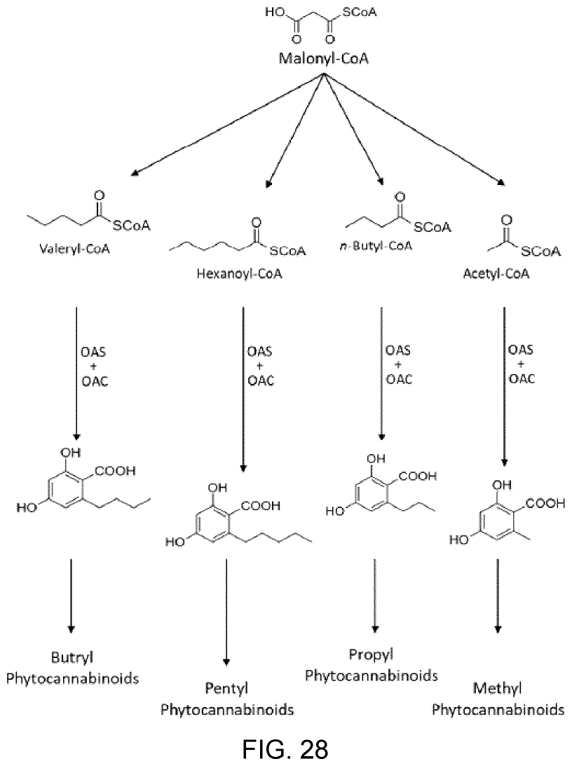

FIG. 28 shows biosynthesis of olivetolic acid from polyketide condensation products of malonyl-CoA and hexanoyl-CoA, as occurs in C. sativa. Olivetolic acid is a metabolic precursor to cannabigerolic acid (“CBGa”). CBGa is a precursor to a large number of downstream phytocannabinoids as described in further detail below. In most varieties of C. sativa, the majority of phytocannabinoids are pentyl-cannabinoids, which are biosynthesized from olivetolic acid, which is in turn synthesized from malonyl-CoA and hexanoyl-CoA at a 3:1 stoichiometric ratio. Some propyl-cannabinoids are observed, and are often identified with a “v” suffix in the widely-used three letter abbreviations (e.g. tetrahydrocannabivarin is commonly referred to as “THCv”, cannabidivarin is commonly referred to as “CBDv”, etc.). Tetrahydrocannabivarin acid may be referred to herein as “THCVa”. FIG. 28 also shows biosynthesis of divarinolic acid from condensation of malonyl-CoA with n-butyl-CoA, which would provide downstream propyl-phytocannabinoids.

FIG. 28 also shows biosynthesis of orsellinic acid from condensation of malonyl-CoA with acetyl-CoA, which would provide downstream methyl-phytocannabinoids. The term “methyl-phytocannabinoids” in this context means their alkyl side chain is a methyl group, where most phytocannabinoids have a pentyl group on the alkyl side chain and varinnic phytocannabinoids have a propyl group on the alkyl side chain.

FIG. 28 also shows biosynthesis of 2,4-diol-6-propylbenzenoic acid from condensation of malonyl-CoA with valeryl-CoA, which would provide downstream butyl-phytocannabinoids.

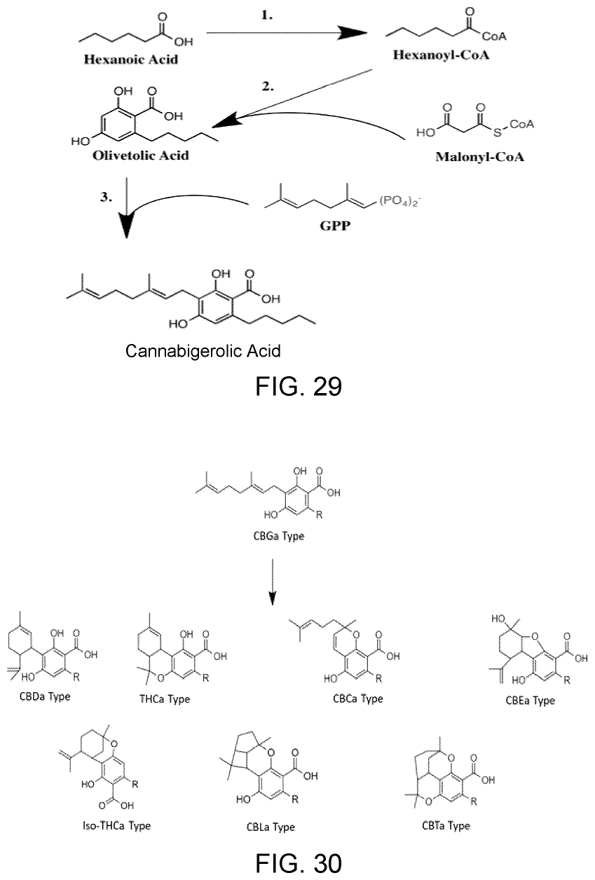

FIG. 29 shows biosynthesis of CBGa from hexanoic acid, malonyl-CoA, and GPP in C. sativa, including the olivetolic acid biosynthesis step shown in FIG. 28. Hexanoic acid is activated with coenzyme A by hexanoyl-CoA synthase (“Hex1; Reaction 1 in FIG. 29). In C. sativa, a type 3 polyketide synthase called olivetolic acid synthase (“csOAS”) and olivetolic acid cyclase (“csOAC”) together catalyze production of olivetolic acid from hexanoyl CoA and malonyl-CoA (Reaction 2 in FIG. 29). Prenyltransferase combines olivetolic acid with GPP, resulting in CBGa (Reaction 3 in FIG. 29).

FIG. 30 shows biosynthesis of downstream acid forms of phytocannabinoids in C. sativa from CBGa. CBGa is oxidatively cyclized into A9-tetrahydrocannabinolic acid (“THCa”) by THCa synthase. CBGa is oxidatively cyclized into cannabidiolic acid (“CBDa”) by CBDa synthase. Other phytocannabinoids are also synthesized in C. sativa, such as cannabichromenic acid (“CBCa”), cannabielsoinic acid (“CBEa”), iso-tetrahydrocannabinolic acid (“iso-THCa”), cannabicyclolic acid (“CBLa”), or cannabicitrannic acid (“CBTa”) by other synthase enzymes, or by changing conditions in the plant cells in a way that affects the enzymatic activity in terms of the resulting phytocannabinoid structure. The acid forms of each of these general phytocannabinoid types are shown in FIG. 30 with a general “R” group to show the alkyl side chain, which would be a 5-carbon chain where olivetolic acid is synthesized from hexanoyl-CoA and malonyl-CoA. In some cases, the carboxyl group is alternatively found on a ring position opposite the R group from the position shown in FIG. 30 (e.g. position 4 of A9-tetrahydrocannabinol (“THC”) rather than position 2 as shown in FIG. 30, etc.).

csOAS uses hexanoyl-CoA as a polyketide substrate. Hexanoic acid is toxic to S. cerevisiae and some other strains of yeast. In addition, synthesis of CBGa from olivetolic acid by the canonical membrane-bound C. sativa prenyltransferase enzyme.

Another prenyltransferase enzymes identified in C. sativa (“PT254”) may also be applied in yeast-based synthesis.

Methods and yeast cells as provided herein for production of phytocannabinoids and phytocannabinoid analogues may apply and include S. cerevisiae transformed with a gene for prenyltransferase PT254 from C. sativa.

Conversion of malonyl-CoA and hexanoyl-CoA to olivetolic acid catalyzed by csOAS at Reaction 2 of FIG. 29 was identified as a metabolic bottleneck in the pathway of FIG. 29. In order to increase yield at Reaction 2 of FIG. 29, multiple enzymes were functionally screened and one enzyme, a polyketide synthase from Dictyostelium discoideum called “DiPKS” was identified that could produce 4-methyl-5-pentylbenzene-1,3 diol (“MPBD”) directly from malonyl-CoA. A CDS for DiPKS is available at the NCBI GenBank online database under Accession Number NC_007087.3.

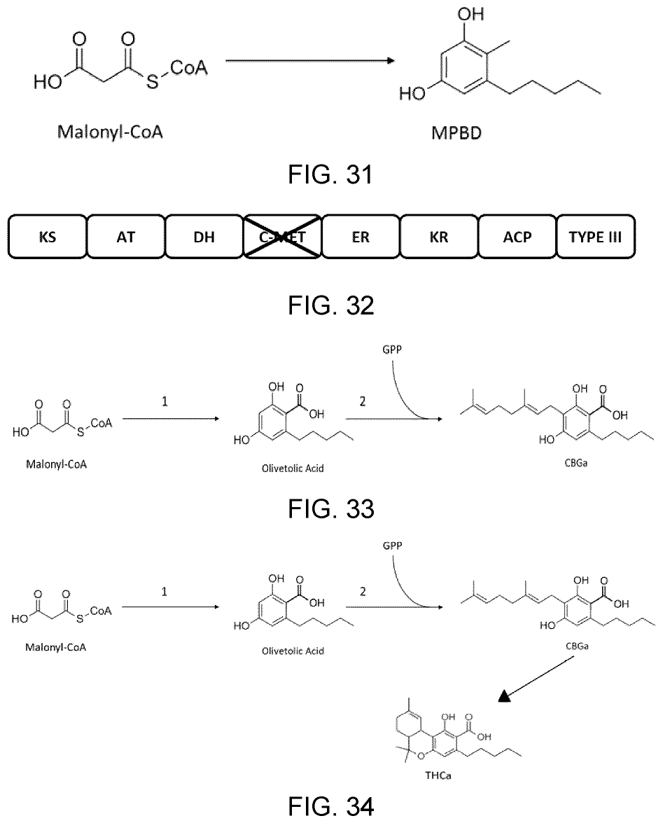

FIG. 31 shows production of MPBD from malonyl-CoA as catalyzed by DiPKS.

FIG. 32 is a schematic of the functional domains of DiPKS. DiPKS includes functional domains similar to domains found in a fatty acid synthase, and in additional includes a methyltransferase domain and a PKS III domain. FIG. 32 shows β-ketoacyl-synthase (“KS”), acyl transacetylase (“AT”), dehydratase (“DH”), C-methyl transferase (“C-Met”), enoyl reductase (“ER”), ketoreductase (“KR”), and acyl carrier protein (“ACP”). The “Type III” domain is a type 3 polyketide synthase. The KS, AT, DH, ER, KR, and ACP portions provide functions typically associated with a fatty acid synthase, speaking to DiPKS being a FAS-PKS protein in this case. The C-Met domain provides catalytic activity for methylating olivetol at carbon 4, providing MPBD.

The C-Met domain is crossed out in FIG. 32, schematically illustrating modifications to DiPKS protein that inactivate the C-Met domain and mitigate or eliminate methylation functionality. The Type III domain catalyzes iterative polyketide extension and cyclization of a hexanoic acid thioester transferred to the Type III domain from the ACP.

The C-Met domain of the DiPKS protein includes amino acid residues 1510 to 1633 of DiPKS. The C-Met domain includes three motifs. The first motif includes residues 1510 to 1518. The second motif includes residues 1596 to 1603. The third motif includes residues 1623 to 1633. Disruption of one or more of these three motifs may result in lowered activity at the C-Met domain. A mutant form of DiPKS in which glycine 1516 is replaced by arginine (“DiPKS^{G1516R}”) disrupts a methylation moiety of DiPKS. DiPKS^{G1516R} does not synthesize MPBD. In the presence of malonyl-CoA from a glucose or other sugar source, and in the absence of csOAC or another olivetolic acid cyclase or other polyketide cyclase, DiPKS^{G1516R} catalyzes synthesis of only olivetol, and not MPBD (Mookerjee et al., WO2018148848; Mookerjee et al. WO2018148849).

Application of DiPKS^{G1516R} rather than csOAS facilitates production of phytocannabinoids and phytocannabinoid analogues without hexanoic acid supplementation. Since hexanoic acid is toxic to S. cerevisiae, eliminating a requirement for hexanoic acid in the biosynthetic pathway for CBGa may provide greater yields of CBGa than the yields of CBGa in a yeast cell expressing csOAS and Hex1.

FIG. 33 is a schematic of biosynthesis of CBGa in a transformed yeast cell by DiPKS^{G1516R}, csOAC and PT254. DiPKS^{G1516R} and csOAC together catalyze reaction 1 in FIG. 33, resulting in olivetolic acid. PT254 catalyzes reaction 2, resulting in production of CBGa. Any downstream reactions to produce other phytocannabinoids or phytocannabinoid analogues would then correspondingly produce the same acid forms of the phytocannabinoids as would be produced in C. sativa or acid forms of phytocannabinoid analogues.

The N-end rule in protein degradation determines the half-life of a protein or other polypeptide as described in Varshavsky, A. (2011). The second residue in any polypeptide is recognized by the cell protein degradation machinery and flagged for degradation. The identity of the second amino acid has a demonstrated impact on the half-life of a polypeptide. It was observed that the second amino acid residue of PT254 was an arginine, which shortens the half-life in yeast relative to the half-life observed when the second residue is serine. Thus, this amino acid residue at position 2 of PT254 was changed to serine, resulting in “PT254^{R2S}”. The presence of the serine was hypothesized to increase the half-life of the protein which would result in greater substrate conversion and production of CBGa. As demonstrated by Example 14, PT254^{R2S} outperformed the wild type PT254.

FIG. 34 shows one example of a downstream phytocannabinoid being produced. In FIG. 34, the pathway of FIG. 33 is extended to include synthesis of THCa by THCa Synthase.

Transforming and Growing Yeast Cells

Details of specific examples of methods carried out and yeast cells produced in accordance with this description are provided below as Examples 12 to 14, below. Each of these three specific examples applied similar approaches to plasmid construction, transformation of yeast, quantification of strain growth, and quantification of intracellular metabolites. These common features across the three examples are described below, followed by results and other details relating to one or more of the examples.

As shown in Table 45, six strains of yeast were prepared. Base strain “HB742” is a uracil and leucine auxotroph CEN PK2 variant of S. cerevisiae with several genetic modifications to increase the availability of biosynthetic precursors and to increase DiPKS^{G1516R} activity. HB742 was prepared from a leucine and uracil auxotroph called “HB42”. In the “Genotype” column, the integration-based modifications are listed in the order they were introduced into the genome. Additional details are in Table 47. Strains “HB801” and “HB814” were based on HB742. Strains “HB861”, “HB862” were based on HB801. Strain HB888 was prepared based on HB814.

TABLE 45
Yeast Strains

| Strain | Background | Plasmids | Genotype | Notes |
|---|---|---|---|---|
| HB742 | -URA, -LEU | None | ΔLEU2 | Base Strain |
| | | | ΔURA3 | |
| | | | NPGA | |
| | | | DiPKS^{G1516R} X 5 | |
| | | | ALD6; ASC1^{L641p} | |
| | | | MAF1 | |
| | | | Erg20^{K197E}::KanMx | |
| | | | UB14p:ERG20 | |
| | | | tHMGR1; IDI | |
| | | | PGK1p:Acc1^{S659A; S1157A} | |
| HB801 | -URA, -LEU | None | (HB742) | Olivetolic acid producing |
| | | | Gal1p:csOAC | strain |
| HB861 | -URA, -LEU | None | (HB801) | CBGA producing strain |
| | | | Gal1p:PT254 | |
| HB862 | -URA, -LEU | None | (HB801) | CBGA producing strain |
| | | | Gal1p:PT254^{R2S} | |
| HB814 | -URA, -LEU | None | (HB742) | Produces neither |
| | | | Gal1p:PT254 | olivetolic acid nor CBGa |
| HB888 | -URA, -LEU | PLAS182 | (HB814) | THCA producing strain |
| | | PLAS251 | | |

Protein sequences and coding DNA sequences used to prepare the strains in Table 45 are provided below in Table 46 and full sequence listings are provided below.

TABLE 46
Protein and DNA Sequences used to Prepare the Yeast Strains

| SEQ ID NO | Description | DNA/Protein | Length | Coding Sequence |
|---|---|---|---|---|
| 412 | csOAC | Protein | 102 | Entire sequence |
| 413 | PT254 | Protein | 323 | Entire sequence |
| 414 | PT254^{R2S} | Protein | 323 | Entire sequence |
| 415 | Gal1p:csOAC:Eno2t | DNA | 2177 | 842 to 1150 |
| | expression/integration cassette | | | |
| 416 | Gal1p:PT254:Cyc1t | DNA | 3097 | 1162 to 2133 |
| | expression/integration cassette | | | |
| 417 | Gal1p:PT254_R2S:Cyc1t | DNA | 3095 | 1162 to 2133 |
| | expression/integration cassette | | | |
| 418 | PLAS182 | DNA | 4995 | 517 to 822 |
| 419 | PLAS251 | DNA | 7432 | 1 to 1626 |
| 420 | PLAS36 | DNA | 8980 | Not applicable |
| 421 | THCA_synthase_aa | Protein | 518 | Entire sequence |
| 422 | Backbone for pHygro | DNA | 3888 | Cassettes added |
| | (PLAS182) | | | before base-pair 1 of |
| | | | | sequence |
| 423 | Expression cassette for csOAC | DNA | 1093 | 511 to 816 |
| | in PLAS182. | | | |
| | Gal1p:csOAC:Cyc1t | | | |
| 424 | Backbone for pGAL (PLAS251) | DNA | 5058 | Cassettes added |
| | | | | before base-pair 1 of |
| | | | | sequence |
| 425 | Expression cassette for THCA | DNA | 2435 | 587 to 2140 |
| | Synthase in PLAS251. | | | |
| | Gal1p:THCA Synthase:Cyc1t | | | |
| 426 | NpgA | DNA | 3564 | 1170 to 2201 |
| 427 | DiPKS-1 | DNA | 11114 | 849 to 10292 |
| 428 | DiPKS-2 | DNA | 10890 | 717 to 10160 |
| 429 | DiPKS-3 | DNA | 11300 | 795 to 10238 |
| 430 | DiPKS-4 | DNA | 11140 | 794 to 10237 |
| 431 | DiPKS-5 | DNA | 11637 | 1172 to 10615 |
| 432 | PDH | DNA | 7114 | Ald6: 1444 to 2949 |
| | | | | ACS: 3888 to 5843 |
| 433 | Maf1 | DNA | 3256 | 936 to 2123 |
| 434 | Erg20K197E | DNA | 4254 | 2683 to 3423 |
| 435 | Erg1p:UB14-Erg20:deg | DNA | 3503 | 1364 to 2701 |
| 436 | tHMGr-IDI | DNA | 4843 | tHMGR1: |
| | | | | 877 to 2385 |
| | | | | IDI1: 3209 to 4075 |
| 437 | PGK1p:ACC1^{S659A, S1157A} | DNA | 7673 | Pgk1p: 222 to 971 |
| | | | | Acc1^{S659A, S1157A}: |
| | | | | 972 to 7673 |

Genome Modification of S. cerevisiae

HB42 was used as a base strain to develop HB742, and in turn all other strains in this experiment. All DNA was transformed into strains using the transformation protocol described in Gietz et al. (2007). Plas 36 was used for the genetic modifications described in this experiment that apply clustered regularly interspaced short palindromic repeats (CRISPR).

The genome of HB42 was iteratively targeted by gRNA's and Cas9 expressed from PLAS36 to make the following genomic modifications in the order of the Table 47 below. Erg20^{K197E} was already included in HB42 and is marked as being order “0”.

TABLE 47
Gene Integration in HB742

| Order | Modification | Integration | Description | Genetic Structure |
|---|---|---|---|---|
| 0 | Erg20^{K197E} | Chromosomal | Mutant of Erg20 protein that | Tpi1p:ERG20K197E: |
| | SEQ ID NO. 434 | modification | diminishes FPP synthase | Cyc1t::Tef1p:KanMX: |
| | | | activity creating greater pool | Tef1t |
| | | | of GPP precursor. | |
| | | | Negatively affects growth | |
| | | | phenotype. (Oswald et al., | |
| | | | 2007) | |
| 1 | PDH bypass | Flagfeldt Site | Acetaldehyde | 19Up::Tdh3p:Ald6: |
| | SEQ ID NO. 432 | 19 integration | dehydrogenase (ALD6) from | Adh1::Tef1p:seACS1^{L641p}: |
| | | | S. cerevisiae and | Prm9t::19Down |
| | | | acetoacetyl coA synthase | |
| | | | (AscL641 P) from Salmonella | |
| | | | enterica. Will allow greater | |
| | | | accumulation of acetyl-coA | |
| | | | in the cell. (Shiba et al., | |
| | | | 2007) | |
| 2 | NpgA | Flagfeldt Site | Phosphopantetheinyl | Site14Up::Tef1p: |
| | SEQ ID NO. 426 | 14 integration | Transferase from Aspergillus | NpgA:Prm9t:Site14Down |
| | | | niger. Accessory Protein for | |
| | | | DiPKS (Kim et al., 2007) | |
| 3 | Maf1 | Flagfeldt Site 5 | Maf1 is a regulator of tRNA | Site5Up::Tef1p: |
| | SEQ ID NO. 433 | integration | biosynthesis. | Maf1:Prm9t:Site5Down |
| | | | Overexpression in S. | |
| | | | cerevisiae has demonstrated | |
| | | | higher monoterpene (GPP) | |
| | | | yields (Liu et al., 2013) | |
| 4 | PGK1p: | Chromosomal | Mutations in the native S. | Pgk1:ACC1^{S659A, S1157A}: |
| | ACC1^{S659A, S1157A} | Modification | cerevisiae acetyl-coA | Acc1t |
| | SEQ ID NO. 437 | | carboxylase that removes | |
| | | | post-translational | |
| | | | modification based down- | |
| | | | regulation. Leads to greater | |
| | | | malonyl-coA pools. The | |
| | | | promoter of Acc1 was also | |
| | | | changed to a constitutive | |
| | | | promoter for higher | |
| | | | expression. (Shi et al., 2014) | |
| 5 | tHMGR-IDI1 | USER Site X-3 | Overexpression of truncated | X3up::Tdh3p:tHMGR1: |
| | SEQ ID NO. 436 | integration | HMGr1 and IDI1 proteins | Adh1t::Tef1p:IDI1: |
| | | | that have been previously | Prm9t::X3down |
| | | | identified to be bottlenecks | |
| | | | in the S. cerevisiae | |
| | | | terpenoid pathway | |
| | | | responsible for GPP | |
| | | | production. (Ro et al., 2006) | |
| 6 | DiPKS^{G1516R}-1 | USER Site XII- | Type 1 FAS fused to Type 3 | XII-1up::Gal1p: |
| | SEQ ID NO. 427 | 1 integration | PKS from D. discoideum. | DiPKSG1516R: |
| | | (Jensen et al., | Produces Olivetol from | Prm9t::XII1-down |
| | | no date) | malonyl-coA | |
| 7 | Erg1p:UB14- | Flagfeldt Site | Sterol responsive promoter | Site18Up::Erg1p: |
| | Erg20:deg | 18 integration | controlling Erg20 protein | UB14deg:ERG20: |
| | SEQ ID NO. 435 | | activity. Allows for regular | Adh1t:Site18down |
| | | | FPP synthase activity and | |
| | | | uninhibited growth | |
| | | | phenotype until | |
| | | | accumulation of sterols | |
| | | | which leads to a suppression | |
| | | | of expression of enzyme. | |
| | | | (Peng et al., 2018) | |
| 8 | DiPKS^{G1516R}-2 | Wu site 1 | Type 1 FAS fused to Type 3 | Wu1up::Gal1p: |
| | SEQ ID NO. 428 | integration | PKS from D. discoideum. | DiPKSG1516R: |
| | | | Produces Olivetol from | Prm9t::Wu1down |
| | | | malonyl-coA | |
| 9 | DiPKS^{G1516R}-3 | Wu site 3 | Type 1 FAS fused to Type 3 | Wu3up::Gal1p: |
| | SEQ ID NO. 429 | integration | PKS from D. discoideum. | DiPKSG1516R: |
| | | | Produces Olivetol from | Prm9t::Wu3down |
| | | | malonyl-coA | |
| 10 | DiPKS^{G1516R}-4 | Wu site 6 | Type 1 FAS fused to Type 3 | Wu6up::Gal1p: |
| | SEQ ID NO. 430 | integration | PKS from D. discoideum. | DiPKSG1516R: |
| | | | Produces Olivetol from | Prm9t::Wu6down |
| | | | malonyl-coA | |
| 11 | DiPKS^{G1516R}-5 | Wu site 18 | Type 1 FAS fused to Type 3 | Wu18up::Gal1p: |
| | SEQ ID NO. 431 | integration | PKS from D. discoideum. | DiPKSG1516R: |
| | | | Produces Olivetol from | Prm9t::Wu18down |
| | | | malonyl-coA | |

The S. cerevisiae strains described herein may be prepared by stable transformation of plasmids, genome integration or other genome modification. Genome modification may be accomplished through homologous recombination, including by methods leveraging CRISPR.

Methods applying CRISPR were applied to delete DNA from the S. cerevisiae genome and introduce heterologous DNA into the S. cerevisiae genome. Guide RNA (“gRNA”) sequences for targeting the Cas9 endonuclease to the desired locations on the S. cerevisiae genome were designed with Benchling online DNA editing software. DNA splicing by overlap extension (“SOEing”) and PCR were applied to assemble the gRNA sequences and amplify a DNA sequence including a functional gRNA cassette.

The functional gRNA cassette, a Cas9-expressing gene cassette, and the pYes2 (URA) plasmid were assembled into the PLAS36 plasmid and transformed into S. cerevisiae for facilitating targeted DNA double-stranded cleavage. The resulting DNA cleavage was repaired by the addition of a linear fragment of target DNA (“Donor DNA”).

Linear Donor DNA for introduction into S. cerevisiae were amplified by polymerase chain reaction (“PCR”) with primers from Operon Eurofins and Phusion HF polymerase (ThermoFisher F-530S) according to the manufacturer's recommended protocols using an Eppendorf Mastercycler ep Gradient 5341. Each genomic integration Donor DNA includes three DNA sequences amplified by PCR. The expression cassette includes part of the homology region of the genome, and is amplified by PCR from that homology region. The genomic homology regions are amplified from the genome with homology to the expression cassette added on by primers. Primers for PCR that amplify the expression cassette also add a homology tail, that adds to the genomic integration region.

Integration site homology sequences for integration into the S. cerevisiae genome using CRISPR may be at Flagfeldt sites. A description of Flagfeldt sites is provided in Bai Flagfeldt, et al., (2009). Other integration sites may be applied as indicated in Table 47.

Increasing Availability of Biosynthetic Precursors

The biosynthetic pathway shown in FIG. 33 and FIG. 34 each require malonyl-CoA and GPP to produce CBGa. Yeast cells may be mutated, genes from other species may be introduced, genes may be upregulated or downregulated, or the yeast cells may be otherwise genetically modified to increase production of olivetolic acid, CBGa or downstream phytocannabinoids. In addition to introduction of a polyketide synthase such as DiPKS^{G1516R}, an olivetolic acid cyclase such as csOAC, and a prenyltransferase such as PT254, additional modifications may be made to the yeast cell to increase the availability of malonyl-CoA, GPP, or other input metabolites to support the biosynthetic pathways of any of FIG. 33 and FIG. 34.

As shown in FIG. 32, DiPKS^{G1516R} includes an ACP domain. The ACP domain of DiPKS^{G1516R} requires a phosphopantetheine group as a co-factor. NpgA is a 4′-phosphopantethienyl transferase from Aspergillus nidulans. A codon-optimized copy of NpgA for S. cerevisiae may be introduced into S. cerevisiae and transformed into the S. cerevisiae, including by homologous recombination. In HB742, an NpgA gene cassette was integrated into the genome of Saccharomyces cerevisiae at Flagfeldt site 14.

Expression of NpgA provides the A. nidulans phosphopantetheinyl transferase for greater catalysis of loading the phosphopantetheine group onto the ACP domain of DiPKS^{G1516R}As a result, the reaction catalyzed by DiPKS^{G1516R} (reaction 1 in FIG. 33 and FIG. 34) may occur at greater rate, providing a greater amount of olivetolic acid for prenylation to CBGa. As shown in Table 45, HB742 includes an integrated polynucleotide including a coding sequence NpgA, as does each modified yeast strain based on HB742 (HB801, HB861, HB862, HB814 and H B888).

The sequence of the integrated DNA coding for NpgA is shown in SEQ ID NO: 426, and includes the Tef1 Promoter, the NpgA coding sequence and the Prm9 terminator. Together the Tef1p, NpgA, and Prm9t are flanked by genomic DNA sequences promoting integration into Flagfeldt site 14 in the S. cerevisiae genome.

SEQ ID NO: 427, SEQ ID NO:428, SEQ ID NO:429, SEQ ID NO:430 and SEQ ID NO:431 each include a copy of DiPKS^{G1516R} flanked by the Gall promoter, the Prm9 terminator, and integration sequences for the sites indicated in Table 47.

The yeast strains may be modified for increasing available malonyl-CoA. Lowered mitochondrial acetaldehyde catabolism results in diversion of the acetaldehyde from ethanol catabolism into acetyl-CoA production, which in turn drives production of malonyl-CoA and downstream polyketides and terpenoids. S. cerevisiae may be modified to express an acetyl-CoA synthase from Salmonella enterica with a substitution modification of Leucine to Proline at residue 641 (“ACS^{L641P}”), and with aldehyde dehydrogenase 6 from S. cerevisiae (“Ald6”). The Leu641 Pro mutation removes downstream regulation of Acs, providing greater activity with the Acs^{L641P} mutant than the wild type Acs. Together, cytosolic expression of these two enzymes increases the concentration of acetyl-CoA in the cytosol. Greater acetyl-CoA concentrations in the cytosol result in lowered mitochondrial catabolism, bypassing mitochondrial pyruvate dehydrogenase (“PDH”), providing a PDH bypass. As a result, more acetyl-CoA is available for malonyl-CoA production.

SEQ ID NO:432 includes coding sequences for the genes for Ald6 and SeAcsL641P, promoters, terminators, and integration site homology sequences for integration into the S. cerevisiae genome at Flagfeldt-site 19. As shown in Table 47 a portion of SEQ ID NO:432 from bases 1444 to 2949 codes for Ald6 under the TDH3 promoter, and bases 3888 to 5843 code for SeAcsL641P under the Tef1 P promoter.

S. cerevisiae may include modified expression of Maf1 or other regulators of tRNA biosynthesis. Overexpressing native Maf1 has been shown to reduce loss of IPP to tRNA biosynthesis and thereby improve monoterpene yields in yeast. IPP is an intermediate in the mevalonate pathway. As shown in Table 45, HB742 includes an integrated polynucleotide including a coding sequence for Maf1 under the Tef1 promoter, as does each modified yeast strain based on HB742 (H1B801, H B861, H B862, H B814 and H B888).

SEQ ID NO:433 is a polynucleotide that was integrated into the S. cerevisiae genome at Flagfeldt-site 5 for genomic integration of Maf1 under the Tef1 promoter. SEQ ID NO: 433 includes the Tef1 promoter, the native Maf1 gene, and the Prm9 terminator. Together, Tef1, Maf1, and Prm9 are flanked by genomic DNA sequences for promoting integration into the S. cerevisiae genome.

The yeast cells may be modified for increasing available GPP. S. cerevisiae may have one or more other mutations in Erg20 or other genes for enzymes that support metabolic pathways that deplete GPP. Erg20 catalyzes GPP production in the yeast cell. Erg20 also adds one subunit of 3-isopentyl pyrophosphate (“IPP”) to GPP, resulting in farnesyl pyrophosphate (“FPP”), a metabolite used in downstream sesquiterpene and sterol biosynthesis. Some mutations in Erg20 have been demonstrated to reduce conversion of GPP to FPP, increasing available GPP in the cell. A substitution mutation Lys197Glu in Erg20 lowers conversion of GPP to FPP by Erg20. As shown in Table 45, base strain HB742 expresses the Erg20^{K197E} mutant protein. Similarly, each modified yeast strain based on any of HB742, (HB801, HB861, HB862, HB814 and HB888) includes an integrated polynucleotide coding for the Erg20^{K197E} mutant integrated into the yeast genome.

SEQ ID NO:434 is a CDS coding for the Erg20^{K197E} protein under control of the Tpi1p promoter and the Cyc1t terminator, and a coding sequence for the KanMX protein under control of the Tef1p promoter and the Tef1t terminator.

SEQ ID NO:435 is a CDS coding for the Erg20 protein under control of the Erg1p promoter and the Adh1t terminator, and flanking sequences for homologous recombination. The Erg1 promoter is downregulated by the presence of large amounts of Ergosterol in the cell. When the cells are growing and there is not much ergosterol in the cell, the Erg1 promoter aids in the expression of the native Erg20 protein that allows the cells to grow without any growth defects associated with the attenuation of FPP synthase activity. When the cells have high amounts of ergosterol present in later stages of growth then the Erg1 promoter is inhibited leading to the cessation of expression of the native Erg20 protein. The extant copies of the native Erg20 protein in the cell are quickly degraded due to the UB14 degradation tag. This allows the mutant Erg20K197E to be functional leading to GPP accumulation.

SEQ ID NO:436 is a CDS coding for the truncated HMGr1 under control of the Tdh3p promoter and the Adh1t terminator, and the IDI1 protein under control of the Tef1p promoter and the Prm9t terminator, and flanking sequences for homologous recombination of both sequences for genome integration. The HMG1 protein catalyzed reduction and the IDI1 catalyzed isomerization have previously been identified as rate limiting steps in the eukaryotic mevalonic pathway. Thus, over-expression of these proteins have been demonstrated to alleviate the bottlenecks in the mevalonate pathway and increase the carbon flux for GPP and FPP production.

Another approach to increasing cytosolic malonyl-CoA is to upregulate Acc1, which is the native yeast malonyl-CoA synthase. In HB742, the promoter sequence of the Acc1 gene was replaced by a constitutive yeast promoter for the PGK1 gene. The promoter from the PGK1 gene allows multiple copies of Acc1 to be present in the cell. The native Acc1 promoter allows only a single copy of the protein to be present in the cell at a time. As shown in Table 45, base strain HB742 includes the Acc1 under the PGK1 promoter, as does each modified yeast strain based on HB742 (H1B801, H B861, H B862, H B814 and H B888).

In addition to upregulating expression of Acc1, S. cerevisiae may include one or more modifications of Acc1 to increase Acc1 activity and cytosolic acetyl-CoA concentrations. Two mutations in regulatory sequences were identified in literature that remove repression of Acc1, resulting in greater Acc1 expression and higher malonyl-CoA production. HB742 includes a coding sequence for the Acc1 gene with Ser659Ala and Ser1157Ala modifications flanked by the PGK1 promoter and the Acc1 terminator. As a result, the S. cerevisiae transformed with this sequence will express Acc1^{S659A; S1157A}. As shown in Table 45, base strain HB742 includes Acc1^{S659A; S1157A}, as does each modified yeast strain based on HB742 (HB801, HB861, HB862, HB814 and HB888).

SEQ ID NO:437 is a polynucleotide that may be used to modify the S. cerevisiae genome at the native Acc1 gene by homologous recombination. SEQ ID NO:437 includes a portion of the coding sequence for the Acc1 gene with Ser659Ala and Ser1167Ala modifications. A similar result may be achieved, for example, by integrating a sequence with the Tef1 promoter, the Acc1 with Ser659Ala and Ser1167Ala modifications, and the Prm9 terminator at any suitable site. The end result would be that Tef1, Acc1^{S659A; S1167A} and Prm9 are flanked by genomic DNA sequences for promoting integration into the S. cerevisiae genome.

Plasmid Construction

Plasmids synthesized to apply and prepare examples of the methods and yeast cells provided herein are shown in Table 48.

TABLE 48
Plasmids and Cassettes Used to Prepare Yeast Strains

| Plasmid Name | Description | Selection |
|---|---|---|
| PLAS182 | pDiddy_hygro_Gal1p-csOAC-Cyc1t | Hygromycin |
| PLAS251 | pGAL_ProA_THCaSynthase | Uracil |
| PLAS36 | pCAS_Hyg_Rnr2p:Cas9: | Hygromycin |
| | Cyc1t::tRNATyr:HDV:gRNA:Snr52t | |

The plasmids PLAS182, PLAS251 and PLAS36 were synthesized using services provided by Twist Bioscience Corporation

Stable Transformation for Strain Construction

Plasmids were transformed into S. cerevisiae using the lithium acetate heat shock method as described by Gietz, et al. (2007). S. cerevisiae HB888 was were prepared by transformation of HB814 with expression plasmids PLAS182 and PLAS251.

To create a stably transformed CBGa producing strain csOAC was first stably transformed. The genome at Flagfeldt position 16 in HB742 was targeted using Cas9 and gRNA expressed from PLAS36. The donor for the recombination was SEQ ID NO.415. Successful integrations were confirmed by colony polymerase chain reaction (“PCR”) and led to the creation of HB801 with a Galactose inducible csOAC encoding gene integrated into the genome of HB742. The genomic region containing SEQ ID NO.415 was also verified by sequencing to confirm the presence of the csOAC encoding gene.

HB801 was used to create HB861 and HB862 in a similar process. PLAS36 expressing the gRNA targeting Flagfeldt position 20 was transformed into strain HB801 along with the donors SEQ ID NO.416 and SEQ ID NO.417. Successful integrations were screened by colony PCR and verified by sequencing the genomic region containing the integrated DNA. All sequencing was performed by Eurofins Genomics. HB861 has SEQ ID NO. 416 integrated into the genome while HB862 has SEQ ID NO. 417 integrated into the genome.

HB742 was also used as the base strain to create a THCa producing strain HB888. PLAS36 expressing a gRNA targeting Flagfeldt position 20 and SEQ ID NO.416 were transformed into HB742 with the aim of integrating galactose inducible PT254 expressing gene into the genome. Successful integrations were screened by colony PCR and verified by sequencing the genomic region containing the integrated DNA. The integration of SEQ ID NO.416 into HB742 created strain HE 814. PLAS182 encodes a galactose inducible csOAC gene and PLAS251 encodes a galactose inducible THCa synthase with a proA tag fused to the N-terminal of the THCa synthase. These two plasmids, PLAS182 and PLAS25, were subsequently transformed into strain HB814 to produce strain HB888.

Yeast Growth and Feeding Conditions

Yeast cultures were grown in overnight cultures with selective media to provide starter cultures. The resulting starter cultures were then used to inoculate experimental replicate cultures to an optical density at having an absorption at 600 nm (“A₆₀₀”) of 0.1.

Table 49 shows the uracil drop out (“URADO”) amino acid supplements that are added to yeast synthetic dropout media supplement lacking leucine and uracil. “YNB” is a nutrient broth including the chemicals listed in the first two columns of Table 49. The chemicals listed in the third and fourth columns of Table 49 are included in the URADO supplement.

TABLE 49
YNB Nutrient Broth and URADO Supplement

| YNB | | | URADO Supplement | | |
|---|---|---|---|---|---|
| Chemical | Concentration | | Chemical | Concentration | |
| Ammonium Sulphate | 5 | g/L | Adenine | 18 | mg/L |
| Biotin | 2 | μg/L | p-Aminobenzoic acid | 8 | mg/L |
| Calcium pantothenate | 400 | μg/L | Alanine | 76 | mg/ml |
| Folic acid | 2 | μg/L | Arginine | 76 | mg/ml |
| Inositol | 2 | mg/L | Asparagine | 76 | mg/ml |
| Nicotinic acid | 400 | μg/L | Aspartic Acid | 76 | mg/ml |
| p-Aminobenzoic acid | 200 | μg/L | Cysteine | 76 | mg/ml |
| Pyridoxine HCl | 400 | μg/L | Glutamic Acid | 76 | mg/ml |
| Riboflavin | 200 | μg/L | Glutamine | 76 | mg/ml |
| Thiamine HCL | 400 | μg/L | Glycine | 76 | mg/ml |
| Citric acid | 0.1 | g/L | Histidine | 76 | mg/ml |
| Boric acid | 500 | μg/L | myo-Inositol | 76 | mg/ml |
| Copper sulfate | 40 | μg/L | Isoleucine | 76 | mg/ml |
| Potassium iodide | 100 | μg/L | Leucine | 152 | mg/ml |
| Ferric chloride | 200 | μg/L | Lysine | 76 | mg/ml |
| Magnesium sulfate | 400 | μg/L | Methionine | 76 | mg/ml |
| Sodium molybdate | 200 | μg/L | Phenylalanine | 76 | mg/ml |
| Zinc sulfate | 400 | μg/L | Proline | 76 | mg/ml |
| Potassium phosphate monobasic | 1.0 | g/L | Serine | 76 | mg/ml |
| Magnesium sulfate | 0.5 | g/L | Threonine | 76 | mg/ml |
| Sodium chloride | 0.1 | g/L | Tryptophan | 76 | mg/ml |
| Calcium chloride | 0.1 | g/L | Tyrosine | 76 | mg/ml |

| | | | | |
|---|---|---|---|---|
| (blank cell) | (blank cell) | Valine | 76 | mg/ml |

Quantification of Metabolites

Metabolite extraction was performed with 300 μl of Acetonitrile added to 100 μl culture in a new 96-well deepwell plate, followed by 30 min of agitation at 950 rpm. The solutions were then centrifuged at 3750 rpm for 5 min. 200 μl of the soluble layer was removed and stored in a 96-well v-bottom microtiter plate. Samples were stored at −20° C. until analysis.

Intracellular metabolites were quantified using high performance liquid chromatography (“HPLC”) and mass spectrometry (“MS”) methods. Quantification of olivetolic acid, CBGa and THCa was performed using HPLC-MS on an Acquity UPLC-TQD MS.

Quantification of CBGa and THCa was performed by HPLC on a Hypersil Gold PFP 100×2.1 mm column with a 1.9 μm particle size. Eluent A—0.1% formic acid in water. Eluent B—0.1% formic acid in acetonitrile. An isocratic mix of 51% eluent B was applied initially and at 2.5 minutes. The column temperature was 45° C. and the flow rate was 0.6 ml/min.

After HPLC separation, samples were injected into the mass spectrometer by electrospray ionization and analyzed in negative mode. The capillary temperature was held at 380° C. The capillary voltage was 3 kV, the source temperature was 150° C., the desolvation gas temperature was 450° C., the desolvation gas flow (nitrogen) was 800 L/hr, and the cone gas flow (nitrogen) was 50 L/hr. Detection parameters for CBGa and THCa are provided in Table 50.

Quantification of olivetolic acid was performed by HPLC on a Waters HSS 1×50 mm column with a 1.8 μm particle size. Eluent A was 0.1% formic acid in water, and eluent B was 0.1% formic acid in acetonitrile. The ratios of A1:B1 were 70/30 at 0.00 min; 50/50 at 1.2 min; 30/70 at 1.70 min, and 70/30 at 1.71 min. The column temperature was 45° C., the flow rate was 0.6 ml/min.

After HPLC separation, samples were injected into the mass spectrometer by electrospray ionization and analyzed in positive mode. The capillary temperature was held at 380° C. The capillary voltage was 3 kV, the source temperature was 150° C., the desolvation gas temperature was 450° C., the desolvation gas flow (nitrogen) was 800 L/hr, and the cone gas flow (nitrogen) was 50 L/hr. A transition of →171 and a collision of 20 V were applied to olivetolic acid. Detection parameters for CBGa and THCa are provided in Table 50.

TABLE 50
Detection parameters for CBGa and THCa

| Parameter | Olivetolic Acid | CBGa | THCa |
|---|---|---|---|
| Retention time | 1.28 min | 1.19 min | 1.50 min |
| Ion | [M − H]⁺ | [M − H]⁻ | [M − H]⁻ |
| Mass (m/z) | 223.01 | 359.2 | 357.2 |
| Mode | ES+, MRM | ES−, SIR | ES−, SIR |
| Span | 0 | 0 | 0 |
| Dwell (s) | 0.2 | 0.2 | 0.2 |
| Cone (V) | 35 | 30 | 30 |

Different concentrations of known standards were injected to create a linear standard curve. Standards for Olivetolic Acid, CBGa and THCa were purchased from Toronto Research Chemicals. Olivetol was not quantified but would have been quantified with a retention time of 1.40 min.

### EXAMPLES—PART 4

### Example 12

Twelve single colony replicates of strains HB861 and HB862 were grown in synthetic complete (“SC”), containing 1.7 g/L YNB without ammonium sulfate, 1.96 g/L URADO supplement, 76 mg/L uracil, 1.5 g/L magnesium L-glutamate, 2% w/v glucose or galactose, 2% w/v raffinose, 200 μg/l geneticin and 200 ug/L ampicillin. Both HB861 and HB862 strains were grown in 1 ml cultures in 96-well deepwell plates. The deepwell plates were incubated at 30° C. and shaken at 250 rpm for 96 hrs.

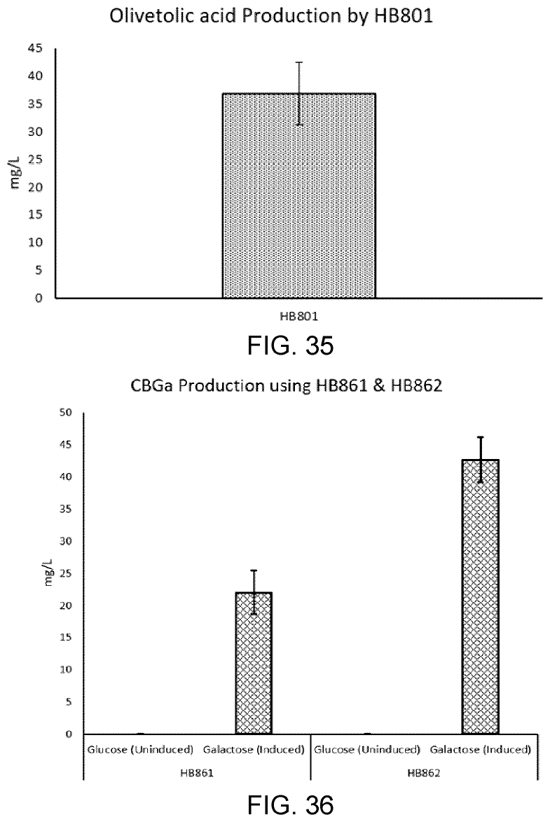

FIG. 35 shows the yields of olivetolic acid from HB801.

FIG. 36 shows production of CBGa by DiPKS^{G1516R}, csOAC and PT254 in two strains of S. cerevisiae.

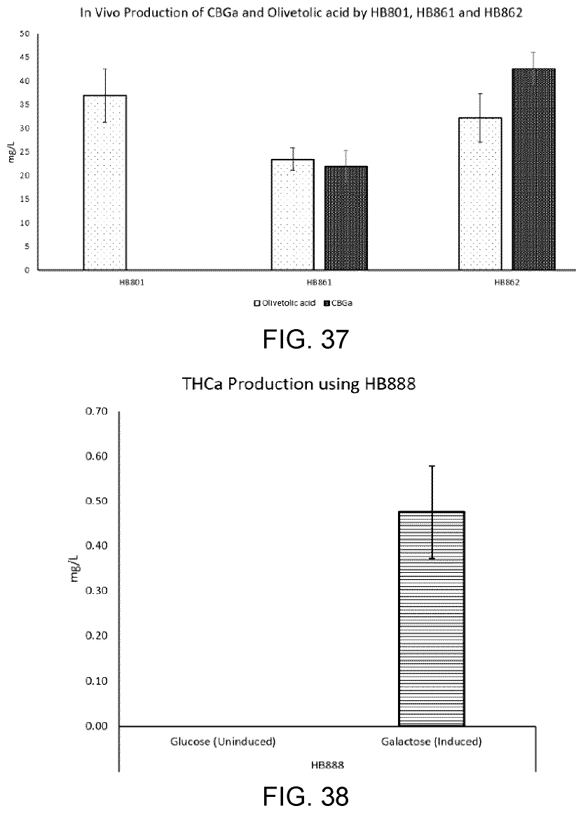

FIG. 37 shows the yield of olivetolic acid from HB801, HB861 and HB862. Production of olivetolic acid from raffinose and galactose was observed, demonstrating direct production in yeast of olivetolic acid without hexanoic acid. Olivetolic acid production was induced by activating the inducible galactose promoter for csOAC in the presence of galactose but not glucose. The olivetolic acid was produced at 36.95+/−5.63 mg/L by HB801, 23.49+/−2.37 mg/L by HB861 and 32.24+/−5.22 mg/L by HB862. The “+/−” indicates standard deviation.

### Example 13

Twelve single colony replicates of strains HB861 and HB862 were grown in SC, containing 1.7 g/L YNB without ammonium sulfate, 1.96 g/L URADO supplement, 76 mg/L uracil, 1.5 g/L magnesium L-glutamate, 2% w/v glucose or galactose, 2% w/v raffinose, 200 μg/l geneticin and 200 ug/L ampicillin. HB861 and HB862 strains were grown in 1 ml cultures in 96-well deepwell plates. Plates were incubated at 30° C. and shaken at 250 rpm for 96 hrs.

FIG. 36 and FIG. 37 each show the yields of CBGa from HB861 and HB862. Production of CBGa from raffinose and galactose was observed, demonstrating direct production in yeast of CBGa without hexanoic acid. CBGa production was induced by activating the inducible galactose promoter for PT254 in the presence of galactose but not glucose. The CBGa was produced at 22.00+/−3.4 mg/L by HB861 and at 42.68+/−3.49 mg/L by HB862. The “+/−” indicates standard deviation. The PT254_R2S mutant outperformed the wild type PT254.

### Example 14

Twelve single colony replicates of strain HB888 was grown in URADO minimal media, containing 1.7 g/L YNB without ammonium sulfate, 1.96 g/L URADO supplement, 1.5 g/L magnesium L-glutamate, 2% w/v glucose or galactose, 2% w/v raffinose, 200 μg/l geneticin, 200 ug/L hygromycin and 200 ug/L ampicillin. HB888 was grown in 1 ml cultures in 96-well deepwell plates. The deepwell plates were incubated at 30° C. and shaken at 250 rpm for 96 hrs.

FIG. 38 shows the yields of THCa by HB888. Production of THCa from raffinose and galactose was observed, demonstrating direct production in yeast of THCa without hexanoic acid. THCa production was induced by activating the inducible galactose promoter for PT254 in the presence of galactose but not glucose. The THCa was produced at 0.48+/−0.10 mg/L by HB888. The “+/−” indicates standard deviation.

Part 5

Prenyltransferases from Stachybotrys for the Production of Phytocannabinoids

The present disclosure relates generally to proteins, and cell lines, and methods for the production of phytocannabinoids in host cells involving prenyltransferases from Stachybotrys.

Overview

Prenyltransferases are provided herein, which may be used in the production of a phytocannabinoid or a phytocannabinoid analogue in a host cell. The production of a phytocannabinoid or a phytocannabinoid analogue in a host cell may be conducted according to a method that comprises transforming the host cell with a sequence encoding the prenyltransferase protein for catalysing the reaction of a polyketide with a prenyl donor. Such a transformed host cell can be cultured to produce the phytocannabinoid or phytocannabinoid analogue.

There is provided herein a method of producing a phytocannabinoid or phytocannabinoid analogue in a host cell that produces a polyketide and a prenyl donor, said method comprising: transforming said host cell with a sequence encoding a prenyltransferase PT72, PT273, and PT296 protein, and culturing the transformed host cell to produce the phytocannabinoid or phytocannabinoid analogue.

There is also provided herein a method of producing a phytocannabinoid or phytocannabinoid analogue, comprising providing a host cell which produces a polyketide precursor and a prenyl donor; introducing into the host cell a polynucleotide encoding a prenyltransferase PT72, PT273, or PT296 protein; and culturing the host cell under conditions sufficient for production of PT72, PT273, or PT296 for producing the phytocannabinoid or phytocannabinoid analogue from the polyketide precursor and the prenyl donor.

Additionally, there is provided herein an expression vector comprising a nucleotide sequence encoding the prenyltransferase PT72, PT273, or PT296 protein, wherein the nucleotide sequence comprises at least 70% identity with a polynucleotide encoding the PT72, PT273, or PT296 protein.

Host cells transformed with the expression vector are also described.

### Detailed Description of Part 5

Generally, there is described herein the production of phyotocannabinoids or phytocannabinoid analogues.

The method described herein produces a phytocannabinoid or a phytocannabinoid analogue in a host cell, which host cell comprises or is capable of producing a polyketide and a prenyl donor. The method comprises transforming the host cell with a sequence encoding a prenyltransferase PT72, PT273, or PT296 protein, and subsequently culturing the transformed cell to produce said phytocannabinoid or phytocannabinoid analogue.

The PT72, PT273, and PT296 proteins may have one of the following characteristics: (a) a protein as set forth in SEQ ID NO:438, SEQ ID NO:439, or SEQ ID NO:440; (b) a prenyltransferase protein with at least 70% identity with SEQ ID NO:438, SEQ ID NO:439, or SEQ ID NO:440; (c) a protein that differs from (a) by one or more residues that are substituted, deleted and/or inserted; or (d) a derivative of (a), (b), or (c).

The nucleotide sequence encoding the prenyltransferase PT72, PT273, or PT296 protein may have one of the following characteristics: (a) a nucleotide sequence encoding a protein as set forth in SEQ ID NO:438, SEQ ID NO:439, or SEQ ID NO:440; or having a sequence according to SEQ ID NO:459, SEQ ID NO:460, or SEQ ID NO:461; (b) a nucleotide sequence encoding a prenyltransferase protein having at least 70% identity with SEQ ID NO:438, SEQ ID NO:439, or SEQ ID NO:440; or having at least 70% identity with SEQ ID NO:459, SEQ ID NO:460, or SEQ ID NO:461; (c) a nucleotide sequence that hybridizes with the complementary strand of the nucleic acid of (a) under conditions of high stringency; (d) a nucleotide sequence that differs from (a) by one or more nucleotides that are substituted, deleted, and/or inserted; or (e) a derivative of (a), (b), (c), or (d).

The polyketide may be one of the following:

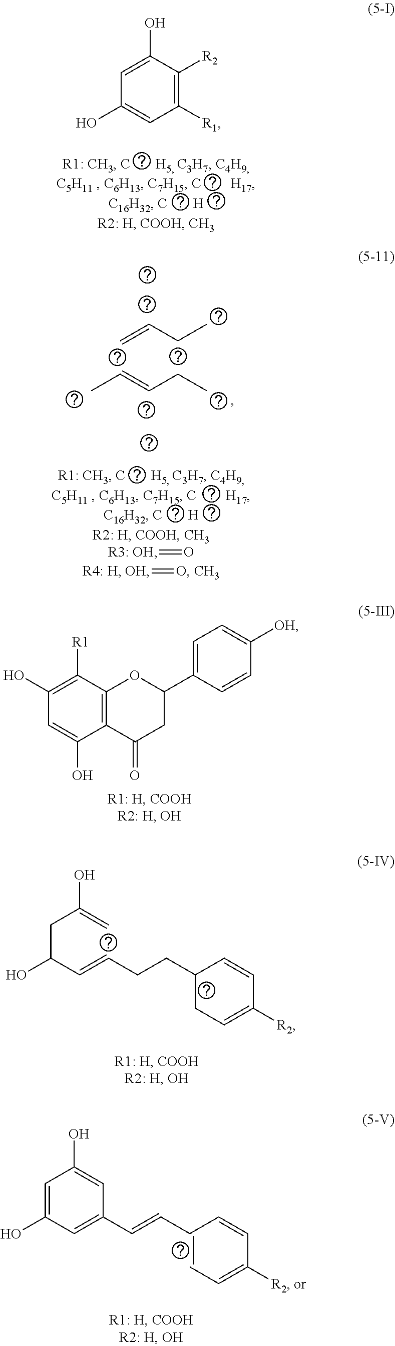

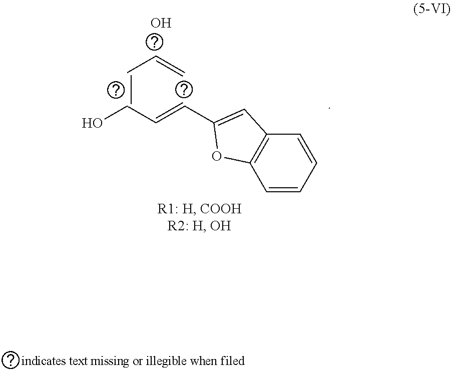

The prenyl donor may have the following structure:

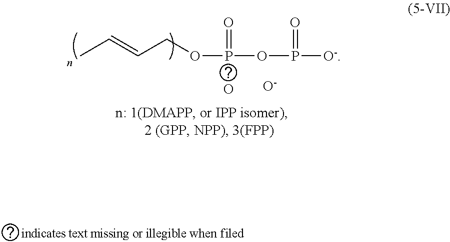

For example, the prenyl donor may be geranyl diphosphate (GPP), farnesyl diphosphate (FPP), or neryl diphosphate (NPP).

The prenylated polyketide structure for the phytocannabinoid or phytocannabinoid analogue formed may be:

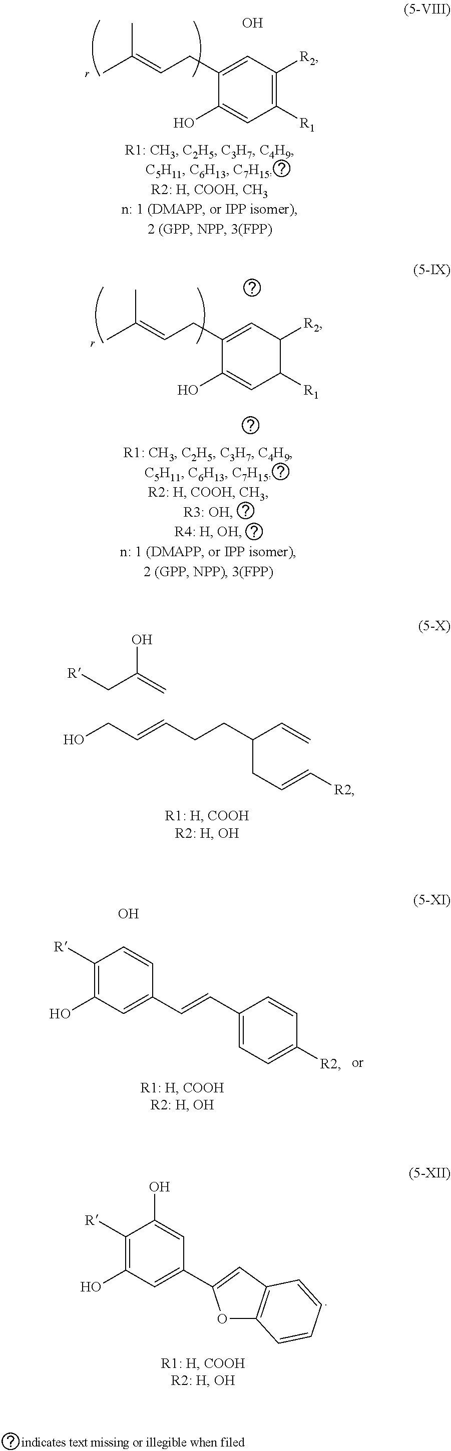

The protein encoded by the nucleotide sequence with which the host cell is transformed may have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the prenyltransferase PT72, PT273, or PT296 protein of SEQ ID NO: 438, SEQ ID NO:439 or SEQ ID NO:440.

The nucleotide sequence may have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:459, SEQ ID NO:460, or SEQ ID NO:4661; or to a polynucleotide encoding any one of SEQ ID NO:438, SEQ ID NO:439 or SEQ ID NO:440.

The polyketide prenylated in the method may be olivetol, olivetolic acid, divarin, divarinic acid, orcinol, or orsellinic acid.

The phytocannabinoid so formed may be cannabigerol (CBG), cannabigerolic acid (CBGa), cannabigerovarin (CBGv), cannabigerovarinic acid (CBGva), cannabigerocin (CBGO), or cannabigerocinic acid (CBGOa).

As exemplary embodiments, when the polyketide is olivetol then the phytocannabinoid formed is cannabigerol (CBG); when the polyketide is olivetolic acid then the phytocannabinoid formed is cannabigerolic acid (CBGa); when the polyketide is divarin then the phytocannabinoid formed is cannabigerovarin (CBGv); when the polyketide is divarinic acid then the phytocannabinoid formed is cannabigerovarinic acid (CBGva); when the polyketide is orcinol then the phytocannabinoid is cannabigerocin (CBGO); and when the polyketide is orsellinic acid then the phytocannabinoid is cannabigerocinic acid (CBGOa).

The host cell can be a fungal cell such as yeast, a bacterial cell, a protist cell, or a plant cell, such as any of the exemplary cell types noted herein. Exemplary host cell types include S. cerevisiae, E. coli, Yarrowia lipolytica, and Komagataella phaffii.

A method is described for producing a phytocannabinoid or phytocannabinoid analogue, comprising: providing a host cell which produces a polyketide precursor and a prenyl donor, introducing into the host cell a polynucleotide encoding a prenyltransferase PT72, PT273, or PT296 protein, and culturing the host cell under conditions sufficient for production of the prenyltransferase PT72, PT273, or PT296 protein for producing the phytocannabinoid or phytocannabinoid analogue from the polyketide precursor and the prenyl donor.

In any of the methods described herein, the host cell may have one or more additional genetic modification, such as for example: (a) a nucleic acid as set forth in any one of SEQ ID NO:441 to SEQ ID NO:453; (b) a nucleic acid having at least 70% identity with the nucleotide sequence of (a); (c) a nucleic acid that hybridizes with the complementary strand of the nucleic acid of (a) under stringent conditions; (d) a nucleic acid encoding a polypeptide with the same enzyme activity as the polypeptide encoded by any one of the nucleic acid sequences of (a); (e) a nucleotide sequence that differs from (a) by one or more nucleotides that are substituted, deleted, and/or inserted; or (f) a derivative of (a), (b), (c), (d), or (e). Such an additional genetic modification may comprise, for example, one or more of NpgA (SEQ ID NO:441), PDH (SEQ ID NO:447), Maf1 (SEQ ID NO:448), Erg20K197E (SEQ ID NO:449), tHMGr-IDI (SEQ ID NO:451), and/or PGK1p:ACC^{1S659A,S1157A} (SEQ ID NO:452).

One or more genetic modification may be made to the host cell in order to increase the available pool of terpenes and/or malonyl-coA in the cell. For example, such a genetic modification may include tHMGr-IDI (SEQ ID NO:451); PGK1p:ACC^{1S659A,S1157A} (SEQ ID NO:452); and/or Erg20K197E (SEQ ID NO:449).

There is described herein an expression vector comprising a nucleotide sequence encoding prenyltransferase PT72, PT273, or PT296 protein, wherein the nucleotide sequence comprises at least 70% identity with SEQ ID NO:459, SEQ ID NO:460, or SEQ ID NO:461; with a polynucleotide encoding PT72, PT273, or PT296; or with a nucleotide encoding prenyltransferase protein that comprises at least 70% identity with SEQ ID NO:438, SEQ ID NO:439, or SEQ ID NO:440.

In such an expression vector, the nucleotide sequence encoding the prenyltransferase PT72, PT273, or PT296 protein may comprises, for example, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with SEQ ID NO:459, SEQ ID NO:460, or SEQ ID NO:461; or with a polynucleotide encoding any one of PT72, PT273, or PT296.

In such an expression vector the prenyltransferase PT72, PT273, or PT296 protein encoded may have at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with SEQ ID NO:438, SEQ ID NO:439, or SEQ ID NO:440.

A host cell is described herein that is transformed with any one of the expression vectors describe, wherein transformation occurs according to any known process. Such a host cell may additionally comprising one or more of: (a) a nucleic acid as set forth in any one of SEQ ID NO:441 to SEQ ID NO:453; (b) a nucleic acid having at least 70% identity with the nucleotide sequence of (a); (c) a nucleic acid that hybridizes with the complementary strand of the nucleic acid of (a), and this hybridization may occur under stringent conditions; (d) a nucleic acid encoding a protein with the same enzyme activity as the protein encoded by any one of the nucleic acid sequences of (a); (e) a nucleic acid that differs from (a) by one or more nucleotides that are substituted, deleted, and/or inserted; or (f) a derivative of (a), (b), (c), (d), or (e).

The host cell may be a fungal cell such as yeast, a bacterial cell, a protist cell, or a plant cell, such as any cell described herein. Exemplary cells include S. cerevisiae, E. coli, Yarrowia lipolytica, and Komagataella phaffii.

The methods, vectors, and cell lines described herein may advantageously be used for the production of phytocannabinoids. By utilizing a protein having prenyltransferase activity, such as PT72, PT273, or PT296, the transformation into a heterologous host cell permits the production of cannabinoids without requiring growth of a whole plant. Cannabinoids such as, but not limited to, CBGa and CBGOa, can be prepared and isolated economically and under controlled conditions. Advantageously, it has been found that PT72, PT273, and PT296 function well in host cells, such as but not limited to yeast, permitting efficient prenylation of aromatic polyketides in the pathway of phytocannabinoid synthesis.

Phytocannabinoids are a large class of compounds with over 100 different known structures that are produced in the Cannabis sativa plant. These bio-active molecules, such as tetrahydrocannabinol (THC) and cannabidiol (CBD), can be extracted from plant material for medical and recreational purposes.

Phytocannabinoids are synthesized from polyketide and terpenoid precursors which are derived from two major secondary metabolism pathways in the cell. For example, the C—C bond formation between the polyketide olivetolic acid and the allylic isoprene diphosphate geranyl pyrophosphate (GPP) produces the cannabinoid cannabigerolic acid (CBGa). This reaction type is catalyzed by enzymes known as prenyltransferases. The Cannabis plant utilizes a membrane-bound prenyltransferase to catalyze the addition of the prenyl moiety to olivetolic acid to form CBGa.

It has been found, as described herein, that olivetolic acid and GPP can be taken as substrates for the PT72, PT273, and PT296 enzymes, which may thus advantageously be used in phytocannabionoid synthesis. As described herein, PT72, PT273, or PT296 may be used to transform a host cell, for use in prenylating polyketides in the pathway to phytocannabinoid synthesis.

In one aspect, there is a method described of producing a phytocannabinoid or phytocannabinoid analogue, comprising: utilizing PT72, PT273, or PT296, a recombinant prenyltransferase, to react a polyketide with a GPP to produce a phytocannabinoid or phytocannabinoid analogue.

In one aspect there is described a method of producing cannabigorcinic acid (CBGOa), comprising: providing a host cell which produces orsellinic acid; introducing a polynucleotide encoding prenyltransferase PT72, PT273, or PT296 polypeptide into said host cell, culturing the host cell under conditions sufficient for PT72, PT273, or PT296 polypeptide production in effective amounts to react with geranyl phyrophosphate to produce CBGOa.

In one aspect there is described a method of producing cannabigorcinic acid (CBGOa), comprising: culturing a host cell which produces orsellinic acid and comprises a polynucleotide encoding prenyltransferase PT72, PT273, or PT296 polypeptide under conditions sufficient for PTase polypeptide production.

Non limiting examples of phytocannabinoids that can be prepared according to the described methods include tetrahydrocannabinol (THC), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), and cannabigerol monomethyl ether (CBGM).

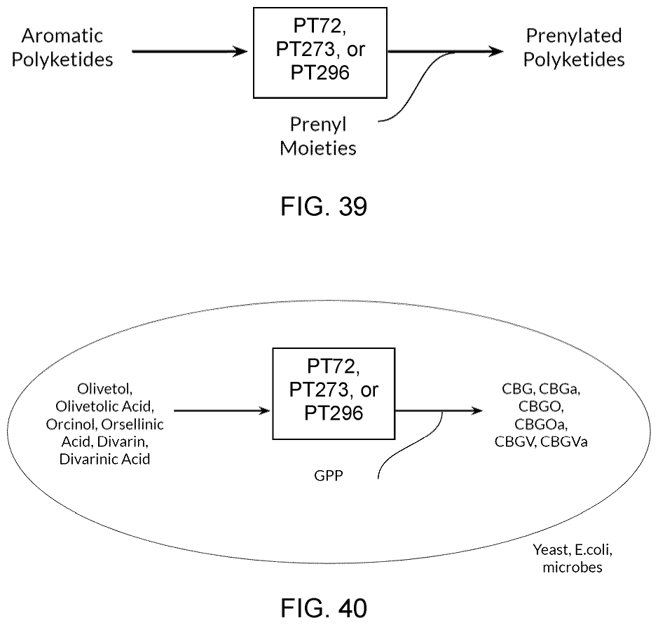

FIG. 39 depicts a general scheme for the use of any one of PT72, PT273, and PT296, as described herein, to attach a prenyl moiety to aromatic polyketides to produce prenylated polyketides.

FIG. 40 depicts examples of specific aromatic polyketides used in the pathway to the production of phytocannabinoids. Further, FIG. 3 is referenced here, depicting structures of phytocannabinoids produced from the C—C bond formation between a polyketide precursor and geranyl pyrophosphate.

In some example, the cannabinoid or phytocannabinoid may have one or more carboxylic acid functional groups. Non limiting examples of such cannabinoids or phytocannabinoids include tetrahydrocannabinolic acid (THCA), cannabidiolic acid (CBDA), and cannabichromenic acid (CBCA).

In some example, the cannabinoid or phytocannabinoid may lack carboxylic acid functional groups. Non limiting examples of such cannabinoids or phytocannabinoids include THC, CBD, CBG, CBC, and CBN.

In some examples of the method described herein, the phytocannabinoid produced is cannabigerol (CBG), cannabigerolic acid (CBGa), cannabigerovarin (CBGv), cannabigerovarinic acid (CBGva), cannabigerocin (CBGo), or cannabigerocinic acid (CBGoa).

In some examples of the method described herein, the polyketide is olivetol, olivetolic acid, divarin, divarinic acid, orcinol, or orsellinic acid.

In some example of the method herein, when the polyketide is olivetol the phytocannabinoid formed is cannabigerol (CBG), when the polyketide is olivetolic acid then the phytocannabinoid is cannabigerolic acid (CBGa), when the polyketide is divarin then the phytocannabinoid is cannabigerovarin (CBGv), when the polyketide is divarinic acid then the phytocannabinoid is cannabigerovarinic acid (CBGva), when the polyketide is orcinol then the phytocannabinoid is cannabigerocin (CBGo), and when the polyketide is orsellinic acid then the phytocannabinoid is cannabigerocinic acid (CBGoa).

A list of polyketides, prenyl donors and resulting prenylated polyketides which may be used or produced according to the methods described is provided in Table 1 above. The following terms are used: DMAPP for dimethylallyl diphosphate; GPP for geranyl diphosphate; FPP for farnesyl diphosphate; NPP for neryl diphosphate; and IPP for isopentenyl diphosphate.

As provided above in Table 2, there are numerous options for host cell organisms which may be used in one or more of the methods described herein

Method of the invention are conveniently practiced by providing the compounds and/or compositions used in such method in the form of a kit. Such kit preferably contains the composition. Such a kit preferably contains instructions for the use thereof.

### EXAMPLES—PART 5

To gain a better understanding of the invention described herein, the following examples are set forth. It should be understood that these examples are for illustrative purposes only. Therefore, they should not limit the scope of this invention in anyway.

### Example 15

Production of Phytocannabinoids in Yeast with Prenyltransferases from Stachybotrys.

Introduction. Phytocannabinoids are naturally produced in Cannabis sativa, other plants, and some fungi. Over 105 phytocannabinoids are known to be biosynthesized in C. sativa, or result from thermal or other decomposition from phytocannabinoids biosynthesized in C. sativa. While the C. sativa plant is also a valuable source of grain, fiber, and other material, growing C. sativa for phytocannabinoid production, particularly indoors, is costly in terms of energy and labour. Subsequent extraction, purification, and fractionation of phytocannabinoids from the C. sativa plant is also labour and energy intensive.

Phytocannabinoids are pharmacologically active molecules that contribute to the medical and psychotropic effects of C. sativa. Biosynthesis in the C. sativa plant scales similarly to other agricultural projects. As with other agricultural projects, large scale production of phytocannabinoids by growing C. sativa requires a variety of inputs (e.g. nutrients, light, pest control, CO2, etc.). The inputs required for cultivating C. sativa must be provided. In addition, cultivation of C. sativa, where allowed, is currently subject to heavy regulation, taxes, and rigorous quality control where products prepared from the plant are for commercial use, further increasing costs. As a result, it may be economical to produce the phytocannabinoids in a robust and scalable, fermentable organism. Saccharomyces cerevisiae has been used to produce industrial scales of similar molecules.

The time, energy, and labour involved in growing C. sativa for phytocannabinoid production provides a motivation to produce transgenic cell lines for production of phytocannabinoids in yeast.

International patent publication WO2018/148848 (Mookerjee et al.,), which is herein incorporated by reference, describes one such method for phytocannabinoid production in a transgenic yeast cell line.

The production of phytocannabinoids in genetically modified strains of Saccharomyces cerevisiae that have been transformed with genes coding for a prenyltransferase (PT72, PT273 or PT296) from Stachybotrys is described. These prenyltransferases catalyze the synthesis of cannabigerolic acid (CBGa) from olivetolic acid (OLA) and geranyl pyrophosphate (GPP). In C. sativa, a prenyltransferase catalyzes the synthesis of CBGa from olivetolic acid and GPP; however, the C. sativa prenyltransferase functions poorly in S. cerevisiae (see, for example, U.S. Pat. No. 8,884,100). The C. sativa prenyltransferase has a native N-terminal chloroplast targeting tag which may complicate expression in fungal hosts. PT72, PT273 and PT296 do not possess this targeting tag and thus may provide a distinct advantage when expressed in S. cerevisiae. This may be useful in creating a consolidated phytocannabinoid producing strain of S. cerevisiae. The S. cerevisiae may also have one or more mutations or modification in genes and metabolic pathways that are involved in OLA and GPP production or consumption.

The modified S. cerevisiae strain may also express genes encoding for DiPKS, a hybrid Type1 FAS-Type 3 PKS from Dictyostelium discoideum (Ghosh et al., 2008) and Olivetolic acid cyclase (OAC) from C. sativa (Gagne et al., 2012). DiPKS allows for the direct production of methyl-Olivetol (meOL) from malonyl-coA, a native yeast metabolite. Certain mutants of DiPKS have been identified that lead to the direct production of olivetol (OL) from malonyl-coA (see WO2018/148848 (2018) to Mookerjee et al.). OAC has been demonstrated to assist in the production of olivetolic acid when a suitable Type 3 PKS is used.

The C. sativa pathway enzymes require hexanoic acid for the production of OLA. However, hexanoic acid is highly toxic to S. cerevisiae and greatly diminishes its growth phenotype. As a result, when using DiPKS and OAC rather than the C. sativa pathway enzymes, hexanoic acid need not be added to the growth media, which may result in increased growth of the S. cerevisiae cultures and greater production of olivetolic acid. The S. cerevisiae may have over-expression of native acetaldehyde dehydrogenase and expression of a modified version of an acetoacetyl-CoA carboxylase or other genes, the modifications resulting in lowered mitochondrial acetaldehyde catabolism. Lowering mitochondrial acetaldehyde catabolism by diverting the acetaldehyde into acetyl-CoA production increases malonyl-CoA available for synthesizing olivetolic acid.

FIG. 4 is referenced here as an outline of the native biosynthetic pathway for cannabinoid production in Cannabis sativa. As expression and functionality of the C. sativa pathway in S. cerevisiae is hindered by problems of toxic precursors and poor expression, this Example utilizes a different biosynthetic route for cannabinoid production to overcome one or more of the above-described detrimental issues. FIG. 5 is referenced here as an outline of the pathway of cannabinoid biosynthesis as described herein. A four enzyme system is described. Dictyostelium polyketide synthase (DiPKS) (1), from D. discoideum and olivetolic acid cyclase (OAC) (2) from C, sativa are used to produce olivetolic acid directly from glucose, via acetyl CoA and malonyl CoA. Geranyl pyrophosphate (GPP) from the yeast terpenoid pathway and olivetolic acid (OLA) are subsequently converted to Cannabigerolic acid using a prenyltransferase (3) which in this example is: PT72, PT273, or PT296. Cannabigerolic acid is then further cyclized to produce THCa or CBDa using C. sativa THCa synthase (5) or CBDa synthase (4) enzymes, respectively.

The prenyltransferases referenced herein as “PT72”, “PT273”, or “PT296”, are previously uncharacterized integral membrane proteins that are derived from Stachybotrys bisbyi (PT72), Stachybotrys chlorohalonata (PT273) and Stachybotrys chartarum (PT296). These proteins are loosely related to PT104, a prenyltransferase from Rhododendron dauricum that had been previously reported to catalyze CBGA biosynthesis, as described in Applicant's own co-pending U.S. Provisional Patent Application No. 62,851,400, which is herein incorporated by reference. Sequence identity between PT72, PT273, PT296, PT104 as well two CBGA prenyltransferases reported from C. sativa (PT85) described in U.S. Pat. No. 8,884,100 and PT254 (Luo et al, 2019) are shown below in Table 51. Note that PT104 is a grifolic acid synthase, an integral membrane protein from Rhododendron dauricum, that has been characterized to convert orsellinic acid and farnesyl pyrophosphate (FPP) to grifolic acid (Saeki et al., 2018).

TABLE 51
Sequence Identity Between PT72, PT273,
PT296 and Other CBGa Prenyltransferases

| | % Identity | % Identity | % Identity |
|---|---|---|---|
| Enzyme | to PT72 | to PT273 | to PT296 |
| PT72 | 100 | 75.5 | 74.5 |
| PT273 | 75.5 | 100 | 97.8 |
| PT296 | 74.5 | 97.8 | 100 |
| PT85 | 20.4 | NA | NA |
| (U.S. Pat. No. 8,884,100) | | | |
| PT104 | 43.7 | 29.7 | 26.1 |
| PT254 | 25.5 | NA | NA |
| (see Luo et al. 2019) | | | |

The in vivo production of CBGa in S. cerevisiae using PT72, PT273 and PT296 as prenyltransferases is described herein. The base strains used in this example have modifications which allow for GPP and olivetolic acid production. The modifications are codified below in Table 52. The modifications made to the base strain are named, and described with reference to a sequence (SEQ ID NO.), the integration region in the genome, and other details such as the genetic structure of the sequence.

TABLE 52
Modifications to Base Strains Used in this Example

| | | | Integration | | |
|---|---|---|---|---|---|
| | Modification | SEQ ID | Region/ | | Genetic Structure |
| # | name | NO. | Plasmid | Description | of Sequence |
| 1 | NpgA | SEQ ID | Flagfeldt Site | Phosphopantetheinyl Transferase | Site14Up::Tef1p: |
| | | NO. 441 | 14 integration | from Aspergillus niger. Accessory | NpgA:Prm9t:Site14Down |
| | | | | Protein for DiPKS (Kim et al., | |
| | | | | 2015) | |
| 2 | DiPKS-1 | SEQ ID | USER Site | Type 1 FAS fused to Type 3 PKS | XII-1up::Gal1p: |
| | | NO. 442 | XII-1 | from D. discoideum. Produces | DiPKSG1516R: |
| | | | integration | Olivetol from malonyl-coA | Prm9t::XII1-down |
| | | | (Jensen et al., | | |
| | | | 2013) | | |
| 3 | DiPKS-2 | SEQ ID | Wu site 1 | Type 1 FAS fused to Type 3 PKS | Wu1up::Gal1p: |
| | | NO. 443 | integration | from D. discoideum. Produces | DiPKSG1516R: |
| | | | | Olivetol from malonyl-coA | Prm9t::Wu1down |
| 4 | DiPKS-3 | SEQ ID | Wu site 3 | Type 1 FAS fused to Type 3 PKS | Wu3up::Gal1p: |
| | | NO. 444 | integration | from D. discoideum. Produces | DiPKSG1516R: |
| | | | | Olivetol from malonyl-coA | Prm9t::Wu3down |
| 5 | DiPKS-4 | SEQ ID | Wu site 6 | Type 1 FAS fused to Type 3 PKS | Wu6up::Gal1p: |
| | | NO. 445 | integration | from D. discoideum. Produces | DiPKSG1516R: |
| | | | | Olivetol from malonyl-coA | Prm9t::Wu6down |
| 6 | DiPKS-5 | SEQ ID | Wu site 18 | Type 1 FAS fused to Type 3 PKS | Wu18up::Gal1p: |
| | | NO. 446 | integration | from D. discoideum. Produces | DiPKSG1516R: |
| | | | | Olivetol from malonyl-coA | Prm9t::Wu18down |
| 7 | PDH | SEQ ID | Flagfeldt Site | Acetaldehyde dehydrogenase | 19Up::Tdh3p:Ald6: |
| | | NO. 447 | 19 integration | (ALD6) from S. cerevisiae and | Adh1::Tef1p:seACS1^{L641P}: |
| | | | | acetoacetyl coA synthase | Prm9t::19Down |
| | | | | (AscL641P) from Salmonella | |
| | | | | enterica. Will allow greater | |
| | | | | accumulation of acetyl-coA in the | |
| | | | | cell. (Shiba et al., 2007) | |
| 8 | Maf1 | SEQ ID | Flagfeldt Site | Maf1 is a regulator of tRNA | Site5Up::Tef1p:Maf1: |
| | | NO. 448 | 5 integration | biosynthesis. Overexpression in S. | Prm9t:Site5Down |
| | | | | cerevisiae has demonstrated | |
| | | | | higher monoterpene (GPP) yields. | |
| | | | | (Liu et al., 2013) | |
| 9 | Erg20K197E | SEQ ID | Chromosomal | Mutant of Erg20 protein that | Tpi1t:ERG20K197E: |
| | | NO. 449 | modification | diminishes FPP synthase activity | Cyc1t::Tef1p:KanMX:Tef1t |
| | | | | creating greater pool of GPP | |
| | | | | precursor. Negatively affects | |
| | | | | growth phenotype.(Oswald et al, | |
| | | | | 2007) | |
| 10 | Erg1p:UB14- | SEQ ID | Flagfeldt Site | Sterol responsive promoter | Site18Up::Erg1p: |
| | Erg20:deg | NO. 450 | 18 integration | controlling Erg20 protein activity. | UB14deg:ERG20: |
| | | | | Allows for regular FPP synthase | Adh1t:Site18down |
| | | | | activity and uninhibited growth | |
| | | | | phenotype until accumulation of | |
| | | | | sterols which leads to a | |
| | | | | suppression of expression of | |
| | | | | enzyme. (Liu et al., 2013) | |
| 11 | tHMGr-IDI | SEQ ID | USER Site X- | Overexpression of truncated | X3up::Tdh3p:tHMGR1: |
| | | NO. 451 | 3 integration | HMGr1 and IDI1 proteins that | Adh1t::Tef1p:IDI1: |
| | | | | have been previously identified to | Prm9t::X3down |
| | | | | be bottlenecks in the S. cerevisiae | |
| | | | | terpenoid pathway responsible for | |
| | | | | GPP production. (Ro et al., 2006) | |
| 12 | PGK1p: | SEQ ID | Chromosomal | Mutations in the native S. | Pgk1:ACC1^{S659A, S1157A}: |
| | ACC1^{S659A, S1157A} | NO. 452 | modification | cerevisiae acetyl-coA carboxylase | Acc1t |
| | | | | that removes post-translational | |
| | | | | modification based down- | |
| | | | | regulation. Leads to greater | |
| | | | | malonyl-coA pools. The promoter | |
| | | | | of Acc1 was also changed to a | |
| | | | | constitutive promoter for higher | |
| | | | | expression (Shi, 2014) | |
| 13 | OAC | SEQ ID | Flagfeldt Site | The Cannabis sativa Olivetolic | FgF16up::Gal1p:csOAC: |
| | | NO. 453 | 16 integration | acid cyclase (OAC) protein allows | Eno2t::FgF16down |
| | | | | the production of olivetolic acid | |
| | | | | from a polyketide precursor. | |

The function of PT104 in the known synthetic pathway to grifolic acid is outlined in FIG. 6. Grifolic acid is an intermediate in the production of daurichromenic acid, an anti-HIV small molecule. This enzyme was previously characterized to strictly prefer orsellinic acid as the polyketide precursor and farnesyl pyrophosphate as the preferred prenyl donor. However, as described herein, that olivetolic acid and GPP can also be taken as substrates for this enzyme, as described in Applicant's own co-pending U.S. Provisional Patent Application No. 62/851,400, which is herein incorporated by reference. This leads to advantages for the use of this enzyme in phytocannabionoid synthesis. PT104, which may also be referred to as d31RdPT1, is a grifolic acid synthase, an integral membrane protein from Rhododendron dauricum, that has been characterized to convert orsellinic acid and farnesyl pyrophosphate (FPP) to grifolic acid (Saeki et al., 2018).

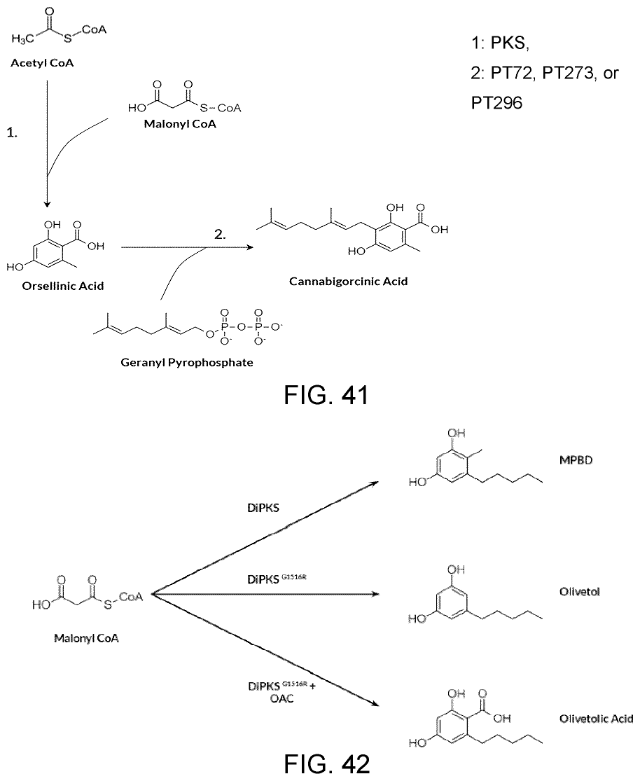

FIG. 41 shows a schematic outline of involvement of PT72, PT273, or PT296 as the prenyltransferase involved in preparing cannabigorcinic acid (CBGa), starting from the reaction of acetyl CoA with malonyl CoA to form orsellinic acid with the involvement of polyketide synthase (PKS). The orsellinic acid, together with geranyl pyrophosphate may then form CBGa, catalyzed by prenyltransferase PT72, PT273 or PT296 as described herein.

This example describes, for the first time, the in vivo production of cannabigerorcinic acid (CBGOa) and CBGa in S. cerevisiae using any one of PT72, PT273 or PT296 as the prenyltransferase.

Table 53 provides information about the plasmids used in this Example.

TABLE 53
Plasmid Information

| # | Plasmid Name | Description | Selection | Backbone |
|---|---|---|---|---|
| 1 | PLAS384 | Gal1p:PT273Cyc1t | Uracil | pYES-URA |
| 2 | PLAS400 | Gal1p:mScarlett:Cyc1t | Uracil | pYES-URA |
| 3 | PLAS411 | Gal1p:PT72:Cyc1t | Uracil | pYES-URA |
| 4 | PLAS413 | Gal1p:PT254:Cyc1t | Uracil | pYES-URA |
| 5 | PLAS414 | Gal1p:PT296:Cyc1t | Uracil | pYES-URA |

Table 54 lists the strains used in this example, providing the features of the strains including background, plasmids if any, genotype, etc.

TABLE 54
Strains Used

| Strain # | Background | Plasmids | Genotype | Notes |
|---|---|---|---|---|
| HB42 | -URA, -LEU | None | Saccharomyces cerevisiae | Base strain |
| | | | CEN.PK2; ΔLEU2; ΔURA3; | |
| | | | Erg20K197E::KanMx | |
| HB144 | -URA, -LEU | None | Saccharomyces cerevisiae | Parent strain |
| | | | CEN.PK2; ΔLEU2; ΔURA3; | for orsellinic |
| | | | Erg20K197E::KanMx; ALD6; ASC1L641P; | acid, divarinic |
| | | | NPGA; MAF1; PGK1p:Acc1; tHMGR1; IDI | acid and |
| | | | | olivetolic acid |
| | | | | feeding assays |
| HB895 | -URA, -LEU | None | Saccharomyces cerevisiae | Parent strain |
| | | | CEN.PK2; ΔLEU2; ΔURA3; | for in vivo |
| | | | Erg20K197E::KanMx; ALD6; ASC1L641P; | CBGA |
| | | | NPGA; MAF1; PGK1p:Acc1; tHMGR1; IDI; | production |
| | | | DiPKS_G1516R X 5; ACC1_S659A_S1157A; | assay with |
| | | | UB14p:ERG20; OAC; | inducible |
| | | | | prenyltransferases |
| HB977 | -URA, -LEU | PLAS400 | Saccharomyces cerevisiae | Expresses a |
| | | | CEN.PK2; ΔLEU2; ΔURA3; | non-cata lytic |
| | | | Erg20K197E::KanMx; ALD6; ASC1L641P; | mScarlett, |
| | | | NPGA; MAF1; PGK1p:Acc1; tHMGR1; IDI; | negative |
| | | | DiPKS_G1516R X 5; ACC1_S659A_S1157A; | control |
| | | | UB14p:ERG20; OAC; Galp:mScarlett | |
| HB1648 | -URA, -LEU | PLAS384 | Saccharomyces cerevisiae | Produces |
| | | | CEN.PK2; ΔLEU2; ΔURA3; | CBGA when |
| | | | Erg20K197E::KanMx; ALD6; ASC1L641P; | fed olivetolic |
| | | | NPGA; MAF1; PGK1p:Acc1; tHMGR1; IDI; | acid |
| | | | Galp:PT273 | |
| HB1649 | -URA, -LEU | PLAS411 | Saccharomyces cerevisiae | Produces |
| | | | CEN.PK2; ΔLEU2; ΔURA3; | CBGA when |
| | | | Erg20K197E::KanMx; ALD6; ASC1L641P; | fed olivetolic |
| | | | NPGA; MAF1; PGK1p:Acc1; tHMGR1; IDI; | acid |
| | | | DiPKS_G1516R X 5; ACC1_S659A_S1157A; | |
| | | | UB14p:ERG20; PT72 | |
| HB1650 | -URA, -LEU | PLAS400 | Saccharomyces cerevisiae | Negative for |
| | | | CEN.PK2; ΔLEU2; ΔURA3; | orsellinic acid, |
| | | | Erg20K197E::KanMx; ALD6; ASC1L641P; | divarinic acid |
| | | | NPGA; MAF1; PGK1p:Acc1; tHMGR1; IDI; | and olivetolic |
| | | | Galp:mScarlett | acid feeding |
| | | | | assays |
| HB1654 | -URA, -LEU | PLAS413 | Saccharomyces cerevisiae | Produces |
| | | | CEN.PK2; ΔLEU2; ΔURA3; | CBGa when |
| | | | Erg20K197E::KanMx; ALD6; ASC1L641P; | induced with |
| | | | NPGA; MAF1; PGK1p:Acc1; tHMGR1; IDI; | galactose. |
| | | | Galp:PT254 | Positive |
| | | | | control. |
| HB1665 | -URA, -LEU | PLAS414 | Saccharomyces cerevisiae | Produces |
| | | | CEN.PK2; ΔLEU2; ΔURA3; | CBGa when |
| | | | Erg20K197E::KanMx; ALD6; ASC1L641P; | induced with |
| | | | NPGA; MAF1; PGK1p:Acc1; tHMGR1; IDI; | galactose |
| | | | DiPKS_G1516R X 5; ACC1_S659A_S1157A; | |
| | | | UB14p:ERG20; OAC; Galp:PT296 | |
| HB1667 | -URA, -LEU | PLAS413 | Saccharomyces cerevisiae | Produces |
| | | | CEN.PK2; ΔLEU2; ΔURA3; | CBGa when |
| | | | Erg20K197E::KanMx; ALD6; ASC1L641P; | induced with |
| | | | NPGA; MAF1; PGK1p:Acc1; tHMGR1; IDI; | galactose. |
| | | | DiPKS_G1516R X 5; ACC1_S659A_S1157A; | Positive |
| | | | UB14p:ERG20; OAC; Galp:PT254 | control. |

Materials and Methods:

Genetic Manipulations:

HB42 was used as a base strain to develop all other strains. All DNA was transformed into strains using the Gietz et al., (2014) transformation protocol. Plas 36 was used for the CRISPR-based genetic modifications described in this experiment (Ryan et al., 2016).

The genome of HB42 was iteratively targeted by gRNA's and Cas9 expressed from PLAS36 to make genomic modifications in the order shown in Table 55.

TABLE 55
Genomic Modifications to Base Strain BH42

| Order | Genomic Region | Modification |
|---|---|---|
| 1 | Flagfeldt Site 19 integration | PDH |
| 2 | Flagfeldt Site 14 integration | NpgA |
| 3 | Flagfeldt Site 5 integration | Maf1 |
| 4 | Chromosomal Modification | PGK1p:ACC1^{S659A, S1157A} |
| 5 | USER Site X-3 integration | tHMGR-IDI1 |
| 6 | USER Site XII-2 integration | DiPKS-1 |
| 7 | Flagfeldt Site 18 integration | Erg1p:UB14-Erg20:deg |
| 8 | Wu site 1 integration | DiPKS-2 |
| 9 | Wu site 3 integration | DiPKS-3 |
| 10 | Wu site 6 integration | DiPKS-4 |
| 11 | Wu site 18 integration | DiPKS-5 |
| 12 | Flagfeldt site 16 integration | OAC |

Strain Growth and Media. HB1648, HB1649, HB1650 and HB1654 were grown in yeast minimal media with a composition of 1.7 g/L YNB without ammonium sulfate+1.96 g/L URA dropout amino acid supplements+1.5 g/L magnesium L-glutamate) with 2% w/v galactose, 2% w/v raffinose, 200 μg/l geneticin, and 200 ug/L ampicillin (Sigma-Aldrich Canada)+100 mg/L Orsellinic acid (Sigma-Aldrich Canada) for 96 hours. This allows the strains to produce CBGOa if the appropriate prenyltransferase is present. HB1650 expressed a non-catalytic mScarlett protein under these conditions and serves as a negative control.

In another embodiment HB1648, HB1649, HB1650 and HB1654 were grown in yeast minimal media with a composition of 1.7 g/L YNB without ammonium sulfate+1.96 g/L URA dropout amino acid supplements+1.5 g/L magnesium L-glutamate) with 2% w/v galactose, 2% w/v raffinose, 200 μg/l geneticin, and 200 ug/L ampicillin (Sigma-Aldrich Canada)+100 mg/L Divarinic acid (Sigma-Aldrich Canada) for 96 hours. This allows the strains to produce CBGVa if the appropriate prenyltransferase is present. HB1650 expressed a non-catalytic mScarlett protein under these conditions and serves as a negative control.

In another embodiment HB1648, HB1649, HB1650 and HB1654 were grown in yeast minimal media with a composition of 1.7 g/L YNB without ammonium sulfate+1.96 g/L URA dropout amino acid supplements+1.5 g/L magnesium L-glutamate) with 2% w/v galactose, 2% w/v raffinose, 200 μg/l geneticin, and 200 ug/L ampicillin+100 mg/L (Sigma-Aldrich Canada)+100 mg/L Olivetolic acid (Sigma-Aldrich Canada) for 96 hours. This allows the strains to produce CBGa if the appropriate prenyltransferase is present. HB1650 expressed a non-catalytic mScarlett protein under these conditions and serves as a negative control.

In another embodiment HB1665, HB997, and HB1667 were grown in yeast minimal media with a composition of 1.7 g/L YNB without ammonium sulfate+1.96 g/L URA dropout amino acid supplements+1.5 g/L magnesium L-glutamate) with 2% w/v galactose, 2% w/v raffinose, 200 μg/l geneticin, and 200 ug/L ampicillin+100 mg/L (Sigma-Aldrich Canada). HB1665, HB997 and HB1667 will produce olivetolic acid upon induction with galactose. CBGA will also be produced if the appropriate prenyltransferase is present.

Experimental Conditions. 3 single colony replicates of strains were tested in this example. All strains were grown in 1 ml media for 96 hours in 96-well deepwell plates. The deepwell plates were incubated at 30° C. and shaken at 950 rpm for 96 hrs.

Metabolite extraction was performed by adding 100 μl of 100% acetonitrile to 100 μl of culture in a new 96-well deepwell plate. An additional 200 μl of 75% acetonitrile was then added, followed by resuspension 10 times with a 200 ul pipette. The solutions were then centrifuged at 3750 rpm for 5 min. 200 μl of the soluble layer was removed and stored in a 96-well v-bottom microtiter plate. Samples were stored at −20° C. until analysis.

Samples were quantified using HPLC-MS analysis.

Quantification Protocol. The quantification of CBGa, CBGVa and CBGOa was performed using HPLC-MS on a Acquity UPLC-TQD MS. The chromatography and MS conditions are described below.

LC conditions. Column: ACQUITY UPLC 50×1 mm, 1.8 μm particle size. Column temperature: 45° C. Flow rate: 0.3 ml/min. Eluent A: Water 0.1% formic acid. Eluent B: Acetontrile 0.1% formic acid.

Table 56 shows the gradient over time.

TABLE 56
Gradient for LC

| Time (min) | % B |
|---|---|
| 0.00 | 10 |
| 0.90 | 90 |
| 1.30 | 90 |
| 1.31 | 10 |
| 2.00 | 10 |

ESI-MS conditions. Capillary: 4.0 kV. Source temperature: 150° C. Desolvation gas temperature: 250° C. Desolvation gas flow (nitrogen): 500 L/hr. Cone gas flow (nitrogen): 50 L/hr.

Table 57 lists detection parameters for ESI-MS.

TABLE 57
Detection Parameters for ESI-MS

| | CBGa |
|---|---|
| Retention time | 1.36 min |
| Transition (m/z) | 359.2 → 341.2 |
| Mode | ES−, MRM |
| Cone | 40 |
| Cone (V) | 25 |
| | CBGOa |
| Retention time | 1.22 min |
| Transition (m/z) | 303.2 → 285.1 |
| Mode | ES−, MRM |
| Cone | 45 |
| Cone (V) | 25 |
| | CBGVa |
| Retention time | 1.28 min |
| Transition (m/z) | 331.2 → 313.2 |
| Mode | ES−, MRM |
| Cone | 45 |
| Cone (V) | 25 |

Results:

The production of CBGOa, CBGVa and CGa in S. cerevisiae by resorcyclic acid feeding was observed.

Strains expressing PT273 (HB1648), PT72 (HB1649), PT254(HB1654) or mScarlett (HB1650) were grown in the presence of resorcylic acid to test prenyltransferase catalytic activity with different substrates. Media was supplemented to a final concentration of 100 mg/L with either orsellinic acid (Cl), divarinic acid (04) or olivetolic acid (06).

Table 58 shows the production of the corresponding C1, 04 and 06 cannabis in HB1648; HB1649, and HB1654 using resorcylic acid feeds, expressed in mg/L.

TABLE 58
Production of CBGOa, CBGVa and CBGa
by Novel Prenyltransferases

| | CBGOa | | CBGa |
|---|---|---|---|
| | (mg/L) | CBGVa(mg/L) | (mg/L) |
| HB1648 (PT273) | 0.70 | 1.14 | 15.67 |
| HB1649 (PT72) | 1.02 | 4.30 | 38.33 |
| HB1654 (PT254) | 0.00 | 8.40 | 15.33 |
| HB1650 (mScarlett) | 0.00 | 0.00 | 0.00 |

The production of CBGa was evaluated in vivo using PT296. PT296 (HB1665), PT254 (HB1667) and mScarlett (HB977) were expressed in an olivetolic acid producing strain of S. cerevisiae. Upon induction with galactose, CBGa production was observed in both HB1665 and HB1667. Values are shown in Table 59.

TABLE 59
In vivo production of CBGA with PT296

| | CBGa (mg/L) |
|---|---|
| HB1665 (PT296) | 6.60 |
| HB977 (mScarlett) | 0.00 |
| HB1667 (PT254) | 5.03 |

These data illustrate that PT72, PT273 and PT296 can act as effective prenyltransferases in the conversion of olivetolic acid to CBGa.

Part 6

PKS, NpgA, OAC and Mutants Thereof in the Production of Polyketides and Phytocannabinoids

The present disclosure relates generally to methods for production of polyketides and phytocannabinoids therefrom in a host cell, utilizing PKS, NpgA, OAC and mutants thereof.

Overview

It is an object of the present disclosure to obviate or mitigate at least one disadvantage of previous approaches to producing polyketides in a host cell, and of previous approaches to producing polyketides.

There is described herein a method of producing polyketides, the method comprising: providing a host cell comprising a polyketide synthase polynucleotide coding for a FaPKS polyketide synthase enzyme from Dictyostelium fasciculatum, wherein: the polyketide synthase enzyme is for producing at least one species of polyketide from malonyl-CoA, the polyketide according to formula 6-I:

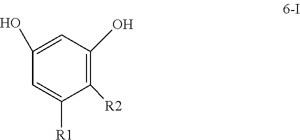

wherein, on formula 6-I, R1 is an alkyl group with a chain length of 1, 2, 3, 4, 5 6, 7, 8, 16 or 18 carbons; and R2 comprises H, carboxyl or methyl; and propagating the host cell for providing a host cell culture.

Further, there is provided a method of producing polyketides, the method comprising: providing a host cell comprising a polyketide synthase polynucleotide coding for a PuPKS polyketide synthase enzyme from Dictyostelium purpureum, wherein: the polyketide synthase enzyme is for producing at least one species of polyketide from malonyl-CoA, the polyketide according to formula 6-II:

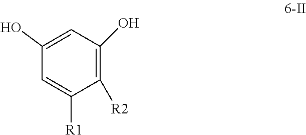

wherein, on formula 6-II, R1 is an alkyl group with a chain length of 1, 2, 3, 4, 5 6, 7, 8, 16 or 18 carbons; and R2 comprises H; wherein the PuPKS polyketide synthase enzyme has a primary structure with between 80% and 100% amino acid residue sequence homology with a protein coded for by a reading frame defined by bases 3486 to 12497 of SEQ ID NO:476, with a charged amino acid residue at amino acid residue position 1452 in place of a glycine residue at position 1452 for mitigating methylation of the at least one species of polyketide; and propagating the host cell for providing a host cell culture.

Additionally, a method of producing polyketides is described, the method comprising: providing a host cell comprising a polyketide synthase polynucleotide coding for at least two copies of a DiPKS polyketide synthase enzyme from Dictyostelium discoideum, wherein: the polyketide synthase enzyme is for producing at least one species of polyketide from malonyl-CoA, the polyketide according to formula 6-III:

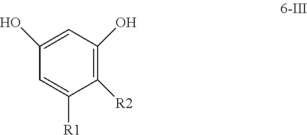

wherein, on formula 6-III, R1 is an alkyl group with a chain length of 1, 2, 3, 4, 5 6, 7, 8, 16 or 18 carbons; and R2 comprises H or carboxyl; and

wherein the DiPKS polyketide synthase enzyme has a primary structure with between 80% and 100% amino acid residue sequence homology with a protein coded for by a reading frame defined by bases selected from the group consisting of bases 849 to 10292 of SEQ ID NO: 477, bases 717 to 10160 of SEQ ID NO:478, bases 795 to 10238 of SEQ ID NO:479, bases 794 to 10237 of SEQ ID NO:480, bases 1172 to 10615 of SEQ ID NO: 481, with a charged amino acid residue at amino acid residue position 1516 in place of a glycine residue at position 1516 for mitigating methylation of the at least one species of polyketide; and propagating the host cell for providing a host cell culture.

Host cells and polynucleotides are described.

### Detailed Description of Part 6

Generally, the present disclosure provides methods and yeast cell lines for producing polyketides Cannabis sativa plant and polyketides with differing side chain lengths. The polyketides are produced in transgenic yeast. The methods and cell lines provided herein include application of genes for enzymes absent from the C. sativa plant. Application of genes other than the complete set of genes in the C. sativa plant that code for enzymes in the biosynthetic pathway resulting in polyketides may provide one or more benefits including biosynthesis of polyketides that are not ordinarily synthesized in C. sativa, biosynthesis of polyketides without input of hexanoic acid, which is toxic to Saccharomyces cerevisiae and other species of yeast, and improved yield.

Many of the 120 phytocannabinoids found in Cannabis sativa may be synthesized from polyketides, and it may be desirable to improve production of polyketides in host cells.

In C. sativa, a type 3 polyketide synthase (“PKS”) enzyme called olivetolic acid synthase (“csOAS”) catalyzes synthesis of olivetolic acid from hexanoyl-CoA and malonyl-CoA in the presence of olivetolic acid cyclase (“csOAC”). Both csOAS and csOAC have been previously characterised as part of the C. sativa phytocannabinoid biosynthesis pathway (Gagne et al., 2012). A prenyltransferase enzyme catalyzes synthesis of cannabigerolic acid (“CBGa”) from olivetolic acid and geranyl pyrophosphate (“GPP”).

PKS enzymes are present across all kingdoms. Dictyostelium discoideum is a species of slime mold that expresses a PKS called “DiPKS”. Wild type DiPKS is a fusion protein consisting of both a type I fatty acid synthase (“FAS”) and a PKS, and is referred to as a hybrid “FAS-PKS” protein. Wild-type DiPKS catalyzes synthesis of 4-methyl-5-pentylbenzene-1,3 diol (“MPBD”) from malonyl-CoA. The reaction has a 6:1 stoichiometric ratio of malonyl-CoA to MPBD.

A mutant form of DiPKS in which glycine 1516 is replaced by arginine (“DiPKS^{G1516R}”) disrupts a methylation moiety of DiPKS. DiPKS^{G1516R} does not synthesize MPBD. In the presence of malonyl-CoA from a glucose source, DiPKS^{G1516R} catalyzes synthesis of only olivetol, and not MPBD (Mookerjee et al., WO2018148848; Mookerjee et al., WO2018148849).

Polyketide synthase enzymes from other species were located in a basic local alignment search tool (“BLAST”) search. The BLAST search showed homology and conservation in the c-methyl transferase domains of PKS enzymes from three additional species: Dictyostelium fasciculatum, Dictyostelium purpureum and Polysphondylium pallidum. The PKS enzymes from D. fasciculatum (“FaPKS”), Dictyostelium purpureum (“PuPKS”), and Polysphondylium pallidum (“PaPKS”) showed between 45.23% and 61.65% overall amino acid sequence homology with DiPKS.

NpgA is a 4′-phosphopantethienyl transferase from Aspergillus nidulans. Expression of NpgA alongside a PKS provides the A. nidulans phosphopantetheinyl transferase for greater catalysis of loading the phosphopantetheine group onto the ACP domain of a PKS. NpgA supports catalysis by DiPKS and homologues of DiPKS, including FaPKS, PuPKS and PaPKS. NpgA also supports catalysis by DiPKS^{G1516R}, and by homologous mutants of FaPKS, PuPKS and PaPKS, respectively including FaPKS^{G1434R} PuPKS^{G1452R} and PaPKS^{G1429R}

The methods and cells lines provided herein may apply and include transgenic cells that have been transformed with nucleotide sequences coding for a PKS and for NpgA. The cells may have also have been transformed with a nucleotide sequence coding for csOAC.

Co-expression of DiPKS^{G1516R}, NpgA and csOAC in S. cerevisiae resulted in production of olivetolic acid in vivo from galactose. Increasing the copy number of DiPKS^{G1516R} increases production of olivetol in the absence of csOAC. In the presence of csOAC, increasing the copy number of DiPKS^{G1516R} increases production of olivetolic acid, and in the ratio of olivetolic acid to olivetol. Strains of S. cerevisiae with csOAC integrated into the genome shows less production of olivetolic acid compared with a strain that expresses csOAC from a plasmid. Plasmid-based expression is associated with a higher copy-number than a typical genome-integrated number of copies. The copy number of both DiPKS^{G1516R} and csOAC affects production of olivetolic acid in S. cerevisiae.

Co-expression of FaPKS with NpgA resulted in production of MPBD. Co-expression of FaPKS^{G1434R} and NpgA resulted in production of olivetol. Co-expression of FaPKS^{G1434R}, NpgA and csOAC resulted in production of olivetol and olivetolic acid.

Co-expression of PuPKS an NpgA did not result in production of MPBD, olivetol or olivetolic acid. Co-expression of PuPKS^{G1452R} and NpgA resulted in production of olivetol. Co-expression of PuPKS^{G1452R}, NpgA and csOAC also resulted in production of olivetol.

Co-expression of PaPKS or PaPKS^{G1429R} and NpgA did not result in production of MPBD, olivetol or olivetolic acid.

Use of DiPKS^{G1516R}, FaPKS^{G1434R} or PuPKS^{G1452R} may provide advantages over csOAS for expression in S. cerevisiae to catalyze synthesis of olivetolic acid, or in the case of PuPKS^{G1452R} olivetol. csOAS catalyzes synthesis of olivetol from malonyl-CoA and hexanoyl-CoA. The reaction has a 3:1:1 stoichiometric ratio of malonyl-CoA to hexanoyl-CoA to olivetol. Olivetol synthesized during this reaction is carboxylated when the reaction is completed in the presence of csOAC, resulting in olivetolic acid. Hexanoic acid is toxic to S. cerevisiae. When applying csOAS and csOAC, hexanoyl-CoA is a necessary precursor for synthesis of olivetolic acid and the presence of hexanoic acid may inhibit proliferation of S. cerevisiae. When using DiPKS^{G1516R} or FaPKS^{G1434R} and csOAC to produce olivetolic acid rather than csOAS and csOAC, the hexanoic acid need not be added to the growth media. The absence of hexanoic acid in growth media may result in increased growth of the S. cerevisiae cultures and greater yield of olivetolic acid compared with S. cerevisiae cultures fed with csOAS.

The S. cerevisiae may have one or more mutations in Erg20, Maf1 or other genes for enzymes or other proteins that support metabolic pathways that deplete GPP, the one or more mutations being for increasing available malonyl-CoA, GPP or both. Alternatively to S. cerevisiae, other species of yeast, including Yarrowia lipolytica, Kluyveromyces marxianus, Kluyveromyces lactis, Rhodosporidium toruloides, Cryptococcus curvatus, Trichosporon pullulan and Lipomyces lipoferetc, may be applied.

Synthesis of olivetolic acid may be facilitated by increased levels of malonyl-CoA in the cytosol. The S. cerevisiae may have overexpression of native acetaldehyde dehydrogenase and expression of a mutant acetyl-CoA synthase or other gene, the mutations resulting in lowered mitochondrial acetaldehyde catabolism. Lowering mitochondrial acetaldehyde catabolism by diverting the acetaldehyde into acetyl-CoA production increases malonyl-CoA available for synthesizing olivetol. Acc1 is the native yeast malonyl CoA synthase. The S. cerevisiae may have over-expression of Acc1 or modification of Acc1 for increased activity and increased available malonyl-CoA. The S. cerevisiae may include modified expression of Maf1 or other regulators of tRNA biosynthesis. Overexpressing native Maf1 has been shown to reduce loss of isopentenyl pyrophosphate (“IPP”) to tRNA biosynthesis and thereby improve monoterpene yields in yeast. IPP is an intermediate in the mevalonate pathway.

In a first aspect, herein provided is a method and cell line for producing polyketides in recombinants organisms. The method applies, and the cell line includes, a host cell transformed with a polyketide synthase CDS and an olivetolic acid cyclase CDS. The polyketide synthase and the olivetolic acid cyclase catalyze synthesis of MPBP, olivetol or olivetolic acid from malonyl CoA. The olivetolic acid cyclase may include Cannabis sativa OAC. The polyketide synthase may include FaPKS, FaPKS^{G1434R}, PuPKS^{G1452R}. Multiple copy numbers of the polyketide synthase may be applied, including multiple copy numbers of DiPKS^{G1516R}. The host cell may include a yeast cell, a bacterial cell, a protest cell or a plant cell.

In a further aspect, here provided is a method of producing polyketides, the method comprising: providing a host cell comprising a polyketide synthase polynucleotide coding for a FaPKS polyketide synthase enzyme from Dictyostelium fasciculatum and propagating the host cell for providing a cell culture. The polyketide synthase enzyme is for producing at least one species of polyketide from malonyl-CoA, having a structure according to formula 6-I:

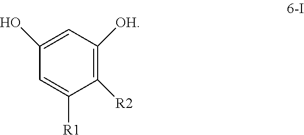

R1 is an alkyl group with a chain length of 1, 2, 3, 4, 5, 6, 7, 8, 16 or 18 carbons; and R2 comprises H, carboxyl or methyl.

In some embodiments, the polyketide synthase comprises a FaPKS polyketide synthase enzyme with a charged amino acid residue at amino acid residue position 1434 in place of a glycine residue at position 1434 for mitigating methylation of the at least one species of polyketide, and R2 comprises H. In some embodiments, the FaPKS polyketide synthase enzyme comprises a FaPKS^{G1434R} polyketide synthase enzyme with a primary structure with between 80% and 100% amino acid residue sequence homology with a protein coded for by a reading frame defined by bases 3486 to 12716 of SEQ ID NO:474. In some embodiments, the host cell further comprises a cyclase polynucleotide coding for an olivetolic acid cyclase enzyme olivetolic acid cyclase enzyme, and R2 comprises H or carboxyl. In some embodiments, the olivetolic acid cyclase enzyme comprises csOAC from C. sativa. In some embodiments, the cyclase polynucleotide comprises a coding sequence for csOAC with a primary structure having between 80% and 100% amino acid residue sequence identity with a protein coded for by a reading frame defined by bases 842 to 1150 of SEQ ID NO:464. In some embodiments, the cyclase polynucleotide has between 80% and 100% base sequence identity with bases 842 to 1150 of SEQ ID NO: 464.

In a further aspect, here provided is a method of producing polyketides, the method comprising: providing a host cell comprising a polyketide synthase polynucleotide coding for a PuPKS polyketide synthase enzyme from Dictyostelium purpureum and propagating the host cell for providing a host cell culture. The polyketide synthase enzyme is for producing at least one species of polyketide from malonyl-CoA, the polyketide having a structure according to formula 6-II:

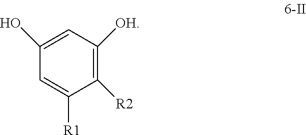

R1 is an alkyl group with a chain length of 1, 2, 3, 4, 5, 6, 7, 8, 16 or 18 carbons; and R2 comprises H. The PuPKS polyketide synthase enzyme has a primary structure with between 80% and 100% amino acid residue sequence homology with a protein coded for by a reading frame defined by bases 3486 to 12497 of SEQ ID NO:476, with a charged amino acid residue at amino acid residue position 1452 in place of a glycine residue at position 1452 for mitigating methylation of the at least one species of polyketide.

In some embodiments, the polyketide synthase comprises a PuPKS^{G1452R} polyketide synthase enzyme, modified relative to PuPKS found from D. discoideum. In some embodiments, the at least one polyketide comprises olivetol and R1 is a pentyl group. In some embodiments, the host cell further comprises a cyclase polynucleotide coding for an olivetolic acid cyclase enzyme olivetolic acid cyclase enzyme. In some embodiments, the olivetolic acid cyclase enzyme comprises csOAC from C. sativa. In some embodiments, the cyclase polynucleotide comprises a coding sequence for csOAC with a primary structure having between 80% and 100% amino acid residue sequence identity with a protein coded for by a reading frame defined by bases 842 to 1150 of SEQ ID NO: 464. In some embodiments, the cyclase polynucleotide has between 80% and 100% base sequence identity with bases 842 to 1150 of SEQ ID NO: 464.

In a further aspect, here provided is a method of producing polyketides, the method comprising: providing a host cell comprising a polyketide synthase polynucleotide coding for at least two copies of a DiPKS polyketide synthase enzyme from Dictyostelium discoideum and propagating the host cell for providing a host cell culture. The polyketide synthase enzyme is for producing at least one species of polyketide from malonyl-CoA, the polyketide having a structure according to formula 6-III:

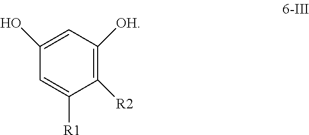

R1 is an alkyl group with a chain length of 1, 2, 3, 4, 5, 6, 7, 8, 16 or 18 carbons; and R2 comprises H or carboxyl. The DiPKS polyketide synthase enzyme has a primary structure with between 80% and 100% amino acid residue sequence homology with a protein coded for by a reading frame defined by bases selected from the group consisting of bases 849 to 10292 of SEQ ID NO: 477, bases 717 to 10160 of SEQ ID NO: 478, bases 795 to 10238 of SEQ ID NO: 479, bases 794 to 10237 of SEQ ID NO: 480, bases 1172 to 10615 of SEQ ID NO: 481, with a charged amino acid residue at amino acid residue position 1516 in place of a glycine residue at position 1516 for mitigating methylation of the at least one species of polyketide.

In some embodiments, the polyketide synthase comprises a DiPKS^{G1516R} polyketide synthase enzyme, modified relative to DiPKS found from D. discoideum. In some embodiments, the host cell further comprises a cyclase polynucleotide coding for an olivetolic acid cyclase enzyme olivetolic acid cyclase enzyme and wherein the at least one polyketide further comprises a polyketide in which R2 comprises a carboxyl group. In some embodiments, the olivetolic acid cyclase enzyme comprises csOAC from C. sativa. In some embodiments, the cyclase polynucleotide comprises a coding sequence for csOAC with a primary structure having between 80% and 100% amino acid residue sequence identity with a protein coded for by a reading frame defined by bases 842 to 1150 of SEQ ID NO: 464. In some embodiments, the cyclase polynucleotide has between 80% and 100% base sequence identity with bases 842 to 1150 of SEQ ID NO: 464.

In some embodiments, the host cell comprises a phosphopantetheinyl transferase polynucleotide coding for a phosphopantetheinyl transferase enzyme for increasing the activity of the polyketide synthase enzyme. In some embodiments, the phosphopantetheinyl transferase comprises NpgA phosphopantetheinyl transferase enzyme from A. nidulans. In some embodiments, the host cell comprises a genetic modification to increase available geranylpyrophosphate. In some embodiments, the genetic modification comprises a partial inactivation of the farnesyl synthase functionality of the Erg20 enzyme. In some embodiments, the host cell comprises an Erg20^{K197E} polynucleotide including a coding sequence for Erg20^{K197E}In some embodiments, the host cell comprises a genetic modification to increase available malonyl-CoA. In some embodiments, the host cell comprises a yeast cell and the genetic modification comprises increased expression of Maf1. In some embodiments, the genetic modification comprises a modification for increasing cytosolic expression of an aldehyde dehydrogenase and an acetyl-CoA synthase. In some embodiments, the host cell comprises a yeast cell and the genetic modification comprises a modification for expressing for Acs^{L641P} from S. enterica and Ald6 from S. cerevisiae. In some embodiments, the genetic modification comprises a modification for increasing malonyl-CoA synthase activity. In some embodiments, the host cell comprises a yeast cell and the genetic modification comprises a modification for expressing Acc1^{S659A; S1157A} from S. cerevisiae. In some embodiments, the host cell comprises a yeast cell comprising an Acc1 polynucleotide including the coding sequence for Acc1 from S. cerevisiae under regulation of a constitutive promoter. In some embodiments, the constitutive promoter comprises a PGK1 promoter from S. cerevisiae.

The host cell can be a bacterial cell, a fungal cell, a protist cell, or a plant cell, such as any of the exemplary cell types noted herein in Table 2. Exemplary host cell types include S. cerevisiae, E. coli, Yarrowia lipolytica, and Komagataella phaffii.

In some embodiments, the method includes extracting the at least one species of polyketide from the host cell culture.

In a further aspect, here provided is a host cell for producing polyketides, the host cell comprising: a first polynucleotide coding for a polyketide synthase enzyme; and a second polynucleotide coding for an olivetolic acid cyclase enzyme.

In some embodiments, the host cell includes the features of one or more of the host cell, the polyketide synthase polynucleotide, the cyclase polynucleotide, the phosphopantetheinyl transferase polynucleotide, the Erg20^{K197E} polynucleotide, the genetic modification to increase available malonyl-CoA or the genetic modification to increase available geranylpyrophosphate.

In a further aspect, herein provided is a method of transforming a host cell for production of polyketides, the method comprising introducing a first polynucleotide coding for a polyketide synthase enzyme into the host cell line; and introducing a second polynucleotide coding for an olivetolic acid cyclase enzyme into the host cell.

In some embodiments, the method includes the features of one or more of the host cell, the polyketide synthase polynucleotide, the cyclase polynucleotide, the phosphopantetheinyl transferase polynucleotide, the Erg20^{K197E} polynucleotide, the genetic modification to increase available malonyl-CoA or the genetic modification to increase available geranylpyrophosphate as described herein.

In a further aspect, herein provided is an FaPKS polyketide synthase enzyme with a charged amino acid residue at amino acid residue position 1434 in place of a glycine residue at position 1434.

In some embodiments, the FaPKS polyketide synthase enzyme has a primary structure with between 80% and 100% amino acid residue sequence homology with a protein coded for by a reading frame defined by bases 3486 to 12716 of SEQ ID NO:474.

In a further aspect, herein provided is an FaPKS polyketide synthase enzyme with a charged amino acid residue at amino acid residue position 1434 in place of a glycine residue at position 1434.

In some embodiments, the polynucleotide has between 80% and 100% nucleotide residue sequence homology with bases 3486 to 12716 of SEQ ID NO: 474.

In a further aspect, herein provided is a PuPKS polyketide synthase enzyme with a charged amino acid residue at amino acid residue position 1452 in place of a glycine residue at position 1452.

In some embodiments, the PuPKS polyketide synthase enzyme has a primary structure with between 80% and 100% amino acid residue sequence homology with a protein coded for by a reading frame defined by bases 3486 to 12497 of SEQ ID NO:476.

In a further aspect, herein provided is a polynucleotide coding for a PuPKS polyketide synthase enzyme with a charged amino acid residue at amino acid residue position 1452 in place of a glycine residue at position 1452.

In some embodiments, the polynucleotide has between 80% and 100% nucleotide residue sequence homology with bases 3486 to 12497 of SEQ ID NO: 476.

FIG. 28 is a schematic of biosynthesis of olivetolic acid and related compounds with different alkyl group chain lengths in C. sativa. FIG. 29 is a schematic of biosynthesis of CBGa from hexanoic acid, malonyl-CoA, and geranyl pyrophosphate in C. sativa. FIG. 30 is a schematic of biosynthesis of downstream phytocannabinoids in acid form CBGa C. sativa. FIG. 31 is a schematic of biosynthesis of MPBD by DiPKS. FIG. 32 is a schematic of functional domains in DiPKS, with mutations to a C-methyl transferase that for lowering methylation of olivetol. FIGS. 28 to 32 are describe in detail above.

Methods and yeast cells as provided herein for production of polyketides may apply and include S. cerevisiae transformed with a gene for csOAS from C. sativa.

DiPKS and Mutants

Conversion of malonyl-CoA and hexanoyl-CoA to olivetolic acid catalyzed by csOAS at Reaction 2 of FIG. 29 was identified as a metabolic bottleneck in the pathway of FIG. 29, as described in further detail above. FIG. 31 shows production of MPBD from malonyl-CoA as catalyzed by DiPKS.

DiPKS Homologues and Mutants

Polyketide synthase enzymes from other species were located in a basic local alignment search tool (“BLAST”) search. The BLAST search showed homology and conservation in the c-methyl transferase domains of PKS enzymes from three additional species: Dictyostelium fasciculatum, Dictyostelium purpureum and Polysphondylium pallidum. The PKS enzymes from D. fasciculatum (“FaPKS”), Dictyostelium purpureum (“PuPKS”), and Polysphondylium pallidum (“PaPKS”) showed overall amino acid sequence homology with DiPKS according to Table 60.

TABLE 60
DiPKS Homologues

| Organism | Name of PKs | % Similarity to DiPKS |
|---|---|---|
| Dictyostelium fasciculatum | FaPKS | 45.23% |
| Dictyostelium purpureum | PuPKS | 61.65% |
| Polysphondylium pallidum | PaPKS | 45.81% |

The primary amino acid sequences of FaPKS, PuPKS and PaPKS were aligned the amino acid with DiPKS to see if there were any conserved residues in the C-methyltransferase domain of the proteins. Molecular Evolutionary Genetic Analysis (“MEGA”) software and Muscle were used to create amino acid sequence alignments and determine the degree of conservation. As shown in Table 61A-610, the alignments showed that the C-methyltransferase domain was highly conserved, including a glycine residue believed to correspond to glycine 1516 in DiPKS.

TABLE 61A
Alignment between DiPKS, FaPKS, PuPKS and PaPKS

| Species | | | | | | | | * | | | | | | | | | | | |
|---|---|---|---|---|---|---|---|---|---|---|---|---|---|---|---|---|---|---|---|
| D. discoideum | S | E | M | V | L | E | S | I | R | P | I | V | R | E | — | — | — | — | — |
| D. fasciculatum | G | S | T | I | Q | K | A | I | G | N | I | V | T | K | S | D | Q | D | C |
| D. purpureum | A | S | L | V | L | E | S | I | K | P | I | V | R | E | — | — | — | — | — |
| P. pallidum | A | D | T | I | Q | H | A | I | T | S | K | L | S | E | — | — | — | — | — |

TABLE 61C
Alignment between DiPKS, FaPKS, PuPKS and PaPKS (con't)

| Species | | * | | | | | | | | | | | | | | | | | |
|---|---|---|---|---|---|---|---|---|---|---|---|---|---|---|---|---|---|---|---|
| D. discoideum | V | L | T | K | L | N | T | Y | L | S | T | L | N | S | N | G | G | S | G |
| D. fasciculatum | L | L | T | K | L | A | S | L | F | — | — | — | — | — | — | E | G | T | T |
| D. purpureum | V | L | E | K | L | N | K | F | L | — | — | — | — | — | — | S | I | N | S |
| P. pallidum | L | L | N | T | F | N | L | I | L | — | — | — | — | — | — | G | G | P | K |

TABLE 61D
Alignment between DiPKS, FaPKS, PuPKS and PaPKS (con't)

| Species | | | | | | | | | | * | | * | * | * | | * | | |
|---|---|---|---|---|---|---|---|---|---|---|---|---|---|---|---|---|---|---|
| D. discoideum | Y | — | — | — | N | I | I | I | E | Y | T | F | T | D | I | S | A | N |
| D. fasciculatum | Y | E | K | S | G | V | E | V | V | Y | T | F | T | D | I | S | A | S |
| D. purpureum | D | K | — | — | N | I | I | V | E | Y | N | F | T | D | I | S | S | S |
| P. pallidum | Q | — | — | — | R | I | E | I | E | Y | T | F | T | D | V | S | A | G |

This conserved domain alignment was further utilized to create mutants of FaPKS, PuPKS and PaPKS to mitigate activity at the c-methyltransferase domain. DiPKS^{G1516R} was used to identify the cognate residue corresponding to conserved glycine 1516 in DiPKS, which in DiPKS is critical for functionality of the C-met Domain. The corresponding residue in each of FaPKS, PuPKS and PaPKS was modified in each case to an arginine residue. Specifically, the residues corresponding to glycine 1516 in DiPKS were mutated to arginine in each of FaPKS, PuPKS and PaPKS, resulting in FaPKS^{G1434R} PuPKS^{G1452R} and PaPKS^{G1429R} The wild-type and mutant homologs of DiPKS were subsequently codon-optimized for S. cerevisiae expression using EMBOSS BACKTRANSSEQ (https: //www.ebi.ac.uk/Tools/st/emboss_backtranseq/) and synthesized by GenScript USA Inc. They were synthesized in the standard yeast expression vector pESC UR.

FIG. 32 is a schematic of the functional domains of PKS enzymes, including DiPKS, FaPKS, PuPKS and PaPKS. FIG. 32 shows functional domains similar to domains found in a fatty acid synthase, and in additional includes a methyltransferase domain and a PKS III domain, and is described in detail above. The “Type III” domain is a type 3 PKS. The KS, AT, DH, ER, KR, and ACP portions provide functions typically associated with a fatty acid synthase, speaking to DiPKS, FaPKS, PuPKS and PaPKS each being a FAS-PKS protein. The C-Met domain provides the catalytic activity for methylating olivetol at carbon 4, providing MPBD. The C-Met domain is crossed out in FIG. 32, schematically illustrating changes to DiPKS, FaPKS, PuPKS and PaPKS that inactivate the C-Met domain and mitigate or eliminate methylation functionality.

A mutant form of DiPKS in which glycine 1516 is replaced by arginine (“DiPKS^{G1516R}”) disrupts a methylation moiety of DiPKS. DiPKS^{G1516R} does not synthesize MPBD. In the presence of malonyl-CoA from a glucose or other sugar source, and in the absence of csOAC or another olivetolic acid cyclase or other polyketide cyclase, DiPKS^{G1516R} catalyzes synthesis of only olivetol, and not MPBD (Mookerjee et al., WO2018148848; Mookerjee et al. WO2018148849). Application of DiPKS^{G1516R} rather than csOAS facilitates production of polyketides without hexanoic acid supplementation. Since hexanoic acid is toxic to S. cerevisiae, eliminating a requirement for hexanoic acid in the biosynthetic pathway for polyketides may provide greater yields of polyketides than the yields of polyketides in a yeast cell expressing csOAS and Hex1.

Through the MEGA search of DiPKS, FaPKS, PuPKS and PaPKS and associated alignment as shown in FIG. 29, FaPKS^{G1434R}, PuPKS^{G1452R} and PaPKS^{G1429R} were each prepared.

Transforming and Growing Yeast Cells

Details of specific examples of methods carried out and yeast cells produced in accordance with this description are provided below as Examples 16, 17, and 18. Each of these three specific examples applied similar approaches to plasmid construction, transformation of yeast, quantification of strain growth, and quantification of intracellular metabolites. These common features across the three examples are described below, followed by results and other details relating to one or more of the examples.

As shown in Table 62, six strains of yeast were prepared. In the “Genotype” column, the integration-based modifications are listed in the order they were introduced into the genome. Base strain “HB42” is a uracil and leucine auxotroph CEN PK2 variant of S. cerevisiae. Modified base strain “HB144” was prepared from HB42 with several genetic modifications to increase the availability of biosynthetic precursors and to increase PKS activity. Additional details are in Table 63.

All subsequent strains were based on HB144. Strains HB259, HB309, HB310 and HB742 each included between one and five copy numbers of DiPKS^{G1516R}. Strain HB801 included five copy numbers of DiPKS^{G1516R} and csOAC. Strains HB865, HB866, HB867, HB868, HB869 and HB870 each included one of FaPKS, PuPKS, PaPKS, FaPKS^{G1434R} PuPKS^{G1452R} and PaPKS^{G1429R}. Strains HB873, HB874, HB875 and HB877 each included between one and five copy numbers of DiPKS^{G1516R} and each included csOAC. Strain HB1030 included csOAC integrated into HB144. Strain HB1113 included PuPKS^{G1452R} and csOAC. Strain HB1114 include FaPKS^{G1434R} and csOAC.

TABLE 62
Yeast Strains

| Strain | Plasmids | Genotype | Notes |
|---|---|---|---|
| HB42 | None | CEN.PK2 | Base Strain |
| | | ΔLEU2 | |
| | | ΔURA3 | |
| | | Erg20K197E::KanMx | |
| HB144 | None | (HB42) | Modified Base Strain |
| | | ALD6; ASC1^{L641P} | |
| | | NPGA | |
| | | MAF1 | |
| | | PGK1p: | |
| | | Acc1^{S659A; S1157A} | |
| | | tHMGR1; IDI | |
| HB259 | None | (HB144) | DiPKS^{G1516R} x 1 |
| | | DiPKS^{G1516R} | Produces Olivetol |
| | | UB14p:ERG20 | |
| HB309 | None | (HB259) | DiPKS^{G1516R} x 3 |
| | | DiPKS^{G1516R} | Produces Olivetol |
| | | DiPKS^{G1516R} | |
| HB310 | None | (HB309) | DiPKS^{G1516R} x 4 |
| | | DiPKS^{G1516R} | Produces Olivetol |
| HB742 | None | (HB310) | DiPKS^{G1516R} x 5 |
| | | DiPKS^{G1516R} | Produces Olivetol |
| HB801 | None | (HB742) | DiPKS^{G1516R} x 5 |
| | | Gal1p:csOAC | Produces Olivetolic Acid |
| HB865 | Plas-43 | HB144 | PaPKS |
| | | | No Production of MPBD, |
| | | | Olivetol or |
| | | | Olivetolic Acid |
| HB866 | Plas-46 | HB144 | PaPKS^{G1429R} |
| | | | No Production of MPBD, |
| | | | Olivetol or |
| | | | Olivetolic Acid |
| HB867 | Plas-47 | HB144 | FaPKS |
| | | | Produces MPBD |
| HB868 | Plas-180 | HB144 | PuPKS^{G1452R} |
| | | | Produces Olivetol |
| HB869 | Plas-191 | HB144 | PuPKS |
| | | | No Production of MPBD, |
| | | | Olivetol or |
| | | | Olivetolic Acid |
| HB870 | Plas-249 | HB144 | FaPKS^{G1434R} |
| | | | Produces Olivetol |
| HB873 | Plas-48 | HB259 | DiPKS^{G1516R} x 1 |
| | | | Produces Olivetol and |
| | | | Olivetolic Acid |
| HB874 | Plas-48 | HB309 | DiPKS^{G1516R} x 3 |
| | | | Produces Olivetol and |
| | | | Olivetolic Acid |
| HB875 | Plas-48 | HB310 | DiPKS^{G1516R} x 4 |
| | | | Produces Olivetol and |
| | | | Olivetolic Acid |
| HB877 | Plas-48 | HB742 | DiPKS^{G1516R} x 5 |
| | | | Produces Olivetol and |
| | | | Olivetolic Acid |
| HB1030 | None | (HB144) | Modified Base Strain |
| | | Gal1p:csOAC | Includes csOAC |
| HB1113 | Plas-180 | HB1030 | PuPKS^{G1452R} |
| | | | Produces Olivetol |
| HB1114 | Pas-249 | HB1030 | FaPKS^{G1434R} |
| | | | Produces Olivetol and |
| | | | Olivetolic Acid |

Protein sequences and coding DNA sequences used to prepare the strains in Table 62 are provided below in Table 63 and full sequence listings are provided below.

TABLE 63
Protein and DNA Sequences used to Prepare the Yeast Strains

| SEQ ID NO | Description | Material | Length | Coding Sequence |
|---|---|---|---|---|
| 462 | csOAC | Protein | 102 | Entire sequence |
| 463 | PLAS48 | DNA | 6094 | 1 to 306 |
| 464 | Gal1p:csOAC:Eno2t | DNA | 2177 | 842 to 1150 |
| | expression/integration cassette | | | |
| 465 | DiPKS | Protein | 3147 | 1 to 3147 |
| 466 | DiPKS^{G1516R} | Protein | 3147 | 1 to 3147 |
| 467 | FaPKS | Protein | 3076 | 1 to 3076 |
| 468 | FaPKS^{G1434R} | Protein | 3076 | 1 to 3076 |
| 469 | PuPKS | Protein | 3003 | 1 to 3003 |
| 470 | PuPKS^{G1452R} | Protein | 3003 | 1 to 3003 |
| 471 | PaPKS | Protein | 3026 | 1 to 3026 |
| 472 | PaPKS^{G1429R} | Protein | 3026 | 1 to 3026 |
| 473 | pESC_Gal1p:FaPKS:Cyc1t | DNA | 16888 | 3486 to 12716 |
| 474 | pESC_Gal1p:FaPKS^{G1434R}:Cyc1t | DNA | 16888 | 3486 to 12716 |
| 475 | pESC_Gal1p:PuDiPKS:Cyc1t | DNA | 16669 | 3486 to 12497 |
| 476 | pESC_Gal1p:PuPKS^{G1452R}:Cyc1t | DNA | 16669 | 3486 to 12497 |
| 477 | pESC_Gal1p:PaPKS:Cyc1t | DNA | 16738 | 3486 to 12566 |
| 478 | pESC_Gal1p:PaPKS^{G1429R}:Cyc1t | DNA | 16738 | 3486 to 12566 |
| 479 | NpgA | DNA | 3564 | 1170 to 2201 |
| 480 | DiPKS-1 | DNA | 11114 | 849 to 10292 |
| 481 | DiPKS-2 | DNA | 10890 | 717 to 10160 |
| 481 | DiPKS-3 | DNA | 11300 | 795 to 10238 |
| 483 | DiPKS-4 | DNA | 11140 | 794 to 10237 |
| 484 | DiPKS-5 | DNA | 11637 | 1172 to 10615 |
| 485 | PDH | DNA | 7114 | Ald6: 1444 to 2949 |
| | | | | ACS: 3888 to 5843 |
| 486 | Maf1 | DNA | 3256 | 936 to 2123 |
| 487 | Erg20K197E | DNA | 4254 | 2683 to 3423 |
| 488 | Erg1p:UB14-Erg20:deg | DNA | 3503 | 1364 to 2701 |
| 489 | tHMGr-IDI1 | DNA | 4843 | tHMGR1: |
| | | | | 877 to 2385 |
| | | | | IDI1: 3209 to 4075 |
| 490 | PGK1p:ACC1^{S659A, S1157A} | DNA | 7673 | Pgk1p: 222 to 971 |
| | | | | Acc1^{S659A, S1157A}: |
| | | | | 972 to 7673 |
| 491 | PLAS36 | DNA | 8980 | Not applicable |

Genome Modification of S. cerevisiae

HB42 was used as a base strain to develop all other strains in this experiment. All DNA was transformed into strains using the transformation protocol described in Gietz et al. (2007). Plas-36 was used for the genetic modifications described in this experiment that apply clustered regularly interspaced short palindromic repeats (CRISPR).

The genome of HB42 was iteratively targeted by gRNA's and Cas9 expressed from PLAS36 to make the following genomic modifications in the order of the Table 64 below. Erg20^{K197E} was already included in HB42 and is marked as being order “0”. The strains resulting from the genomic integrations are listed in Table 62.

TABLE 64
Gene Integration in HB742

| Order | Modification | Integration | Description | Genetic Structure |
|---|---|---|---|---|
| 0 | Erg20^{K197E} | Chromosomal | Mutant of Erg20 protein that | Tpi1p:ERG20K197E: |
| | SEQ ID NO. 487 | modification | diminishes FPP synthase | Cyc1t::Tef1p:KanMX:Tef1t |
| | | | activity creating greater pool | |
| | | | of GPP precursor. | |
| | | | Negatively affects growth | |
| | | | phenotype. (Oswald et al., | |
| | | | 2007) | |
| 1 | PDH bypass | Flagfeldt Site | Acetaldehyde | 19Up::Tdh3p:Ald6: |
| | SEQ ID NO. 485 | 19 integration | dehydrogenase (ALD6) from | Adh1::Tef1p:seACS1^{L641P}: |
| | | | S. cerevisiae and | Prm9t::19Down |
| | | | acetoacetyl coA synthase | |
| | | | (AscL641P) from Salmonella | |
| | | | enterica. Will allow greater | |
| | | | accumulation of acetyl-coA | |
| | | | in the cell. (Shiba et al., | |
| | | | 2007) | |
| 2 | NpgA | Flagfeldt Site | Phosphopantetheinyl | Site14Up::Tef1p:NpgA: |
| | SEQ ID NO. 479 | 14 integration | Transferase from Aspergillus | Prm9t:Site14Down |
| | | | niger. Accessory Protein for | |
| | | | DiPKS (Kim et al., 2007) | |
| 3 | Maf1 | Flagfeldt Site 5 | Maf1 is a regulator of tRNA | Site5Up::Tef1p:Maf1: |
| | SEQ ID NO. 486 | integration | biosynthesis. | Prm9t:Site5Down |
| | | | Overexpression in S. | |
| | | | cerevisiae has demonstrated | |
| | | | higher monoterpene (GPP) | |
| | | | yields (Liu et al., 2013) | |
| 4 | PGK1p:ACC1^{S659A, S1157A} | Chromosomal | Mutations in the native S. | Pgk1:ACC1^{S659A, S1157A}: |
| | SEQ ID NO. 490 | Modification | cerevisiae acetyl-coA | Acc1t |
| | | | carboxylase that removes | |
| | | | post-translational | |
| | | | modification based down- | |
| | | | regulation. Leads to greater | |
| | | | malonyl-coA pools. The | |
| | | | promoter of Acc1 was also | |
| | | | changed to a constitutive | |
| | | | promoter for higher | |
| | | | expression. (Shi et al., 2014) | |
| 5 | tHMGR-IDI1 | USER Site X-3 | Overexpression of truncated | X3up::Tdh3p:tHMGR1: |
| | SEQ ID NO. 489 | integration | HMGr1 and IDI1 proteins | Adh1t::Tef1p:IDI1: |
| | | | that have been previously | Prm9t::X3down |
| | | | identified to be bottlenecks | |
| | | | in the S. cerevisiae | |
| | | | terpenoid pathway | |
| | | | responsible for GPP | |
| | | | production. (Ro et al., 2006) | |
| 6 | DiPKS^{G1516R}-1 | USER Site XII- | Type 1 FAS fused to Type 3 | XII-1up::Gal1p: |
| | SEQ ID NO. 480 | 1 integration | PKS from D. discoideum. | DiPKS^{G1516R}: |
| | | (Jensen et al., | Produces Olivetol from | Prm9t::XII1-down |
| | | no date) | malonyl-coA | |
| 7 | Erg1p:UB14- | Flagfeldt Site | Sterol responsive promoter | Site18Up::Erg1p: |
| | Erg20:deg | 18 integration | controlling Erg20 protein | UB14deg:ERG20: |
| | SEQ ID NO. 488 | | activity. Allows for regular | Adh1t:Site18down |
| | | | FPP synthase activity and | |
| | | | uninhibited growth | |
| | | | phenotype until | |
| | | | accumulation of sterols | |
| | | | which leads to a suppression | |
| | | | of expression of enzyme. | |
| | | | (Peng et al., 2018) | |
| 8 | DiPKS^{G1516R}-2 | Wu site 1 | Type 1 FAS fused to Type 3 | Wu1up::Gal1p: |
| | SEQ ID NO. 481 | integration | PKS from D. discoideum. | DiPKS^{G1516R}: |
| | | | Produces Olivetol from | Prm9t::Wu1down |
| | | | malonyl-coA | |
| 9 | DiPKS^{G1516R}-3 | Wu site 3 | Type 1 FAS fused to Type 3 | Wu3up::Gal1p: |
| | SEQ ID NO. 482 | integration | PKS from D. discoideum. | DiPKS^{G1516R}: |
| | | | Produces Olivetol from | Prm9t::Wu3down |
| | | | malonyl-coA | |
| 10 | DiPKS^{G1516R}-4 | Wu site 6 | Type 1 FAS fused to Type 3 | Wu6up::Gal1p: |
| | SEQ ID NO. 483 | integration | PKS from D. discoideum. | DiPKS^{G1516R}: |
| | | | Produces Olivetol from | Prm9t::Wu6down |
| | | | malonyl-coA | |
| 11 | DiPKS^{G1516R}-5 | Wu site 18 | Type 1 FAS fused to Type 3 | Wu18up::Gal1p: |
| | SEQ ID NO. 484 | integration | PKS from D. discoideum. | DiPKS^{G1516R}: |
| | | | Produces Olivetol from | Prm9t::Wu18down |
| | | | malonyl-coA | |
| 12 | csOAC | Flagfeldt Site | C. sativa Olivetolic acid | Site16Up::Gal1p: |
| | SEQ ID NO. 464 | 16 integration | cyclase | csOAC:Eno2t:Site16down |

To create HB1030, HB144 was modified with SEQ ID NO. 464 in a similar fashion to that applied to HB742 to create HB801.

The S. cerevisiae strains described herein may be prepared by stable transformation of plasmids, genome integration or other genome modification. Genome modification may be accomplished through homologous recombination, including by methods leveraging CRISPR.

Methods applying CRISPR were applied to delete DNA from the S. cerevisiae genome and introduce heterologous DNA into the S. cerevisiae genome, as described above in PART 4.

Integration site homology sequences for integration into the S. cerevisiae genome using CRISPR may be at Flagfeldt sites. A description of Flagfeldt sites is provided in Bai Flagfeldt, et al., (2009). Other integration sites may be applied as indicated in Table 64.

Increasing Availability of Biosynthetic Precursors

The biosynthetic pathways shown in FIG. 42 each require malonyl-CoA to produce MPBD, olivetol or olivetolic acid. Yeast cells may be mutated, genes from other species may be introduced, genes may be upregulated or downregulated, or the yeast cells may be otherwise genetically modified to increase production of olivetolic acid, CBGa or downstream phytocannabinoids. In addition to introduction of a PKS and an olivetolic acid cyclase such as csOAC, additional modifications may be made to the yeast cell to increase the availability of malonyl-CoA, GPP, or other input metabolites to support the biosynthetic pathways of any of FIG. 42.

As shown in FIG. 32, DiPKS^{G1516R} includes an ACP domain. The ACP domain of DiPKS^{G1516R} requires a phosphopantetheine group as a co-factor. NpgA is a 4′-phosphopantethienyl transferase from Aspergillus nidulans. A codon-optimized copy of NpgA for S. cerevisiae may be introduced into S. cerevisiae and transformed into the S. cerevisiae, including by homologous recombination. In HB144, an NpgA gene cassette was integrated into the genome of Saccharomyces cerevisiae at Flagfeldt site 14.

Expression of NpgA provides the A. nidulans phosphopantetheinyl transferase for greater catalysis of loading the phosphopantetheine group onto the ACP domain of a PKS. As a result, the reaction catalyzed by DiPKS^{G1516R} (FIG. 42) or the other PKS enzymes may occur at greater rate, providing a greater amount of olivetolic acid. As shown above in Table 62, HB144 includes an integrated polynucleotide including a coding sequence NpgA, as does each modified yeast strain based on HB144 (HB259, HB309, HB310, HB742, HB801, HB865, HB866, HB867, HB868, HB869, HB870, HB873, HB874, HB875, HB877, HB1030, HB1113 and HB1114).

The sequence of the integrated DNA coding for NpgA is shown in SEQ ID NO: 479, and includes the Tef1 Promoter, the NpgA coding sequence and the Prm9 terminator. Together the Tef1p, NpgA, and Prm9t are flanked by genomic DNA sequences promoting integration into Flagfeldt site 14 in the S. cerevisiae genome.

The yeast strains may be modified for increasing available malonyl-CoA. Lowered mitochondrial acetaldehyde catabolism results in diversion of the acetaldehyde from ethanol catabolism into acetyl-CoA production, which in turn drives production of malonyl-CoA and downstream polyketides and terpenoids. S. cerevisiae may be modified to express an acetyl-CoA synthase from Salmonella enterica with a substitution modification of Leucine to Proline at residue 641 (“ACs^{L641P}”), and with aldehyde dehydrogenase 6 from S. cerevisiae (“Ald6”). The Leu641 Pro mutation removes downstream regulation of Acs, providing greater activity with the Acs^{L641P} mutant than the wild type Acs. Together, cytosolic expression of these two enzymes increases the concentration of acetyl-CoA in the cytosol. Greater acetyl-CoA concentrations in the cytosol result in lowered mitochondrial catabolism, bypassing mitochondrial pyruvate dehydrogenase (“PDH”), providing a PDH bypass. As a result, more acetyl-CoA is available for malonyl-CoA production.

SEQ ID NO:485 includes coding sequences for the genes for Ald6 and SeAcsL641P, promoters, terminators, and integration site homology sequences for integration into the S. cerevisiae genome at Flagfeldt-site 19. As shown in Table 64 a portion of SEQ ID NO:485 from bases 1444 to 2949 codes for Ald6 under the TDH3 promoter, and bases 3888 to 5843 code for SeAcsL641P under the Tef1P promoter.

S. cerevisiae may include modified expression of Maf1 or other regulators of tRNA biosynthesis. Overexpressing native Maf1 has been shown to reduce loss of IPP to tRNA biosynthesis and thereby improve monoterpene yields in yeast. IPP is an intermediate in the mevalonate pathway. As shown in Table 62, HB742 includes an integrated polynucleotide including a coding sequence for Maf1 under the Tef1 promoter, as does each modified yeast strain based on HB742 (H1B801, H B861, H B862, H B814 and H B888).

SEQ ID NO:486 is a polynucleotide that was integrated into the S. cerevisiae genome at Flagfeldt-site 5 for genomic integration of Maf1 under the Tef1 promoter. SEQ ID NO: 486 includes the Tef1 promoter, the native Maf1 gene, and the Prm9 terminator. Together, Tef1, Maf1, and Prm9 are flanked by genomic DNA sequences for promoting integration into the S. cerevisiae genome.

The yeast cells may be modified for increasing available GPP. S. cerevisiae may have one or more other mutations in Erg20 or other genes for enzymes that support metabolic pathways that deplete GPP. Erg20 catalyzes GPP production in the yeast cell. Erg20 also adds one subunit of 3-isopentyl pyrophosphate (“IPP”) to GPP, resulting in farnesyl pyrophosphate (“FPP”), a metabolite used in downstream sesquiterpene and sterol biosynthesis. Some mutations in Erg20 have been demonstrated to reduce conversion of GPP to FPP, increasing available GPP in the cell. A substitution mutation Lys197Glu in Erg20 lowers conversion of GPP to FPP by Erg20. As shown in Table 62, base strain HB742 expresses the Erg20^{K197E} mutant protein. Similarly, each modified yeast strain based on any of HB742, (HB801, HB861, HB862, HB814 and HB888) includes an integrated polynucleotide coding for the Erg20^{K197E} mutant integrated into the yeast genome.

SEQ ID NO:487 is a CDS coding for the Erg20^{K197E} protein under control of the Tpi1p promoter and the Cyc1t terminator, and a coding sequence for the KanMX protein under control of the Tef1p promoter and the Tef1t terminator.

SEQ ID NO:488 is a CDS coding for the Erg20 protein under control of the Erg1p promoter and the Adh1t terminator, and flanking sequences for homologous recombination. The Erg1 promoter is downregulated by the presence of large amounts of Ergosterol in the cell. When the cells are growing and there is not much ergosterol in the cell, the Erg1 promoter aids in the expression of the native Erg20 protein that allows the cells to grow without any growth defects associated with the attenuation of FPP synthase activity. When the cells have high amounts of ergosterol present in later stages of growth then the Erg1 promoter is inhibited leading to the cessation of expression of the native Erg20 protein. The extant copies of the native Erg20 protein in the cell are quickly degraded due the UB14 degradation tag. This allows the mutant Erg20K197E to be functional leading to GPP accumulation.

SEQ ID NO:489 is a CDS coding for the truncated HMGr1 under control of the Tdh3p promoter and the Adh1t terminator, and the IDI1 protein under control of the Tef1p promoter and the Prm9t terminator, and flanking sequences for homologous recombination of both sequences for genome integration. The HMG1 protein catalyzed reduction and the IDI1 catalyzed isomerization have previously been identified as rate limiting steps in the eukaryotic mevalonic pathway. Thus, over-expression of these proteins have been demonstrated to alleviate the bottlenecks in the mevalonate pathway and increase the carbon flux for GPP and FPP production.

Another approach to increasing cytosolic malonyl-CoA is to upregulate Acc1, which is the native yeast malonyl-CoA synthase. In HB742, the promoter sequence of the Acc1 gene was replaced by a constitutive yeast promoter for the PGK1 gene. The promoter from the PGK1 gene allows multiple copies of Acc1 to be present in the cell. The native Acc1 promoter allows only a single copy of the protein to be present in the cell at a time. As shown in Table 62, base strain HB742 includes the Acc1 under the PGK1 promoter, as does each modified yeast strain based on HB742 (H1B801, H B861, H B862, H B814 and H B888).

In addition to upregulating expression of Acc1, S. cerevisiae may include one or more modifications of Acc1 to increase Acc1 activity and cytosolic acetyl-CoA concentrations. Two mutations in regulatory sequences were identified in literature that remove repression of Acc1, resulting in greater Acc1 expression and higher malonyl-CoA production. HB742 includes a coding sequence for the Acc1 gene with Ser659Ala and Ser1157Ala modifications flanked by the PGK1 promoter and the Acc1 terminator. As a result, the S. cerevisiae transformed with this sequence will express Acc1^{S659A; S1157A}. As shown in Table 62, base strain HB742 includes Acc1^{S659A; S1157A}, as does each modified yeast strain based on HB742 (HB801, HB861, HB862, HB814 and HB888).

SEQ ID NO:490 is a polynucleotide that may be used to modify the S. cerevisiae genome at the native Acc1 gene by homologous recombination. SEQ ID NO:490 includes a portion of the coding sequence for the Acc1 gene with Ser659Ala and Ser1167Ala modifications. A similar result may be achieved, for example, by integrating a sequence with the Tef1 promoter, the Acc1 with Ser659Ala and Ser1167Ala modifications, and the Prm9 terminator at any suitable site. The end result would be that Tef1, Acc1^{S659A; S1167A} and Prm9 are flanked by genomic DNA sequences for promoting integration into the S. cerevisiae genome.

Plasmid Construction

Plasmids synthesized to apply and prepare examples of the methods and yeast cells provided herein are shown in Table 65.

TABLE 65
Plasmids and Cassettes Used to Prepare Yeast Strains

| Plasmid Name | Description | Selection |
|---|---|---|
| PLAS-36 | pCAS_Hyg_Rnr2p:Cas9:Cyc1t::tRNATyr:HDV:gRNA:Snr52t | Hygromycin |
| PLAS-43 | pESC_Gal1p:PaPKS:Cyc1t | Uracil |
| PLAS-46 | pESC_Gal1p:PaPKS^{G1429R}:Cyc1t | Uracil |
| PLAS-47 | pESC_Gal1p:FaPKS:Cyc1t | Uracil |
| PLAS-48 | pGAL_Gal1p:csOAC:Cyc1t | Uracil |
| PLAS-180 | pESC_Gal1p:PuPKS^{G1452R}:Cyc1t | Uracil |
| PLAS-191 | pESC_Gal1p:PuPKS:Cyc1t | Uracil |
| PLAS-249 | pESC_Gal1p:FaPKS^{G1434R}:Cyc1t | Uracil |

The plasmids PLAS-36 and PLAS-48 were synthesized using services provided by Twist Bioscience Corporation. PLAS-43, PLAS-46, PLAS-47, PLAS-180, PLAS-191 and PLAS-249 were synthesized using services provided by Genscript.

Stable Transformation for Strain Construction

SEQ ID NO:480, SEQ ID NO: 481, SEQ ID NO: 482, SEQ ID NO:483 and SEQ ID NO:484 each include a copy of DiPKS^{G1516R} flanked by the Gall promoter, the Prm9 terminator, and integration sequences for the sites indicated above in Table 64.

Plasmids were transformed into S. cerevisiae using the lithium acetate heatshock method as described by Gietz, et al. (2007). S. cerevisiae HB865, HB866, HB867, HB868, HB869, HB870 were prepared by transformation of HB144 with expression plasmids Plas-43, Plas-46, Plas-47, Plas-180, Plas-191 and Plas-249, respectively, for stable expression of, respectively, PaPKS, PaPKS^{G1429R}, FaPKS, PuPKS^{G1452R}, PuPKS and FaPKS^{G1434R}

To create olivetolic acid producing strains, Plas-48 was stably transformed into HB259, HB309, HB310, HB742 to express csOAC at varying copy numbers of DiPKS^{G1516R}.

HB1030 was created to provide a base strain with genomic integration of csOAC. Successful integrations were confirmed by colony polymerase chain reaction (“PCR”) and led to the creation of HB1030 with a Galactose inducible csOAC encoding gene integrated into the genome of HB144. The genomic region containing SEQ ID NO.464 was also verified by sequencing to confirm the presence of the csOAC encoding gene. HB1113 was transformed by introduction of Plas-180 into HB1030, resulting in expression of PuPKS^{G1452R} and production of olivetol. HB1114 was transformed by introduction of Plas-249 into HB1030, resulting in expression of FaPKS^{G1434R} and production of olivetol and olivetolic acid.

Yeast Growth and Feeding Conditions

Yeast cultures were grown in overnight cultures with selective media to provide starter cultures. The resulting starter cultures were then used to inoculate experimental replicate cultures to an optical density at having an absorption at 600 nm (“A₆₀₀”) of 0.1.

Table 66 shows the uracil drop out (“URADO”) amino acid supplements that are added to yeast synthetic dropout media supplement lacking leucine and uracil. “YNB” is a nutrient broth including the chemicals listed in the first two columns of Table 66. The chemicals listed in the third and fourth columns of Table 66 are included in the URADO supplement.

TABLE 66
YNB Nutrient Broth and URADO Supplement

| YNB | | | URADO Supplement | | |
|---|---|---|---|---|---|
| Chemical | Concentration | | Chemical | Concentration | |
| Monosodium Glutamate | 1.5 | g/L | Adenine | 18 | mg/L |
| Biotin | 2 | μg/L | p-Aminobenzoic acid | 8 | mg/L |
| Calcium pantothenate | 400 | μg/L | Alanine | 76 | mg/ml |
| Folic acid | 2 | μg/L | Arginine | 76 | mg/ml |
| Inositol | 2 | mg/L | Asparagine | 76 | mg/ml |
| Nicotinic acid | 400 | μg/L | Aspartic Acid | 76 | mg/ml |
| p-Aminobenzoic acid | 200 | μg/L | Cysteine | 76 | mg/ml |
| Pyridoxine HCl | 400 | μg/L | Glutamic Acid | 76 | mg/ml |
| Riboflavin | 200 | μg/L | Glutamine | 76 | mg/ml |
| Thiamine HCL | 400 | μg/L | Glycine | 76 | mg/ml |
| Citric acid | 0.1 | g/L | Histidine | 76 | mg/ml |
| Boric acid | 500 | μg/L | myo-Inositol | 76 | mg/ml |
| Copper sulfate | 40 | μg/L | Isoleucine | 76 | mg/ml |
| Potassium iodide | 100 | μg/L | Leucine | 152 | mg/ml |
| Ferric chloride | 200 | μg/L | Lysine | 76 | mg/ml |
| Magnesium sulfate | 400 | μg/L | Methionine | 76 | mg/ml |
| Sodium molybdate | 200 | μg/L | Phenylalanine | 76 | mg/ml |
| Zinc sulfate | 400 | μg/L | Proline | 76 | mg/ml |
| Potassium phosphate monobasic | 1.0 | g/L | Serine | 76 | mg/ml |
| Magnesium sulfate | 0.5 | g/L | Threonine | 76 | mg/ml |
| Sodium chloride | 0.1 | g/L | Tryptophan | 76 | mg/ml |
| Calcium chloride | 0.1 | g/L | Tyrosine | 76 | mg/ml |

| | | | | |
|---|---|---|---|---|
| (blank cell) | (blank cell) | Valine | 76 | mg/ml |

Quantification of Metabolites

Metabolite extraction was performed with 300 μl of Acetonitrile added to 100 μl culture in a new 96-well deepwell plate, followed by 30 min of agitation at 950 rpm. The solutions were then centrifuged at 3,750 rpm for 5 min. 200 μl of the soluble layer was removed and stored in a 96-well v-bottom microtiter plate. Samples were stored at −20° C. until analysis.

Intracellular metabolites were quantified using high performance liquid chromatography (“HPLC”) and mass spectrometry (“MS”) methods. Quantification of olivetolic acid, CBGa and THCa was performed using HPLC-MS on an Acquity UPLC-TQD MS.

Quantification of olivetolic acid was performed by HPLC on a Waters HSS 1×50 mm column with a 1.8 μm particle size. Eluent A1 was 0.1% formic acid in water, and eluent El was 0.1% formic acid in acetonitrile. The ratios of A1:B1 were 70/30 at 0.00 min, 50/50 at 1.2 min, 30/70 at 1.70 min and 70/30 at 1.71 min. The column temperature was 45° C., the flow rate was 0.6 ml/min.

After HPLC separation, samples were injected into the mass spectrometer by electrospray ionization and analyzed in positive mode. The capillary temperature was held at 380° C. The capillary voltage was 3 kV, the source temperature was 150° C., the desolvation gas temperature was 450° C., the desolvation gas flow (nitrogen) was 800 L/hr, and the cone gas flow (nitrogen) was 50 L/hr.

TABLE 67
Detection parameters for CBGa and THCa

| Parameter | MPBD | Olivetol | Olivetolic Acid |
|---|---|---|---|
| Retention time | 1.35 min | 1.40 min | 1.28 min |
| Ion | [M − H]⁺ | [M − H]⁺ | [M − H]⁺ |
| Mass (m/z) | 195.1 | 181.1 | 223.01 |
| Mode | ES+, MRM | ES+, MRM | ES+, MRM |
| Transition | → 125 | → 71 | → 171 |
| Collision | 15 | 15 | 20 |
| Span | 0 | 0 | 0 |
| Dwell (s) | 0.2 | 0.2 | 0.2 |
| Cone (V) | 26 | 26 | 35 |

Different concentrations of known standards were injected to create a linear standard curve. Standards for MPBD, Olivetol and Olivetolic Acid were purchased from Toronto Research Chemicals.

### EXAMPLES—PART 6

### Example 16

The homologs of DiPKS were synthesized by GenScript and subsequently transformed into HB144. Twelve single colony replicates of each of HB144, HB259, HB867, HB870, HB869, HB868, HB865 and HB866 were grown in 1 ml of YNB-URA media (2.1 g/L of YNB+1.8 g/L of URADO+20 g/L glucose+200 ug/L geneticin+50 ug/L ampicillin) in 96-well deepwell plates. Twelve single colony replicates of HB144 and HB259 were grown in SC Media (2.1 g/L of YNB+1.8 g/L of URADO+20 g/L glucose+76 mg/l uracil+200 ug/l geneticin+50 ug/l ampicillin). The cultures were incubated for 96 hours at 30° C. at 950 RPM. After 96 hours the metabolites are extracted and quantified using HPLC-MS.

Only HB867 (FaPKS) produced MPBD. The other homologs of DiPKS did not show any MPBD production.

HB870 and HB868, produced olivetol from glucose. HB870 (FaPKS^{G1434R}) demonstrated that mutation of the c-met domain of FaPKS shifted the product profile completely from MPBD to olivetol. The mutation in the c-met domain of HB868 (PuPKS^{G1425R}) also led to the production of olivetol. This data demonstrates that PuPKS^{G1425R} is functional in yeast, and raises the possibility that its wild type product, which may be a methylated analogue of olivetol with a structure different than that of MPBD, is not being measured.

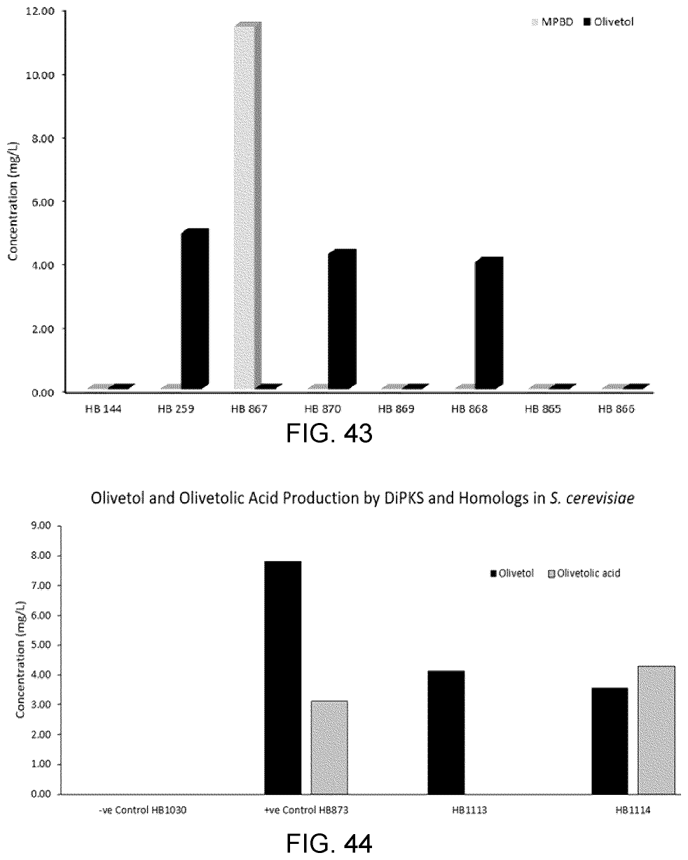

FIG. 43 shows the yields of MPBD and olivetol. Production of MPBD and olivetol from raffinose and galactose was observed, demonstrating direct production in yeast of MPBD and olivetol without hexanoic acid. The data from FIG. 43 is tabulated in Table 68.

TABLE 68
Production data for MPBD and olivetol
in eight strains of S. cerevisia

| | | MPBD | | olivetol | |
|---|---|---|---|---|---|
| Variable | Strain# | (mg/)l | STDEV | (mg/l) | STDEV |
| −ve Control | HB144 | 0.00 | 0.00 | 0.00 | 0.00 |
| DiPKS^{G1516R} | HB259 | 0.00 | 0.00 | 4.89 | 0.35 |
| FaPKS | HB867 | 11.38 | 3.22 | 0.00 | 0.00 |
| FaPKS^{G1434R} | HB870 | 0.00 | 0.00 | 4.23 | 0.95 |
| PuPKS | HB869 | 0.00 | 0.00 | 0.00 | 0.00 |
| PuPKS^{G1452R} | HB868 | 0.00 | 0.00 | 3.98 | 0.49 |
| PaPKS | HB865 | 0.00 | 0.00 | 0.00 | 0.00 |
| PaPKS^{G1429R} | HB866 | 0.00 | 0.00 | 0.00 | 0.00 |

### Example 17

FaPKS^{G1434R} and PuPKS^{G1452R} were assessed for production of olivetol and olivetolic acid in the presence of csOAC.

Twelve single colony replicates of HB873, HB1113 and HB1114 were grown in 1 ml of YNB-URA media (2.1 g/L of YNB+1.8 g/L of URADO+20 g/L glucose+200 ug/l geneticin+50 ug/l ampicillin) in 96-well deepwell plates. Twelve single colony replicates of HB 1030 were grown in SC Media (2.1 g/L of YNB+1.8 g/L of URADO+20 g/L Glucose+76 mg/L uracil+200 ug/l geneticin+50 ug/l ampicillin). The cultures were incubated for 96 hours at 30° C. at 950 RPM. After 96 hours the metabolites are extracted and quantified using HPLC-MS.

The expression of the csOAC in a strain expressing FaPKS^{G1434R} let to the simultaneous production of both Olivetol and Olivetolic acid. PuPKS^{G1452R} did not produce any olivetolic acid when expressed with csOAC, however, its Olivetol production was maintained.

FIG. 44 shows the yields of olivetol and olivetolic acid from HB873, HB1113 and HB1114, with HB1030 as a negative control. Production of olivetol and olivetolic acid from raffinose and galactose was observed, demonstrating direct production in yeast of olivetol and olivetolic acid without hexanoic acid. The data from FIG. 44 is tabulated in Table 69.

TABLE 69
Production data for olivetol and olivetolic
acid in four strains of S. cerevisia

| | | olivetol | | Olivetolic | |
|---|---|---|---|---|---|
| Variable | Strain# | (mg/)l | STDEV | acid (mg/l) | STDEV |
| −ve Control | HB1030 | 0.00 | 0.00 | 0.00 | 0.00 |
| +ve Control | HB873 | 7.80 | 1.85 | 3.13 | 0.74 |
| PuPKS^{G1452R} | HB1113 | 4.13 | 0.65 | 0.00 | 0.00 |
| FaPKS^{G1434R} | HB1114 | 3.58 | 1.08 | 4.30 | 1.57 |

### Example 18

Strains HB259, HB309, HB310, HB742 were cultured to assess DiPKS^{G1516R} activity at copy numbers of 1, 3, 4 and 5 for production of olivetol. Strains HB873, HB874, HB875, HB877, were cultured to assess DiPKS^{G1516R} activity at copy numbers of 1, 3, 4 and 5 for production of olivetolic acid in the presence of plasmid-expressed csOAC. Strain HB801 was cultured for expression of DiPKS^{G1516R} at a copy number of 5 in the presence of genome-integrated csOAC.

Twelve single colony replicates of strains HB144, HB259, HB309, HB310 and HB752 were grown in 1 ml of SC Media (2.1 g/L of YNB+1.8 g/L of URADO+20 g/L glucose+76 mg/l uracil+200 ug/l geneticin+50 ug/l ampicillin) each in 96-well deepwell plates. Strains HB873, HB874, HB875 and HB877 were grown in 1 ml of YNB-URA media (2.1 g/L of YNB+1.8 g/L of URADO+20 g/L glucose+200 ug/l geneticin+50 ug/l ampicillin). The cultures were incubated for 96 hours at 30° C. at 950 RPM. After 96 hours the metabolites are extracted and quantified using HPLC-MS.

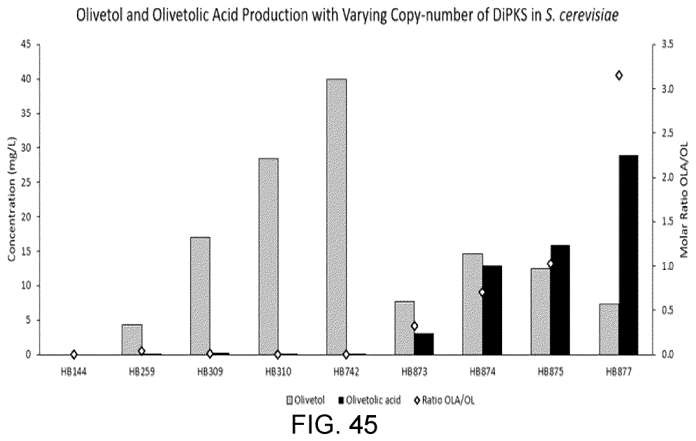

FIG. 45 shows the yields of olivetol and olivetolic acid from HB259, HB309, HB310, HB742, HB873, HB874, HB875, HB877 and HB801. Production from raffinose and galactose was observed, demonstrating direct production in yeast of olivetol and olivetolic acid without hexanoic acid. The data from FIG. 45 is tabulated in Table 70.

TABLE 70
Production data for olivetol and olivetolic
acid in nine strains of S. cerevisia

| | olivetol | | Olivetolic | | olivetolic |
|---|---|---|---|---|---|
| Strain# | (mg/)l | STDEV | acid (mg/l) | STDEV | acid:olivetol |
| HB144 | 0.00 | 0.0000 | 0.00 | 0.0000 | 0.0000 |
| HB259 | 4.38 | 0.0243 | 0.20 | 0.0009 | 0.0367 |
| HB309 | 17.07 | 0.0947 | 0.30 | 0.0013 | 0.0141 |
| HB310 | 28.47 | 0.1580 | 0.15 | 0.0007 | 0.0042 |
| HB742 | 40.00 | 0.2220 | 0.10 | 0.0004 | 0.0020 |
| HB873 | 7.80 | 0.0433 | 3.13 | 0.0140 | 0.3225 |
| HB874 | 14.63 | 0.0812 | 12.90 | 0.0575 | 0.7087 |
| HB875 | 12.47 | 0.0692 | 15.93 | 0.0711 | 1.0268 |
| HB877 | 7.38 | 0.0410 | 28.97 | 0.1292 | 3.1551 |
| HB801 | 27.26 | 0.1513 | 6.15 | 0.0274 | 0.1813 |

As the copy number of DiPKS^{G1516R} increases in the strain, the olivetol production also increases. This same effect was also seen with olivetolic acid production. As the copy-number of DiPKS^{G1516R} increases in the presence of OAC expressed from a high-copy plasmid, the amount of olivetolic acid produced also increases. The molar ratio between olivetolic acid and olivetol also increases as the copy number of DiPKS increases. This copy-number effect is also seen with the copy-number of csOAC. csOAC expressed from a high-copy plasmid in HB742 (HB877) has a greater olivetolic acid to olivetol production profile than a strain with a single copy of csOAC integrated into HB742 (HB801). HB801 has a lower production of olivetolic acid and a molar ratio of olivetolic acid to olivetol. This implies an effect of copy-number of csOAC on olivetolic acid production.

Part 7

Methods and Cells for Production of Phytocannabinoids or Phytocannabinoid Precursors Incorporating Aspects of Part 1 to Part 6

Combinations of the methods, nucleotides, and expression vectors described herein in PARTS 1 to 6 may be employed together to produce phytocannabinoids, phytocannabinoid precursors such as polyketides. Depending on the desired product, selections of characteristics of the cells and methods employed may be selected to achieve production of the cannabinoid, cannabinoid precursor, or intermediate of interest. Particular exemplary methods and cells are described hereinbelow.

Overview

A method of producing a phytocannabinoid is described, comprising culturing a host cell under suitable culture conditions to form a phytocannabinoid, said host cell comprising: (a) a polynucleotide encoding a polyketide synthase (PKS) enzyme; (b) a polynucleotide encoding an olivetolic acid cyclase (OAC) enzyme; and (c) a polynucleotide encoding a prenyltransferase (PT) enzyme; and optionally comprising: (d) a polynucleotide encoding an acyl-CoA synthase (Alk) enzyme; (e) a polynucleotide encoding a fatty acyl CoA activating (CsAAE) enzyme; and/or (f) a polynucleotide encoding a THCa synthase (OXC) enzyme.

A method of producing CBGOa via an orsellinic acid intermediate is also described, comprising culturing a host cell under suitable culture conditions to form said CBGOa, said host cell comprising a polynucleotide encoding polyketide synthase PKS110 and prenyltransferase PT72.

Methods of transforming host cells, expression vectors, and host cells comprising said polynucleotides are also described.

### Detailed Description of Part 7

A method of producing a phytocannabinoid comprising culturing a host cell under suitable culture conditions to form a phytocannabinoid is described. The host cell comprises a polynucleotide encoding a polyketide synthase (PKS) enzyme; a polynucleotide encoding an olivetolic acid cyclase (OAC) enzyme; and a polynucleotide encoding a prenyltransferase (PT) enzyme. Optionally, the host cell may also comprise a polynucleotide encoding an acyl-CoA synthase (Alk) enzyme; a polynucleotide encoding a fatty acyl CoA activating (CsAAE) enzyme; and/or a polynucleotide encoding a THCa synthase (OXC) enzyme, as well as any other polynucleotide described in any one of PARTS 1 to 6 herein.

A method is described for transforming a host cell for production of a phytocannabinoid comprising: introducing into the host cell line a polynucleotide encoding a polyketide synthase (PKS) enzyme; an olivetolic acid cyclase (OAC) enzyme; and a prenyltransferase (PT) enzyme; and optionally including said polynucleotide additionally encoding: (d) a polynucleotide encoding an acyl-CoA synthase (Alk) enzyme; (e) a polynucleotide encoding a fatty acyl CoA activating (CsAAE) enzyme; and/or (f) a polynucleotide encoding a THCa synthase (OXC) enzyme.

For example, the PKS may comprise DiPKS-1 to DiPKS-5 bearing G1516R, PKS73, or PKS80 to PKS110; the OAC may comprise csOAC or PC20; the PT may comprise PT72, PT104, PT129, PT211, PT254, PT273, or PT296; the CsAAE may comprise CsAAE1; the Alk may comprise Alk1-Alk30; and the OXC comprises OXC52; OXC53; or OXC155. Mutations of these as described herein with regard to PARTS 1-6 are encompassed.

A method of producing CBGOa via an orsellinic acid intermediate is described, comprising culturing a host cell under suitable culture conditions to form said orsellinic acid, wherein said host cell can then convert said orsellinic acid to CBGOa, said host cell comprising a polynucleotide encoding polyketide synthase PKS110 and prenyltransferase PT72.

An expression vector is described comprising: a polynucleotide encoding a polyketide synthase (PKS) enzyme; a polynucleotide encoding an olivetolic acid cyclase (OAC) enzyme; and a polynucleotide encoding a prenyltransferase (PT) enzyme. The expression vector optionally comprises a polynucleotide encoding an acyl-CoA synthase (Alk) enzyme; a polynucleotide encoding CsAAE1; and/or a polynucleotide encoding a THCa synthase (OXC) enzyme. Further, any polynucleotide as described in any one of PARTS 1-6 may be included in the expression vector.

An expression vector is described comprising a polynucleotide encoding polyketide synthase PKS110 and encoding prenyltransferase PT72. Optionally other polynucleotides may be included.

A host cell comprising these expression vectors is encompassed herein. The host cell is a bacterial cell, a fungal cell, a protist cell, or a plant cell, and may for example be a cell of a species selected from the group consisting of S. cerevisiae, E. coli, Yarrowia lipolytica, and Komagataella phaffii.

Table 71 outlines certain exemplary cells transformed with a combination of nucleic acids encoding enzymes for preparation of phytocannabinoids or precursors/intermediates in the production thereof. The enzyme names, strains, products formed, and feed used for the host cells in Examples 19-35. Briefly, host cells may be transformed with specific nucleic acids encoding enzymes permitting the cells to form a product, such as a phytocannabinoid, or an intermediate or precursor such as an aromatic polyketide. These examples are not limited to particular strains, nor are the named enzymes exhaustive of all possible enzymes such host cells may be transformed to contain.

TABLE 71
Exemplary Cells Transformed with a Combination of Enzymes
For Examples 19 to 35, The SEQ ID NO for the enzymes described in each combination is preceded by a number
in parentheses indicating in which of PART 1 to PART 7 the sequence is described

| | EX | Enzyme Name and SEQ ID Nos Provided for | | | | | Strain | | |
|---|---|---|---|---|---|---|---|---|---|
| | # | Specific Examples Described Herein | | | | | # | Product | Feed |
| Enzymes | 19 | | DiPKSG1516R | OAC | PT254 | OXC53 | HB888 | THCa | |
| | | | | (PC20) | | | | | |
| SEQ ID # | | | (1.)16 | (4.)412 | (4.)413 | (4.)421 | | | |
| Enzymes | 20 | CsAAE1 | PKS73 | OAC | PT254 | OXC155 | HB1775 | THCva | Butyric |
| | | | | (PC20) | | | | | acid |
| SEQ ID # | | (3.)405* | (3.)267 | (3.)406 | (4)413 | (3.)411* | | | |
| Enzymes | 21 | | DiPKSG1516R | OAC | PT296 | OXC53 | | THCa | |
| | | | | (PC20) | | | | | |
| SEQ ID # | | | (1.)16 | (4.)412 | (5.)440 | (4.)421 | | | |
| Enzymes | 22 | | DiPKSG1516R | OAC | PT72 | OXC53 | | THCa | |
| | | | | (PC20) | | | | | |
| SEQ ID # | | | (1.)16 | (4.)412 | (5.)438 | (4.)421 | | | |
| Enzymes | 23 | | DiPKSG1516R | OAC | PT273 | OXC53 | | THCa | |
| | | | | (PC20) | | | | | |
| SEQ ID # | | | (1.)16 | (4.)412 | (5.)439 | (4.)421 | | | |
| Enzymes | 24 | | PKS110 | [OAC | PT72 | | | CBGOa | |
| | | | | (PC20)]** | | | | | |
| SEQ ID # | | | (7.)514 | [(3.)406]* | (5.)438 | | | | |
| Enzymes | 25 | CsAAE1 | PKS73 | OAC | PT254 | | | CBGVa | Butyric |
| | | | | (PC20) | | | | | acid |
| SEQ ID # | | (3.)405* | (3.)267 | (3.)406 | (4.)413 | | | | |
| Enzymes | 26 | CsAAE1 | PKS73 | OAC | PT72 | | | CBGVa | Butyric |
| | | | | (PC20) | | | | | acid |
| SEQ ID # | | (3.)405* | (3.)267 | (3.)406 | (5.)438 | | | | |
| Enzymes | 27 | CsAAE1 | PKS73 | OAC | PT72 | OXC155 | | THCVa | Butyric |
| | | | | (PC20) | | | | | acid |
| SEQ ID # | | (3.)405* | (3.)267 | (3.)406 | (5.)438 | (3.)411* | | | |
| Enzymes | 28 | CsAAE1 | PKS73 | OAC | PT273 | OXC155 | | THCVa | Butyric |
| | | | | (PC20) | | | | | acid |
| SEQ ID # | | (3.)405* | (3.)267 | (3.)406 | (5.)439 | (3.)411* | | | |
| Enzymes | 29 | CsAAE1 | PKS73 | OAC | PT296 | OXC155 | | THCVa | Butyric |
| | | | | (PC20) | | | | | acid |
| SEQ ID # | | (3.)405* | (3.)267 | (3.)406 | (5.)440 | (3.)411* | | | |
| Enzymes | 30 | CsAAE1 | PKS73 | OAC | PT211 | OXC155 | | THCVa | Butyric |
| | | | | (PC20) | | | | | acid |
| SEQ ID # | | (3.)405* | (3.)267 | (3.)406 | (2.) 89 | (3.)411* | | | |
| Enzymes | 31 | CsAAE1 | PKS73 | OAC | PT129 | OXC155 | | THCVa | Butyric |
| | | | | (PC20) | | | | | acid |
| SEQ ID # | | (3.)405* | (3.)267 | (3.)406 | (2.) 78 | (3.)411* | | | |
| Enzymes | 32 | | DiPKSG1516R | OAC | PT254 | OXC52- | HB1890 | CBDa | |
| | | | | (PC20) | | S88A/L450G/P224- | | | |
| | | | | | | Serine insertion | | | |
| SEQ ID # | | | (1.)16 | (4.)412 | (4.)413 | (7.)500 | | | |
| Enzymes | 33 | | DiPKSG1516R | OAC | PT296 | OXC52- | | CBDa | |
| | | | | (PC20) | | S88A/L450G/P224- | | | |
| | | | | | | Serine insertion | | | |
| SEQ ID # | | | (1.)16 | (4.)412 | (5.)440 | (7.)500 | | | |
| Enzymes | 34 | | DiPKSG1516R | OAC | PT72 | OXC52- | | CBDa | |
| | | | | (PC20) | | S88A/L450G/P224- | | | |
| | | | | | | Serine insertion | | | |
| SEQ ID # | | | (1.)16 | (4.)412 | (5.)438 | (7.)500 | | | |
| Enzymes | 35 | | DiPKSG1516R | OAC | PT273 | OXC52- | | CBDa | |
| | | | | (PC20) | | S88A/L450G/P224- | | | |
| | | | | | | Serine insertion | | | |
| SEQ ID # | | | (1.)16 | (4.)412 | (5.)439 | (7.)500 | | | |

*Note on OXC notation: OXC155 and OXC53 are interchangeable in that OXC155 is defined as OstI-pro-alpha-f(I)-OXC53. The OstI-pro-alpha-f(I) tag is always used in examples where the product is THCa
**optional. but not required in Example 24

### Example 19

THCa Production

Host cell S. cerevisiae strain HB888 is transformed with the following enzymes: DiPKS G1516R (PART 1, SEQ ID NO:16); OAC (PC20) (see PART 4. SEQ ID NO:412); PT254 (see PART 4, SEQ ID NO:413); and OXC53 (see PART 4, SEQ ID NO:421) and under suitable culture and growth conditions forms THCa.

### Example 20

THCva Production with Butyric Acid Feed

Host cell S. cerevisiae strain HB1775 is transformed with the following enzymes: CsAAE1 (see PART 3, SEQ ID NO:405) PKS73 (PART 3, SEQ ID NO:267); OAC (PC20) (see PART 3, SEQ ID NO:406); PT254 (see PART 4, SEQ ID NO:413); and OXC155 (see PART 3, SEQ ID NO:411) and together with a butyric acid feed under suitable culture and growth conditions, forms THCva.

### Example 21

THCa Production

A S. cerevisiae host cell is transformed with the following enzymes: DiPKS G1516R (PART 1, SEQ ID NO:16); OAC (PC20) (see PART 4, SEQ ID NO:412); PT296 (see PART 5, SEQ ID NO:440); and OXC53 (see PART 4, SEQ ID NO:421) and is cultured under suitable culture and growth conditions to form THCa.

### Example 22

THCa Production

A S. cerevisiae host cell is transformed with the following enzymes: DiPKS G1516R (PART 1, SEQ ID NO:16); OAC (PC20) (see PART 4, SEQ ID NO:412); PT72 (see PART 5, SEQ ID NO:438); and OXC53 (see PART 4, SEQ ID NO:421) and is cultured under suitable culture and growth conditions to form THCa.

### Example 23

THCa Production

A S. cerevisiae host cell is transformed with the following enzymes: DiPKS G1516R (PART 1, SEQ ID NO:16); OAC (PC20) (see PART 4, SEQ ID NO:412); PT273 (see PART 5, SEQ ID NO:439); and OXC53 (see PART 4, SEQ ID NO:421) and is cultured under suitable culture and growth conditions to form THCa.

### Example 24

Cannabigiorcins: Cannabigiorcinic Acid Production (CBGOa)

Cannabigiorcins are cannabinoids built using an orsellinic acid polyketide. As a result of using orsellinic acid in place of olivetolic acid, cannbigiorcins have a C1 alkyl tail instead of the C5 tail found in most well-known cannabinoids, as shown below with regard to CBGOa, CBGa, THCOad THCa.

A S. cerevisiae host cell is transformed with the following enzymes: PKS110 (PART 7, SEQ ID NO:514) and PT72 (see PART 5, SEQ ID NO:438), and is cultured under suitable culture and growth conditions to form CBGOa.

Orsellenic acid may be produced in yeast using PKS110 (data shown in Table 72) and thus, the method of producing CBGOa using PKS110 and PT72 is encompassed herein.

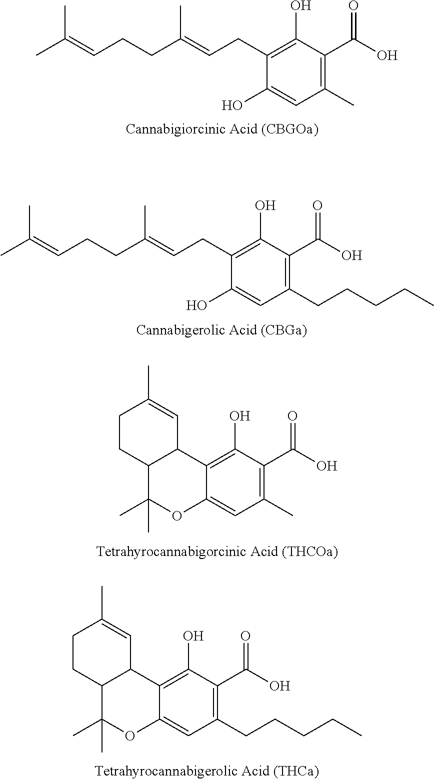 [SMILES: CC(C)=CCC/C(C)=C/Cc1c(O)cc(C)c(C(=O)O)c1O ; CCCCCc1cc(O)c(C/C=C(\C)CCC=C(C)C)c(O)c1C(=O)O ; CC1=CC2c3c(cc(C)c(C(=O)O)c3O)OC(C)(C)C2CC1 ; CCCCCc1cc2c(c(O)c1C(=O)O)C1C=C(C)CCC1C(C)(C)O2]

TABLE 72
In vivo production of orsellinic acid using PKS110

| Strain | Enzyme | Orsellenic acid (mg/L) |
|---|---|---|
| HB959 | PKS110 | 43.5 |
| HB144 | None | 0 |

### Example 25

CBGVa Production with Butyric Acid Feed

Host cell S. cerevisiae is transformed with the following enzymes: CsAAE1 (see PART 3, SEQ ID NO:405) PKS73 (PART 3, SEQ ID NO:267); OAC (PC20) (see PART 3. SEQ ID NO:406); and PT254 (see PART 4, SEQ ID NO:413); and together with a butyric acid feed under suitable culture and growth conditions, forms CBGVa.

### Example 26

CBGVa Production with Butyric Acid Feed

Host cell S. cerevisiae is transformed with the following enzymes: CsAAE1 (see PART 3, SEQ ID NO:405) PKS73 (PART 3, SEQ ID NO:267); OAC (PC20) (see PART 3, SEQ ID NO:406); and PT72 (see PART 5, SEQ ID NO:438); and together with a butyric acid feed under suitable culture and growth conditions, forms CBGVa.

### Example 27

THCVa Production with Butyric Acid Feed

Host cell S. cerevisiae is transformed with the following enzymes: CsAAE1 (see PART 3, SEQ ID NO:405) PKS73 (PART 3, SEQ ID NO:267); OAC (PC20) (see PART 3. SEQ ID NO:406); PT72 (see PART 5, SEQ ID NO:438); and OXC155 (PART 3, SEQ ID NO:411), and together with a butyric acid feed under suitable culture and growth conditions, forms THCVa.

### Example 28

THCVa Production with Butyric Acid Feed

Host cell S. cerevisiae is transformed with the following enzymes: CsAAE1 (see PART 3, SEQ ID NO:405) PKS73 (PART 3, SEQ ID NO:267); OAC (PC20) (see PART 3. SEQ ID NO:406); PT273 (see PART 5, SEQ ID NO:439); and OXC155 (PART 3, SEQ ID NO:411), and together with a butyric acid feed under suitable culture and growth conditions, forms THCVa.

### Example 29

THCVa Production with Butyric Acid Feed

Host cell S. cerevisiae is transformed with the following enzymes: CsAAE1 (see PART 3, SEQ ID NO:405) PKS73 (PART 3, SEQ ID NO:267); OAC (PC20) (see PART 3. SEQ ID NO:406); PT296 (see PART 5, SEQ ID NO:440); and OXC155 (PART 3, SEQ ID NO:411), and together with a butyric acid feed under suitable culture and growth conditions, forms THCVa.

### Example 30

THCVa Production with Butyric Acid Feed

Host cell S. cerevisiae is transformed with the following enzymes: CsAAE1 (see PART 3, SEQ ID NO:405) PKS73 (PART 3, SEQ ID NO:267); OAC (PC20) (see PART 3. SEQ ID NO:406); PT211 (see PART 2, SEQ ID NO:89); and OXC155 (PART 3, SEQ ID NO:411), and together with a butyric acid feed under suitable culture and growth conditions, forms THCVa.

### Example 31

THCVa Production with Butyric Acid Feed

Host cell S. cerevisiae is transformed with the following enzymes: CsAAE1 (see PART 3, SEQ ID NO:405) PKS73 (PART 3, SEQ ID NO:267); OAC (PC20) (see PART 3. SEQ ID NO:406); PT129 (see PART 2, SEQ ID NO:78); and OXC155 (PART 3, SEQ ID NO:411), and together with a butyric acid feed under suitable culture and growth conditions, forms THCVa.

Strain, Growth and Media: As pertaining to Examples 19 to 31, strains HB959, HB144 and others described herein, were grown on yeast minimal media with a composition of 1.7 g/L YNB without ammonium sulfate+1.4 g/L amino acid supplement dropout supplement lacking URA, HIS, LEU and TRP+1.5 g/L magnesium L-glutamate) with 2% w/v galactose, 2% w/v raffinose, 200 μg/l geneticin, and 200 ug/L ampicillin (Sigma-Aldrich Canada).

Experimental Conditions: 3-6 single colony replicates of strains were tested in this study. All strains were grown in 1 ml media for 96 hours in 96-well deepwell plates. The deepwell plates were incubated at 30° C. and shaken at 950 rpm for 96 hrs. Metabolite extraction was performed by adding 270 μl of 56% acetonitrile to 30 μl of culture in a fresh 96-well deepwell plates. The plates were then centrifuged at 3750 rpm for 5 min. 200 μl of the soluble layer was removed and stored in a 96-well v-bottom microtiter plate. Samples were stored at −20° C. until analysis.

Samples were quantified using HPLC-MS analysis

Table 73 lists and describes the strains used in Examples 19-31.

TABLE 73
Strains used in this study

| Strain # | Background | Plasmids | Genotype | Notes |
|---|---|---|---|---|
| HB144 | -URA, -LEU | None | Saccharomyces cerevisiae | Base |
| | | | CEN.PK2; ΔLEU2; ΔURA3; | strain |
| | | | Erg20K197E::KanMx; ALD6; ASC1L641P; | |
| | | | NPGA; MAF1; PGK1p:Acc1; tHMGR1; IDI | |
| HB965 | -URA, -LEU | None | Saccharomyces cerevisiae CEN.PK2; | Base |
| | | | ΔLEU2; ΔURA3; Erg20K197E::KanMx; | strain |
| | | | ALD6; ASC1L641P; NPGA; MAF1; | |
| | | | PGK1p:Acc1; tHMGR1; IDI; | |
| | | | DiPKS_G1516R X 5; ACC1_S659A_S1157A; | |
| | | | UB14p:ERG20; pGAL:OAC; pGAL:PT254 | |
| HB1202 | -URA, -LEU | None | Saccharomyces cerevisiae CEN.PK2; | Base |
| | | | ΔLEU2; ΔURA3; Erg20K197E::KanMx; | strain |
| | | | ALD6; ASC1L641P; NPGA; MAF1; | |
| | | | PGK1p:Acc1; tHMGR1; IDI; | |
| | | | DiPKS_G1516R X 5; ACC1_S659A_S1157A; | |
| | | | UB14p:ERG20; Tef1p:OAC; Tef1p:PT254 | |
| HB1740 | -URA, -LEU | PLAS415 | Saccharomyces cerevisiae CEN.PK2; | HB965 |
| | | | ΔLEU2; ΔURA3; Erg20K197E::KanMx; | |
| | | | ALD6; ASC1L641P; NPGA; MAF1; | |
| | | | PGK1p:Acc1; tHMGR1; IDI; | |
| | | | DiPKS_G1516R X 5; ACC1_S659A_S1157A; | |
| | | | UB14p:ERG20; pGAL:OAC; pGAL:PT254 | |
| HB1955 | -URA, -LEU | PLAS458 | Saccharomyces cerevisiae | HB965 |
| | | | CEN.PK2; ΔLEU2; ΔURA3; Erg20K197E::KanMx; | |
| | | | ALD6; ASC1L641P; NPGA; MAF1; | |
| | | | PGK1p:Acc1; tHMGR1; IDI; | |
| | | | DiPKS_G1516R X 5; ACC1_S659A_S1157A; | |
| | | | UB14p:ERG20; pGAL:OAC; pGAL:PT254 | |
| HB1956 | -URA, -LEU | PLAS459 | Saccharomyces cerevisiae | HB965 |
| | | | CEN.PK2; ΔLEU2; ΔURA3; Erg20K197E::KanMx; | |
| | | | ALD6; ASC1L641P; NPGA; MAF1; | |
| | | | PGK1p:Acc1; tHMGR1; IDI; | |
| | | | DiPKS_G1516R X 5; ACC1_S659A_S1157A; | |
| | | | UB14p:ERG20; pGAL:OAC; pGAL:PT254 | |
| HB2020 | -URA, -LEU | PLAS510 | Saccharomyces cerevisiae | HB965 |
| | | | CEN.PK2; ΔLEU2; ΔURA3; Erg20K197E::KanMx; | |
| | | | ALD6; ASC1L641P; NPGA; MAF1; | |
| | | | PGK1p:Acc1; tHMGR1; IDI; | |
| | | | DiPKS_G1516R X 5; ACC1_S659A_S1157A; | |
| | | | UB14p:ERG20; pGAL:OAC; pGAL:PT254 | |
| HB2021 | -URA, -LEU | PLAS511 | Saccharomyces cerevisiae | HB965 |
| | | | CEN.PK2; ΔLEU2; ΔURA3; Erg20K197E::KanMx; | |
| | | | ALD6; ASC1L641P; NPGA; MAF1; | |
| | | | PGK1p:Acc1; tHMGR1; IDI; | |
| | | | DiPKS_G1516R X 5; ACC1_S659A_S1157A; | |
| | | | UB14p:ERG20; pGAL:OAC; pGAL:PT254 | |
| HB1792 | -URA, -LEU | PLAS512 | Saccharomyces cerevisiae | HB965 |
| | | | CEN.PK2; ΔLEU2; ΔURA3; Erg20K197E::KanMx; | |
| | | | ALD6; ASC1L641P; NPGA; MAF1; | |
| | | | PGK1p:Acc1; tHMGR1; IDI; | |
| | | | DiPKS_G1516R X 5; ACC1_S659A_S1157A; | |
| | | | UB14p:ERG20; pGAL:OAC; pGAL:PT254 | |
| HB2010 | -URA, -LEU | PLAS513 | Saccharomyces cerevisiae | HB965 |
| | | | CEN.PK2; ΔLEU2; ΔURA3; Erg20K197E::KanMx; | |
| | | | ALD6; ASC1L641P; NPGA; MAF1; | |
| | | | PGK1p:Acc1; tHMGR1; IDI; | |
| | | | DiPKS_G1516R X 5; ACC1_S659A_S1157A; | |
| | | | UB14p:ERG20; pGAL:OAC; pGAL:PT254 | |
| HB990 | -URA, -LEU | PLAS416 | Saccharomyces cerevisiae | HB965 |
| | | | CEN.PK2; ΔLEU2; ΔURA3; Erg20K197E::KanMx; | |
| | | | ALD6; ASC1L641P; NPGA; MAF1; | |
| | | | PGK1p:Acc1; tHMGR1; IDI; | |
| | | | DiPKS_G1516R X 5; ACC1_S659A_S1157A; | |
| | | | UB14p:ERG20; pGAL:OAC; pGAL:PT254 | |
| HB1971 | -URA, -LEU | PLAS460 | Saccharomyces cerevisiae | HB965 |
| | | | CEN.PK2; ΔLEU2; ΔURA3; Erg20K197E::KanMx; | |
| | | | ALD6; ASC1L641P; NPGA; MAF1; | |
| | | | PGK1p:Acc1; tHMGR1; IDI; | |
| | | | DiPKS_G1516R X 5; ACC1_S659A_S1157A; | |
| | | | UB14p:ERG20; pGAL:OAC; pGAL:PT254 | |
| HB1973 | -URA, -LEU | PLAS462 | Saccharomyces cerevisiae | HB965 |
| | | | CEN.PK2; ΔLEU2; ΔURA3; Erg20K197E::KanMx; | |
| | | | ALD6; ASC1L641P; NPGA; MAF1; | |
| | | | PGK1p:Acc1; tHMGR1; IDI; | |
| | | | DiPKS_G1516R X 5; ACC1_S659A_S1157A; | |
| | | | UB14p:ERG20; pGAL:OAC; pGAL:PT254 | |
| HB1254 | -URA, -LEU | None | Saccharomyces cerevisiae | HB1202 |
| | | | CEN.PK2; ΔLEU2; ΔURA3; Erg20K197E::KanMx; | |
| | | | ALD6; ASC1L641P; NPGA; MAF1; | |
| | | | PGK1p:Acc1; tHMGR1; IDI; | |
| | | | DiPKS_G1516R X 5; ACC1_S659A_S1157A; | |
| | | | UB14p:ERG20; pGAL:OAC; | |
| | | | pGAL:PT254; Ostl-pro-alpha-f(I)-OXC52 | |
| HB1890 | -URA, -LEU | None | Saccharomyces cerevisiae | HB1202 |
| | | | CEN.PK2; ΔLEU2; ΔURA3; Erg20K197E::KanMx; | |
| | | | ALD6; ASC1L641P; NPGA; MAF1; | |
| | | | PGK1p:Acc1; tHMGR1; IDI; | |
| | | | DiPKS_G1516R X 5; ACC1_S659A_S1157A; | |
| | | | UB14p:ERG20; pGAL:OAC; pGAL:PT254; | |
| | | | Ostl-pro-alpha-f(I)-OXC52-S225del | |
| HB959 | -URA, -LEU | None | Saccharomyces cerevisiae | HB144 |
| | | | CEN.PK2; ΔLEU2; ΔURA3; Erg20K197E::KanMx; | |
| | | | ALD6; ASC1L641P; NPGA; MAF1; | |
| | | | PGK1p:Acc1; tHMGR1; IDI; PKS110 | |

Table 74 lists the plasmids used in this example.

TABLE 74
Description of Plasmids

| | Plasmid | | | |
|---|---|---|---|---|
| # | Name | Description | Selection | Backbone |
| 1 | PLAS415 | Ostl-pro-alpha- | Uracil | pGREG- |
| | | f(I)-OXC52-VB40 | | URA |
| 2 | PLAS459 | Ostl-pro-alpha-f(I)- | Uracil | pGREG- |
| | | OXC52-L450G-VB40 | | URA |
| 3 | PLAS458 | Ostl-pro-alpha-f(I)- | Uracil | pGREG- |
| | | OXC52-S88A-VB40 | | URA |
| 4 | PLAS510 | Ostl-pro-alpha-f(I)- | Uracil | pGREG- |
| | | OXC52-A386V-VB40 | | URA |
| 5 | PLAS511 | Ostl-pro-alpha-f(I)- | Uracil | pGREG- |
| | | OXC52-G350I-VB40 | | URA |
| 6 | PLAS512 | Ostl-pro-alpha-f(I)- | Uracil | pGREG- |
| | | OXC52-R3W-VB40 | | URA |
| 7 | PLAS513 | Ostl-pro-alpha-f(I)- | Uracil | pGREG- |
| | | OXC52-P224_225insSer | | URA |
| 8 | PLAS460 | Ostl-pro-alpha-f(I)- | Uracil | pGREG- |
| | | OXC52-S88A/L450G/R3W | | URA |
| 9 | PLAS462 | Ostl-pro-alpha-f(I)- | Uracil | pGREG- |
| | | OXC52-S88A/450G/Serine | | URAHB |
| | | insertion at P224 | | |
| 11 | PLAS416 | RFP | RFP | pGREG- |
| | | | | URA |
| 12 | PLAS400 | RFP | RFP | pYES-URA |

TABLE 75
Description of Sequences

| | | | | Position of |
|---|---|---|---|---|
| SEQ ID | | | Length of | coding |
| NO: | Description | DNA/Protein | sequence | sequence |
| SEQ ID | Ostl-pro-alpha-f(I)- | Protein | 609 | all |
| NO. 492 | OXC52 | | | |
| SEQ ID | Ostl-pro-alpha-f(I)- | Protein | 609 | all |
| NO. 493 | OXC52-S88A | | | |
| SED ID | Ostl-pro-alpha-f(I)- | Protein | 609 | all |
| NO. 494 | OXC52-A386V | | | |
| SEQ ID | Ostl-pro-alpha-f(I)- | Protein | 609 | all |
| NO. 495 | OXC52-L450G | | | |
| SEQ ID | Ostl-pro-alpha-f(I)- | Protein | 609 | all |
| NO. 496 | OXC52-G350I | | | |
| SEQ ID | Ostl-pro-alpha-f(I)- | Protein | 609 | all |
| NO.497 | OXC52-R3W | | | |
| SEQ ID | Ostl-pro-alpha-f(I)- | Protein | 610 | all |
| NO. 498 | OXC52-Serine | | | |
| | insertion at P224 | | | |
| SEQ ID | Ostl-pro-alpha-f(I)- | Protein | 609 | all |
| NO. 499 | OXC52- | | | |
| | S88A/L450G/R3W | | | |
| SEQ ID | Ostl-pro-alpha-f(I)- | Protein | 610 | all |
| NO. 500 | OXC52- | | | |
| | S88A/450G/Serine | | | |
| | insertion at P224 | | | |
| SEQ ID | Ostl-pro-alpha-f(I)- | Protein | 610 | all |
| NO. 501 | OXC53 | | | |
| SEQ ID | Ostl-pro-alpha-f(I)- | Protein | 609 | all |
| NO. 502 | OXC53 - S225 del | | | |
| SEQ ID | PKS110 | Protein | 2098 | all |
| NO. 503 | | | | |
| SEQ ID | RFP | Protein | 232 | all |
| NO. 504 | | | | |
| SEQ ID | PLAS415 | DNA | 7615 | 2890-4719 |
| NO. 505 | | | | |
| SEQ ID | PLAS459 | DNA | 7615 | 2890-4719 |
| NO. 506 | | | | |
| SEQ ID | PLAS458 | DNA | 7615 | 2890-4719 |
| NO. 507 | | | | |
| SEQ ID | PLAS510 | DNA | 7615 | 2890-4719 |
| NO. 508 | | | | |
| SEQ ID | PLAS511 | DNA | 7615 | 2890-4719 |
| NO. 509 | | | | |
| SEQ ID | PLAS512 | DNA | 7615 | 2890-4719 |
| NO. 510 | | | | |
| SEQ ID | PLAS513 | DNA | 7618 | 2890-4721 |
| NO. 511 | | | | |
| SEQ ID | Ostl-pro-alpha-f(I)- | DNA | 4137 | 1339-3189 |
| NO. 512 | OXC53 | | | |
| SEQ ID | Ostl-pro-alpha-f(I)- | DNA | 4134 | 1339-3186 |
| NO. 513 | OXC53 - S225 del | | | |
| SEQ ID | PKS110 | DNA | 7717 | 728-7024 |
| NO. 514 | | | | |

TABLE 76
Modifications to base strains used in this experiment:

| | | | Integration | | |
|---|---|---|---|---|---|
| | Modification | | Region/ | | Genetic Structure of |
| # | name | SEQ ID NO. | Plasmid | Description | Sequence |
| 1 | Ostl-pro- | SEQ ID | Apel-3 | d28 THC synthase | Apel-3up::Tef1p:Ostl- |
| | alpha-f(I)- | NO: 512 | | fused with a 5′ Ostl- | pro-alpha-f(I)- |
| | OXC53 | | | pro-alpha-f(I) tag | OXC53::cyct:Apel- |
| | | | | | 3down |
| 2 | Ostl-pro- | SEQ ID | Apel-3 | d28 THC synthase | Apel-3up::Tef1p:Ostl- |
| | alpha-f(I)- | NO: 513 | | fused with a 5′ OST- | pro-alpha-f(I)-OXC53- |
| | OXC53 - | | | Proaftag. S225 is | S225del::cyct:Apel- |
| | S225 del | | | deleted. | 3down |
| 3 | PKS110 | SEQ ID | X-4 | Orsellinic acid | X-4up::pGAL:PKS110::cyct:X- |
| | | NO: 514 | | synthase | 4-3down |

### Examples 32-35

Examples are provided herein in which aspects of the above-noted details of PART 1-PART 6 are utilized in combination to produce phytocannabinoids or intermediates in the production thereof, specifically with regard to CBDa production in the following examples. Transformed cells are also described.

Method and Cells for CBDa Production

The terminal step in CBDa biosynthesis is the cyclization of CBGa by CBDa synthase. Modified CBDAs are used, which is hereafter referred to as Ostl-pro-alpha-f(I)-OXC52. When expressed inside yeast, Ostl-pro-alpha-f(I)-OXC52 has limited activity and is a bottleneck in the pathway. Through an in house protein engineering program we have discovered mutants of Ostl-pro-alpha-f(I)-OXC52 that show increased CBDAs activity in yeast. These include point mutations and single amino acid insertions. We would like to claim the process of producing CBDa in a modified yeast cell using these enzymes. A list of the best performing mutations is shown below in Table 77, which lists OXC52 mutants with improved activity in yeast.

TABLE 77
OXC52 mutants with improved activity in yeast

| Strain | Mutation | Activity relative to wild type |
|---|---|---|
| HB1668 | OXC52 | 1.00 |
| HB1955 | OXC52-S88A | 4.50 |
| HB2020 | OXC52-A386V | 3.08 |
| HB1956 | OXC52-L450G | 3.12 |
| HB2021 | OXC52-G350I | 2.44 |
| HB1792 | OXC52-R3W | 2.45 |
| HB2010 | OXC52-Serine | 5.76 |
| | insertion at P224 | |
| HB990 | RFP (negative) | 0 |

Combinations of these mutations can also be used to create enzymes with even greater activity. We would like to claim the use of a CEO synthase with any of the above listed mutations in any combination. A list of the top performing combinations discovered so far is shown below in Table 78, which shows OXC52 mutant combinations with improved activity in yeast.

TABLE 78
OXC52 mutant combinations with improved activity in yeast

| | | CBGa | CBDa | % CBGA |
|---|---|---|---|---|
| Strain | Mutation | (mg/L) | (mg/L) | turnover |
| HB1668 | OXC52 | 23.1 | 1.3 | 0.05 |
| HB1971 | OXC52-S88A/L450G/R3W | 4.9 | 12.6 | 0.38 |
| HB1973 | OXC52- S88A/450G/Serine | 3.8 | 15.4 | 0.29 |
| | insertion at P224 | | | |
| HB990 | RFP (negative) | 18.4 | 0 | 0.0 |

An interesting finding from this work is that the insertion of a serine after residue 224 greatly increases Ostl-pro-alpha-f(I)-OXC52 activity. Alternatively, if serine 225 is deleted from THCAs (OXC53) the enzyme switches its activity from producing THCA to primarily produced CBDA. We would like to claim the use of Ostl-pro-alpha-f(I)-OXC53-S225 del for producing CBDa in a modified yeast cell. Table 79 shows production of CBDa using the mutant THCa synthase described herein.

TABLE 79
Production of CBDa using a mutant THCa synthase

| | | CBGa | CBDa | THCa |
|---|---|---|---|---|
| Strain | Mutation | (mg/L) | (mg/L) | (mg/L) |
| HB1254 | Ostl-pro-alpha- | 20.9 | 0.0 | 1.4 |
| | f(I)-OXC53 | | | |
| HB1890 | Ostl-pro-alpha- | 12.6 | 2.1 | 0.1 |
| | f(I)-OXC53 - | | | |
| | S225 del | | | |

Strain Growth and Media. Strains H B1668, H B1955, H B2020, H B1956, H B2021, HB1792, HB2010, HB990, HB1668, HB1971, HB1973, and HB990 were grown on yeast minimal media with a composition of 1.7 g/L YNB without ammonium sulfate+1.96 g/L URA dropout amino acid supplement+1.5 g/L magnesium L-glutamate) with 2% w/v galactose, 2% w/v raffinose, 200 μg/l geneticin, and 200 ug/L ampicillin (Sigma-Aldrich Canada).

HB1890 and HB1254 were grown on yeast minimal media with a composition of 1.7 g/L YNB without ammonium sulfate+1.4 g/L amino acid supplement dropout supplement lacking URA, HIS, LEU and TRP+1.5 g/L magnesium L-glutamate) with 2% w/v galactose, 2% w/v raffinose, 200 μg/l geneticin, and 200 ug/L ampicillin (Sigma-Aldrich Canada).

Experimental Conditions. 3-6 single colony replicates of strains were tested in this study. All strains were grown in 1 ml media for 96 hours in 96-well deepwell plates. The deepwell plates were incubated at 30° C. and shaken at 950 rpm for 96 hrs. Metabolite extraction was performed by adding 270 μl of 56% acetonitrile to 30 μl of culture in a fresh 96-well deepwell plate. The plates were then centrifuged at 3750 rpm for 5 min. 200 μl of the soluble layer was removed and stored in a 96-well v-bottom microtiter plate. Samples were stored at −20° C. until analysis. Samples were quantified using HPLC-MS analysis

Quantification Protocol. The quantification of CBDa was performed using HPLC-MS on a Acquity UPLC-TQD MS. The chromatography and MS conditions are described below

LC conditions: Column: Waters Acquity UPLC C18 column 1×50 mm, 1.8 um. Column temperature: 45. Flow rate: 0.35 mL/min. Eluent A: H2O 0.1% Formic Acid. Eluent B: ACN 0.1% Formic Acid.

Gradient:

| Time (min) | % B | Flow rate (ml/min) |
|---|---|---|
| 0 | 90 | 0.35 |
| 1.20 | 10 | 0.35 |
| 1.21 | 90 | 0.35 |
| 2.00 | 90 | 0.35 |

ESI-MS conditions: Capillary: 4 kV. Source temperature: 150° C. Desolvation gas temperature: 400° C. Drying gas flow (nitrogen): 500 L/hr. Collision gas flow (argon): 0.10 mL/min

MRM Transition: CBDa (negative ionisation): m/z 357.5->245.1.

### Example 32

CBDa Production

A S. cerevisiae host cell is transformed with the following enzymes: DiPKS G1516R (PART 1, SEQ ID NO:16); OAC (PC20) (see PART 4, SEQ ID NO:412); PT254 (see PART 4, SEQ ID NO:413); and OXC52-S88A/L450G/P224-Serine insertion (see PART 7, SEQ ID NO:500) and is cultured under suitable culture and growth conditions to form CBDa.

### Example 33

CBDa Production

A S. cerevisiae host cell is transformed with the following enzymes: DiPKS G1516R (PART 1, SEQ ID NO:16); OAC (PC20) (see PART 4, SEQ ID NO:412); PT296 (see PART 5, SEQ ID NO:440); and OXC52-S88A/L450G/P224-Serine insertion (see PART 7, SEQ ID NO:500) and is cultured under suitable culture and growth conditions to form CBDa.

### Example 34

CBDa Production

A S. cerevisiae host cell is transformed with the following enzymes: DiPKS G1516R (PART 1, SEQ ID NO:16); OAC (PC20) (see PART 4, SEQ ID NO:412); PT72 (see PART 5, SEQ ID NO:438); and OXC52-S88A/L450G/P224-Serine insertion (see PART 7, SEQ ID NO:500) and is cultured under suitable culture and growth conditions to form CBDa.

### Example 35

CBDa Production

A S. cerevisiae host cell is transformed with the following enzymes: DiPKS G1516R (PART 1, SEQ ID NO:16); OAC (PC20) (see PART 4, SEQ ID NO:412); PT273 (see PART 5, SEQ ID NO:439); and OXC52-S88A/L450G/P224-Serine insertion (see PART 7, SEQ ID NO:500) and is cultured under suitable culture and growth conditions to form CBDa.

### Examples Only

In the preceding description, for purposes of explanation, numerous details are set forth in order to provide a thorough understanding of the embodiments. However, it will be apparent to one skilled in the art that these specific details are not required.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

### REFERENCES

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

### Patent Publications

U.S. Pat. No. 7,361,482
U.S. Pat. No. 8,884,100 (Page et al.) Aromatic Prenyltransferase from Cannabis.
WO2018148848 (Mookerjee et al.) publication of PCT/CA2018/050189, METHOD AND CELL LINE FOR PRODUCTION OF PHYTOCANNABINOIDS AND PHYTOCANNABINOID ANALOGUES IN YEAST
WO2018148849 (Mookerjee et al.) publication of PCT/CA2018/050190, METHOD AND CELL LINE FOR PRODUCTION OF POLYKETIDES IN YEAST

### Non-Patent Literature

Bai Flagfeldt, D., Siewers, V., Huang, L. and Nielsen, J. (2009) “Characterization of chromosomal integration sites for heterologous gene expression in Saccharomyces cerevisiae” Yeast, 26, 545-551.
Gagne, S. J., et al. (2012) “Identification of Olivetolic Acid Cyclase from Cannabis Sativa Reveals a Unique Catalytic Route to Plant Polyketides.” Proceedings of the National Academy of Sciences, vol. 109, no. 31, 2012, pp. 12811-12816. doi:10.1073/pnas.1200330109.
Ghosh, R., A. Chhabra, P. A. Phatale, S. K. Samrat, J. Sharma, A. Gosain, D. Mohanty, S. Saran and R. S. Gokhale (2008) “Dissecting the Functional Role of Polyketide Synthases in Dictyostelium discoideum biosynthesis of the differentiation regulating factor 4-methyl-5-pentylbenzene-1,3-diol” Journal of Biological Chemistry, 283(17), 11348-11354.
Gietz, R. D. and Schiestl, R. H., (2007) “High-efficiency yeast transformation using the LiAc/SS carrier DNA/PEG method.” Nat. Protoc. 2, 31-34.
Gietz R. D. (2014) Yeast Transformation by the LiAc/SS Carrier DNA/PEG Method (pp 1-12). In: Smith J., Burke D. (eds) Yeast Genetics. Methods in Molecular Biology (Methods and Protocols), vol 1205. Humana Press, New York, N. Y. https://doi.org/10.1007/978-1-4939-1363-3_1.
Jensen, N. B., Strucko, T., Kildegaard, K. R., David, F., Er\hat{ }Ome Maury, J., Mortensen, U. H., et al., (2014). EasyClone: method for iterative chromosomal integration of multiple genes in Saccharomyces cerevisiae. FEMS Yeast Research, Volume 14, Issue 2, pages 238-248; https://doi.org/10.1111/1567-1364.12118.
Kim, J.-M., Song, H.-Y., Choi, H.-J., So, K.-K., Kim, D.-H., Chae, K.-S., . . . Jahng, K.-Y. (2015). “Characterization of NpgA, a 4′-phosphopantetheinyl transferase of Aspergillus nidulans, and evidence of its involvement in fungal growth and formation of conidia and cleistothecia for development.” Journal of Microbiology, 53(1), 21-31 https: //doi.org/10.1007/s12275-015-4657-8.
Kuzuyama et al. (2005) Structural basis for the promiscuous biosynthetic prenylation of aromatic natural products, Nature, volume 435, pages 983-987; doi: 10.1038/nature03668.
Liu, J., Zhang, W., Du, G., Chen, J., & Zhou, J. (2013). “Overproduction of geraniol by enhanced precursor supply in Saccharomyces cerevisiae.” Journal of Biotechnology, 168(4), 446-451. https://doi.org/10.1016/J.JBIOTEC.2013.10.017.
Luo, X., Reiter, M., d'Espaux, L., Wong, J., Denby, C., Lechner, A., Zhang, Y., Grzybowski, A., Harth, S., Lin, W., Lee, H., Yu, C., Shin, J., Deng, K., Benites, V., Wang, G., Baidoo, E., Chen, Y., Dev, I., Petzold, C. and Keasling, J. (2019). “Complete biosynthesis of cannabinoids and their unnatural analogues in yeast.” Nature, 567(7746), pp. 123-126.
Oswald, Marilyne; Marc Fischer, Nicole Dirninger, Francis Karst, (2007) “Monoterpenoid biosynthesis in Saccharomyces cerevisiae.” FEMS Yeast Research, 7(3), 413-421. https://doi.org/10.1111/j.1567-1364.2006.00172.x
Peng, B., Nielsen, L. K., Kampranis, S. C., & Vickers, C. E. (2018). Engineered protein degradation of farnesyl pyrophosphate synthase is an effective regulatory mechanism to increase monoterpene production in Saccharomyces cerevisiae. Metabolic Engineering, 47, 83-93. https://doi.org/10.1016/J.YMBEN.2018.02.005.
Ro, D.-K., Paradise, E. M., Ouellet, M., Fisher, K. J., Newman, K. L., Ndungu, J. M., Keasling, J. D. (2006). Production of the antimalarial drug precursor artemisinic acid in engineered yeast. Nature, 440(7086), 940-943. JOUR. https://doi.org/10.1038/nature04640.
Ryan, O. W., Poddar, S., & Cate, J. H. D. (2016). CRISPR-Cas9 Genome Engineering in Saccharomyces cerevisiae Cells. Cold Spring Harbor Protocols, 2016(6), pdb.prot086827. https://doi.org/10.1101/pdb.prot086827.
Saeki, H., Hara, R., Takahashi, H., lijima, M., Munakata, R., Kenmoku, H., . . . Taura, F. (2018). An Aromatic Farnesyltransferase Functions in Biosynthesis of the Anti-HIV Meroterpenoid Daurichromenic Acid. Plant Physiology, 178(2), 535-551; https: //doi.org/10.1104/PP.18.00655.
Shi, S., Chen, Y., Siewers, V., & Nielsen, J. (2014). “Improving Production of Malonyl Coenzyme A-Derived Metabolites by Abolishing Snf1-Dependent Regulation of Acc1.” mBio, 5(3), e01130-14. https://doi.org/10.1128/mBio.01130-14.
Shiba, Y., Paradise, E. M., Kirby, J., Ro, D.-K., & Keasling, J. D. (2007). “Engineering of the pyruvate dehydrogenase bypass in Saccharomyces cerevisiae for high-level production of isoprenoids.” Metabolic Engineering, 9(2), 160-168. https: //doi.org/10.1016/J.YMBEN.2006.10.005.
Stout, J. M., Boubakir, Z., Ambrose, S. J., Purves, R. W., & Page, J. E. (2012). The hexanoyl-CoA precursor for cannabinoid biosynthesis is formed by an acyl-activating enzyme in Cannabis sativa trichomes. The Plant Journal, 71(3), 353-365.
Taura, Futoshi, et al. (2009) “Characterization of olivetol synthase, a polyketide synthase putatively involved in cannabinoid biosynthetic pathway.” FEBS letters 583.12 (2009): 2061-2066.
Varshavsky, A. (2011). The N-end rule pathway and regulation by proteolysis. Protein Science 20(8):1285-1476. https://doi.org/10.1002/pro.666.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. A method of producing a phytocannabinoid or phytocannabinoid analogue in a host cell that produces a polyketide and a prenyl donor, said method comprising:
transforming said host cell with a sequence encoding a prenyltransferase PT296 protein, and
culturing said transformed host cell under conditions sufficient for production of the prenyltransferase PT296 protein to produce said phytocannabinoid or phytocannabinoid analogue;
wherein the wherein the prenyltransferase PT296 protein comprises a prenyltransferase protein as set forth in SEQ ID NO:440.

135. The method of claim 134, wherein the prenyltransferase PT296 protein comprises or consists of
a prenyltransferase protein with at least 95% identity with SEQ ID NO:440.

136. The method of claim 134, wherein the sequence encoding the prenyltransferase PT296 protein comprises or consists of:
(a) a nucleotide sequence encoding the protein of SEQ ID NO:440; or a nucleotide having a sequence according to SEQ ID NO:461;
(b) a nucleotide sequence having at least 85% identity with the nucleotide sequence of (a); or having at least 85% identity with SEQ ID NO461; or
(c) a nucleotide sequence that hybridizes with the complementary strand of the nucleic acid of (a) under conditions of high stringency.

137. The method of claim 134, wherein said polyketide is:

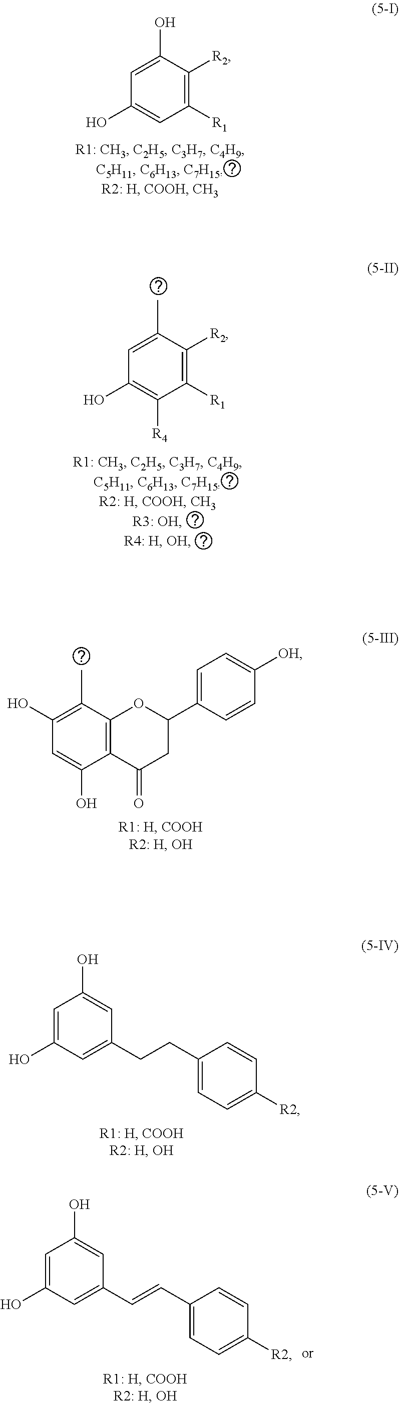

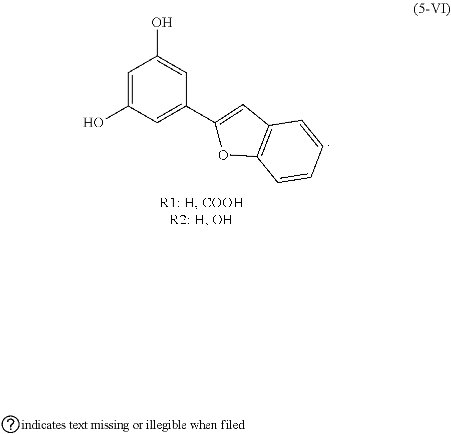 [SMILES: Oc1cc(O)cc(-c2cc3ccccc3o2)c1]

138. The method of any one of claim 134, wherein said prenyl donor is:

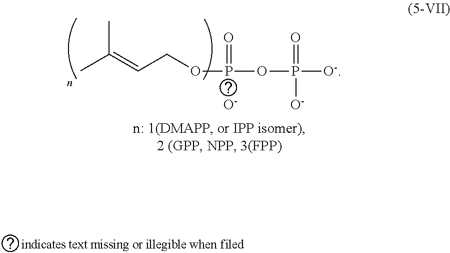

139. The method of claim 138, wherein the prenyl donor is geranyl diphosphate (GPP), farnesyl diphosphate (FPP), or neryl diphosphate (NPP).

140. The method of claim 134, wherein said phytocannabinoid or phytocannabinoid analogue is:

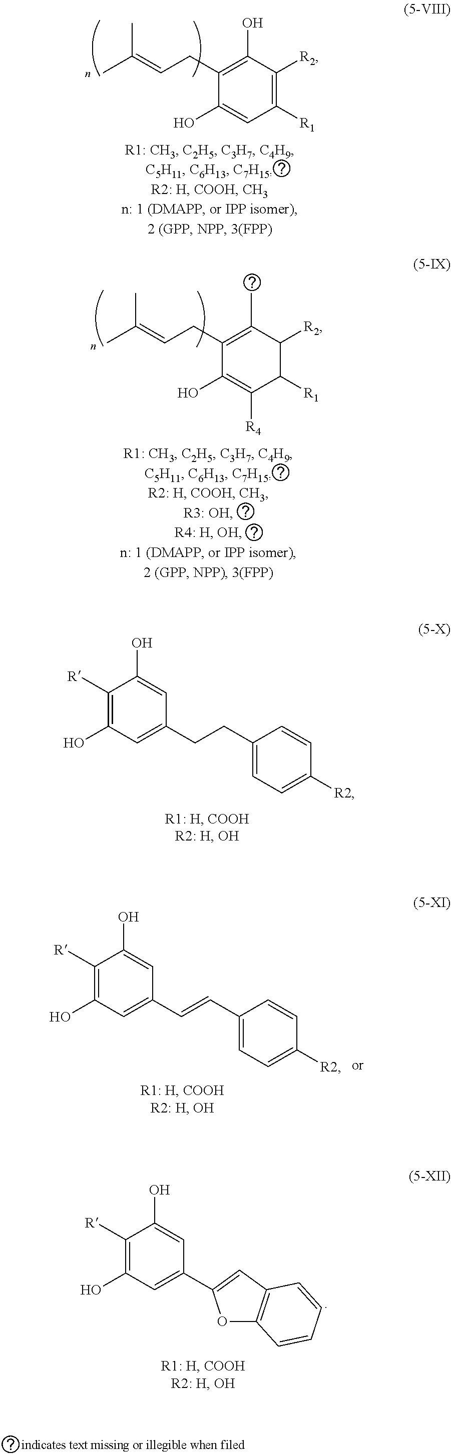

141. (canceled)

142. (canceled)

143. (canceled)

144. The method of claim 134, wherein said polyketide is olivetolic acid, and said phytocannabinoid is cannabigerolic acid (CBGa).

145. (canceled)

146. (canceled)

147. (canceled)

148. (canceled)

149. (canceled)

150. (canceled)

151. (canceled)

152. (canceled)

153. (canceled)

154. (canceled)

155. (canceled)

156. An expression vector comprising a nucleotide sequence encoding prenyltransferase PT296 protein, wherein said nucleotide sequence comprises:
at least 85% identity with a nucleotide sequence encoding SEQ ID NO:440; or
at least 85% identity with a nucleotide having a sequence according to SEQ ID NO:461.

157. (canceled)

158. A host cell transformed with the expression vector according to claim 156.

159. The host cell of claim 158, additionally comprising one or more of:
(a) a nucleic acid as set forth in any one of SEQ ID NO:441 to SEQ ID NO:453;
(b) a nucleic acid having at least 85% identity with the nucleotide sequence of (a);
(c) a nucleic acid that hybridizes with the complementary strand of the nucleic acid of (a) under stringent conditions; or
(d) a nucleic acid encoding a protein with the same enzyme activity as the protein encoded by any one of the nucleic acid sequences of (a).

160. (canceled)

161. (canceled)

162. (canceled)

163. (canceled)

164. (canceled)

165. (canceled)

166. (canceled)

167. (canceled)

168. (canceled)

169. (canceled)

170. (canceled)

171. (canceled)

172. (canceled)

173. (canceled)

174. (canceled)

175. (canceled)

176. (canceled)

177. (canceled)

178. (canceled)

179. (canceled)

180. (canceled)

181. (canceled)

182. (canceled)

183. (canceled)

184. (canceled)

185. (canceled)

186. (canceled)

187. (canceled)

188. (canceled)

189. (canceled)

190. (canceled)

191. (canceled)

192. (canceled)

193. (canceled)

194. (canceled)

195. (canceled)

196. (canceled)

197. (canceled)

198. (canceled)

199. (canceled)

200. (canceled)

201. (canceled)

202. (canceled)

203. (canceled)

204. (canceled)

205. (canceled)

206. (canceled)

207. (canceled)

208. (canceled)

209. (canceled)

210. (canceled)

211. (canceled)

212. (canceled)

213. A method of producing a phytocannabinoid comprising culturing a host cell under suitable culture conditions to form a phytocannabinoid, said host cell comprising:
(a) a polynucleotide encoding a polyketide synthase (PKS) enzyme;
(b) a polynucleotide encoding an olivetolic acid cyclase (OAC) enzyme;
(c) a polynucleotide encoding a prenyltransferase (PT) enzyme; and
(d) a polynucleotide encoding a THCa synthase (OXC) enzyme:
wherein the OXC enzyme comprises a protein with at least 85% sequence identity to any one of SEQ ID NO. 498-501:
and optionally comprising:
(e) a polynucleotide encoding an acyl-CoA synthase (Alk) enzyme; and/or
(f) polynucleotide encoding a fatty acyl CoA activating (CsAAE) enzyme.

214. The method of claim 213, wherein:
PKS comprises DiPKS-1-DiPKS-5 bearing G1516R, PKS73, or PKS80-PKS110;
OAC comprises csOAC or PC20;
PT comprises PT296;
CsAAE comprises CsAAE1; and/or
Alk comprises Alk1-Alk30.

215. The method of claim 213, wherein the host cell is cultured together with a butyric acid feed.

216. A method of transforming a host cell for production of a phytocannabinoid comprising:
introducing into the host cell line a polynucleotide encoding:
(a) a polyketide synthase (PKS) enzyme;
(b) an olivetolic acid cyclase (OAC) enzyme;
(c) a prenyltransferase (PT) enzyme; and
(d) a polynucleotide encoding a THCa synthase (OXC) enzyme:
wherein the OXC enzyme comprises a protein with at least 85% sequence identity to any one of SEQ ID NO. 498-501:
and optionally said polynucleotide additionally encoding:
(e) a polynucleotide encoding an acyl-CoA synthase (Alk) enzyme; and/or
(f) a polynucleotide encoding a fatty acyl CoA activating (CsAAE) enzyme.

217. The method of claim 216, wherein:
PKS comprises DiPKS-1-DiPKS-5 bearing G1516R, PKS73, or PKS80-PKS110;
OAC comprises csOAC or PC20;
PT comprises PT296;
CsAAE comprises CsAAE1; and/or
Alk comprises Alk1-Alk30.

218. (canceled)

219. An expression vector comprising:
a polynucleotide encoding a polyketide synthase (PKS) enzyme;
a polynucleotide encoding an olivetolic acid cyclase (OAC) enzyme;
a polynucleotide encoding a prenyltransferase (PT) enzyme; and
a polynucleotide encoding a THCa synthase (OXC) enzyme of at least 85% sequence identity with SEQ ID NO. 498-501.

220. The expression vector of claim 219, additionally comprising:
a polynucleotide encoding an acyl-CoA synthase (Alk) enzyme; and/or
a polynucleotide encoding CsAAE1.

221. AR The expression vector of claim 219, wherein
the polynucleotide encoding the OXC enzyme encodes a protein according to SEQ ID NO: 500; and/or
the polynucleotide encoding the prenyltransferase enzyme encodes a protein having at least 85% sequence identity to SEQ ID NO:440.

222. A host cell comprising the expression vector of claim 219.

223. (canceled)

224. (canceled)

225. The host cell of claim 222, wherein said host cell comprises a nucleotide encoding: SEQ ID NOs: 440 and 500.

226. The expression vector of claim 156, additionally comprising a nucleotide sequence encoding an OXC enzyme according to SEQ ID NO. 498-501.

227. A host cell comprising the expression vector of claim 226.

228. A prenyltransferase (PT) enzyme having a sequence according to SEQ ID NO:440, prepared by the host cell of claim 158.

229. A THCa synthase (OXC) enzyme having a sequence according to any one of SEQ ID NO:498-501, prepared by the host cell of claim 222.
